SEQUENCE LISTING (118 sequences)

SEQ ID NO: 1 - DNA, 1540 bp, Pediococcus acidilactici
  source 1..1540: mol_type = genomic DNA
caagtcgaacgaacttccgttaattgattatgacgtgcttgcactgaatgagattttaacacgaagtgagtggcggacgggtgagtaacacgtgggtaacctgcccagaagcaggggataacacctggaaacagatgctaataccgtataacagagaaaaccgcctggttttcttttaaaagatggctctgctatcacttctggatggacccgcggcgcattagctagttggtgaggtaacggctcaccaaggcgatgatgcgtagccgacctgagagggtaatcggccacattgggactgagacacggcccagactcctacgggaggcagcagtagggaatcttccacaatggacgcaagtctgatggagcaacgccgcgtgagtgaagaagggtttcggctcgtaaagctctgttgttaaagaagaacgtgggtgagagtaactgttcacccagtgacggtatttaaccagaaagccacggctaactacgtgccagcagccgcggtaatagggtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctgttagtaccctgctgtcaacgataagagtgattactaagtgttggagggtttccgcccttcagtgctgcagctaacgcattaagttatccgcctggggagtacgaccgcaaggttgaaactcaaagaattgacgggggccgcacaagcgtggagcatgtggttaattcgaagtacgcgaagaacttaccaggtcttgacatctttgccaacctaagagattaggcgttccttcggggacagaatgacaggtggtgcatggttgtcgtcagctcgtgtcgtgagatgttgggttaagtcccgcaacgagcgcaacccttattactagttgccagcattcagttgggcactctagtgagactgccggtgacaaaccggaggaaggtggggacgacgtcaaatcatcatgccccttatgacctgggctacacacgtgctacaatggatggtacaacgagtcgcgaaaccgcgaggtttagctaatctcttaaaaccattctcagttcggactgtaggctgcaactcgcctacacgaagtcggaatcgctagtaatcgcggatcagcatgccgcggtgaatacgttcccgggccttgtacacaccgcccgtcacaccatgagagtttgtaacacccaaagccggtggggtaaccttttaggagctagccgtctaaggtgggacagatgattagggtgaagtcgtaacaaggtagccgtaggagaacctgcggctggatcacctcctttctaaggaataatacggaacc

SEQ ID NO: 2 - DNA, 1433 bp, Akkermansia muciniphila
  source 1..1433: mol_type = genomic DNA
aacgaacgctggcggcgtggataagacatgcaagtcgaacgagagaattgctagcttgctaataattctctagtggcgcacgggtgagtaacacgtgagtaacctgcccccgagagcgggatagccctgggaaactgggattaataccgcatagtatcgaaagattaaagcagcaatgcgcttggggatgggctcgcggcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagtaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtgaaggtggggactctggcgagactgcccagatcaactgggaggaaggtggggacgacgtcaggtcagtatggcccttatgcccagggctgcacacgtactacaatgcccagtacagagggggccgaagccgcgaggcggaggaaatcctaaaaactgggcccagttcggactgtaggctgcaacccgcctacacgaagccggaatcgctagtaatggcgcatcagctacggcgccgtgaatacgttcccgggtcttgtacacaccgcccgtcacatcatggaagctggtcgcacccgaagtatctgaagccaaccgcaaggaggcagggtcctaaggtgagactggtaactgggatg

SEQ ID NO: 3 - DNA, 1450 bp, Escherichia coli
  variation 392: note = a, c, t, g, unknown or other
  variation 852..853: note = a, c, t, g, unknown or other
  source 1..1450: mol_type = genomic DNA
agtttgatcatggctcagattgaacgctggcggcaggcctaacacatgcaagtcgaacggtaacaggaagcagcttgctgctttgctgacgagtggcggacgggtgagtaatgtctgggaaactgcctgatggagggggataactactggaaacggtagctaataccgcataacgtcgcaagcacaaagagggggaccttagggcctcttgccatcggatgtgcccagatgggattagctagtaggtggggtaacggctcacctaggcgacgatccctagctggtctgagaggatgaccagcaacactggaactgagacacggtccagactcctacgggaggcagcagtggggaatattgcacaatgggcgcaagcctgatgcagccatgcngcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccgganntaacgcgttaagtcgaccgcctggggagtacggccgcaaggttaaaactcaaatgaattgacgggggccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaacttcgggagggcg

SEQ ID NO: 4 - DNA, 1520 bp, Streptococcus mitis
  source 1..1520: mol_type = genomic DNA
ttttgagtttgatcctggctcaggacgaacgctggcggcgtgcctaatacatgcaagtagaacgctgaaggaggagcttgcttctccggatgagttgcgaacgggtgagtaacgcgtaggtaacctgcctggtagcgggggataactattggaaacgatagctaataccgcataagagtagatgttgcatgacatttgcttaaaaggtgcaattgcatcactaccagatggacctgcgttgtattagctagttggtggggtaacggctcaccaaggcgacgatacatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtacgctttggaaactgtttaacttgagtgcaagaggggagagtggaattccatgtgtagcggtgaaatgcgtagatatatggaggaacaccggtggcgaaagcggctctctggcttgtaactgacgctgaggctcgaaagcgtggggagcaaacaggattagataccctggtagtccacgccgtaaacgatgagtgctaggtgttagaccctttccggggtttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagttttccttcgggacagaggtgacaggtggtgcatggttgtcgtcagctcgtgtcgtgagatgttgggttaagtcccgcaacgagcgcaacccctattgttagttgccatcatttagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctcttaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttttaggagccagccgcctaaggtgggatagatgattggggtgaagtcgtaacaagtcagccgtttgggaa

SEQ ID NO: 5 - DNA, 1546 bp, Streptococcus salivarius
  source 1..1546: mol_type = genomic DNA
atgggagagtttgatcctggctcaggacgaacgctggcggcgtgcctaatacatgcaagtagaacgctgaagagaggagcttgctcttcttggatgagttgcgaacgggtgagtaacgcgtaggtaacctgccttgtagcgggggataactattggaaacgatagctaataccgcataacaatggatgacacatgtcatttatttgaaaggggcaattgctccactacaagatggacctgcgttgtattagctagtaggtgaggtaacggctcacctaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagtcaagaacgagtgtgagagtggaaagttcacactgtgacggtagcttaccagaaagggacggctaactacgtgccagcagccgcggtaatacgtaggtcccgagcgttgtccggatttattgggcgtaaagcgagcgcaggcggtttgataagtctgaagttaaaggctgtggctcaaccatagttcgctttggaaactgtcaaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttggatcctttccgggattcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctatttctagagatagaaagttacttcggtacatcggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggttggtacaacgagttgcgagtcggtgacggcaagctaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggtagccgtatcggaaggtgcggctggatcactccctta

SEQ ID NO: 6 - DNA, 1511 bp, Phascolarctobacterium faecium
  variation 50: note = a, c, t, g, unknown or other
  variation 1382: note = a, c, t, g, unknown or other
  variation 1477: note = a, c, t, g, unknown or other
  source 1..1511: mol_type = genomic DNA
atcctggctcaggacgaacgctggcggcgtgcctaacacatgcaagtcgnacggagaattttatttcggtagaattcttagtggcgaacgggtgagtaacgcgtaggcaacctaccctttagacggggacaacattccgaaaggagtgctaataccggatgtgatcatcgtgccgcatggcaggatgaagaaagatggcctctacaagtaagctatcgctaaaggatgggcctgcgtctgattagctagttggtagtgtaacggactaccaaggcgatgatcagtagccggtctgagaggatgaacggccacattgggactgagacacggcccaaactcctacgggaggcagcagtggggaatcttccgcaatggacgaaagtctgacggagcaacgccgcgtgagtgatgaaggatttcggtctgtaaagctctgttgtttatgacgaacgtgcagtgtgtgaacaatgcattgcaatgacggtagtaaacgaggaagccacggctaactacgtgccagcagccgcggtaatacgtaggtggcgagcgttgtccggaattattgggcgtaaagagcatgtaggcggcttaataagtcgagcgtgaaaatgcggggctcaaccccgtatggcgctggaaactgttaggcttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacgggattagataccccggtagtcctggccgtaaacgatgggtactaggtgtaggaggtatcgaccccttctgtgccggagttaacgcaataagtaccccgcctggggagtacggccgcaaggttgaaactcaaaggaattgacgggggcccgcacaagcggtggagtatgtggtttaattcgacgcaacgcgaagaaccttaccaaggcttgacattgattgaacgctctagagatagagatttcccttcggggacaagaaaacaggtggtgcatggctgtcgtcagctcgtgtcgtgagatgttgggttaagtcccgcaacgagcgcaacccctatcctatgttaccagcaagtaaagttggggactcatgggagactgccagggacaacctggaggaaggcggggatgacgtcaagtcatcatgccccttatgtcttgggctacacacgtactacaatggtcggaaacagagggaagcgaagccgcgaggcagagcaaaccccagaaacccgatctcagttcggatcgcaggctgcaacccgcctgcgtgaagtcggaatcgctagtaatcgcaggtcagcatactgcggtgaatacgtncccgggccttgtacacaccgcccgtcacaccacgaaagttggtaacacccgaagccggtgaggtaacctattaggagccagccgtctaaggtggngccgatgattggggtgaagtcgtaacaaggtagc

SEQ ID NO: 7 - DNA, 1517 bp, Lactobacillus plantarum
  source 1..1517: mol_type = genomic DNA
ckaacgcyggcggcgtgcctaatacatgcaagtcgaacgaactctggtattgattggtgcttgcatcatgatttacatttgagtgagtggcgaactggtgagtaacacgtgggaaacctgcccaggaagcgggggataacacctggaaacakatgctaataccgcataacaacttggaccgcatggtccgagtttgaaagatggcttcggctatcacttttggatggycccgcggcgtattakctakatggtggggtaacggctcaccatggcaatgatacgtacccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtatatatatggaasaacaccagtggcgaaggcggctgtctggttctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaacaaggtagccgtaggagaa

SEQ ID NO: 8 - DNA, 1479 bp, Clostridium tertium
  source 1..1479: mol_type = genomic DNA
caggacgaacgctggcggcgtgcctaacacatgcaagtcgagcgagaggagttccttcgggaacaaatctagcggcggacgggtgagtaacacgtgggcaacctgccttgtagaggggaatagccttccgaaaggaagattaataccgcataacattacattttcgcatgaagaagtaattaaaggagcaatccgctacaagatgggcccgcggcgcattagctagttggtgaggtaacggctcaccaaggcgacgatgcgtagccgacctgagagggtgatcggccacattgggactgagacacggcccagactcctacgggaggcagcagtggggaatattgcacaatgggggaaaccctgatgcagcaacgccgcgtgagtgatgaaggttttcggatcgtaaagctctgtcttcagggacgataatgacggtacctgaggaggaagccacggctaactacgtgccagcagccgcggtaatacgtaggtggcgagcgttgtccggatttactgggcgtaaagggagcgtaggcggatttttaagtgagatgtgaaatacccgggctcaacttgggtgctgcatttcaaactggaagtctagagtgcaggagaggagagtggaattcctagtgtagcggtgaaatgcgtagagattaggaagaacaccagtggcgaaggcgactctctggactgtaactgacgctgaggctcgaaagcgtggggagcaaacaggattagataccctggtagtccacgccgtaaacgatgaatactaggtgtaggggttgtcatgacctctgtgccgccgcaaacgcattaagtattccgcctggggagtacggtcgcaagattaaaactcaaaggaattgacgggggcccgcacaagcagcggagcatgtggtttaattcgaagcaacgcgaagaaccttacctagacttgacatctcctgcattactcttaatcgaggaagtcccttcggggacaggatgacaggtggtgcatggttgtcgtcagctcgtgtcgtgagatgttgggttaagtcccgcaacgagcgcaacccttattgttagttgctaccatttagttgagcactctagcgagactgcccgggttaaccgggaggaaggtggggatgacgtcaaatcatcatgccccttatgtctagggctacacacgtgctacaatggcaagtacaaagagatgcgataccgcgaggtggagctaaactataaaacttgtctcagttcggattgtaggctgaaactcgcctacatgaagctggagttgctagtaatcgcgaatcagaatgtcgcggtgaatacgttcccgggccttgtacacaccgcccgtcacaccatgagagttggcaatacccaaagttcgtgagctaacccgtaagggaggcagcgacctaaggtagggtcagcgattggggtgaagtcgtaacaaggtagccgtaggagaacctgcggctg

SEQ ID NO: 9 - DNA, 1487 bp, Holdemanella biformis
  variation 31: note = a, c, t, g, unknown or other
  variation 120: note = a, c, t, g, unknown or other
  variation 242: note = a, c, t, g, unknown or other
  variation 341..342: note = a, c, t, g, unknown or other
  variation 579: note = a, c, t, g, unknown or other
  variation 928..929: note = a, c, t, g, unknown or other
  variation 933: note = a, c, t, g, unknown or other
  variation 968: note = a, c, t, g, unknown or other
  variation 1229: note = a, c, t, g, unknown or other
  variation 1385: note = a, c, t, g, unknown or other
  source 1..1487: mol_type = genomic DNA
caaatggagagtttgatcctggctcaggatnaacgctggcggcatgcctaatacatgcaagtcgaacgagaggaaggaaagcttgcttttctgaatctagtggcgaacgggtgagtaacncgtaggtaacctgcccatgtgcccgggataacttctggaaacggatgctaaaaccggataggtagcagacaagcatttgactgctattaaagtggctaaggccatgaacatggatggacctncggtgcattagctagttggtgaggtaacggcccaccaaggcgacgatgcatagccggcctgagagggcggacggccacattgggactgagacacggccnnaactcctacgggaggcagcagtagggaattttcgtcaatggggggaaccctgaacgagcaatgccgcgtgagtgaggaaggtcttcggatcgtaaagctctgttgtaagagaaaaacgacattcatagggaatgatgagtgagtgatggtatcttaccagaaagtcacggctaactacgtgccagcagccgcggtaatacgtaggtggcgagcgttatccggaatgattgggcgtanagggtgcgtaggtggcagaacaagtctggagtaaaaggtatgggctcaacccgtactggctctggaaactgttcagctagagaacagaagaggacggcggaactccatgtgtagcggtaaaatgcgtagatatatggaagaacaccggtggcgaaggcggccgtctggtctgttgctgacactgaagcacgaaagcgtggggagcaaataggattagataccctagtagtccacgccgtaaacgatgagaactaggtgttgggggaataactcagtgccgcagttaacgcaataagttctccgcctggggagtatgcacgcaagtgtgaaactcaaaggaattgacggnngccngcacaagcggtggagtatgtggtttaattcgaagnaacgcgaagaaccttaccaggccttgacatcccttgcagagrtatagagatatatccgaggttaacaaggagacaggtggtgcatggttgtcgtcagctcgtgtcgtgagatgttgggttaagtcccgcaacgagcgcaacccttgtgatatgttactaacattgagttgaggactcatatcagactgccggtgataaaccggaggaaggtggggatgacgtcaaatcatcatgccccttatggcctgggctacacacgtnctacaatggcgtctacaaagagcggcaagcctgtgaaggcaagcgaatctcataaaggacgtctcagttcggattgaagtctgcaacccgacttcatgaagctggaatcgctagtaatcgcgaatcagcatgtcgcggtgaatacgttctcgggcnttgtacacaccgcccgtcaaaccatgggagttggtaatgcccgaagccggtggcataacctttcgaggagtgagccgtcgaaggcaggaccgatgactgggg

SEQ ID NO: 10 - DNA, 1537 bp, Gemella haemolysans
  source 1..1537: mol_type = genomic DNA
gatcctggctcaggacgaacgctggcggcgtgcctaatacatgcaagtcgagcgaagtttttctggtgcttgcaccggaaaaacttagcggcgaacgggtgagtaacacgtaaagaacctgcctcatagactgggacaactattggaaacgatagctaataccggataacagcattaactgcatggttgatgtttaaaagttggttttgctaacactatgagatggctttgcggtgcattagctagttggtggggtaaaggcctaccaaggcgacgatgcatagccgacctgagagggtgatcggccacactgggactgagacacggcccagactcctacgggaggcagcagtagggaatcttccgcaatgggcgaaagcctgacggagcaacgccgcgtgagtgaagaaggatttcggttcgtaaagctctgttgttagggaagaatgattgtgtagtaactatacacagtagagacggtacctaaccagaaagccacggctaactacgtgccagcagccgcggtaatacgtaggtggcaagcgttgtccggaattattgggcgtaaagcgcgcgcaggtggtttaataagtctgatgtgaaagcccacggctcaaccgtggagggtcattggaaactgttaaacttgagtgcaggagagaaaagtggaattcctagtgtagcggtgaaatgcgtagagattaggaggaacaccagtggcgaaggcggctttttggcctgtaactgacactgaggcgcgaaagcgtggggagcaaacaggattagataccctggtagtccacgccgtaaacgatgagtgctaagtgttggtctcataagagatcagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaagtcttgacatactgtgaggacacaagagattgtgttgttctgacctttggttagacacagatacaggtggtgcatggttgtcgtcagctcgtgtcgtgagatgttgggttaagtcccgcaacgagcgcaacccttatatctagttgccagcagtaagatggggactctagatagactgccagtgataaactggaggaaggtggggatgacgtcaaatcatcatgccccttatgacttgggctacacacgtgctacaatggataggaacaaagagaagcgagctcgcgagagtaagcaaacctcacaaaactattctcagttcggattgtagtctgcaactcgactacatgaagctggaatcgctagtaatcgcgaatcagaatgtcgcggtgaatacgttcccgggtcttgtacacaccgcccgtcacaccacgagagtttgtaacacccgaagacggtggcctaaccttttaggagggagccggtcacgtgggacagatgattggggtgaagtcgtaacaaggtagccgtatcggaaggtgcggctggatcacct

SEQ ID NO: 11 - DNA, 1461 bp, Terrisporobacter glycolicus
  source 1..1461: mol_type = genomic DNA
aacgctggcggcgtgcctaacacatgcaagtcgagcgattcacttcggtgaagagcggcggacgggtgagtaacgcgtgggtaacctgcctcatacacatggataacataccgaaaggtatgctaatacaggataatataagagattcacatgtatttcttatcaaagctccggcggtatgagatggacccgcgtctgattagctagttggtaaggtaacggcttaccaaggcgacgatcagtagccgacctgagagggtgatcggccacattggaactgagacacggtccaaactcctacgggaggcagcagtggggaatattgcacaatgggcgaaagcctgatgcagcaacgccgcgtgagtgatgaaggccttcgggtcgtaaaactctgtcctcaaggaagataatgacggtacttgaggaggaagccccggctaactacgtgccagcagccgcggtaatacgtagggggctagcgttatccggatttactgggcgtaaagggtgcgtaggtggtttcttaagtcaggagtgaaaggctacggctcaaccgtagtaagctcttgaaactgggagacttgagtgcaggagaggaaagtggaattcctagtgtagcggtgaaatgcgtagatattaggaggaacaccagtagcgaaggcggctttctggactgtaactgacactgaggcacgaaagcgtggggagcgaacaggattagataccctggtagtccacgccgtaaacgatgagtactaggtgtcgggggttacccccctcggtgccgcacgtaacgcattaagtactccgcctggggagtacgctcgcaagagtgaaactcaaaggaattgacggggacccgcacaagtagcggagcatgtggtttaattcgaagcaacgcgaagaaccttacctaagcttgacatccttttgaccgatgcctaatcgcatttttcccttcggggacagaagtgacaggtggtgcatggttgtcgtcagctcgtgtcgtgagatgttgggttaagtcccgcaacgagcgcaacccttgcctttagttgccagcattaagttgggcactctagagggactgccagggataacctggaggaaggtggggatgacgtcaaatcatcatgccccttatgcttagggctacacacgtgctacaatgggtggtacagagggcagccaagtcgtgaggccgagctaatcccttaaatgccattctcagttcggattgtaggctgaaactcgcctacatgaagctggagttactagtaatcgcagatcagaatgctgcggtgaatgcgttcccgggtcttgtacacaccgcccgtcacaccacggaagttgggggcgcccgaagccacttagctaacccttttgggaagcgagtgtcgaaggtgaaatcaataactggggtgaagtcgtaacaaggtagccgtatcggaaggtgcggct

SEQ ID NO: 12 - DNA, 1453 bp, Coprococcus comes
  source 1..1453: mol_type = genomic DNA
gatgaacgctggcggcgtgcttaacacatgcaagtcgaacgaagcacttatmcttgattcttcggatgaagrggtttgtgactgagtggcggacgggtgagtaacgcgtgggtaacctgcctcatacagggggataacagttagaaatgactgctaataccgcataagaccacrgagccgcatggctcggtgggaaaaactccggtggtatgagatggacccgcgtctgattaggtagttggtggggtaacggcctaccaagccaacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaaggcggcttactggacgatgactgacgttgaggctcgaaagcgtggggagcaaacaggattagataccctggtagtccacgccgtaaacgatgactactaggtgtcggggagcaaagctcttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccggtgaccggcgtgtaatgacgccttttcttcggaacaccggtgacaggtggtgcatggttgtcgtcagctcgtgtcgtgagatgttgggttaagtcccgcaacgagcgcaacccttatcttcagtagccagcaattcggatgggcactctggagagactgccagggataacctggaggaaggtggggatgacgtcaaatcatcatgccccttatgagcagggctacacacgtgctacaatggcgtaaacaaagggaagcgagcctgcgagggtaagcaaatctcaaaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggtaacgcccgaagtcagtgacccaaccgtaaggagggagctgccgaaggtgggaccgataactggggtg

SEQ ID NO: 13 - DNA, 1388 bp, Clostridium spiroforme
  source 1..1388: mol_type = genomic DNA
gatcctggctcaggatgaacgctggcggcgtgcctaacacatgcaagtcgagcgagcagcaatgctcgagtggcgaacgggtgagtaatacataagtaacctgccctagacagggggataactgctggaaacggcagctaagaccgcataggtatggacactgcatggtgaccatattaaaatgccaaggcactggtagaggatggacttatggcgcattagctggttggtgaggtaacagctcaccaaggcgacgatgcgtagccgacctgagagggtgaccggccacactgggactgagacacggcccagactcctacgggaggcagcagtagggaattttcggcaatggggggaaccctgaccgagcaacgccgcgtgaaggaagaaggaattcgttctgtaaacttctgttataaaggaagaacggcggatatagggaatgatatccgagtgacggtactttatgagaaagccacggctaactacgtgccagcagccgcggtaatacgtaggtggcgagcgttatccggaattattgggcgtaaagagggagcaggcggcggcagaggtctgtggtgaaagactgaagcttaacttcagtaagccatagaaaccgggctgctagagtgcaggagaggatcgtggaattccatgtgtagcggtgaaatgcgtagatatatggaggaacaccagtggcgaaggcgacggtctggcctgtaactgacgcacattcccgaaagcgtggggagcaaataggattagataccctagtagtccacgccgtaaacgatgagtactaagtgttgggagtcaaatttcagtgctgcagttaacgcaataagtactccgcctgagtagtacgttcgcaagaatgaaactcaaaggaattgacgggggccgcacaagcggtggagcatgtggtttaattcgaagcaacgcgaagaaccttaccaggtcttgacatccggataaagacctcgagagatgaggggatagatatatccgagacaggtggtgcatggttgtcgtcagctcgtgtcgtgagatgttgggttaagtcccgcaacgagcgcaacccttgtcgctagttaccatcattaagttggggactctagcgagactgccagtgacaagctggaggaaggcggggatgacgtcaaatcatcatgccccttatgacctgggctacacacgtgctacaatggatggagcagagggaagcgaagccgcgaggtgaagcaaaacccataaaaccattctcagttcggattgtagtctgcaactcgactacatgaagttggaatcgctagtaatcgcgaatcagcatgtcgcggtgaatacgttcccgggccttgtacacaccgcccgtcacaccatgagagttg

SEQ ID NO: 14 - DNA, 1502 bp, Lactobacillus fermentum
  source 1..1502: mol_type = genomic DNA
cggcggtgtgcctaatacatgcaagtcgaacgcgttggcccaattgattgatggtgcttgcacctgattgattttggtcgccaacgagtggcggacgggtgagtaacacgtaggtaacctgcccagaagcgggggacaacatttggaaacagatgctaataccgcataacaacgttgttcgcatgaacaacgcttaaaagatggcttctcgctatcacttctggatggacctgcggtgcattagcttgttggtggggtaacggcctaccaaggcgatgatgcatagccgagttgagagactgatcggccacaatgggactgagacacggcccatactcctacgggaggcagcagtagggaatcttccacaatgggcgcaagcctgatggagcaacaccgcgtgagtgaagaagggtttcggctcgtaaagctctgttgttaaagaagaacacgtatgagagtaactgttcatacgttgacggtatttaaccagaaagtcacggctaactacgtgccagcagccgcggtaatacgtaggtggcaagcgttatccggatttattgggcgtaaagagagtgcaggcggttttctaagtctgatgtgaaagccttcggcttaaccggagaagtgcatcggaaactggataacttgagtgcagaagagggtagtggaactccatgtgtagcggtggaatgcgtagatatatggaagaacaccagtggcgaaggcggctacctggtctgcaactgacgctgagactcgaaagcatgggtagcgaacaggattagataccctggtagtccatgccgtaaacgatgagtgctaggtgttggagggtttccgcccttcagtgccggagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgccaaccctagagatagggcgtttccttcgggaacgcaatgacaggtggtgcatggtcgtcgtcagctcgtgtcgtgagatgttgggttaagtcccgcaacgagcgcaacccttgttactagttgccagcattaagttgggcactctagtgagactgccggtgacaaaccggaggaaggtggggacgacgtcagatcatcatgccccttatgacctgggctacacacgtgctacaatggacggtacaacgagtcgcgaactcgcgagggcaagcaaatctcttaaaaccgttctcagttcggactgcaggctgcaactcgcctgcacgaagtcggaatcgctagtaatcgcggatcagcatgccgcggtgaatacgttcccgggccttgtacacaccgcccgtcacaccatgagagtttgtaacacccaaagtcggtggggtaaccttttaggagccagccgcctaaggtgggacagatgattagggtgaagtcgtaacaag

SEQ ID NO: 15 - DNA, 1433 bp, Clostridium sp.
  source 1..1433: mol_type = genomic DNA
agagtttgatcctggctcaggatgaacgctggcggcgtgcctaatacatgcaagtcgaacgcttcacttcggtgaagagtggcgaacgggtgagtaatacataagtaacctggcatctacagggggataactgatggaaacgtcagctaagaccgcataggtgtagagatcgcatgaactctatatgaaaagtgctacgggactggtagatgatggacttatggcgcattagctggttggtagggtaacggcctaccaaggcgacgatgcgtagccgacctgagagggtgaccggccacactgggactgagacacggcccagactcctacgggaggcagcagtagggaattttcggcaatgggggaaaccctgaccgagcaacgccgcgtgaaggaagaagtaattcgttatgtaaacttctgtcatagaggaagaacggtggatatagggaatgatatccaagtgacggtactctataagaaagccacggctaactacgtgccagcagccgcggtaatacgtaggtggcgagcgttatccggaattattgggcgtaaagagggagcaggcggcactaagggtctgtggtgaaagatcgaagcttaacttcggtaagccatggaaaccgtagagctagagtgtgtgagaggatcgtggaattccatgtgtagcggtgaaatgcgtagatatatggaggaacaccagtggcgaaggcgacgatctggcgcataactgacgctcagtcccgaaagcgtggggagcaaataggattagataccctagtagtccacgccgtaaacgatgagtactaagtgttgggagtcaaatctcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatcgatctaaaggctccagagatggagagatagctatagagaagacaggtggtgcatggttgtcgtcagctcgtgtcgtgagatgttgggttaagtcccgcaacgagcgcaacccctgttgccagttgccagcattaagttggggactctggcgagactgccggtgacaagccggaggaaggcggggatgacgtcaaatcatcatgccccttatgacctgggctacacacgtgctacaatggacagagcagagggaagcgaagccgcgaggtggagcgaaacccataaaactgttctcagttcggactgcagtctgcaactcgactgcacgaagatggaatcgctagtaatcgcgaatcagcatgtcgcggtgaatacgttctcgggccttgtacacaccgcccgtcacaccatgagagtcggtaacacccgaagccggtggcctaaccgcaaggaa

SEQ ID NO: 16 - DNA, 1489 bp, Lactobacillus animalis
  variation 1259: note = a, c, t, g, unknown or other
  source 1..1489: mol_type = genomic DNA
gatgaacgctggcggcgtgcctaatacatgcaagtcgaacgaaacttctttatcaccgagtgcttgcactcaccgataaagagttgagtggcgaacgggtgagtaacacgtgggcaacctgcccaaaagagggggataacacttggaaacaggtgctaataccgcataaccatagttaccgcatggtaactatgtaaaaggtggctatgctaccgcttttggatgggcccgcggcgcattagctagttggtgaggtaaaggcttaccaaggcaatgatgcgtagccgaactgagaggttgatcggccacattgggactgagacacggcccaaactcctacgggaggcagcagtagggaatcttccacaatgggcgaaagcctgatggagcaacgccgcgtgggtgaagaaggtcttcggatcgtaaaaccctgttgttagagaagaaagtgcgtgagagtaactgttcacgtttcgacggtatctaaccagaaagccacggctaactacgtgccagcagccgcggtaatacgtaggtggcaagcgttatccggatttattgggcgtaaagggaacgcaggcggtcttttaagtctgatgtgaaagccttcggcttaaccggagtagtgcattggaaactgggagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgaatgctaagtgttggagggtttccgcccttcagtgctgcagctaacgcaataagcattccgcctggggagtacgaccgcaaggttgaaactcaaaggaattgacgggggcccgcacaagcggtggagcatgtggtttaattcgaagcaacgcgaagaaccttaccaggtcttgacatcttctgacaatcctagagataggactttcccttcggggacagaatgacaggtggtgcatggttgtcgtcagctcgtgtcgtgagatgttgggttaagtcccgcaacgagcgcaacccttattgttagttgccagcattaagttgggcactctagcaagactgccggtgacaaaccggaggaaggtggggatgacgtcaaatcatcatgccccttatgacctgggctacacacgtgctacaatggacggtacaacgagtcgcaagancgcgaggtttagcaaatctcttaaagccgttctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagccggtggggtaaccttttggagccagccgtctaaggtgggacagatgattggggtgaag

SEQ ID NO: 17 - DNA, 1513 bp, Streptococcus parasanguinis
  source 1..1513: mol_type = genomic DNA
ttgatcctggctcaggatgaacgctggcggcgtgcctaatacatgcaagtagaacgctgaagcttggtgcttgcaccgagcggaggagttgcgaacgggtgagtaacgcgtaggtaacctgcctcttagcgggggataactattggaaacgatagctaataccgcataaaagtcgacattgcatgatgttgacttgaaaggtgcaaatgcatcactaagagatggacctgcgttgtattagctagttggtgaggtaacggctcaccaaggcgacgatmcatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtaacttaccagaaagggacggctaactacgtgccagcagccgcggtaatacgtaggtcccgagcgttrtccggatttattgggcgtaaagcgagcgcaggcggttagataagtctgaagttaaaggctgtggcttaaccatagtacgctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttgggtcctttccgggactcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagctttccttcgggacagaggtgacaggtggtgcatggttgtcgtcagctcgtgtcgtgagatgttgggttaagtcccgcaacgagcgcaacccctattgttagttgccatcatttagttgggcactctagcgagactgccggtaataaaccggaggaaggtggggatgacgtcaaatcatcatgccccttatgacctgggctacacacgtgctacaatggctggtacaacgagtcgcgagtcggtgacggcaagctaatctcttaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaagtagccgtatcggaag

SEQ ID NO: 18 - DNA, 1499 bp, Clostridium sp.
  source 1..1499: mol_type = genomic DNA
tcgcccttagagtttgatcgtggctcaggatgaacgctggcggcgtgcttaacacatgcaagtcgaacgaagcgcctcggattgatttcttcggattgatatctctggtgactgagtggcggacgggtgagtaacgcgtgggtaacctgcctcatacagggggataacagttagaaatgactgctaataccgcataagcgcacagtaccgcatggtacagtgtgaaaaactccggtggtatgagatggacccgcgtctgattagcttgttggcggggtaacggcccaccaaggcgacgatcagtagccgacctgagagggtgatcggccacattgggactgagacacggcccaaactcctacgggaggcagcagtggggaatattgcacaatgggggaaaccctgatgcagcgacgccgcgtgaaggaagacgtatttcggtatgtaaacttctatcagcaaggaagatgatgacggtacttgactaagaagccccggctaactacgtgccagcagccgcggtaatacgtagggggcaagcgttatccggatttactgggtgtaaagggagcgtaggcggccatgcaagtcagaagtgaaagcccggggctcaaccccgggactgcttttgaaactgtagggctagattgccggagaggtgagcggaattcctagtgtagcggtgaaatgcgtagatattaggaggaacaccagtggcgaaggcggctcactggacggtaaatgacgctgaggcttgaaagcgtggggagcaaacaggattagataccctggtagtccacgccgtaaacgatgaatactaggtgtcggggcccagaagggcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaggtcttgacatctgggcgaccggagagtaatgtctcctttcctacgggacgcccaagacaggtggtgcatggttgtcgtcagctcgtgtcgtgagatgttgggttaagtcccgcaacgagcgcaacccctatttttagtagccagcggtccggccgggcactctagagagactgccagggataacctggaggaaggtggggatgacgtcaaatcatcatgccccttatgatctgggctacacacgtgctacaatggtggtaacaaagtgaggcgaacctgtgagggggagcgaatcacataaaagccatctcagttcggattgtagtctgcaactcgactacatgaagctggaatcgctagtaatcgcgaatcagcatgtcgcggtgaatacgttcccgggtcttgtacacaccgcccgtcacaccatgggagttggatatgcccgaagtcagtgacccaaccgagaggagggagctgccgaaggtggagaacgatgactggggtgaagtcgtaacaatt

SEQ ID NO: 19 - DNA, 1406 bp, Granulicatella adiacens
  variation 63: note = a, c, t, g, unknown or other
  variation 134: note = a, c, t, g, unknown or other
  variation 388: note = a, c, t, g, unknown or other
  variation 588: note = a, c, t, g, unknown or other
  variation 867: note = a, c, t, g, unknown or other
  variation 1265: note = a, c, t, g, unknown or other
  source 1..1406: mol_type = genomic DNA
agagtttgatcatggctcaggacgaacgctggcggcgtgcctaatacatgcaagtcgaacganagcgaccggtgcttgcactggtcaatctagtggcgaacgggtgagtaacacgtgggtaacctgcccatcanagggggataacatccggaaacggatgctaaaaccgcataggtcttcgaaccgcatggtttgaagaggaaaagaggcgcaagcttctgctgatggatggacccgcggtgcattagctagttggtgaggtacggctcaccaaggccgtgatgcatagccgacctgagagggtgatcggccacattgggactgagacacggcccaaactcctacgggaggcagcagtagggaatcttccgcaatggacgcaagtctnacggagcaacgccgcgtgagtgaagaaggttttcggatcgtaaaactctgttgttagagaagaacaagtgctagagtaactgttagcgccttgacggtatctaaccagaaagccacggctaactacgtgccagcagccgcggtaatacgtaggtggcaagcgttgtccggatttattgggcgtaaagcgagcgcaggcgnttccttaagtctgatgtgaaagcccccggctcaaccggggagggtcattggaaactggggaacttgagtgcagaagaggagagtggaattccatgtgtagcggtgaaatgcgtagatatatggaggaacaccagtggcgaaggcgactctctggtctgtaactgacgctgaggctcgaaagcgtgggtagcaaacaggattagataccctggtagtccacgccgtaaacgatgagtgctaagtgttggagggtttccgcccttcagtgctgcagttaangcattaagcactccgcctggggagtacgaccgcaaggttgaaactcaaaggaattgacggggacccgcacaagcggtggagcatgtggtttaattcgaagcaacgcgaagaaccttaccaagtcttgacatcctttgaccactctagagatagagctttcccttcggggacaaagtgacaggtggtgcatggttgtcgtcagctcgtgtcgtgagatgttgggttaagtcccgcaacgagcgcaacccttattactagttgccagcattcggttgggcactctagtgagactgccggtgacaaaccggaggaaggtggggatgacgtcaaatcatcatgccccttatgacttgggctacacacgtgctacaatggatggtacaacgagcagcgaactngcgagggtaagcgaatctcttaaagccattctcagttcggattgtaggctgcaactcgcctacatgaagccggaatcgctagtaatcgcggatcagcacgccgcggtgaatacgttcccgggtcttgtacacaccgcccgt

SEQ ID NO: 20 - DNA, 1413 bp, Actinomyces graevenitzii
  source 1..1413: mol_type = genomic DNA
ggcggcgtgcttaacacatgcaagtcgaacgaaaataccaagcttgcttggttgattagtggcgaacgggtgagtaacacgtgagtaacctgccccttactttcctggataactgctcgaaagggtagctaatacgggatattctgaccagcctgcatgggttggtttggaaaggttgtttttctggtaagggatgggctcgcggcctatcagcttgttggtggggtgatggcctaccaaggctttgacgggtagccggcctgagagggtggacggtcacactgggactgagatacggcccagactcctacgggaggcagcagtggggaatattgcacaatgggcgcaagcctgatgcagcgacgccgcgtgagggatgaaggccttcgggttgtaaacctctttcgtcagggaacaaggcactgcttttttggtggtgttgagggtacctggataagaagcgccggctaactacgtgccagcagccgcggtaatacgtagggcgcaagcgttgtccggaattattgggcgtaaagagctcgtaggcggctggtcgcgtctgtcgtgaaaacttccggctcaaccgggggcttgcggtgggtacgggccggctagagtgcggtaggggtaactggaactcctggtgtagcggtggaatgcgcagatatcaggaagaacaccgatggcgaaggcaggttactgggccgttactgacgctgaggagcgaaagcgtggggagcgaacaggattagataccctggtagtccacgctgtaaacgttgggcactaggtgtgaggctcctttccgggggttttgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttaagcgtggctctagagatagggtttacccttttgggtggttttcacaggtggtgcatggttgtcgtcagctcgtgtcgtgagatgttgggttaagtcccgcaacgagcgcaacccttgtcccgtgttgccagcacgttgtggtggggactcgcgggagactgccggggtcaactcggaggaaggtggggatgacgtcaaatcatcatgccccttatgtcttgggcttcacgcatgctacaatggctggtacagagggttgcgatactgtgaggtggagctaatcccttaaagctggtctcagttcggattggtgtctgcaactcgacaccatgaaggtggagtcgctagtaatcgcagatcagcaacgctgcggtgaatacgttctcgggccttgtacacaccgcccgtcacgtcacgaaagttggcaacaccccaagcccgtggctct

SEQ ID NO: 21 - DNA, 1429 bp, Clostridium bartlettii
  variation 47: note = a, c, t, g, unknown or other
  source 1..1429: mol_type = genomic DNA
aggatgaacgctggcggcgtgcctaacacatgcaagtcgagcgattntcttcggagaagagcggcggacgggtgagtaacgcgtgggtaacctgccctgtacacacggataacataccgaaaggtatgctaatacgggataacataagaaattcgcatgtttttcttatcaaagctccggcggtacaggatggacccgcgtctgattagctagttggtgaggtaacggctcaccaaggcgacgatcagtagccgacctgagagggtgatcggccacattggaactgagacacggtccaaactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggatttactgggcgtaaagggtgcgtaggcggtcttttaagtcaggagtgaaaggctacggctcaaccgtagtaagctcttgaaactggaggacttgagtgcaggagaggagagtggaattcctagtgtagcggtgaaatgcgtagatattaggaggaacaccagtagcgaaggcggctctctggactgtaactgacgctgaggcacgaaagcgtggggagcaaacaggattagataccctggtagtccacgccgtaaacgatgagtactagctgtcggaggttacccccttcggtggcgcagctaacgcattaagtactccgcctggggagtacgctcgcaagagtgaaactcaaaggaattgacggggacccgcacaagtagcggagcatgtggtttaattcgaagcaacgcgaagaaccttacctaagcttgacatccttttgaccgatgcctaatcgcatctttcccttcggggacagaagtgacaggtggtgcatggttgtcgtcagctcgtgtcgtgagatgttgggttaagtcccgcaacgagcgcaacccttgcctttagttgccatcattaagttgggcactctagagggactgccagggataacctggaggaaggtggggatgacgtcaaatcatcatgccccttatgcttagggctacacacgtgctacaatgggtggtacagagggcagcgaagtcgtgaggccaagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatgggagttgggggcgcccgaagccggctagctaaccttttggaagcggtcgtcgaaggtgaaaccaataactggggtgaa

SEQ ID NO: 22 - DNA, 1502 bp, Clostridium algidixylanolyticum
  source 1..1502: mol_type = genomic DNA
agagtttgatcctggctcaggatgaacgctggcggcgtgcttaacacatgcaagtcgagcgaagcgattttaaggaagccttcgggtggaattagaattgacttagcggcggacgggtgagtaacgcgtgggtaacctgcctcatacagggggataacagttagaaatgactgctaataccgcataagcgcacagtgctgcatagcacagtgtgaaaaactccggtggtatgagatggacccgcgtctgattaggtagttggtgaggtaacggcccaccaagccgacgatcagtagccgacctgagagggtgaccggccacattgggactgagacacggcccaaactcctacgggaggcagcagtggggaatattggacaatgggggaaaccctgatccagcgacgccgcgtgactgaagaagtatttcggtatgtaaaggtctatcagcagggaagaaaatgacggtacctgactaagaagccccggctaactacgtgccagcagccgcggtaatacgtagggggcaagcgttatccggatttactgggtgtaaagggagcgtagacggctctgcaagtctggagtgaaagcccggggctcaaccccgggactgctttggaaactgtggagctagagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgtcggggagcaaagctcttcggtgccgtcgctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcggaatgacagccttataactaaggtctccctacggggcattctagacaggtggtgcatggttgtcgtcagctcgtgtcgtgagatgttgggttaagtcccgcaacgagcgcaacccttatccttagtagccagcagttcggctgggcactctggggagactgccagggataacctggaggaaggtggggatgacgtcaaatcatcatgccccttatgatttgggctacacacgtgctacaatggcgtaaacaaagggaagcgaaggagcgatctggagcaaaccccaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagttggtaacgcccgaagtcagtgacccaaccgtaaggagggagctgccgaaggcgggactgataactggggtgaagtcgtaacaaggtagccgtatcggaga

SEQ ID NO: 23 - DNA, 1506 bp, Lactobacillus homohiochii
  source 1..1506: mol_type = genomic DNA
gacgaacgctggcggcatgcctaatacatgcaagtcgaacgagctgcgcctaatgatagttgatgcttgcattagcttgacttaagttagcagcgagtggcgaactggtgagtaacacgtggataacctgcccagaagaaggggataacacctggaaacagatgctaataccgtataacaacgaaaaccacatggttttcgtttgaaagatggcctttgtgctatcgcttttggatggatccgcggcgcattagctagttggtgagataaaggctcaccaaggcaatgatgcgtagccgacctgagagggtaatcggccacattgggactgagacacggcccagactcctacgggaggcagcagtagggaatcttccacaatggacgaaagtctgatggagcaatgccgcgtgagtgaagaagggtttcggctcgtaaaactctgttgttagagaagaacgggcgtgagagtaaytgctcacgtcgcgacggtatctaaccagaaagtcacggctaactacgtgccagcagccgcggtaatacgtaggtggcaagcgttgtccggatttattgggcgtaaagcgagcgcaggcggtcttctaagtctgatgtgaaagccttcggcttaaccggagaagtgcatcggaaactgggaaacttgagtgcagaagaggacagtggaactccatgtgtagcggtgaaatgcgtagatatatggaggaacaccagtggcgaaggcggctgtctagtctgtaactgacgctgaggctcgaaagcatgggtagcaaacaggattagataccctggtagtccatgccgtaaacgatgagtgctaggtgttggagggtttccgcccttcagtgccgcagctaacgcattaagcactccgcctggggagtacgaccgcaaggttgaaactcaaaggaattgacggggacccgcacaagcggtggagcatgtggtttaattcgatgctacgcgaagaaccttaccaggtcttgacatcttctgttaacctaagagattaggtgtccccttcgggggcagaatgacaggtggtgcatggttgtcgtcagctcgtgtcgtgagatgttgggttaagtcccgcaacgagcgcaacccttgtcattagttgccagcattaagttgggcactctagtgagactgccggtgacaaaccggaggaaggtggggatgacgtcaaatcatcatgccccttatgacctgggctacacacgtgctacaatggacggtacaacgagttgcgaaaccgcgaggtcaagctaatctcttaaagccgttctcagttcggattgcaggctgcaactcgcctgcatgaagttggaatcgctagtaatcgtggatcagcatgccacggtgaatacgttcccgggtcttgtacacaccgcccgtcacaccatgagagtttgtaacacccaaagtcggttaggtaaccttttggagcctgccgcctaaggtgggacagatgattagggtgaagtcgta

SEQ ID NO: 24 - DNA, 1517 bp, Tyzzerella nexilis
  variation 184..185: note = a, c, t, g, unknown or other
  variation 341: note = a, c, t, g, unknown or other
  variation 430: note = a, c, t, g, unknown or other
  source 1..1517: mol_type = genomic DNA
gagatttgatcctggctcaggatgaacgctggccggccgtgcttacacatgcagtcgaacgaagcgcttaaactggatttcttcggattgaagtttttgctgactgagtggcggacgggtgagtaacgcgtgggtaacctgcctcatacagggggataacagttagaaatgactgctaataccnnataagcgcacagtgctgcatggcacagtgtaaaaactccggtggtatgagatggacccgcgtctgattagctagttggtggggtaacggcctaccaaggcgacgatcagtagccggcctgagagggtgaacggccacattgggactgagacacggnccaaactcctacgggaggcagcagtggggaatattgcacaatgggggaaaccctgatgcagcgacgccgcgtgagcgaagaagtatttnggtatgtaaagctctatcagcagggaagaaaatgacggtacctgactaagaagcaccggctaaatacgtgccagcagccgcggtaatacgtaggtgcaagcgttatccggatttactgggtgtaaagggagcgtagacggttgtgtaagtctgatgtgaaagcccggggctcaaccccggactgcattggaaactatgtaactagagtgtcggagaggtaagcggaattcctagtgtagcggtgaaatgcgtagatattaggaggaacaccagtggcgaaggcggcttactggacgatcactgacgttgaggctcgaaagcgtggggagcaaacaggattagataccctggtagtccacgccgtaaacgatgactactaggtgtcggggagcaaagctcttcggtgccgcagcaaacgcaataagtagtccacctggggagtacgttcgcaagaatgaaactcaaaggaattgacggggacccgcacaagcgtggagcatgtggtttaattcgagcaacgcgaagaccttacctggtcttgacatcccggtgaccggtccagtaatgggacctttccttcgggacacggtgacaggtggtgcatggttgtcgtcagctcgtgtcgtgagatgttgggttaagtcccgcaacgagcgcaacccctatcttcagtagccagcatttaaggtgggcactctggagagactgccagggataacctggaggaaggtggggatgacgtcaaatcatcatgccccttatgaccagggctacacacgtgctacaatggcgtaaacaaagggaagcgaacctgtgaggggaagcaaatctcaaaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagtcagtaacgcccgaagtcagtgacccaaccgtaaggagggagctgccgaaggtgggaccgataactggggtgaagtcgtaacaaggtagccgtatcggaaggtgcggctggatcacctcc

SEQ ID NO: 25 - DNA, 1478 bp, Mogibacterium neglectum
  source 1..1478: mol_type = genomic DNA
agtttgatcatggctcaggatgaacgctggcggcgtgcctaatacatgcaagtcgagcgagatgttagcgcatgaaccttcgggggattatgctaacggaaagcggcggacgggtgagtaacgcgtaggcaacctgcccctgacagagggatagccattggaaacgatgattaaaacctcatgacaccgatgaagcacatgcttcatcggtcaaagatttatcggtcagggatgggcctgcgtctgattaactagttggtgaggtaacggctcaccaaggtgacgatcagtagccgacctgagagggtgatcggccacattggaactgagacacggtccaaacttctacggaaggcagcagtagggaatcttgcacaatgggcgaaagcctgatgcagcaacgccgcgtgaaggatgaaggccttcgggttgtaaacttctgttctaagggaagaaacaaatgacggtaccttaggagcaagccccggctaactacgtgccagcagccgcggtaatacgtagggggcaagcgttatccggaattattgggcgtaaagagtgcgtaggtggttacctaagcgcaaggtttaatttagaggctcaacctctacttgccttgcgaactgggctacttgagtgcaggaggggaaagcggaattcctagtgtagcggtgaaatgcgtagatattaggaggaacaccagcggcgaaggcggctttctggactgtaactgacactgaggcacgaaagcgtgggtagcaaacaggattagataccctggtagtccacgccgtaaacgatgagcactaggtgttgggcccgttaggctcagtgccgcagttaacgcaataagtgctccgcctggggagtacgctcgcaagagtaaaactcaaaggaattgacggggacccgcacaagcagcggagcatgtggtttaattcgaagcaacgcgaagaaccttaccagggcttgacatcctgctgacaggaccttaaccggtcccttcttcggacagcagagacaggtggtgcatggttgtcgtcagctcgtgtcgtgagatgttgggttaagtcccgcaacgagcgcaacccttgtcgctagttactaacattcagttgaggactctagcgagactgccgaggtcaactcggaggaaggtggggatgacgtcaaatcatcatgccccttatgttctgggctacacacgtgctacaatggtcggtacaatgggatgcaatactgtgaagtggagcaaatcaccaaaaccgatcccagttcggattgtaggctgcaactcgcctacatgaagttggagttgctagtaatcgcagatcagaatgctgcggtgaatgcgttcccgggtcttgtacacaccgcccgtcacaccatgggaagttgggggtgcccaaagtcggttaattaatctatcgcctaaggcaaaaccaatgactggggtgaagtcgtaacaaggtaaccgt

SEQ ID NO: 26 - DNA, 1535 bp, Lactobacillus reuteri
  source 1..1535: mol_type = genomic DNA
agagtttgatcctggctcaggatgaacgccggcagtgtgcctaatacatgcaagtcgtacgcactggcccaactaattgatggtgcttgctgaattgacgatggatcaccagtgagtggcggacgggtgagtaacacgtaggtaacctgccccggagcggggaataacatttggaaacagatgctaataccgcataacaacaaaagccgcatggtttttctggaaagatggctttggctatcactctgggatggacctgcggtgcatttagctagttggtaaggtaacggcttacccaaggcgatgatgcatagccgagttgagagactgatcggccacaatgggaactgagacacggtccataacttctacgggaggcagcagtagggaatcttccacaatgggcgcaagctgatggagcaacaccgcgttattaagaaagggtttcggccgcttaaactctgttgttggagaagaacgtgcgttagagtaactgttacgcagtgacggtatccaaccagaaagtcacggctaactacgtgccagcagccgcggtaatacgtaggtggcaagcgttatccggatttattgggcgtaaagcgagcgcaggcggttgcttaggtctgatgtggaaactcggcttaaccgaagaagtgcatcggaaaccgggcgacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtccatgccgtaaacgatgagtgctaggtgttggagggtttccgcccttcagtgcctgttctaacgcattaatgcactccgcctggggagtacgaccgcaaggttgaaactcaaaggaattgacgggggcccgcacaagcggtgaagcatgtggtttaattcgaagctacgcgaagaaccttaccaggtcttgacatcttgcgctaaccttagagataaggcgttcccttcggggacgttaatgacaggtggtgcatggtcgtcgtcagctcgtgtcgtgagatgttgggttaagtcccgcaacgagcgcaacccttgttactagttgccagcattaagttggggactctagtgagactgccggtgacaaaccggaggaaggtggggacgacgtcagatcatcatgccccttatgaccctgggctacacacgtgctacaatggacggtacaacgagtcgcaaactcgcgagagtaagctaatctcttaaagccgttctcagttcggactgtaggctgcaactcgcctacacgaagtcggaatcgctagtaatcgcggatcagcatgccgcggtgaatacgttcccgggccttgtacacaccgcccgtcacaccatgggagtttgtaacgcccaaagttcggtggcctaacctttatggacgggtaccctaaggcgggacagatgatctggggtgaagtcgtaacaaggta

SEQ ID NO: 27 - DNA, 1484 bp, Clostridium saccharogumia
  source 1..1484: mol_type = genomic DNA
aagagtttgatcctggctcaggatgaacgctggcggcgtgcctaatacatgcaagtcgaacgcgggcagtaatgtccgagtggcgaacgggtgagtaagacataagtaacctgccctttacagggggataactattggaaacgatagctaagaccgcataggtaaagataccgcatggtaggtttattaaaagtgccaaggcactggtagaggatggacttatggcgcattagctggttggtgaggtaacggctcaccaaggcgacgatgcgtagccgacctgagagggtgaccggccacactgggactgagacacggcccagactcctacgggaggcagcagtagggaattttcggcaatggggggaaccctgaccgagcaacgccgcgtgaaggaggaaggtcttcggactgtaaacttctgttataaaggaagaaaggcggatacagggaatggtatccgagtgacggtactttatgaggaagccacggctaactacgtgccagcagccgcggtaatacgtaggtggcaagcgttatccggaattattgggcgtaaagagggagcaggcggcagcaagggtctgtggtgaaagactgaagcttaacttcagtaagccatagaaaccgggcagctagagtgcaggagaggatcgtggaattccatgtgtagcggtgaaatgcgtagatatatggaggaacaccagtggcgaaggcgacgatctggcctgcaactgacgctcagtcccgaaagcgtggggagcaaataggattagataccctagtagtccacgccgtaaacgatgagtactaagtgttgggagtcaaatctcagtgctgcagttaacgcagtaagtactccgcctgagtagtacgttcgcaagaatgaaactcaaaggaattgacgggggcccgcacaagcggtggagcatgtggtttaattcgaagcaacgcgaagaaccttaccaggtcttgacatactcataaaggctccagagatggagagataggtatatgggatacaggtggtgcatggttgtcgtcagctcgtgtcgtgagatgttgggttaagtcccgcaacgagcgcaacccttatcgttagttaccatcattaagttggggactctagcgagactgccagtgacaagctggaggaaggcggggatgacgtcaaatcatcatgccccttatgacctgggctacacacgtgctacaatggatggagcagagggaagcgaagccgcgaggtggagcgaaacccagaaaaccattctcagttcggattgtagtctgcaactcgactacatgaagttggaatcgctagtaatcgcgaatcagcatgtcgcggtgaatacgttctcgggccttgtacacaccgcccgtcacaccatgagagttgataacacccgaagccggtggcctaaccgcaaggagggagctgtctaaggtgggattgatgattggggtgaagtcgtaacaagggt

SEQ ID NO: 28 - DNA, 1479 bp, Eubacterium hallii
  variation 617: note = a, c, t, g, unknown or other
  variation 955: note = a, c, t, g, unknown or other
  variation 1087: note = a, c, t, g, unknown or other
  variation 1098: note = a, c, t, g, unknown or other
  variation 1184..1185: note = a, c, t, g, unknown or other
  variation 1227: note = a, c, t, g, unknown or other
  variation 1259: note = a, c, t, g, unknown or other
  variation 1269: note = a, c, t, g, unknown or other
  variation 1374..1375: note = a, c, t, g, unknown or other
  variation 1391: note = a, c, t, g, unknown or other
  source 1..1479: mol_type = genomic DNA
tttatttgagagtttgatcctggctcaggatgaacgctggcggcgtgcctaacacatgcaagtcgaacgaagcaccttaccwgattcttcggatgaaagwytggtgactgagtggcggacgggtgagtaacgcgtgggtaacctgccctgtacagggggataacagctggaaacggctgctaataccgcataagcgcacgaggagacatctccttgtgtgaaaaactccggtggtacaggatgggcccgcgtctgattagctggttggcagggtaacggcctaccaaggcaacgatcagtagccggtctgagaggatgaacggccacattggaactgagacacggtccaaactcctacgggaggcagcagtggggaatattgcacaatgggggaaaccctgatgcagcaacgccgcgtgagtgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggtgcgtaggtggcagtgcaagtcagatgtgaaaggccggggctcaaccccggngctgcatttgaaactgcwyrgctagagtacaggagaggcaggcggaattcctagtgtagcggtgaaatgcgtagatattaggaggaacaccagtggcgaaggcggcctgctggactgttactgacactgaggcacgaaagcgtggggagcaaacaggattagataccctggtagtccacgccgtaaacgatgaatactaggtgtcggggccgtataggctycggtgccgccgctaacgcagtaagtattccacctggggagtacgttcgcaagaatgaaactcaaaggaattgacggggacccgcacaagcggtggagcatgtggtttaattcgaagnaacgcgaagaaccttaccaggtcttgacatccttctgaccgcaccttaatcggtgctttccttcgggacagaagagacaggtggtgcatggttgtcgtcagctcgtgtcgtgagatgttgggttaagtcccncaacgagcgcnacccctatcttcagtagccagcaggtaaggctgggcactctggagagactgccagggataacctggaggaaggtggggacgacgtnnaatcatcatgccccttatgatctgggcgacacacgtgctacnatggcggtcacagagtgaggcgaaccygcganggggagcaanccacaaaaaggccgtcccagttcggactgtagtctgcaacccgactacacgaagctggaatcgctagtaatcgcgaatcagaatgtcgcggtgaatacgttcccnngtcttgtacacaccgnccgtcacaccatgggagtcggaaatgcccgaagccagtgacccaacctttatggagggagctgtcgaaggtggagccggtaactgggg

SEQ ID NO: 29 - DNA, 1476 bp, Drancourtella massiliensis
  source 1..1476: mol_type = genomic DNA
cctggctcaggatgaacgctggcggcgtgcctaacacatgcaagtcgagcgaagcgctgttttcagaatcttcggaggaagaggacagtgactgagcggcggacgggtgagtaacgcgtgggcaacctgcctcatacagggggataacagttagaaatgactgctaataccgcataagcgcacaggaccgcatggtccagtgtgaaaaactccggtggtatgagatggacccgcgtctgattaggtagttggtggggtaaaggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgaaggaagaagtatttcggtatgtaaacttctatcagcagggaagaaratgacggtacctgagtaagaagcaccggctaaatacgtgccagcagccgcggtaatacgtatggtgcaagcgttatccggatttactgggtgtaaagggagcgtagacggataggcaagtctggagtgaaaacccagggctcaaccctgggactgctttggaaactgcagatctggagtgccggagaggtaagcggaattcctagtgtagcggtgaaatgcgtagatattaggaggaacaccagtggcgaaggcggcttactggacggtgactgacgttgaggctcgaaagcgtggggagcaaacaggattagataccctggtagtccacgccgtaaacgatgactactaggtgtcggtgtgcaaagcacatcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctggtcttgacatccggatgacgggcgagtaatgtcgccgtcctttcggggcatccgagacaggtggtgcatggttgtcgtcagctcgtgtcgtgagatgttgggttaagtcccgcaacgagcgcaacccttatcttcagtagccagcatataaggtgggcactctggagagactgccagggagaacctggaggaaggtggggatgacgtcaaatcatcatgccccttatggccagggctacacacgtgctacaatggcgtaaacaaagggaagcgagagggtgacctgaagcgaatcccaaaaataacgtctcagttcggattgtagtctgcaactcgactacatgaagctggaatcgctagtaatcgcggatcagcatgccgcggtgaatacgttcccgggtcttgtacacaccgcccgtcacaccatgggagtcagtaacgcccgaagccagtgacccaacctttgaggagggagctgtcgaaggcgggagggataactggggtgaagtcgtaacaa

SEQ ID NO: 30 - DNA, 1513 bp, Clostridium perfringens
  source 1..1513: mol_type = genomic DNA
taaattgagagtttgatcctggctcaggatgaacgctggcggcgtgcttaacacatgcaagtcgagcgatgaagtttccttcgggaaacggattagcggcggacgggtgagtaacacgtgggtaacctgcctcatagagtggaatagccttccgaaaggaagattaataccgcataacgttgaaagatggcatcatcattcaaccaaaggagcaatccgctatgagatggacccgcggcgcattagctagttggtggggtaacggcctaccaaggcgacgatgcgtagccgacctgagagggtgatcggccacattgggactgagacacggcccagactcctacgggaggcagcagtggggaatattgcacaatgggggaaaccctgatgcagcaacgccgcgtgagtgatgaaggttttcggatcgtaaagctctgtctttggggaagataatgacggtacccaaggaggaagccacggctaactacgtgccagcagccgcggtaatacgtaggtggcgagcgttatccggatttactgggcgtaaagggagcgtaggcggatgattaagtgggatgtgaaatacccgggctcaacttgggtgctgcattccaaactggttatctagagtgcaggagaggagagtggaattcctagtgtagcggtgaaatgcgtagagattaggaagaacaccagtggcgaaggcgactctctggactgtaactgacgctgaggctcgaaagcgtggggagcaaacaggattagataccctggtagtccacgccgtaaacgatgaatactaggtgtgggggtttcaacacctccgtgccgccgctaacgcattaagtattccgcctggggagtacggtcgcaagattaaaactcaaaggaattgacggggacccgcacaagtagcggagcatgtggtttaattcgaagcaacgcgaagaaccttacctacacttgacatcccttgcattactcttaatcgaggaaatcccttcggggacaaggtgacaggtggtgcatggttgtcgtcagctcgtgtcgtgagatgttgggttaagtcccgcaacgagcgcaacccttgtcgttagttactaccattaagttgaggactctagcgagactgcctgggttaaccaggaggaaggtggggatgacgtcaaatcatcatgccccttatgtgtagggctacacacgtgctacaatggctggtacagagagatgcaataccgcgaggtggagccaaacttaaaaaccagtctcagttcggattgtaggctgaaactcgcctacatgaagctggagttactagtaatcgcgaatcagaatgtcgcggtgaatacgttcccgggtcttgtacacaccgcccgtcacaccatgagagttggcaatacccgaagtccgtgagctaaccgcaaggaggcagcggccgaaggtagggtcagcgattggggtgaagtcgtaacaaggtagccgtaggagaacctgcggctggatcacctccttt

SEQ ID NO: 31 - DNA, 1926 bp, Bacteroides stercoris
  source 1..1926: mol_type = genomic DNA
gtttgatcctggctcaggatgaacgctagctacaggcttaacacatgcaagtcgaggggcagcatcatcaaagcttgctttgatggagggcgaccggcgcacgggtgagtaacacgtatccaacctgccgacaacactgggatagcctttcgaaagaaagattaataccggatggcatagttttcccgcatgggttgactattaaagaatttcggttgtcgatggggatgcgttccattaggcagttggcggggtaacggcccaccaaacctacgatggataggggttctgagaggaaggtcccccacattggaactgagacacggtccaaactcctacgggaggcagcagtgaggaatattggtcaatggacgagagtctgaaccagccaagtagcgtgaaggatgactgcccgaatttaaaaacttcttttatacgggaataaagtgagccacgtgtggctttttgtatgtaccgtatgaataaggatcggctaactccgtgccagcagccgcggtaatacggaggattcgagcgttatccggatttattgggtttaaagggagcgtaggcgggttgttaagtcagttgtgaaagtttgcggctcaaccgtaaaattgcagttgatactggcgaccttgagtgcaacagaggtaggcggaattcgtggtgtagcggtgaaatgcttagatatcacgaagaactccgattgcgaaggcagcttactggattgtaactgacgctgatgctcgaaagtgtgggtatcaaacaggattagataccctggtagtccacacagtaaacgatgaatactcgctgttggcgatatacagtcagcggccaagtgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaactgacggaaccggaaacggttctttcttcggacagttgtgaaggtgctgcatggttgtcgtcagctcgtgccgtgaggtgtcggcttaagtgccataacgagcgcaacccttacgggtagttaccatcaggttatgctggggactctacccggactgccgtcgtaagatgtgaggaaggtggggatgacgtcaaatcagcacggcccttacgtccggggctacacacgtgttacaatggggggtacagaaggcagctacacggcgacgtggtgctaatcccgaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcgaggagcgtcctagggtaaaactggtgattggggctaagtcgtaacaaggtagccgtaccggaaggtgcggctggaacacctcctttctggagcgacgctcagaaaaaagacgaaaagttggttctaaagagactacctccataagaacctcttgtactgcgaagaattgtttattgataatataaagagaaagtagaagccgagccttcgggctagggtttgactgacagtcctatagctcagttggttagagcgctacactgataatgtagaggtcggcagttcaactctgcctgggactacgacatagtcttggtgagagacacagacgattttaattactaattctaattactaattaccaatcactgaccgtctattactaaccactatctcgggggattagctcagctggctagagcacctgctttgcacgcagggggtcaacggttcgaatccgttattctccactcttgagattccgttaaatcggaata

SEQ ID NO: 32 - DNA, 1404 bp, Ruminococcus faecis
  source 1..1404: mol_type = genomic DNA
atgcaagtcgaacgaagcaccttgatttgattcttcggatgaagatcttggtgactgagtggcggacgggtgagtaacgcgtgggtaacctgcctcatacagggggataacagttagaaatgactgctaataccgcataagaccacagcaccgcatggtgcaggggtaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggagtggcaagtctgatgtgaaaacccggggctcaaccccgggactgcattggaaactgtcaatctagagtaccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgactactaggtgtcgggcagcaaagctgttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatctccctgaccggcaagtaatgttgcctttccttcgggacagggatgacaggtggtgcatggttgtcgtcagctcgtgtcgtgagatgttgggttaagtcccgcaacgagcgcaacccctatctttagtagccagcggtttggccgggcactctagagagactgccagggataacctggaggaaggtggggatgacgtcaaatcatcatgccccttatgagcagggctacacacgtgctacaatggcgtaaacaaagggaggcagaaccgcgaggtcgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggaggagctgccgaag

SEQ ID NO: 33 - DNA, 1256 bp, Clostridium sp.
  variation 942: note = a, c, t, g, unknown or other
  variation 944: note = a, c, t, g, unknown or other
  source 1..1256: mol_type = genomic DNA
ggcgtgcctaacacatgcaagtcgagcggtatatacttaaacaaatcttcggaagagtgagagtatagagagcggcggacgggtgagtaacgcgtaggcaacctgccccatacagagggatagcctcgggaaaccgggattaaaacctcataacgcgaaagagtcacatggctttttcgccaaagatttatcggtatgggatgggcctgcgtctgattagctagttggtggggtaacggctcaccaaggcaacgatcagtagccgacctgagagggtaatcggccacattggaactgagacacggtccaaactcctacgggaggcagcagtggggaatattgcacaatgggcgaaagcctgatgcagcaacgccgcgtgagcgatgaaggcctttgggtcgtaaagctctgtcctaggggaagaataaatgacggtacccttggaggaagccccggctaactacgtgccagcagccgcggtaatacgtagggggcaagcgttatccggaattattgggcgtaaagagtgcgtaggtggttttgtaagcgcggggtgaaaggcaatagctcaactattgtaagccctgcgaactgtgagacttgagtgcaggagaggaaagcggaattcctagtgtagcggtgaaatgcgtagatattaggaggaacaccagtggcgaaggcggctttctggactgtaactgacactgaggcacgaaagcgtggggagcaaacaggattagataccctggtagtccacgccgtaaacgatgagcactaggtgtcggggccgcaaggtttcggtgccgcagttaacgcattaagtgctccgcctggggagtacgcacgcaagtgtgaaactcaaaggaattgacggggacccgcacaagcagcggagcatgtggtttaattcgaagcaacgcgaagaaccttaccagggcttgncntccctctgaccggcgcgtaacggtgcctttccttcgggacagaggagacaggtggtgcatggttgtcgtcagctcgtgtcgtgagatgttgggttaagtcccgcaacgagcgcaacccttgtcattagttgccagcagttcggctgggcactctagtgagactgccggggataactcggaggaaggtggggatgacgtcaaatcatcatgccccttatgttctgggctacacacgtgctacaatggctggtacaaagagaagcgagacggtgacgtggagcaaatctcaaaaaccagtcccagttcggattg

SEQ ID NO: 34 - DNA, 1488 bp, Bacteroides uniformis
  source 1..1488: mol_type = genomic DNA
agagtttgatcctggctcaggatgaacgctagctacaggcttaacacatgcaagtcgaggggcatcaggaagaaagcttgctttctttgctggcgaccggcgcacgggtgagtaacacgtatccaacctgccgatgactcggggatagcctttcgaaagaaagattaatacccgatggtatatctgaaaggcatctttcagctattaaagaatttcggtcattgatggggatgcgttccattaggttgttggcggggtaacggcccaccaagccgtcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagttaggcacgtgtgcctttttgtatgtaccgtatgaataaggatcggctaactccgtgccagcagccgcggtaatacggaggatccgagcgttatccggatttattgggtttaaagggagcgtaggcggatgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcaaatgaatgttctggaaacagatcagccgcaaggcatttgtgaaggtgctgcatggttgtcgtcagctcgtgccgtgaggtgtcggcttaagtgccataacgagcgcaacccttatcgatagttaccatcaggttatgctggggactctgtcgagactgccgtcgtaagatgtgaggaaggtggggatgacgtcaaatcagcacggcccttacgtccggggctacacacgtgttacaatggggggtacagaaggcagctacacggtgacgtgatgctaatccctaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtgcgtaaccgcgaggagcgccctagggtaaaactggtgattggggctaagtcgtaacaaggtaacc

SEQ ID NO: 35 - DNA, 1499 bp, Weissella confusa
  source 1..1499: mol_type = genomic DNA
gatgaacgctggcggcgtgcctaatacatgcaagtcgaacgctttgtggttcaactgatttgaagagcttgctcagatatgacgatggacattgcaaagagtggcgaacgggtgagtaacacgtgggaaacctacctcttagcaggggataacatttggaaacagatgctaataccgtataacaatgacaaccgcatggttgttatttaaaagatggttctgctatcactaagagatggtcccgcggtgcattagctagttggtaaggtaatggcttaccaaggcgatgatgcatagccgagttgagagactgatcggccacaatgggactgagacacggcccatactcctacgggaggcagcagtagggaatcttccacaatgggcgaaagcctgatggagcaacgccgcgtgtgtgatgaagggtttcggctcgtaaaacactgttgtaagagaagaatgacattgagagtaactgttcaatgtgtgacggtatcttaccagaaaggaacggctaaatacgtgccagcagccgcggtaatacgtatgttccaagcgttatccggatttattgggcgtaaagcgagcgcagacggttatttaagtctgaagtgaaagccctcagctcaactgaggaattgctttggaaactggatgacttgagtgcagtagaggaaagtggaactccatgtgtagcggtgaaatgcgtagatatatggaagaacaccagtggcgaaggcggctttctggactgtaactgacgttgaggctcgaaagtgtgggtagcaaacaggattagataccctggtagtccacaccgtaaacgatgagtgctaggtgtttgagggtttccgcccttaagtgccgcagctaacgcattaagcactccgcctggggagtacgaccgcaaggttgaaactcaaaggaattgacggggacccgcacaagcggtggagcatgtggtttaattcgaagcaacgcgaagaaccttaccaggtcttgacatcccttgacaactccagagatggagcgttcccttcggggacaaggtgacaggtggtgcatggttgtcgtcagctcgtgtcgtgagatgttgggttaagtcccgcaacgagcgcaacccttattactagttgccagcattcagttgggcactctagtgagactgccggtgacaaaccggaggaaggtggggatgacgtcaaatcatcatgccccttatgacctgggctacacacgtgctacaatggcgtatacaacgagttgccaacccgcgagggtgagctaatctcttaaagtacgtctcagttcggattgtaggctgcaactcgcctacatgaagtcggaatcgctagtaatcgcggatcagcacgccgcggtgaatacgttcccgggtcttgtacacaccgcccgtcacaccatgagagtttgtaacacccaaagccggtggggtaaccttcgggagccagccgtctaaggtgggacagatgattagggtg

SEQ ID NO: 36 - DNA, 1411 bp, Megasphaera micronuciformis
  source 1..1411: mol_type = genomic DNA
gaaagcttgctttttctacaatctagtggcaaacgggtgagtaacgcgtaaacaacctgccctacggatggggacaacagctggaaacggctgctaataccgaatacgttccgaaagtcgcatgactgtcggaagaaaggatggcctctatttataagctatcgccggaggaggggtttgcgtctgattagctagttggaggggtaacggcccaccaaggcgacgatcagtagccggtctgagaggatgaacggccacattggaactgagacacggtccagactcctacgggaggcagcagtggggaatcttccgcaatgggcgaaagcctgacggagcaacgccgcgtgagtgaagacggccttcgggttgtaaagctctgttatacgggacgaataatcttgtggttaatacccataagaagtgacggtaccgtaagagaaagccacggctaactacgtgccagcagccgcggtaatacgtaggtggcaagcgttgtccggaattattgggcgtaaagggcgcgcaggcggcttcttaagtctgtcttaaaagtgcggggcttaaccccgtgatgggatggaaactgggaagctcagagtatcggagaggaaagcggaattcctagtgtagcggtgaaatgcgtagatattaggaggaacaccagtggcgaaagcggcttttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacggccgcaaggttgaaactcaaaggaattgacgggggcccgcacaagcggtggagtatgtggtttaattcgacgcaacgcgaagaaccttaccaagccttgacattgatcgcaaggagtagagatactctgttcttcttcggaagacgagaaaacaggtggtgcacggctgtcgtcagctcgtgtcgtgagatgttgggttaagtcccgcaacgagcgcaacccctatcttctgttaccagcacgtaaaggtggggactcaggagagactgccgcagacaatgcggaggaaggcggggatgacgtcaagtcatcatgccccttatggcttgggctacacacgtactacaatggctcttaatagagggaagcgaaggagcgatccggagcaaaccccaaaaacagagtctcagttcggattgtaggctgcaactcgcctacatgaagcaggaatcgctagtaatcgcaggtcagcatactgcggtgaatacgttcccgggccttgtacacaccgcccgtcacaccacgaaagtcattcacacccgaagccggtgaggtaaccgcaaggagcca

SEQ ID NO: 37 - DNA, 1493 bp, Peptostreptococcus sp.
  source 1..1493: mol_type = genomic DNA
ttgtgcgtgcttacacacatgcaagtcgaacgtgatttttatggaaagcccttcggggatggaaattaaatgaaagtggcgaacgggtgagtaacacgtgagcaacctaccttacacagggggatagccgttggaaacgacgattaataccacatgagaccactaagacacatgttttggaggtaaaagatttatcggtgtaagatgggctcgcgtctgattagctagttggaagggtaaaggcctaccaaggcgacgatcagtagccggtctgagaggatgaacggccacattggaactgagacacggtccaaactcctacgggaggcagcagtggggaatattgcacaatggggggaaccctgatgcagcgacgccgcgtgagcgaagaaggttttcgaatcgtaaagctctgtcctatgagaagataatgacggtatcataggaggaagccccggctaaatacgtgccagcagccgcggtaatacgtatggggcgagcgttgtccggaattattgggcgtaaagggtacgtaggcggccttttaagtcaggtgtgaaagcgtgaggcttaacctcattaagcacttgaaactggaaggcttgagtgaaggagaggaaagtggaattcctagtgtagcggtgaaatgcgtagatattaggaggaataccggtggcgaaggcgactttctggacttttactgacgctcaggtacgaaagcgtggggagcaaacaggattagataccctggtagtccacgccgtaaacgatgaatgctaggtgttgggagtcaaatctcggtgccgaagttaacacattaagcattccgcctggggagtacggtggcaacactgaaactcaaaggaattgacggggacccgcacaagcagcggagcatgtggtttaattcgaagcaacgcgaagaaccttaccaaggcttgacatatagttgagttattgagaaattgataagtccctcgggacagctatacaggtggtgcatggttgtcgtcagctcgtgtcgtgagatgttgggttaagtcccgcaacgagcgcaacccttatcttcagttaccatcatgtaatgatggggactctggagagactgccgatgacaaatcggaggaaggtggggatgacgtcaaatcatcatgccctttatgtcttgggctacacacgtgctacaatggttggtacaacgagaagctagacagtgatgttaagcaaaactctaaaaaccaatctcagttcggattgtaggctgcaactcgcctacatgaagtcggagttgctagtaatcgcgaatcagaatgtcgcggtgaatgcgttcccgggtcttgtacacaccgcccgtcacaccatgggagttggcaatacccgaagccgccgatctaaccgcaaggaggaaggcgtcgaaggtagggttaatgactggggtgaagtcgtaacaaggtagccgtatcggaaggtgcggttggatcacctcctataaa

SEQ ID NO: 38 - DNA, 1529 bp, Bacteroides fragilis
  source 1..1529: mol_type = genomic DNA
acaatgaagagtttgatcctggctcaggatgaacgctagctacaggcttaacacatgcaagtcgaggggcatcaggaagaaagcttgctttctttgctggcgaccggcgcacgggtgagtaacacgtatccaacctgccctttactcggggatagcctttcgaaagaaagattaatacccgatagcataatgattccgcatggtttcattattaaaggattccggtaaaggatggggatgcgttccattaggttgttggtgaggtaacggctcaccaagccttcgatggataggggttctgagaggaaggtcccccacattggaactgagacacggtccaaactcctacgggaggcagcagtgaggaatattggtcaatgggcgctagcctgaaccagccaagtagcgtgaaggatgaaggctctatgggtcgtaaacttcttttatataagaataaagtgcagtatgtatactgttttgtatgtattatatgaataaggatcggctaactccgtgccagcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagtggaatgatgtggaaacatgtcagtgagcaatcaccgctgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctagcgggtgaccgtatgctaatcccaaaatcctctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacctgaagtacgtaaccgcaaggatcgtcctagggtaaaactggtgactggggctaagtcgtaacaaggtagccgtaccggaaggtgcggctggaacacctccttt

SEQ ID NO: 39 - DNA, 1573 bp, Lactobacillus gasseri
  source 1..1573: mol_type = genomic DNA
gaaaatgagagtttgatcctggctcaggacgaacgctggcggcgtgcctaatacatgcaagtcgagcgagcttgcctagatgaatttggtgcttgcaccagatgaaactagatacaagcgagcggcggacgggtgagtaacacgtgggtaacctgcccaagagactgggataacacctggaaacagatgctaataccggataacaacactagacgcatgtctagagtttaaaagatggttctgctatcactcttggatggacctgcggtgcattagctagttggtaaggtaacggcttaccaaggcaatgatgcatagccgagttgagagactgatcggccacattgggactgagacacggcccaaactcctacgggaggcagcagtagggaatcttccacaatggacgcaagtctgatggagcaacgccgcgtgagtgaagaagggtttcggctcgtaaagctctgttggtagtgaagaaagatagaggtagtaactggcctttatttgacggtaattacttagaaagtcacggctaactacgtgccagcagccgcggtaatacgtaggtggcaagcgttgtccggatttattgggcgtaaagcgagtgcaggcggttcaataagtctgatgtgaaagccttcggctcaaccggagaattgcatcagaaactgttgaacttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccagtgcaaacctaagagattaggtgttcccttcggggacgctgagacaggtggtgcatggctgtcgtcagctcgtgtcgtgagatgttgggttaagtcccgcaacgagcgcaacccttgtcattagttgccatcattaagttgggcactctaatgagactgccggtgacaaaccggaggaaggtggggatgacgtcaagtcatcatgccccttatgacctgggctacacacgtgctacaatggacggtacaacgagaagcgaacctgcgaaggcaagcggatctctgaaagccgttctcagttcggactgtaggctgcaactcgcctacacgaagctggaatcgctagtaatcgcggatcagcacgccgcggtgaatacgttcccgggccttgtacacaccgcccgtcacaccatgagagtctgtaacacccaaagccggtgggataacctttataggagtcagccgtctaaggtaggacagatgattagggtgaagtcgtaacaaggtagccgtaggagaacctgcggctggatcacctccttt

SEQ ID NO: 40 - DNA, 1515 bp, Rothia dentocariosa
  source 1..1515: mol_type = genomic DNA
agagtttgattctggctcaggacgaacgctggcggcgtgcttaacacatgcaagtcgaacgatgaagcctagcttgctaggtggattagtggcgaacgggtgagtaatacgtgagtgacctacctttgactctgggataagcctgggaaactgggtctaataccggatacgaccaatctccgcatggggtgttggtggaaagcgttatggagtggttttagatgggctcacggcctatcagcttgttggtgaggtaatggcttaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctgttagcatcgaagaagcgaaagtgacggtaggtgcagagaaagcgccggctaactacgtgccagcagccgcggtaatacgtagggcgcgagcgttgtccggaattattgggcgtaaagagcttgtaggcggttggtcgcgtctgctgtgaaaggctggggcttaaccctggttttgcagtgggtacgggctaactagagtgcagtaggggagactggaattcctggtgtagcggtggaatgcgcagatatcaggaggaacaccgatggcgaaggcaggtctctgggctgtaactgacgctgagaagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaagaaattgacgggggcccgcacaagcggcggagcatgcggattaattcgatgcaacgcgaagaaccttaccaaggcttgacatatactggactgcgtcagagatggcgtttcccttcggggctggtatacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggatgacgtcaaatcatcatgccccttatgtcttgggcttcacgcatgctacaatggccggtacagagggttgcgatactgtgaggtggagctaatccctaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaaccctggtggggggagccgtcgaaggtgggactggcgattgggactaagtcgtaacaaggtagccgtaccggaaggtgcggctggatcacct

SEQ ID NO: 41 - DNA, 1398 bp, Clostridium sporosphaeroides
  variation 830: note = a, c, t, g, unknown or other
  variation 834: note = a, c, t, g, unknown or other
  variation 860: note = a, c, t, g, unknown or other
  variation 920: note = a, c, t, g, unknown or other
  variation 924: note = a, c, t, g, unknown or other
  variation 1139: note = a, c, t, g, unknown or other
  variation 1167: note = a, c, t, g, unknown or other
  variation 1270..1272: note = a, c, t, g, unknown or other
  variation 1276: note = a, c, t, g, unknown or other
  variation 1291: note = a, c, t, g, unknown or other
  variation 1373: note = a, c, t, g, unknown or other
  variation 1378..1379: note = a, c, t, g, unknown or other
  source 1..1398: mol_type = genomic DNA
tgaacttagcttagtggcggacgggtgagtaacgcgtgagtawcctgcctttcrgagggggatamcgttcctgaaaacaacgctaataccgcataatatattggtgtcgcatggcaccggtatcaaaggagcaatccgctgcaagatggactcgcgtccgatyagccagtkgatkgggtaaaggcccaccaaagcgacgatcggtagccgggttgagagactgaacggccacattgggactgagacacggcccagactcctacgggaggcagcagtgggggatattgcacaatggaggaaactctgatgcagcaatgccgcgtgagggaagacggtcttcggattgtaaacctctgtccttggtgaagataatgacggtagccaaggaggaagctccggctaactacgtgccagcagccgcggtaatacgtagggagcaagcgttgtccggatttactgggtgtaaagggtgcgtaggcggctctttaagtcgggcgtgaaagctgtgggcttaacccacaaattgcgttcgaaactggagggcttgagtgaagtagaggtaggcggaattcccggtgtagcggtgaaatgcgtagagatcgggaggaacaccagtggcgaaggcggcctactgggctttaactgacgctgaggcacgaaagcatgggtagcaaacaggattagataccctggtagtccatgccgtaaacgatgattactaggtgtggggggtctgaccccttccgtgccggagttaacacaataagtaatccacctggggagtacggccgcaaggttgaaactcaaaggaattgacgggggcccgcacnagcngtggagtatgtggtttaattcgaagnaacgcgaagaaccttaccaggtgttgacatccaactaacgaggcagagatgcattaggtngctncggggaaagttgagacaggtggtccatggttgtcgtcagctcgtgtcgtgagatgttgggttaagtcccgcaacgagcgcaacccttgtgattagttstacgcaagagcactctaatcagactgccgttgacaaaacggaggaaggtggggacgacgtcaaatcatcatgccccttatgacctgggctacacacgtactacaatggtcgccaacngagggaagccaagccgcgaggtggagcnaacccccaaaagcgatctcagttcggattgtaggctgcaacccgcctacatgaagttggaattgctagtaatcgcggatcagcatgccgcggcgaatacgttnnngggncttgtacacaccgcncgtcacaccatgggagccggtaatacccgaagccaatagtctaaccgcaaggaggacgttgtcgaaggtaggattggcgacnggggnnaagtcgtaacaaggtaacc

SEQ ID NO: 42 - DNA, 1431 bp, Hungatella hathewayi
  variation 191: note = a, c, t, g, unknown or other
  variation 211: note = a, c, t, g, unknown or other
  variation 248: note = a, c, t, g, unknown or other
  variation 979: note = a, c, t, g, unknown or other
  source 1..1431: mol_type = genomic DNA
ctcaggatgaacgctggcggcgtgcttaacacatgcaagtcgagcgaagcggtttcaatgaagttttcggatggatttgaaattgacttagcggcggacgggtgagtaacgcgtgggtaacctgccttacactgggggataacagttagaaatgactgctaataccgcataagcgcacagggccgcatggnctggtgtgaaaaactccggnggtgtaagatggacccgcgtctgattaggtagttggnggggtaacggcccaccaagccgacgatcagtagccgacctgagagggtgaccggccacattgggactgagacacggcccaaactcctacgggaggcagcagtggggaatattggacaatgggcgaaagcctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtttagcaagtctgaagtgaaagcccggggctcaaccccggtactgctttggaaactgttagacttgagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgtcggggggcaaagcccttcggtgccgccgcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgaaaacacnttaaccgtgatccctcttcggagcagtggagacaggtggtgcatggttgtcgtcagctcgtgtcgtgagatgttgggttaagtcccgcaacgagcgcaacccttatccttagtagccagcgagtagagtcgggcactctggggagactgccagggataacctggaggaaggtggggatgacgtcaaatcatcatgccccttatgatttgggctacacacgtgctacaatggcgtaaacaaagggaggcaaaggagcgatctggagcaaaccccaaaaataacgtctcagttcggattgcaggctgcaactcgcctgcatgaagctggaatcgctagtaatcgcgaatcagaatgtcgcggtgaatacgttcccgggtcttgtacacaccgcccgtcacaccatgggagttggtaacgcccgaagtcagtgacccaaccgaaaggagggagct

SEQ ID NO: 43 - DNA, 1435 bp, Clostridium methylpentosum
  source 1..1435: mol_type = genomic DNA
aggacgaacgctggcggcgcgcctaacacatgcaagtcgaacggagttgttttggagaagcccttcggggtggaactgattcaacttagtggcggacgggtgagtaacacgtgagcaacctgccttacagaggggaataacgtttggaaacgaacgctaataccgcataacataacggaatcgcatggttttgttatcaaagattatatcgctgtaagatgggctcgcgtctgattagatagttggtgaggtaatggctcaccaagtcgacgatcagtagccggactgagaggttgaacggccacattgggactgagacacggcccagactcctacgggaggcagcagtggggaatattgcacaatgggggaaaccctgatgcagcgacgccgcgtgaaggaagaaggccttcgggttgtaaacttctgtcttcagggacgataatgacggtacctgaggaggaagctccggctaactacgtgccagcagccgcggtaatacgtagggagcgagcgttgtccggaattactgggtgtaaagggagcgtaggcgggattgcaagttgaatgtgaaatctatgggcttaacccataaactgcgttcaaaactgcagttcttgagtgaagtagaggcaggcggaattcctagtgtagcggtgaaatgcgtagatattaggaggaacaccagtggcgaaggcggcctgctgggctttaactgacgctgaggctcgaaagcgtgggtagcaaacaggattagataccctggtagtccacgccgtaaacgatgattactaggtgtaggggggtcaaccttctgtgccggagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccaactaacgaagtagagatacattaggtgcccttcggggaaagttgagacaggtggtgcatggttgtcgtcagctcgtgtcgtgagatgttgggttaagtcccgcaacgagcgcaacccttacatttagttgctacgcagagcactctagatggactgccgttgacaaaacggaggaaggtggggatgacgtcaaatcatcatgccccttatgacctgggctacacacgtactacaatggctattaacagaggaagcaaaacagtgatgtggagcaaacccctaaaaatagtctcagttcggattgtaggctgcaactcgcctacatgaagccggaattgctagtaatcgcggatcagcatgccgcggtgaatacgttcccgggccttgtacacaccgcccgtcacaccatgagagttggcaacacccgaagtcagtagtctaaccgcaaggaggacgctgccgaaggtgggg

SEQ ID NO: 44 - DNA, 1500 bp, Clostridium sp.
  source 1..1500: mol_type = genomic DNA
cttagattgatcatggctagggatgaacgctggcggcgtgcttaacacatgcaagtcgaacgaagcacttaagttgatttcttcggattgattcttgagtgactgagtggcggacgggtgagtaacgcgtgggtaacctgcctcatacagggggataacagtcagaaatggctgctaataccgcataagcgcacagtaccgcatggtacagcgtgaaaaactccggtggtatgagatggacccgcgtctgattagctggttggtggggtaacggcccaccaaggcgacgatcagtagccggcctgasagggtgaacggccacattgggactgagacacggcccaaactcctacgggaggcagcagtggggaatattgcacaatgggggaaaccctgatgcagtgacgccgcgtgasaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggactggcaagtctgatgtgaaaacccggggctcaaccccgggactgcattggaaactgtcagtctagagtgtcggaggggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgagtactaggtgtcggggagcaaagctcttcggtgccgcagcaaacgcaataagtattccacctggggagtacgttcgcaagaatgaaactcaaaggaattgacggggaccgcacaagcggtggagcatgtggtttaattcgaagcaacgcgaagarccttaccaagtcttgacatcgatctgaccggatagtaatgtatccttttcttcggggacagagaagacaggtggtgcatggttgtcgtcagctcgtgtcgtgagatgttgggttaagtcccgcaacgagcgcaacccttatccttagtagccagcatttcggatgggcactctggggagactgccagggataacctggaggaaggtggggatgacgtcaaatcatcatgccccttatgatttgggctacacacgtgctacaatggcgtaaacaaagggaagcagtcccgcgagggtgagcaaatcccaaaaataacgtctcagttcggattgtagtctgcaactcgactacatgaagctggaatcgctagtaatcgcaggtcagcatactgcggtgaatacgttcccgggtcttgtacacaccgcccgtcacaccatgggagtcggatatgcccgaagccagtgacccaactacgggagggagctgtcgaaggtggagccgataactggggtgaagtcgtaacaaggtaaccgtaaagggcg

SEQ ID NO: 45 - DNA, 1366 bp, Clostridium sp.
  source 1..1366: mol_type = genomic DNA
tgcagtcgagcgattctcttcggagaagagcggcggacgggtgagtaacgcgtgggtaacctgccctgtacacacggataacataccgaaaggtatgctaatacgggataatatgttttgatcgcatggtcgaaatatcaaagctccggcggtacaggatggacccgcgtctgattagctagttggtgaggtaatggctcaccaaggcgacgatcagtagccgacctgagagggtgatcggccacattggaactgagacacggtccaaactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggtggtttcttaagtcagaggtgaaaggctacggctcaaccgtagtaagcctttgaaactgagaaacttgagtgcaggagaggagagtagaattcctagtgtagcggtgaaatgcgtagatattaggaggaataccagttgcgaaggcggctctctggactgtaactgacactgaggcacgaaagcgtggggagcaaacaggattagataccctggtagtccacgccgtaaacgatgagtactagctgtcggaggttacccccttcggtggcgcagctaacgcattaagtactccgcctgggaagtacgctcgcaagagtgaaactcaaaggaattgacggggacccgcacaagtagcggagcatgtggtttaattcgaggcaacgcgaagaaccttacctaagcttgacatccttctgacctctctctaatcggagattacccttcggggacagaagtgacaggtggtgcatggttgtcgtcagctcgtgtcgtgagatgttgggttaagtcccgcaacgagcgcaacccttgcccttagttgccagcattaagttgggcactctagagggactgccagggataacctggaggaaggtggggatgacgtcaaatcatcatgccccttatgcttagggctacacacgtgctacaatgggtggtacagagggttgccaaaccgtgaggtggagctaatcccttaaagccattctcagttcggattgtaggctgaaactcgcctacatgaagctggagttactagtaatcgcagatcagaatgctgcggtgaatgcgttcctgggtcttgtacacaccgcccgtcacaccacggaagttgggggcgcccgaagccagatagctaaccttttgaagcgtc

SEQ ID NO: 46 - DNA, 1418 bp, Ruminococcus torques
  variation 724: note = a, c, t, g, unknown or other
  variation 771: note = a, c, t, g, unknown or other
  variation 776: note = a, c, t, g, unknown or other
  variation 807: note = a, c, t, g, unknown or other
  source 1..1418: mol_type = genomic DNA
ctcaggatgaacgctggcggcgtgcctaacacatgcaagtcgagcgaagcactttgcttagattcttcggatgaagaggattgtgactgagcggcggacgggtgagtaacgcgtgggtaacctgcctcatacagggggataacagttagaaatgactgctaataccgcataagaccacagcaccgcatggtgcgggggtaaaaactccggtggtatgagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggatgggcaagtctgatgtgaaaacccggggctcaaccccgggactgcattggaaactgttcatctagagtgctggagaggtaagtggaattcctagtgtagcggtgaaatgcgtagatattaggaggaacaccagtggcgaaggcggcttactggacagtaactgacgtngaggctcgaaagcgtggggagcacacaggattagataccctggtagnccacnccgtaaacgatgactactaggtgtcgggtgncaaagccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccgctgaccggacggtaatgcgtccttcccttcggggcagcggagacaggtggtgcatggttgtcgtcagctcgtgtcgtgagatgttgggttaagtcccgcaacgagcgcaacccctatctttagtagccagcggccaggccgggcactctagagagactgccggggataacccggaggaaggtggggatgacgtcaaatcatcatgccccttatgagcagggctacacacgtgctacaatggcgtaaacaaagggaagcgagaccgcgaggtggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgt

SEQ ID NO: 47 - DNA, 1475 bp, Clostridium sp.
  source 1..1475: mol_type = genomic DNA
ccttaacacatgcaagtcgaacgaagatatttagaatgagagcttcggcaggattttatcatcttagtggcggacgggtgagtaacgtgtgggcaacctgccctgtactggggaataatcattggaacgatgactaataccgcatgtggtcctcggaaggcatcttctgaggaagaaaggatttattcggtacaggatgggcccgcatctgattagctagttggtgagataacagcccaccaaggcgacgatcatagccgacctgagagggtgatcggccacattgggactgagacacggcccaaactcctacgggaggcagcagtggggaatattgcacaatgggcgaaagcctgatgcagcaacgccgcgtgaaggatgagggtttcggctcgtaaacttctatcaatagggaagaaagaaatgacggtacctaaataagaagccccggctaactacgtgccagcagccgcggtaatacgtagggggcaagcgttatccggaattatgggtgtaaagggagcgtaggcggcatggtaagccagatgtgaaagccttgggcttaacccgaggattgcatttggaactatcaagctagagtacaggatgaggaaagcggaattcctagtgtagcgtgaaatgcgtagatattaggaagaacaccagtggcgaaggcggctttctggactgaaactgacgctgaggctcgaaagcgtggggagcaaacaggattagataccctggtagtccacgccgtaaacgtgagtgctaggtgtcggggaggaatcctcggtgccgcagctaacgcaataagcactccacctggggagtacgaccgcaaggttgaaactcaaaggaattgacgggggcccgcacaagcggtggagcagtggtttaattcgaagcaacgcgaagaaccttaccaaggcttgacatcccgatgaccgctctagagatagagcttctcttcggagcatcggtgacaggtggtgcatggttgtcgtcagctcgtgcgtagatgttgggttaagtcccgcaacgagcgcaacccttatcttcagtagccatcattcagttgggcactctggagagactgccgtggataacacggaggaaggtggggatgacgtcaaatcatcatgcccttatgtctgggctacacacgtgctacaatggctggtaacaaagtgacgcaagacggcgacgttaagcaaatcacaaaaacccagtcccagttcggattgtagtctgcaactcgactacatgaagcggaatcgctagtaatcgcgaatcagcatgtcgcggtgaatacgttcccgggccttgtacacaccgcccgtcacaccatgggagttggaagcacccgaagtcggtgacctaaccgtaaggaaggagccccgaaggtgaagccagtgactggggtgaagtcgtaacaaggtagccgtatcggaaggtgcggctggtcccccctttta

SEQ ID NO: 48 - DNA, 1542 bp, Lactobacillus buchneri
  source 1..1542: mol_type = genomic DNA
tttgatcctggctcaggacgaacgctggcggcgtgcctaatacatgcaagtcgaacgcgtctccgttgatgattttaggtgcttgcacttgaaagatttaacattgagacgagtggcgaactggtgagtaacacgtgggtaacctgcccttgaagtaggggataacacttggaaacaggtgctaataccgtataacaaccaaaaccacctggttttggtttaaaagacggcttcggctgtcactttaggatggacccgcggcgtattagcttgttggtaaggtaacggcctaccaaggcgatgatacgtagccgacctgagagggtaatcggccacattgggactgagacacggcccaaactcctacgggaggcagcagtagggaatcttccacaatggacgaaagtctgatggagcaacgccgcgtgagtgatgaagggtttcggctcgtaaaactctgttgttggagaagaacaggtgtcagagtaactgttgacatcttgacggtatccaaccagaaagccacggctaactacgtgccagcagccgcggtaatacgtaggtggcaagcgttgtccggatttattgggcgtaaagcgagcgcaggcggttttttaggtctgatgtgaaagccttcggcttaaccggagaagtgcatcggaaaccgggagacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgatgctacgcgaagaaccttaccaggtcttgacatcttctgccaacctaagagattaggcgttcccttcggggacagaatgacaggtggtgcatggttgtcgtcagctcgtgtcgtgagatgttgggttaagtcccgcaacgagcgcaacccttattgttagttgccagcattcagttgggcactctagcaagactgccggtgacaaaccggaggaaggtggggatgacgtcaaatcatcatgccccttatgacctgggctacacacgtgctacaatggacggtacaacgagtcgcgaaaccgcgaggtcaagctaatctcttaaagccgttctcagttcggattgtaggctgcaactcgcctacatgaagttggaatcgctagtaatcgtggatcagcatgccacggtgaatacgttcccgggccttgtacacaccgcccgtcacaccatgagagtttgtaacacccaaagccggtgaggtaaccttcggggaccagccgtctaaggtgggacagatgattagggtgaagtcgtaacaaggtagccgtaggagaac

SEQ ID NO: 49 - DNA, 703 bp, Fusobacterium nucleatum
  variation 409: note = a, c, t, g, unknown or other
  variation 458: note = a, c, t, g, unknown or other
  variation 614: note = a, c, t, g, unknown or other
  source 1..703: mol_type = genomic DNA
cttgaatttgggtttttaacttagatttgggtggcggacgggtgagtaacgcgtaaagaacttgcctcacagctagggacaacatttagaaatgaatgctaatacctgatattatgattttaaggcatcttagaattatgaaagctataagcactgtgagagagctttgcgtcccattagctagttggagaggtaacagctcaccaaggcgatgatgggtagccggcctgagagggtgaacggccacaaggggactgagacacggcccttactcctacgggaggcagcagtggggaatattggacaatggaccgagagtctgatccagcaattctgtgtgcacgatgaagtttttcggaatgtaaagtgctttcagttgggaagaaagaaatgacggtaccaacagaanaagtgacggctaaatacgtgccagcagccgcggtaatacgtatgtcacnagcgttatccggatttattgggcgtaaagcgcgtctaggtggttatgtaagtctgatgtgaaaatgcagggctcaactctgtattgcgttggaaactgtataactagagtactggagaggtaagcggaactacaagtgtagaggtgaaattcgtanatatttgtaggaatgccgatggggaagccagcttactggacagatactgacgctaaagcgcgaaagcgtgggtagcaaacaggattaga

SEQ ID NO: 50 - DNA, 1497 bp, Eggerthella lenta
  source 1..1497: mol_type = genomic DNA
agagtttgatcctggctcaggatgaacgctggcggcgtgcctaacacatgcaagtcgaacgatgaaaccgccctcgggcggacatgaagtggcgaacgggtgagtaacacgtgaccaacctgccccttgctccgggacaaccttgggaaaccgaggctaataccggatactcctcgcccccctcctggggggcccgggaaagcccagacggcaagggatggggtcgcggcccattaggtagtaggcggggtaacggcccacctagcccgcgatgggtagccgggttgagagaccgaccggccacattgggactgagatacggcccagactcctacgggaggcagcagtggggaattttgcgcaatgggggaaaccctgacgcagcaacgccgcgtgcgggacgacggccttcgggttgtaaaccgctttcagcagggaagaaattcgacggtacctgcagaagaagctccggctaactacgtgccagcagccgcggtaatacgtagggagcgagcgttatccggattcattgggcgtaaagagcgcgtaggcggcctctcaagcgggatctctaatccgagggctcaacccccggccggatcccgaactgggaggctcgagttcggtagaggcaggcggaattcccggtgtagcggtggaatgcgcagatatcgggaagaacaccgatggcgaaggcagcctgctgggccgcaactgacgctgaggcgcgaaagctaggggagcgaacaggattagataccctggtagtcctagccgtaaacgatggatactaggtgtggggggctccgccctccgtgccgcagccaacgcattaagtatcccgcctggggagtacggccgcaaggctaaaactcaaaggaattgacgggggcccgcacaagcagcggagcatgtggcttaattcgaagcaacgcgaagaaccttaccagggcttgacatggacgtgaagccggggaaacccggtggccgagaggagcgtccgcaggtggtgcatggctgtcgtcagctcgtgtcgtgagatgttgggttaagtcccgcaacgagcgcaacccctgccccatgttgccagcattaggttggggactcatgggggactgccggcgtcaagccggaggaaggtggggacgacgtcaagtcatcatgccctttatgccctgggctgcacacgtgctacaatggccggtacaacgggctgcgagaccgcgaggtcgagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagccgccggccgaacccgcaaggggcggaggcgtcgaaggtgtggagggtaaggggggtgaagtcgtaacaaggtagccgtaccggaaggtgcggctggatcacct

SEQ ID NO: 51 - DNA, 1530 bp, Clostridium sp.
  source 1..1530: mol_type = genomic DNA
gagtttgatcatggctcaggatgaacgctggcggcgtgcttaacacgagtgcaaggtcgaacgaagcgcttaaggaccattcttcggaggcgtacttttgcggactgcagtggcggacgggtgagtaactgtgggtaactgccctatacagggggataacagttagaaatgactgctaataccgcataagcgcactagaaccgcatggttcggtgtgaaaaactgaggtggtataggatggacccgcgtctgattagcttgttggtggggtaacggctcaccaaggcgacgatcagtagccggcctgagagggcgaccggccacattgggactgagacacggcccaaactctacgggaggcagcagtggggaatattgcacaatgggggaaaccctgatgcagcgacgccgcgtgagtgaagaagtatttcggtatgttaaagctctatcagcagggaagaaaatgacggtacctgactaagatgccccggctaactacgtgccagcagccgcggtaatacgtagggggcaagcgttatccggatttactgggtgtaaaagggagcgtaggtggcatggtaagccagaagtgaaaacccagggctcaactctgtggattgcttttggaactatcaagctagagtgctggaggggtaagcggaattcctagtgtagcggtgaaatgcgtagatattaggaggaacaccggtggcgaaggcggcttactggacagaaactgacactgaggctcgaaagcgtggggagcaaacaggattagataccctggtagtccacgccgtaaacgatgaatactaggtgtcggggtacaaagtacctcggtgccgccgcaaacgcattaagtattccacctggggagtacgttcgcaagaatgaaactcaaaggaattgacggggaccgcacaagcggtggagcatgtggtttaattcgaagcaacgcgaagaaccttaccaagtcttgacatccctctgaataacaggtaatgctgttaggcttcgggacagaggagacaggtggtgcatggttgtcgtcagctcgtgtcgtgagatgttgggttaagtcccgcaacgagcgcaacccttgtccttagtagccatcaggtaaagctgggcactctatggagactgccggggataacccggaggaaggtggggatgacgtcaaatcatcatgccccttatgacttgggctacacacgtgctacaatggcgtaaacaaagggaagcaagcatgtgagtgtaagcaaatctcaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcggtaacgcccgaagcctgtgacccaaccttaagaggagggagcatgccgaaggttgggcccggtaactggggtgaagtcgtaacaaggtagccgtatcggaaggtgcggctggatcacctcct

SEQ ID NO: 52 - DNA, 1505 bp, Anaerostipes caccae
  variation 614: note = a, c, t, g, unknown or other
  source 1..1505: mol_type = genomic DNA
ttagagtttgatccatggctcaggatgaacgctggcggcgtgcttaacacatgcaagtcgaacgaagcatttaggattgaagttttcggatggatttcctatatgactgagtggcggacgggtgagtaacgcgtggggaacctgccctatacagggggataacagctggaaacggctgctaataccgcataagcgcacagaatcgcatgattcagtgtgaaaagccctggcagtataggatggtcccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaacagacggtacctgactaagaagccccggctaactacgtgccagcagccgcggtaatacgtagggggcaagcgttatccggaattactgggtgtaaagggtgcgtaggtggcatggtaagtcagaagtgaaagcccggggcttaacccngggactgcttttgaaactgtcatgctggagtgcaggagaggtaagcggaattcctagtgtagcggtgaaatgcgtagatattaggaggaacaccagtggcgaaggcggcttactggactgtcactgacactgatgcacgaaagcgtggggagcaaacaggattagataccctggtagtccacgccgtaaacgatgaatactaggtgtcggggccgtagaggcttcggtgccgcagcaaacgcagtaagtattccacctggggagtacgttcgcaagaatgaaactcaaaggaattgacggggacccgcacaagcggtggagcatgtggtttaattcgaagcaacgcgaagaaccttacctggtcttgacatcccaatgaccgaaccttaaccggttttttctttcgagacattggagacaggtggtgcatggttgtcgtcagctcgtgtcgtgagatgttgggttaagtcccgcaacgagcgcaacccctatctttagtagccagcatttgaggtgggcactctagagagactgccagggataacctggaggaaggtggggacgacgtcaaatcatcatgccccttatggccagggctacacacgtgctacaatggcgtaaacaaagggaagcgaagtcgtgaggcgaagcaaatcccagaaataacgtctcagttcggattgtagtctgcaactcgactacatgaagctggaatcgctagtaatcgtgaatcagaatgtcacggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcgg

SEQ ID NO: 53 - DNA, 1262 bp, Blautia faecis
  source 1..1262: mol_type = genomic DNA
ataacagccagaaatgactgctaataccgcataagcgcacagaaccgcatggttcggtgtgaaaaactccggtggtataagatggacccgcgttggattagctagttggcagggcagcggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtagacggcgcagcaagtctgatgtgaaaggcaggggcttaacccctggactgcattggaaactgctgtgcttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcagggagcacagctctttggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaaatcttgacatccctctgaccgggacttaaccgtccctttccttcgggacaggggagacaggtggtgcatggttgtcgtcagctcgtgtcgtgagatgttgggttaagtcccgcaacgagcgcaacccctatccttagtagccagcacgcartggtgggcactctgaggagactgccagggataacctggaggaaggcggggatgacgtcaaatcatcatgccccttatgatttgggctacacacgtgctacaatggcgtaaacaaagggaagcgaacccgcgagggtgggcaaatctcaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccg

SEQ ID NO: 54 - DNA, 1427 bp, Actinobaculum massiliense
  variation 18: note = a, c, t, g, unknown or other
  variation 82: note = a, c, t, g, unknown or other
  source 1..1427: mol_type = genomic DNA
cctttttttggttgggtnctcgagtggcgaacggtgagtattacgtgagtaacctgccctcaactgtgggataagcctgggnaactgggtctaatactgcatattcttctttcaccgcatggtgggggttggaaagttttttcggttggggatgggctcgcggcctatcagcttgttggtggggtgatggcctaccaaggcggtgacgggtagccggcctgagagggtggtcggccacattgggactgagatacggcccagactcctacgggaggcagcagtgggggatattgcacaatgggcgcaagcctgatgcagcgacgccgcgtgagggatgaaggctttcgggttgtaaacctctttcggtacagaacaaggcacgttttttgcgtgtggagggtatgtgcagaagaagcgccggctaactacgtgccagcagccgcggtaatacgtagggcgcgagcgttgtccggaattattgggcgtaaagggctcgtaggcggcttgttgcgcctgctgtgaaaacgcggggcttaactccgcgcgtgcagtgggtacgggcaggcttgagtgtggtaggggtgactggaattccaggtgtagcggtggaatgcgcagatatctggaggaacaccgatggcgaaggcaggtcactgggccattactgacgctgaggagcgaaagcgtgggtagcgaacaggattagataccctggtagtccacgctgtaaacgttgggaactaggtgtggggcccttttccggggttctgcgccgtagctaacgcattaagttccccgcctggggagtacggccgcaaggctaaaactcaaaggaattgacgggggcccgcacaagcggcggagcatgcggattaattcgatgcaacgcgaagaaccttaccaaggcttgacatacaccgggagcatgcagagatgtgtgtgcctttttggctggtgtacaggtggtgcatggttgtcgtcagctcgtgtcgtgagatgttgggttaagtcccgcaacgagcgcaacccttgtctcgtgttgccagcgcgttatggtggggactcacgagagactgccggggttaactcggaggaaggtggggatgacgtcaaatcatcatgccccttatgtcttgggcttcacgcatgctacaatggccggtacagtgggttgcgatactgtgaggtggagcgaatctctgaaagccggtctcagttcggatcggggtctgcaactcgacctcgtgaagtcggagtcgctagtaatcgcggatcagcattgccgcggtgaatacgttcccgggccttgtacacaccgcccgtcacgtcacgaaagttggcaacacccgaagcccgtggcctaaccttttggggggagcggtcgaaggtggggttggcgattgggacgaagtcgtaacaaggtagcc

SEQ ID NO: 55 - DNA, 1484 bp, Enterococcus gallinarum
  source 1..1484: mol_type = genomic DNA
acgaacgctggcggcgtgcctaatacatgcaagtcgaacgctttttctttcaccggagcttgctccaccgaaagaaaaagagtggcgaacgggtgagtaacacgtgggtaacctgcccatcagaaggggataacacttggaaacaggtgctaataccgtataacactattttccgcatggaagaaagttgaaaggcgcttttgcgtcactgatggatggacccgcggtgcattagctagttggtgaggtaacggctcaccaaggccacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggatgagagtaaaacgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcgaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaacctttttggagccagccgcctaaggtgggatagatgattggggtgaag

SEQ ID NO: 56 - DNA, 1417 bp, Clostridium sp.
  source 1..1417: mol_type = genomic DNA
tcaggacgaacgctggcggcatgcctaacacatgcaagtcgaacggagtaagagagaagcttgcttagctcttacttagtggcggacgggtgagtaacgcgtgagtaacctgcctttcagagggggataacgttctgaaaagaacgctaataccgcatgatatattggggtcgcatggccttggtatcaaaggagcaatccgctgaaagatggactcgcgtccgattagccagttggcggggtaaaggcccaccaaagcgacgatcggtagccgggttgagagactgaacggccacattgggactgagacacggcccagactcctacgggaggcagcagtgggggatattgcacaatggaggaaactctgatgcagcaatgccgcgtgagggaagacggtcttcggattgtaaacctctgtccttggtgaagataatgacggtagccaaggaggaagctccggctaactacgtgccagcagccgcggtaatacgtagggagcaagcgttgtccggatttactgggtgtaaagggtgcgtaggcggctttttaagtcgggcgtgaaagctgtgggcttaacccacaaattgcgttcgaaactggaaggcttgagtgaagtagaggtaggcggaattcccggtgtagcggtgaaatgcgtagagatcgggaggaacaccagtggcgaaggcggcctactgggctttaactgacgctgaggcacgaaagcatgggtagcaaacaggattagataccctggtagtccatgccgtaaacgatgattactaggtgtggggggtctgaccccttccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccaactaacgaggcagagatgcattaggtgcccttcggggaaagttgagacaggtggtgcatggttgtcgtcagctcgtgtcgtgagatgttgggttaagtcccgcaacgagcgcaacccttgtgattagttgctacgcaagagcactctaatcagactgccgttgacaaaacggaggaaggtggggacgacgtcaaatcatcatgccccttatgacctgggctacacacgtactacaatggtcgctaacagagggaagccaagccgcgaggtggagcaaacccccaaaagcgatctcagttcggattgtaggctgcaacccgcctacatgaagttggaattgctagtaatcgcggatcagcatgccgcgggtgaatacgttcccgggccttgtacacaccgcccgtcacaccatgggagccggtaatacccgaagccaatagtctaaccgcaagggggacgt

SEQ ID NO: 57 - DNA, 332 bp, Leuconostoc sp.
  source 1..332: mol_type = genomic DNA
agcgcagacggttgattaagtctgatgtgaaagcccggagctcaactccggaaaggcattggaaactggtcaacttgagtgcagtagaggtaagtggaactccatgtgtagcggtggaatgcgtagatatatggaagaacaccagcggcgaaggcggcttactggactgtaactgacgttgaggctcgaaagtgtgggtagcaaacaggattagataccctggtagtccacaccgtaaacgatgaacactaggtgttaggaggtttccgcctcttagtgccgaagctaacgcattaagtgttccgcctggggagtacgaccgcaaggttgaa

SEQ ID NO: 58 - DNA, 1445 bp, Atopobium rimae
  source 1..1445: mol_type = genomic DNA
gatgaacgctggcggcgcgcctaacacatgcaagtcgaacggttaaagcaccttttaggtgtgtataaagtggcgaacggctgagtaacacgtgggcaacctgccctcctcttggggatagcctcgggaaaccgaggataatacccgatacttcaatgtggccgcatgacgacattgagaaagcttttgcggagaaggatgggcccgcggcctgttagcttgttggtggggtagaggcctaccaaggcaatgatgggtagctgggttgagagaccgaccagccagattgggactgagacacggcccagactcctacgggaggcagcagtggggaatcttgcacaatgggcgaaagcctgatgcagcgacgccgcgtgcgggacgaaggccttcgggttgtaaaccgctttcagcagggacgaggcgaaagtgacggtacctgcagaagaagccccggctaactacgtgccagcagccgcggtaatacgtagggggcaagcgttatccggattcattgggcgtaaagcgctcgtaggcggtctgttaggtcgggagttaaatccggaggctcaacctccgcccgctcccgataccggcagacttgagtttggtaggggaaggtggaattcctagtgtagcggtggaatgcgcagatattaggaagaacaccagtggcgaaggcggccttctgggccataactgacgctgaggagcgaaagctaggggagcaaacaggattagataccctggtagtcctagctgtaaacgatggacactaggtgtgggagaatacgtcttccgtgccgcagctaacgcattaagtgtcccgcctggggagtacggccgcaaggctaaaactcaaaggaattgacggggacccgcacaagcagcggagcatgtggtttaattcgaagcaacgcgaagaaccttaccagggcttgacatgtaggtgaagcggcggaaacgtcgtggccgaaaggagcctacacaggtggtgcatggctgtcgtcagctcgtgtcgtgagatgttaggttaagtcctgcaacgagcgcaaccctcgtcgtatattgccagcggttcggccgggcacctatacgagaccgccggcgtcaagccggaggaaggtggggacgacgtcaagtcatcatgccccttatgtcctgggctacacacgtgctacaatggccggcacaatgagctgccaacccgcaagggcgagcgaatctctaaagccggtcccagttcggattggaggctgcaacccgcctccatgaagtcggagttgctagtaatcgcggatcagcacgccgcggtgaatgcgttcccgggtcttgtacacaccgcccgtcacaccacccgagtcgattgcacccgaagtcgtcggcctaaccttttaggagggagacgccgaaggtgtggttggtaaggggggtgaagtcgtaac

SEQ ID NO: 59 - DNA, 1308 bp, Blautia luti
  variation 702..705: note = a, c, t, g, unknown or other
  source 1..1308: mol_type = genomic DNA
gtgggtaacctgccttatacagggggataacagtcagaaatgactgctaataccgcataagcgcacagagctgcatggctccggtgtgaaaaactccggtggtataagatggacccgcgttggattagctagttggtgaggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcatggacaagtctgatgtgaaaggctggggctcaaccccgggactgcattggaaactgcccgtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgcggtaaacgatgaatcctaggtgtcggggagcaaannnnttcggtgccgccgcaaacgcattaagcattccacctggggagtacgttcgcaagaatgaaactcaaaggaattgacggggacccgcacaagcggtggagcatgtggtttaattcgaagcaacgcgaagaaccttaccaagtcttgacatccctctgaccgagtatgtatggtacttttccttcgggagagagaggagacaggtggtgcatggttgtcgtcagctcgtgtcgtgagatgttgggttaagtcccgcaacgagcgcaacccctatccccagtagccagcggttcggccgggcactctgaggagactgccagggataacctggaggaaggcggggatgacgtcaaatcatcatgccccttatgatttgggctacacacgtgctacaatggcgtaaacaaagggaagcaagcctgcgagggtgggcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaact

SEQ ID NO: 60 - DNA, 1506 bp, Pseudopropionibacterium propionicum
  source 1..1506: mol_type = genomic DNA
tcaggacgaacgctggcggcgtgcttaacacatgcaagtcgaacggaaaggcccctttgggggtactcgagtggcgaacgggtgagtaacacgtgagtaacctgcccttgacttcgggataacttcaggaaactggggctaataccggatagacatccttgctgcatggtgggggttggaaagcttcggcggttttggatggactcgcggcttatcagcttgttggtggggtagtggcttaccaaggctttgacgggtagccggcctgagagggcgaccggccacattgggactgagatacggcccagactcctacgggaggcagcagtggggaatattgcacaatgggcggaagcctgatgcagcaacgccgcgtgcgggatgacggcttcgggttgtaaacccgctttcagcaggggcgaagcttttgtgacggtacctgcagaagaagcaccggctaactacgtgccagcagccgcggtgatacgtagggtgcgagcgttgtccggatttattgggcgtaaagagctcgtaggtggttgattgcgtcggaagtgaaaacttggggcttaaccctgagcgtgctttcgatacgggttgacttgaggaaggtaggggagaatggaattcctggtggagcggtggaatgcgcagatatcaggaggaacaccagtggcgaaggcggttctctggacctttcctgacgctgaggagcgaaagcgtggggagcgaacaggcttagataccctgggtagtccacgctgtaaacggtgggtactaggtgtggggtccattccacggattccgtgccgtagctaacgcattaagtaccccgcctggggagtacggccgcaaggctaaaactcaaaggaattgacggggccccgcacaagcggcggagcatgcggattaattcgatgcaacgcgaagaaccttacctgggtttgacatggactgggagtgctcagagatgggtatgcctccttgtggggctggttcacaggtggtgcatggctgtcgtcagctcgtgtcgtgagatgttgggttaagtcccgcaacgagcgcaaccctcgttcactgttgccagcacgttatggtggggactcagtggagaccgccggggtcaactcggaggaaggtggggatgacgtcaagtcatcatgccccttatgtccagggcttcacgcatgctacaatggccggtacaaagagttgcgagcctgtgagggtgagcgaatctcggaaagccggtctcagttcggattggggtctgcaactcgaccctatgaagtcggagtcgctagtaatcgcagatcagcaacgctgcggtgaatacgttcccggggcttgtacacaccgcccgtcaagtcatgaaagtcggtaacacccgaagccggtggcctaacctgtgtgggggagccgtcgaaggtgggactggtaattaggactaagtcgtaacaaggtagccgtaccggaaggtgcggctggatcacctcctttctaagg

SEQ ID NO: 61 - DNA, 1473 bp, Streptococcus lutetiensis
  source 1..1473: mol_type = genomic DNA
tgaacgctggcggcgtgcctaatacatgcaagtagaacgctgaagactttagcttgctaaagttggaagagttgcgaacgggtgagtaacgcgtaggtaacctgcctactagcgggggataactattggaaacgatagctaataccgcataacagcatttaacccatgttagatgcttgaaaggagcaattgcttcactagtagatggacctgcgttgtattagctagttggtgaggtaacggctcaccaaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagagaagaacgtgtgtgagagtggaaagttcacacagtgacggtaacttaccagaaagggacggctaactacgtgccagcagccgcggtaatacgtaggtcccgagcgttgtccggatttattgggcgtaaagcgagcgcaggcggtttaataagtctgaagttaaaggcagtggcttaaccattgttcgctttggaaactgttagacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttaggccctttccggggcttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctattcctagagataggaagtttcttcggaacatcggtgacaggtggtgcatggttgtcgtcagctcgtgtcgtgagatgttgggttaagtcccgcaacgagcgcaacccctattgttagttgccatcattaagttgggcactctagcgagactgccggtaataaaccggaggaaggtggggatgacgtcaaatcatcatgccccttatgacctgggctacacacgtgctacaatggttggtacaacgagtcgcgagtcggtgacggcaagcaaatctcttaaagccaatctcagttcggattgtaggctgcaactcgcctacatgaagtcggaatcgctagtaatcgcggatcagcacgccgcggtgaatacgttcccggccttgtacacaccgcccgtcacaccacgagagtttgtaacacccgaagtcggtgaggtaaccttttaggagccagccgcctaaggtgggatagatgattggggtgaagtc

SEQ ID NO: 62 - DNA, 1532 bp, Scardovia inopinata
  source 1..1532: mol_type = genomic DNA
tttcgattctggctcaggatgaacgctggcggcgtgcttaacacatgcaagtcgaacgggatcctgcttggtttttgctgggtgggtgagagtggcgaacgggtgagtaatgcgtgactaacctgccgtgtagttgggtatagctcctggaaacgggtggtaataccgaatggtccactgggctgcatggtctggtgggaaagttttgtgtgctatgcgatggggtcgcgtcctatcagcttgttggtggggtgatggcctaccaaggcttcgacgggtagccggcctaagagggcgaccggccacattgggactgagatacggcccagactcctacgggaggcagcagtggggaatattgcacaatggggggaaccctgatgcagcgacgccgcgtgcgggatggaggccttcgggttgtgaaccgcttttattggggagcaagctggccctgtgtgggttggtgagtgtacttggtgaataagcaccggctaactacgtgccagcagccgcggtaatacgtagggtgcaagcgttgtccggatttattgggcgtaaagggctcgtaggcggttcgtcgcgtctggtgtgaaagcttactgcttaacggtgggtttgcgttggatacgggcgggcttgagtgcagtaggggagactggaattctcggtgtaacggtggaatgtgtagatatcgggaagaacacctatggcgaaggcaggtctctgggctgttactgacgctgaggagcgaaagcgtggggagcgaacaggattagataccctggtagtccacgctgtaaacggtggacgctggatgtggggcctatttccacgggttctgtgtcggagctaacgcgttaagcgtcccgcctggggagtacggccgcaaggttaaaactcaaagaaattgacgggggcccgcacaagcggcggagcatgcggattaattcgatgcaacgcgaagaaccttaccagggcttgacatgtgagcgttgatcttagagatagggtttcccttcggggcgctttcacaggtggtgcatggtcgtcgtcagctcgtgtcgtgagatgttgggttaagtcccgcaacgagcgcaacccttgccttgtgttgccagcgggtcgtgccggggactcacaagggaccgccggggttaactcggaggaaggtggggatgacgtcagatcatcatgccccttacgtcctgggcttcacgcatgctacaatggctggtacagcgggatgcgatacggtgacgtggagcggatcctgtaaaaccggtctcagttcggatcggagcctgcaactcggctccgtgaaggtggagtcgctagtaatcgcggatcagcagtgccgcggtgaatgcgttcccgggccttgtacacaccgcccgtcaagtcatgaaagtgggcagcacccgaagccggtggcctaacccttgtggggagagccgtctaaggtgaggttcgcgattgggactaagtcgtaacaaggtagccgtaccggaaggtgcggctggatcacctcctta

SEQ ID NO: 63 - DNA, 1479 bp, Dorea formicigenerans
  variation 123..125: note = a, c, t, g, unknown or other
  variation 220..222: note = a, c, t, g, unknown or other
  variation 287: note = a, c, t, g, unknown or other
  variation 349..350: note = a, c, t, g, unknown or other
  variation 517: note = a, c, t, g, unknown or other
  variation 534..535: note = a, c, t, g, unknown or other
  variation 725: note = a, c, t, g, unknown or other
  variation 918: note = a, c, t, g, unknown or other
  variation 921: note = a, c, t, g, unknown or other
  variation 955..956: note = a, c, t, g, unknown or other
  variation 1184..1185: note = a, c, t, g, unknown or other
  source 1..1479: mol_type = genomic DNA
ttaaacgagagtttgatcctggctcaggatgaacgctggcggcgtgcttaacacatgcaagtcgagcgaagcacataagtttgattcttcggatgaagacttttgtgactgagcggcggacgnnngagtaacgcgtgggtaacctgcctcatacagggggataacagytagaaatggctgctaataccgcataagaccacagtactgcatggtacagtgnnnaaaactccggtggtatgagatggacccgcgtctgattaggtagttggtgaggtaacggcccaccnagccgacgatcagtagccgacctgagagggtgaccggccacattgggactgagacacggccnngactcctacgggaggcagcagtggggaatattgcacaatgggcgaaagcctgatgcagcgacgccgcgtgaaggatgaagtatttcggtatgtaaacttctatcagcagggaagaaaatgacggtacctgactaagaagccccggctaactacgtgccagcagccgnggtaatacgtagggggnnagcgttatccggatttactgggtgtaaagggagcgtagacggctgtgcaagtctgaagtgaaaggcatgggctcaacctgtggactgctttggaaactgtgcagctagagtgtcggagaggtaagtggaattcctagtgtagcggtgaaatgcgtagatattaggaggaacaccagtggcgaaggcggcntactggacgatgactgacgttgaggctcgaaagcgtggggagcaaacaggattagataccctggtagtccacgccgtaaacgatgactgctaggtgtcgggtagcaaagctattcggtgccgcagctaacgcaataagcagtccacctggggagtacgttcgcaagaatgaaactcaaaggaattgacggggnccngcacaagcggtggagcatgtggtttaattcgaannaacgcgaagaaccttacctgatcttgacatcccgatgaccgcttcgtaatggaagyttttcttcggaacatcggtgacaggtggtgcatggttgtcgtcagctcgtgtcgtgagatgttgggttaagtcccgcaacgagcgcaacccttatcttcagtagccagcatttaggatgggcactctggagagactgccagggataacctggaggaaggtggggatgacgtnnaatcatcatgccccttatgaccagggctacacacgtgctacaatggcgtaaacagagggaggcagagccgcgaggccgagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgaaaggagggagctgccgaaggtgggaccgataactggggt

SEQ ID NO: 64 - DNA, 1430 bp, Corynebacterium pseudogenitalium
  variation 1341: note = a, c, t, g, unknown or other
  source 1..1430: mol_type = genomic DNA
ggctcaggatgaacgctggcggcgtgcttaacacatgcaagtcgaacggaaaggccagtgcttgcactggtactcgagtggcgaacgggtgagtaacacgtgggtgatctgccctgtacttcgggataagcttgggaaactgggtctaataccggataggacaactttttggatattgttgtggaaagcttttgcggtatgggatgagctcgcggcctatcagcttgttggtggggtaatggcctaccaaggcgtcgacgggtagccggcctgagagggtgtacggccacattgggactgagatacggcccagactcctacgggaggcagcagtggggaatattgcacaatgggcgcaagcctgatgcagcgacgccgcgtgggggatgaaggccttcgggttgtaaactcctttcgctagggacgaagctttttgtgacggtacctggagaagaagcaccggctaactacgtgccagcagccgcggtaatacgtagggtgcgagcgttgtccggaattactgggcgtaaagagctcgtaggtggtttgtcgcgtcgtttgtgtaagtccacagcttaactgtgggactgcaggcgatacgggcataacttgagtgctgtaggggagactggaattcctggtgtagcggtgaaatgcgcagatatcaggaggaacaccgatggcgaaggcaggtctctgggcagtaactgacgctgaggagcgaaagcatgggtagcgaacaggattagataccctggtagtccatgccgtaaacggtgggcgctaggtgtgagtcccttccacgggatttgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggcttgacatgtagcggatcgccgcagagatgtggtttcccttgtggtcgctatacaggtggtgcatggttgtcgtcagctcgtgtcgtgagatgttgggttaagtcccgcaacgagcgcaacccttgtcttatgttgccagcatgtaatggtggggactcatgagagactgccggggttaactcggaggaaggtggggatgacgtcaaatcatcatgccccttatgtccagggcttcacacatgctacaatggtcggtacagcgcgtttgctaccttgtgaggggatgctaatcgcacaaagccggccttagttcggattggggtctgcaactcgaccccatgaagtcggagtcgctagtaatcgcagatcagcaacgctgcggtgaatacgtncccagccagtggcctgtcatgggagctgtcgaaggtgggatcggcgattgggacgaagtcgtaacaaggtagccgtaccggaaggtgcg

SEQ ID NO: 65 - DNA, 1508 bp, Turicibacter sanguinis
  source 1..1508: mol_type = genomic DNA
agagtttgatcatggctcaggatgaacgctggcggcgtgcctaatacatgcaagtcgagcgaaccacttcggtggtgagcggcgaacgggtgagtaacacgtaggttatctgcccatcagacggggacaacgattggaaacgatcgctaataccggataggacgaaagtttaaaggtgcttcggcaccactgatggatgagcctgcggcgcattagctagttggtagggtaaaggcctaccaaggcgacgatgcgtagccgacctgagagggtgaacggccacactgggactgagacacggcccagactcctacgggaggcagcagtagggaatcttcggcaatgggcgaaagcctgaccgagcaacgccgcgtgaatgatgaaggccttcgggttgtaaaattctgttataagggaagaatggctctagtaggaaatggctagagtgtgacggtaccttatgagaaagccacggctaactacgtgccagcagccgcggtaatacgtaggtggcgagcgttatccggaattattgggcgtaaagagcgcgcaggtggttgattaagtctgatgtgaaagcccacggcttaaccgtggagggtcattggaaactggtcaacttgagtgcagaagagggaagtggaattccatgtgtagcggtgaaatgcgtagagatatggaggaacaccagtggcgaaggcggcttcctggtctgtaactgacactgaggcgcgaaagcgtggggagcaaacaggattagataccctggtagtccacgccgtaaacgatgagtgctaagtgttgggggtcgaacctcagtgctgaagttaacgcattaagcactccgcctggggagtacggtcgcaagactgaaactcaaaggaattgacggggacccgcacaagcggtggagcatgtggtttaattcgaagcaacgcgaagaaccttaccaggtcttgacataccagtgaccgtcctagagataggattttcccttcggggacaatggatacaggtggtgcatggttgtcgtcagctcgtgtcgtgagatgttgggttaagtcccgcaacgagcgcaacccctgtcgttagttgccagcattcagttggggactctaacgagactgccagtgacaaactggaggaaggtggggatgacgtcaaatcatcatgccccttatgacctgggctacacacgtgctacaatggttggtacaaagagaagcgaagcggtgacgtggagcaaacctcataaagccaatctcagttcggattgtaggctgcaactcgcctacatgaagttggaatcgctagtaatcgcgaatcagcatgtcgcggtgaatacgttcccgggtcttgtacacaccgcccgtcacaccacgagagtttacaacacccgaagtcagtggcctaaccgcaaggagggagctgcctaaggtggggtagatgattggggtgaagtcgtaacaaggtatccctaccggaaggtggggttggatcacctcctt

SEQ ID NO: 66 - DNA, 1456 bp, Roseburia faecis
  source 1..1456: mol_type = genomic DNA
gatgaacgctggcggcgtgcttaacacatgcaagtcgaacgaagcactctatttgattttcttcggaaatgaagattttgtgactgagtggcggacgggtgagtaacgcgtgggtaacctgcctcatacagggggataacagttggaaacgactgctaataccgcataagcgcacaggatcgcatgatccggtgtgaaaaactccggtggtatgagatggacccgcgtctgattagccagttggcagggtaacggcctaccaaagcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagtggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgtcggggagcattgctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccgatgacagagtatgtaatgtacyttctcttcggagcatcggtgacaggtggtgcatggttgtcgtcagctcgtgtcgtgagatgttgggttaagtcccgcaacgagcgcaacccctgtccttagtagccagcggttcggccgggcactctagggagactgccagggataacctggaggaaggcggggatgacgtcaaatcatcatgccccttatgacttgggctacacacgtgctacaatggcgtaaacaaagggaagcggagccgtgaggccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttggaaatgcccgaagtcagtgacccaaccgcaaggagggagctgccgaaggcaggttcgataactggggtg

SEQ ID NO: 67 - DNA, 1467 bp, Clostridium paraputrificum
  variation 982..983: note = a, c, t, g, unknown or other
  source 1..1467: mol_type = genomic DNA
gatcctggctcaggatgaacgctggcggcgtgcctaacacatgcaagtcgagcgatgaagttccttcgggaacggattagcggcggacgggtgagtaacacgtgggcaacctgccttatagaggggaatagccttccgaaaggaagattaataccgcataagattgtagcttcgcatgaagtagcaattaaaggagcaatccgctataagatgggcccgcggcgcattagctagttggtaaggtaacggctcaccaaggcgacgatgcgtagccgacctgagagggtgatcggccacattgggactgagacacggcccagactcctacgggaggcagcagtggggaatattgcacaatgggggaaaccctgatgcagcaacgccgcgtgagtgatgacggcttcgggttgtaaagctctgtctttggggacgataatgacggtacccaaggaggaagccacggctaactacgtgccagcagccgcggtaatacgtaggtggcaagcgttgtccggatttactgggcgtaaagggagcgtaggcggatttttaagtgggatgtgaaatacccgggctcaacctgggtgctgcattccaaactggaaatctagagtgcaggaggggaaagtggaattcctagtgtagcggtgaaatgcgtagagattaggaagaacaccagtggcgaaggcgactttctggactgtaactgacgctgaggctcgaaagcgtggggagcaaacaggattagataccctggtagtccacgccgtaaacgatgaatactaggtgtaggggttgtcatgacctctgtgccgccgctaacgcattaagtattccgcctggggagtacggtcgcaagattaaaactcaaaggaattgacgggggcccgcacaagtagcggagcatgtggtttaattcgaagcaacgcgaagaaccttacctagacttgacatctcctgaattaccatgtaatgtggaagnnccttcgggaacaggaagacaggtggtgcatggttgtcgtcagctcgtgtcgtgagatgttgggttaagtcccgcaacgagcgcaacccttattgttagttgctaccatttagttgagcactctagcgagactgccgggttaaccgggaggaaggtggggatgacgtcaaatcatcatgccccttatgtctagggctacacacgtgctacaatggccggtacaacgagatgcaataccgtgaggtggagcaaaactataaaaccggtctcagttcggattgtaggctgaaactcgcctacatgaagctggagttactagtaatcgcgaatcagaatgtcgcggtgaatacgttcccgggccttgtacacaccgcccgtcacaccatgagagttggcaatacccaaagttggtgatctaacccgtaagggaggaagccacctaaggtagggtcagcgattggggtgaagtcgtaacaaggtatccg

SEQ ID NO: 68 - DNA, 1478 bp, Clostridium symbiosum
  variation 349..350: note = a, c, t, g, unknown or other
  variation 534..535: note = a, c, t, g, unknown or other
  variation 606: note = a, c, t, g, unknown or other
  variation 618..619: note = a, c, t, g, unknown or other
  variation 709: note = a, c, t, g, unknown or other
  variation 921: note = a, c, t, g, unknown or other
  variation 955..956: note = a, c, t, g, unknown or other
  variation 1012..1013: note = a, c, t, g, unknown or other
  variation 1015: note = a, c, t, g, unknown or other
  variation 1081: note = a, c, t, g, unknown or other
  variation 1185: note = a, c, t, g, unknown or other
  variation 1240: note = a, c, t, g, unknown or other
  variation 1391..1392: note = a, c, t, g, unknown or other
  variation 1470: note = a, c, t, g, unknown or other
  variation 1474..1475: note = a, c, t, g, unknown or other
  source 1..1478: mol_type = genomic DNA
aaacatgagagtttgatcctggctcaggatgaacgctggcggcgtgcctaacacatgcaagtcgaacgaagcgatttaacggaagttttcggatggaagttgaattgactgagtggcggacgggtgagtaacgcgtgggtaacctgccttgtactgggggacaacagttagaaatgactgctaataccgcataagcgcacagtattgcatgatacagtgtgaaaaactccggtggtacaagatggacccgcgtctgattagctagttggtaaggtaacggcttaccaaggcgacgatcagtagccgacctgagagggtgaccggccacattgggactgagacacggccnnaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggnnagcgttatccggatttactgggtgtaaagggagcgtagacggtaaagcaagtctgaagtgaaagcccgcgnctcaactgcggnnctgctttggaaactgtttaactggagtgtcggagaggtaagtggaattcctagtgtagcggtgaaatgcgtagatattaggaggaacacnagtggcgaaggcgacttactggacgataactgacgttgaggctcgaaagcgtggggagcaaacaggattagataccctggtagtccacgccgtaaacgatgaatactaggtgttggggagcaaagctcttcggtgccgtcgcaaacgcagtaagtattccacctggggagtacgttcgcaagaatgaaactcaaaggaattgacggggaccngcacaagcggtggagcatgtggtttaattcgaannaacgcgaagaaccttaccaggtcttgacatcgactcgacgggggagtaacgtcccnntnccttcggggcggagaagacaggtggtgcatggttgtcgtcagctcgtgtcgtgagatgttgggttnagtcccgcaacgagcgcaacccttattctaagtagccagcggttcggccgggaactcttgggagactgccagggataacctggaggaaggtggggatgacgtcnaatcatcatgccccttatgatctgggctacacacgtgctacaatggcgtaaacanagagaagcaagaccgcgaggtggagcaaatctcaaaaataacgtctcagttcggactgcaggctgcaactcgcctgcacgaagctggaatcgctagtaatcgcgaatcagaatgtcgcggtgaatacgttcccgggtcttgtacacaccgnncgtcacaccatgggagtcagtaacgcccgaagtcagtgacccaaccgcaaggagggagctgccgaaggcgggaccganaacnnggg

SEQ ID NO: 69 - DNA, 1462 bp, Propionibacterium freudenreichii
  variation 17: note = a, c, t, g, unknown or other
  variation 41: note = a, c, t, g, unknown or other
  variation 74: note = a, c, t, g, unknown or other
  variation 83..86: note = a, c, t, g, unknown or other
  variation 93: note = a, c, t, g, unknown or other
  variation 187: note = a, c, t, g, unknown or other
  variation 196: note = a, c, t, g, unknown or other
  variation 223: note = a, c, t, g, unknown or other
  variation 250: note = a, c, t, g, unknown or other
  variation 334: note = a, c, t, g, unknown or other
  variation 360: note = a, c, t, g, unknown or other
  variation 387: note = a, c, t, g, unknown or other
  variation 396: note = a, c, t, g, unknown or other
  variation 461: note = a, c, t, g, unknown or other
  variation 470: note = a, c, t, g, unknown or other
  variation 560: note = a, c, t, g, unknown or other
  variation 813: note = a, c, t, g, unknown or other
  variation 844: note = a, c, t, g, unknown or other
  variation 947: note = a, c, t, g, unknown or other
  variation 1140: note = a, c, t, g, unknown or other
  variation 1173: note = a, c, t, g, unknown or other
  variation 1367: note = a, c, t, g, unknown or other
  variation 1429: note = a, c, t, g, unknown or other
  source 1..1462: mol_type = genomic DNA
ttttcaacggagagttngatcctggctcaggacgaacgctngcggcgtgcttaacacatgcaagtcggacggtnaggcctttnnnngggggtnctcgagtggcgaacgggtgagtaacacgtgaggaacgtgcccttgacttcgggatagctccaggaaactggtggtaatcccgaatatgagcctngcctgcatnggtcgggttggaaagctttatgcggtnagggatcgtctcgcggcctatcagctngttggtggggtaatggcctaccaaggcagcgacgggtagccggcctgagagggtgaccggccacattgggactgagatacggcncagactcctacgggaggcagcagtgnggaatattgcacaatgggcgcaagccngatgcagcnacgccgcgtgcgggatgacggccttcgggttgtgaaccgctttcatccatgacgaagcgcaagtnacggtagtnggagaagaagcaccggctaactacgtgccagcagccgcggtgatacgtagggtgcgagcgttgtccggaattattgggcgtaaagagctngtaggcggttgtcacgtcggaagtgaaattccagggcttaactctgggcttgctttcgatacgggttgacttgaggaatgtaggggagaatggaactctcggtggagcggtggaatgcgcagatatcgggaagaacaccagtggcgaaggcggttctctggacatttcctgacgctgagaagcgaaagcgtggggagcaaacaggcttagataccctggtagtccacgccgtaaacggtgggtactaggtgtnggtcccttccacggggtccgtgccgtagctnacgcattaagtaccccgcctggggagtacggccgcaaggctaaaactcaaaggaattgacgggggcccgcacaagcggcggagcatgcggattaattcgatgnaacgcgaagaaccttacctgggtttgacatgtactggaagcgttcagagatgggcgtgcctttttggctggtacacaggtggtgcatggctgtcgtcagctcgtgtcgtgagatgttgggttaagtcccgcaacgagcgcaaccctcgtccaatgttgccagcagttcggctggggactcattggagaccgcnggggccaactcggaggaaggtggggatgaggtnaagtcatcatgccccttatgtccagggcttcacgcatgctacaatggccggtacaaagagttgcgagcctgtgagggtgagcgaatctcagaaagccggtctcagttcggattggggtctgcaactcgaccccatgaagtcggagtcgctagtaatcgcagatcagcaacgctgcggtgaatacgttcccgggncttgtacacaccgcccgtcaagtcatgaaagtcggtaacacccgaaccggtggcccaacctnttggggggagccgtcgaaggtgggactggtgat

SEQ ID NO: 70 - DNA, 331 bp, Klebsiella sp.
  source 1..331: mol_type = genomic DNA
cacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgatttggaggttgtgcccttgaggcgtggcttccggagctaacgcgttaaatcgaccgcctggggagtacgttcgcaagaatgaa

SEQ ID NO: 71 - DNA, 1561 bp, Lactobacillus delbrueckii
  source 1..1561: mol_type = genomic DNA
gagagtttgatcctggctcaggacgaacgctggcggcgtgcctaatacatgcaagtcgagcgagctgaattcaaagattccttcgggatgatttgttggacgctagcggcggatgggtgagtaacacgtgggcaatctgccctaaagactgggataccacttggaaacaggtgctaataccggataacaacatgaatcgcatgattcaagtttgaaaggcggcgtaagctgtcactttaggatgagcccgcggcgcattagctagttggtggggtaaaggcctaccaaggcaatgatgcgtagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggatagaggcagtaactggtctttatttgacggtaatcaaccagaaagtcacggctaactacgtgccagcagccgcggtaatacgtaggtggcaagcgttgtccggatttattgggcgtaaagcgagcgcaggcggaatgataagtctgatgtgaaagcccacggctcaaccgtggaactgcatcggaaactgtcattcttgagtgcagaagaggagagtggaattccatgtgtagcggtgtaatgcgtagatatatggaagaacaccagtggcgaaggcggctctctggtctgcaactgacgctgaggctcgaaagcatgggtagcgaacaggattagataccctggtagtccatgccgtaaacgatgagcgctaggtgttggggactttccggtcctcagtgccgcagcaaacgcattaagcgctccgcctggggagtacgaccgcaaggttgaaactcaaaggaattgacgggggcccgcacaagcggtggagcatgtggtttaattcgaagcaacgcgaagaaccttaccaggtcttgacatcctgtgctacacctagagataggtggttcccttcggggacgcagagacaggtggtgcatggctgtcgtcagctcgtgtcgtgagatgttgggttaagtcccgcaacgagcgcaacccttgtctttagttgccatcattaagttgggcactctaaagagactgccggtgacaaaccggaggaaggtggggatgacgtcaagtcatcatgccccttatgacctgggctacacacgtgctacaatgggcagtacaacgagaagcgaacccgcgagggtaagcggatctcttaaagctgttctcagttcggactgcaggctgcaactcgcctgcacgaagctggaatcgctagtaatcgcggatcagcacgccgcggtgaatacgttcccgggccttgtacacaccgcccgtcacaccatggaagtctgcaatgcccaaagtcggtgggataacctttataggagtcagccgcctaaggcagggcagatgactggggtgaagtcgtaacaaggtagccgtaggagaacctgcggctggatcacctcctttcta

SEQ ID NO: 72 - DNA, 1545 bp, Streptococcus mutans
  source 1..1545: mol_type = genomic DNA
ggagagtttgatcctggctcaggacgaacgctggcggcgtgcctaatacatgcaagtgggacgcaagggaacacactgtgcttgcacaccgtgttttcttgagtcgcgaacgggtgagtaacgcgtaggtaacctgcctattagcgggggataactattggaaacgatagctaataccgcataatattaattattgcatgataattgattgaaagatgcaagcgcatcactagtagatggacctgcgttgtattagctagttggtaaggtaagagcttaccaaggcgacgatacatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaagctctgttgtaagtcaagaacgtgtgtgagagtggaaagttcacacagtgacggtagcttaccagaaagggacggctaactacgtgccagcagccgcggtaatacgtaggtcccgagcgttgtccggatttattgggcgtaaagggagcgcaggcggtcaggaaagtctggagtaaaaggctatggctcaaccatagtgtgctctggaaactgtctgacttgagtgcagaaggggagagtggaattccatgtgtagcggtgaaatgcgtagatatatggaggaacaccagtggcgaaagcggctctctggtctgtcactgacgctgaggctcgaaagcgtgggtagcgaacaggattagataccctggtagtccacgccgtaaacgatgagtgctaggtgttaggccctttccggggcttagtgccggagctaacgcaataagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctattcttagagataggaagttacttcggtacatcggagacaggtggtgcatggttgtcgtcagctcgtgtcgtgagatgttgggttaagtcccgcaacgagcgcaacccttattgttagttgccatcattaagttgggcactctagcgagactgccggtaataaaccggaggaaggtggggatgacgtcaaatcatcatgccccttatgacctgggctacacacgtgctacaatggtcggtacaacgagttgcgagccggtgacggcaagctaatctctgaaagccgatctcagttcggattggaggctgcaactcgcctccatgaagtcggaatcgctagtaatcgcggatcagcacgccgcggtgaatacgttcccgggccttgtacacaccgcccgtcacaccacgagagtttgtaacacccgaagtcggtgaggtaaccttttaggggccagccgcctaaggtgggatggatgattggggtgaagtcgtaacaaggtagccgtatcggaaggtgcggctggatcac

SEQ ID NO: 73 - DNA, 1479 bp, Enterococcus rivorum
  variation 428: note = a, c, t, g, unknown or other
  source 1..1479: mol_type = genomic DNA
gacgaacgctggcggcgtgcctaatacatgcaagtcgaacgcttctttccttccgagtgcttgcactcaactggaaagaggagtggcggacgggtgagtaacacgtgggtaacctacccatcagagggggataacacttggaaacaggtgctaataccgcataacagtctgtaccgcatggtacaggagtgaaagacgctttcgggtgtcactgatggatggacccgcggtgcattagctagttggtgaggtaacggctcaccaaggccacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactcngttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattgttagttgccatcattcagttgggcactctagcgagactgccggtgacaaaccggaggaaggtggggatgacgtcaaatcatcatgccccttatgacctgggctacacacgtgctacaatgggaagtacaacgagtcgctaggccgcgaggtcatgcaaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaacctttatggagccagccgcctaaggtgggatagatgattgg

SEQ ID NO: 74 - DNA, 1311 bp, Enterobacter ludwigii
  source 1..1311: mol_type = genomic DNA
cgaaggcacggaatagcttggctctgttgtgacgagagcgggagaggtgagaattgtgtgggggactccctgatggagggggatatctcctgtgaacagtgtctcttaccccgtatcatctcacgaccccaaagagggacattctcgcctctcgtgctctcatatgcccccataggattttctcttaggtggggtaacggctccacctaggcgacgatccctagctggtctgagaggatgaccagcacactggactgagacacggtccagactctacgggaggcagcagtggggaatatgcacaatgggcgcaagcctgatgcagccatgccgcgtgtatgaagaaggccttcgggttgtaaagtacttcagcggggaggaaggtgttgaggttaataacctcagcaattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttaccagagatggattggtgccttcgggaactctgagacaggtgctgcatggctgtcgtcagctcgtgttgtgaaatgttgggttaagtcccgcaacgagcgcaacccttatcctttgttgccagcggttcggccgggaactcaaaggagactgccagtgataaactggaggaaggtggggatgacgtcaagtcatcatggcccttacgagtagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtagatcaagaatgcttaccgaaaccccc

SEQ ID NO: 75 - DNA, 1505 bp, Clostridium leptum
  source 1..1505: mol_type = genomic DNA
cgacgctggcggcgtgcctaacacatgcaagtcgaacggagttaaattcgacacccgagtatccggccgggaggcggggtgctgggggttggatttaacttagtggcggacgggtgagtaacgcgtgagtaacctgcctttcagagggggataacgttctgaaaagaacgctaataccgcataacatcaatttatcgcatgataggttgatcaaaggagcaatccgctggaagatggactcgcgtccgattagccagttggcggggtaacggcccaccaaagcgacgatcggtagccggactgagaggttgaacggccacattgggactgagacacggcccagactcctacgggaggcagcagtgggggatattgcacaatgggggaaaccctgatgcagcaacgccgcgtgagggaagaaggttttcggattgtaaacctctgttcttagtgacgataatgacggtagctaaggagaaagctccggctaactacgtgccagcagccgcggtaatacgtagggagcgagcgttgtccggatttactgggtgtaaagggtgcgtaggcggcgaggcaagtcaggcgtgaaatctatgggcttaacccataaactgcgcttgaaactgtcttgcttgagtgaagtagaggtaggcggaattcccggtgtagcggtgaaatgcgtagagatcgggaggaacaccagtggcgaaggcggcctactgggctttaactgacgctgaagcacgaaagcatgggtagcaaacaggattagataccctggtagtccatgccgtaaacgatgattactaggtgtggggggtctgaccccctccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccgtctaacgaagcagagatgcattaggtgcccttcggggaaaggcgagacaggtggtgcatggttgtcgtcagctcgtgtcgtgagatgttgggttaagtcccgcaacgagcgcaacccttgtttctagttgctacgcaagagcactctagagagactgccgttgacaaaacggaggaaggtggggacgacgtcaaatcatcatgccccttatgacctgggccacacacgtactacaatggctgtaaacagagggaagcaaagccgcgaggtggagcaaaaccctaaaagcagtcccagttcggatcgcaggctgcaacccgcctgcgtgaagtcggaattgctagtaatcgcggatcagcatgccgcggtgaatacgttcccgggccttgtacacaccgcccgtcacaccatgggagccggtaatacccgaagccagtagttcaaccgcaaggagagcgctgtcgaaggtaggattggcgactggggtgaagtcgtaacaaggtagccgtatcggaaggtgcggc

SEQ ID NO: 76 - DNA, 1437 bp, Enterococcus lactis
  source 1..1437: mol_type = genomic DNA
ctataatgcagtcgtacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaataaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacactctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaa

SEQ ID NO: 77 - DNA, 1518 bp, Bifidobacterium dentium
  source 1..1518: mol_type = genomic DNA
tttgatcatggctcaggatgaacgctggcggcgtgcttaacacatgcaagtcgaacgggatcccgggggttcgcctccgggtgagagtggcgaacgggtgagtaatgcgtgaccgacctgccccatacaccggaatagctcctggaaacgggtggtaatgccggatgctccggttggatgcatgtccttccgggaaaggttccatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatggaggccttcgggttgtaaaccgcttttgatcgggagcaagcccttcggggtgagtgtacccttcgaataagcaccggctaactacgtgccagcagccgcggtaatacgtagggtgcaagcgttatccggaattattgggcgtaaagggctcgtaggcggttcgtcgcgtccggtgtgaaagcccatcgcttaacggtgggtctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggccgtagagatacggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacagcgggatgcgacatggcgacatggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggcagcacccgaagccggtggcctaacccttctgtgggatggagccgtctaaggtgaggctcgtgattgggactaagtcgtaacaaggtagccgtaccggaaggtgcggctggatcacctcct

SEQ ID NO: 78 - DNA, 1484 bp, Eubacterium limosum
  source 1..1484: mol_type = genomic DNA
agagtttgatcctggctcaggacgaacgctggcggtatgcttaacacatgcaagtcgaacgagaaggttttgatggatccttcgggtgacattagaactggaaagtggcgaacgggtgagtaacgcgtgggtaacctgccctatggaaaggaatagcctcgggaaactgggagtaaagccttatattatggttttgtcgcatggcaagatcatgaaaactccggtgccataggatggacccgcgtcccattagctagttggtgagataacagcccaccaaggcgacgatgggtaaccggtctgagagggcgaacggtcacactggaactgagacacggtccagactcctacgggaggcagcagtggggaatattgcgcaatgggggcaaccctgacgcagcaataccgcgtgagtgaagaaggttttcggatcgtaaagctctgttattggggaagaagaatgacggtacccaatgaggaagtcccggctaactacgtgccagcagccgcggtaatacgtaggggacaagcgttgtccggaatgactgggcgtaaagggcgcgtaggcggtctattaagtctgatgtgaaaggtaccggctcaaccggtgaagtgcattggaaactggtagacttgagtattggagaggcaagtggaattcctagtgtagcggtgaaatgcgtagatattaggaggaacaccagtggcgaaggcggcttgctggacaaatactgacgctgaggtgcgaaagcgtggggagcgaacaggattagataccctggtagtccacgccgtaaacgatgaatgctaggtgttggggaaactcagtgccgcagttaacacaataagcattccgcctggggagtacgaccgcaaggttgaaactcaaaggaattgacggggacccgcacaagcagcggagcatgtggtttaattcgaagcaacgcgaagaaccttaccaggtcttgacatcctctgacgagcctagagataggaagtttccttcgggaacagagagacaggtggtgcatggttgtcgtcagctcgtgtcgtgagatgttgggttaagtcccgcaacgagcgcaacccctgcctttagttgccagcattaagttgggcactctagagggactgccgtagacaatacggaggaaggtggggacgacgtcaaatcatcatgccccttatgacctgggctacacacgtgctacaatggtctgaacagagggccgcgaagccgcgaggtgaagcaaatcccttaaaacagatcccagttcggattgcaggctgcaactcgcctgcatgaagttggagttgctagtaatcgcggatcagaatgccgcggtgaatgcgttcccgggtcttgtacacaccgcccgtcacaccacgagagttggcaacacccgaagcctgtgagagaaccgcaaggactcagcagtcgaaggtggggctagtaattggggtgaagtcgtaacaaggtaacc

SEQ ID NO: 79 - DNA, 1391 bp, Holdemania filiformis
  variation 1024: note = a, c, t, g, unknown or other
  source 1..1391: mol_type = genomic DNA
ggagtggcgaacgggtgagtaatacataagcaatctgcccatcggcctgggataacagttggaaacgactgctaataccggataggtgatgaagaggcatctctcgatcattaaagttgggatacaacacggatggatgagcttatggcgtattagctagtaggtgaggtaacggctcacctaggcgatgatacgtagccgacctgagagggtgaccggccacattgggactgagacacggcccaaactcctacgggaggcagcagtagggaattttcggcaatgggcgaaagcctgaccgagcaacgccgcgtgagtgaagaaggccttcgggttgtaaagctctgttgtgaaggaagaacggctcatacagggaatggtatgggagtgacggtactttaccagaaagccacggctaactacgtgccagcagccgcggtaatacgtaggtggcgagcgttatccggaattattgggcgtaaagggtgcgcaggcggtttgttaagtttaaggtgaaagcgtggggcttaaccccatatagccttagaaactgacagactagagtacaggagagggcaatggaattccatgtgtagcggtaaaatgcgtagatatatggaggaacaccagtggcgaaggcggttgcctggcctgtaactgacgctcatgcacgaaagcgtggggagcaaataggattagataccctagtagtccacgccgtaaacgatgagaactaagtgttggggaaactcagtgctgcagttaacgcaataagttctccgcctggggagtatgcacgcaagtgtgaaactcaaaggaattgacgggggcccgcacaagcggtggagtatgtggtttaattcgacgcaacgcgaagaaccttaccaggtcttgacatcccctgcaaagacatagagatatgttggaggttatcagggagacaggtggtgcatggttgtcgtcagctcgtgtcgtgagatgttgggttaagtcccgcaacgagcgcaacccttgtctttagttnctaacattaagttgaggactctagagagactgccggtgacaaaccggaggaaggtggggatgacgtcaaatcatcatgccccttatgacctgggctacacacgtactacaatggcggatacaacgagaagcaagacagtgatgtggagcaaaactcagaaagtccgtctcagttcggattgaagtctgcaacccgacttcatgaagccggaatcgctagtaatcgcggatcagcatgccgcggtgaatacgttctcgggccttgtacacaccgcccgtcaaaccatgagagttggcaatacccgaagccggtggcctaacctcgcaagaggagggagccgtcgaaggtagggctgatgattggggtt

SEQ ID NO: 80 - DNA, 1522 bp, Lactococcus piscium
  source 1..1522: mol_type = genomic DNA
tcctggctgaggacgaacgctggcggcgtgcctaatacatgcaagttgaacgatgatttctggtacttgtatcagaatgaagagtagcgaacgggtgagtaacgcgtgggtaacctacctcttagcgggggacaactattggaaacgatagctaataccgcataacaatgttaaacccatgtttaacatttgaaagtaccaattggtacactaggagatggacccgcgttgtattagctagttggtagtgtaatggactaccaaggcgatgatacatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacgttgtgtagagtggaaaattacacaagtgacggtatctaaccagaaagggacggctaactacgtgccagcagccgcggtaatacgtaggtcccgagcgttgtccggatttattgggcgtaaagcgagcgcaggtggtttaataagtctgatgtaaaaggcagtggctcaaccattgtgtgcattggaaactgttagacttgagtgcagtagaggagagtggaattccatgtgtagcggtgaaatgcgtagatatatggaggaacaccggtggcgaaagcggctctctggactgtaactgacactgaggctcgaaagcgtgggtagcaaacaggattagataccctggtagtccacgccgtaaacgatgagtgctagttgtttggggctatccagccctaagtgacgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacataccagtgctattcttagagataagaagttacttcggtacactggatacaggtggtgcatggttgtcgtcagctcgtgtcgtgagatgttgggttaagtcccgcaacgagcgcaacccctattgttagttgccatcattaagttgggcactctagcgagactgccggtaataaaccggaggaaggtggggatgacgtcaaatcatcatgccccttatgacctgggctacacacgtgctacaatggttggtacaacgagtcgcaagctagtgatagcaagctaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaatacccaaagccggtgagctaaccttttaggaggcagccgtctaaggtaggatagatgattggggtgaagtcgtaacaaggtagccgtatcggaaggtgcggctgga

SEQ ID NO: 81 - DNA, 1483 bp, Bacteroides thetaiotaomicron
  source 1..1483: mol_type = genomic DNA
cttttacaatgaagagtttgatcctggctcaggatgaacgctagctacaggcttaacacatgcaagtcgaggggcagcatttcagtttgcttgcaaactggagatggcgaccggcgcacgggtgagtaacacgtatccaacctgccgataactcggggatagcctttcgaaagaaagattaatacccgatggtataatcagaccgcatggtcttgttattaaagaatttcggttatcgatggggatgcgttccattaggcagttggtgaggtaacggctcaccaaaccttcgatggataggggttctgagaggaaggtcccccacattggaactgagacacggtccaaactcctacgggaggcagcagtgaggaatattggtcaatgggcgcaggcctgaaccagccaagtagcgtgaaggatgactgccctatgggttgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacgggtgaaactcaaaggaattgacgggggcccgtacaagcggaggaacatgtggtttaattcgatgatacgcgaggaaccttacccgggcttaaattgcatttgaatatattggaaacagtatagccgtaaggcaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtcaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggc

SEQ ID NO: 82 - DNA, 1487 bp, Massiliomicrobiota timonensis
  source 1..1487: mol_type = genomic DNA
agagtttgatcctggctcaggatgaacgctggcggcgtgcctaatacatgcaagtcggacgcaatgcttcggcattgagtggcgaacgggtgagtaagacataagcaacctgcccctgtgagggggataactgctggaaacggcagctaagaccgcataggcatagaggacgcatgtcgactatgttaaatatcccacgggatagcacagggatgggcttatgacgcattagccagctggtgaggtaacggctcaccagggcgacgatgcgtagccggcctgagagggtggacggccacactgggactgagacacggcccagactcctacgggaggcagcagtagggaattttcggcaatgggcgaaagcctgaccgagcaacgccgcgtgaaggaagaagtcattcgtgatgtaaacttctgttatgaaggaagaacggcagatggagggaatgccatgtgcgtgacggtacttcatgaggaagccacggctaactacgtgccagcagccgcggtaatacgtaggtggcgagcgttatccggaatcattgggcgtaaagagggagcaggcggcagtgcaggtctgcggtgaaagaccggagctaaacttcggtaagccgtggaaaccgcacagctagagagcatcagaggatcgcggaattccatgtgtagcggtgaaatgcgtagatatatggaggaacaccagtggcgaaggcggcggtctggggtgcagctgacgctcagtcccgaaagcgtggggagcaaataggattagataccctagtagtccacgccgtaaacgatgagtgctaagtgttgggggtcagacctcagtgctggagttaacgcaataagcactccgcctgagtagtacgttcgcaagaatgaaactcaaaggaattgacgggggcccgcacaagcggtggagcatgtggtttaattcgaagcaacgcgaagaaccttaccaggtcttgacatggagataaaggccctggagacagggagatagatatatctcacacaggtggtgcatggttgtcgtcagctcgtgtcgtgagatgttgggttaagtcccgcaacgagcgcaacccctgttgccagttgccagcattaggttggggactctggcgagactgcctctgcaaggaggaggaaggcggggatgacgtcaaatcatcatgccccttatgacctgggctacacacgtgctacaatggacggatcagagggaggcgaagccgcgaggtggagcgaaacccagaaacccgttcacagttcggactgcagtctgcaactcgactgcacgaagctggaatcgctagtaatcgcgaatcagcatgtcgcggtgaatacgttctcgggccttgtacacaccgcccgtcacaccatgagagttggtaacacccgaagccggtggcccaaccgcaaggagggagctgtctaaggtgggactgatgattggggtgaagtcgtaacaaggtagccgt

SEQ ID NO: 83 - DNA, 1458 bp, Blautia hydrogenotrophica
  variation 1416: note = a, c, t, g, unknown or other
  source 1..1458: mol_type = genomic DNA
gatgaacgctggcggcgtgcttaacacatgcaagtcgaacgaagcgatagagaacggagatttcggttgaagttttctattgactgagtggcggacgggtgagtaacgcgtgggtaacctgccctatacagggggataacagttagaaatgactgctaataccgcataagcgcacagcttcgcatgaagcggtgtgaaaaactgaggtggtataggatggacccgcgttggattagctagttggtgaggtaacggcccaccaaggcgacgatccatagccggcctgagagggtgaacggccacattgggactgagacacggcccaaactcctacgggaggcagcagtggggaatattgcacaatgggggaaaccctgatgcagcgacgccgcgtgaaggaagaagtatctcggtatgtaaacttctatcagcagggaagaaagtgacggtacctgactaagaagccccggctaattacgtgccagcagccgcggtaatacgtaaggggcaagcgttatccggatttactgggtgtaaagggagcgtagacggtttggcaagtctgatgtgaaaggcatgggctcaacctgtggactgcattggaaactgtcagacttgagtgccggagaggcaagcggaattcctagtgtagcggtgaaatgcgtagatattaggaggaacaccagtggcgaaggcggcctgctggacggtaactgacgttgaggctcgaaagcgtggggagcaaacaggattagataccctggtagtccacgctgtaaacgatgaatactaggtgtcgggtggcaaagccattcggtgccgcagcaaacgcaataagtattcccacctggggagtacgttcgcaagaatgaaactcaaaggaattgacggggacccgcacaagcggtggagcatgtggtttaattcgaagcaacgcgaagaaccttaccaaatcttgacatccctctgaccgggaagtaatgttcccttttcttcggaacagaggagacaggtggtgcatggttgtcgtcagctcgtgtcgtgagatgttgggttaagtcccgcaacgagcgcaacccttattcttagtagccagcaggtagagctgggcactctagggagactgccagggataacctggaggaaggtggggatgacgtcaaatcatcatgccccttatgatttgggctacacacgtgctacaatggcgtaaacaaagggaagcgaaggggtgacctggagcaaatctcaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccnaaaggagggagctgccgaaggtgggactgataactggggtga

SEQ ID NO: 84 - DNA, 1529 bp, Enterococcus mundtii
  source 1..1529: mol_type = genomic DNA
acgaacgctggcggcgtgcctaatacatgcaagtcgaacgcttcttttcccaccggagcttgctccaccgggaaaagaggagtggcgaacgggtgagtaacacgtgggtaacctgcccatcagaaggggataacacttggaaacaggtgctaataccgtataacaatcgaaaccgcatggtttcgttttgaaaggcgctttacggtgccgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaagggtgagagtaactgttcaccccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaacctttttggagccagccgcctaaggtgggatagatgattggggtgaagtcgtaacaaggtagccgtatcggaaggtgcggctggatcacctc

SEQ ID NO: 85 - DNA, 1480 bp, Prevotella melaninogenica
  source 1..1480: mol_type = genomic DNA
tggctcaggatgaacgctagctacaggcttaacacatgcaagttgaggggaaacggcattgagtgcttgcactctttggacgtcgaccggcgcacgggtgagtaacgcgtatccaaccttcccattactgtgggataacctgccgaaaggcagactaataccgcatagtcttcgatgacggcatcagatttgaagtaaagatttatcggtaatggatggggatgcgtctgattagcttgttggcggggtaacggcccaccaaggcaacgatcagtaggggttctgagaggaaggtcccccacattggaactgagacacggtccaaactcctacgggaggcagcagtgaggaatattggtcaatggacggaagtctgaaccagccaagtagcgtgcaggatgacggccctatgggttgtaaactgcttttgtatggggataaagttagggacgtgtccctatttgcaggtaccatacgaataaggaccggctaattccgtgccagcagccgcggtaatacggaaggtccaggcgttatccggatttattgggtttaaagggagcgtaggctggagattaagtgtgttgtgaaatgtagacgctcaacgtctgaattgcagcgcatactggtttccttgagtacgcacaacgttggcggaattcgtcgtgtagcggtgaaatgcttagatatgacgaagaactccgattgcgaaggcagctgacgggagcgcaactgacgcttaagctcgaaggtgcgggtatcaaacaggattagataccctggtagtccgcacagtaaacgatggatgcccgctgttggtacctggtatcagcggctaagcgaaagcattaagcatcccacctggggagtacgccggcaacggtgaaactcaaaggaattgacgggggcccgcacaagcggaggaacatgtggtttaattcgatgatacgcgaggaaccttacccgggcttgaattgcagaggaaggatttagagataatgacgcccttcggggtctctgtgaaggtgctgcatggttgtcgtcagctcgtgccgtgaggtgtcggcttaagtgccataacgagcgcaacccctctcttcagttgccatcaggttaagctgggcactctggagacactgccaccgtaaggtgtgaggaaggtggggatgacgtcaaatcagcacggcccttacgtccggggctacacacgtgttacaatggccggtacagagggacggtgtaatgcaaattgcatccaatcttgaaagccggtcccagttcggactggggtctgcaacccgaccccacgaagctggattcgctagtaatcgcgcatcagccatggcgcggtgaatacgttcccgggccttgtacacaccgcccgtcaagccatgaaagccgggggtgcctgaagtccgtgaccgcaaggatcggcctagggcaaaactggtgattggggctaagtcgtaacaaggtaacc

SEQ ID NO: 86 - DNA, 1537 bp, Erwinia chrysanthemi
  source 1..1537: mol_type = genomic DNA
tagagttttgatcatggctcagattgaacgctggcggcaggcctaacacatgcaagtcgagcggcagcggggggaagcttgcttccccgccggcgagcggcggacgggtgagtaatgtctggggatctgcctgatggagggggataactactggaaacggtagctaataccgcataacgtcgcaagaccaaagtgggggaccttcgggcctcacgccatcggatgaacccagatgggattagctagtaggtgaggtaacggctcacctaggcgacgatccctagctggtctgagaggatgaccagccacactggaactgagacacggtccagactcctacgggaggcagcagtggggaatattgcacaatgggggaaaccctgatgcagccatgccgcgtgtgtgaagaaggccttcgggttgtaaagcactttcagcggggaggaagggagcagggttaataaccttgttcattgacgttacccgcagaagaagcaccggctaactccgtgccagcagccgcggtaatacggagggtgcaagcgttaatcggaatgactgggcgtaaagcgcacgcaggcggtctgttaagttggatgtgaaatccccgggcttaacctgggaactgcattcaaaactgacaggctagagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgctgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccagcgaagcctgtagagatacgggtgtgccttcgggagcgctgagacaggtgctgcatggctgtcgtcagctcgtgttgtgaaatgttgggttaagtcccgcaacgagcgcaacccttatcctctgttgccagcacatcgggtgggaactcaggggagactgccggtgataaaccggaggaaggtggggatgacgtcaagtcatcatggcccttacgagtagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 87 - DNA, 1517 bp, Tyzzerella nexilis; identical to SEQ ID NO: 24
  variation 184..185: note = a, c, t, g, unknown or other
  variation 341: note = a, c, t, g, unknown or other
  variation 430: note = a, c, t, g, unknown or other
  source 1..1517: mol_type = genomic DNA

SEQ ID NO: 88 - DNA, 1464 bp, Clostridioides difficile
  source 1..1464: mol_type = genomic DNA
agagtttgatcctggctcaggatgaacgctggcggcgtgcctaacacatgcaagttgagcgatttacttcggtaaagagcggcggacgggtgagtaacgcgtgggtaacctaccctgtacacacggataacataccgaaaggtatgctaatacgggataatatatttgagaggcatctcttgaatatcaaaggtgagccagtacaggatggacccgcgtctgattagctagttggtaaggtaacggcttaccaaggcgacgatcagtagccgacctgagagggtgatcggccacattggaactgagacacggtccaaactcctacgggaggcagcagtggggaatattgcacaatgggcgaaagcctgatgcagcaacgccgcgtgagtgatgaaggccttcgggtcgtaaaactctgtcctcaaggaagataatgacggtacttgaggaggaagccccggctaactacgtgccagcagccgcggtaatacgtagggggctagcgttatccggatttactgggcgtaaagggtgcgtaggcggtctttcaagtcaggagtgaaaggctacggctcaaccgtagtaagctcttgaaactgggagacttgagtgcaggagaggagagtggaattcctagtgtagcggtgaaatgcgtagatattaggaggaacaccagttgcgaaggcggctctctggactgtaactgacgctgaggcacgaaagcgtggggagcaaacaggattagataccctggtagtccacgctgtaaacgatgagtactaggtgtcgggggttacccccttcggtgccgcagctaacgcattaagtactccgcctgggaagtacgctcgcaagagtgaaactcaaaggaattgacggggacccgcacaagtagcggagcatgtggtttaattcgaagcaacgcgaagaaccttacctaagcttgacatcccaatgacatctccttaatcggagagttcccttcggggacattggtgacaggtggtgcatggttgtcgtcagctcgtgtcgtgagatgttgggttaagtcccgcaacgagcgcaacccttgtctttagttgccatcattaagttgggcactctagagagactgccagggataacctggaggaaggtggggatgacgtcaaatcatcatgccccttatgcttagggctacacacgtgctacaatgggtagtacagagggttgccaagccgtaaggtggagctaatcccttaaagctactctcagttcggattgtaggctgaaactcgcctacatgaagctggagttactagtaatcgcagatcagaatgctgcggtgaatgcgttcccgggtcttgtacacaccgcccgtcacaccacgggagttggagacgcccgaagccgattatctaaccttttggaagaagtcgtcgaaggtggaatcaataactggggtgaagtcgtaacaaggtaacc

SEQ ID NO: 89 - DNA, 1398 bp, Blautia obeum
  variation 1083: note = a, c, t, g, unknown or other
  variation 1207: note = a, c, t, g, unknown or other
  variation 1233: note = a, c, t, g, unknown or other
  source 1..1398: mol_type = genomic DNA
ggcgtgcttaacacatgcaagtcgaacgggaaacttttcattgaagcttcggcagatttggtctgtttctagtggcggacgggtgagtaacgcgtgggtaacctgccttatacagggggataacaaccagaaatggttgctaataccgcataagcgcacaggaccgcatggtctggtgtgaaaaactccggtggtataagatggacccgcgttggattagctagttggcagggtaacggcctaccaaggcgacgatccatagccggcctgagagggtgaacggccacattgggactgagacacgccccagactcctcgggaggcagcagtggggaatattgcacaatgggggaaaccctgatgcagcgacgccgcgtgaaggaagaagtatctcggtatgtaaacttctatcagcagggaagatagtgacggtacctgactaagaagccccgkctaactacgtgccagcagccgcggtaatacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgcaaacgatgaatactaggtgttggggagcaaagctcttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccctctgaccgtcccttaaccggatctttccttcgggacaggggagacaggtggtgcatggttgtcgtcagctcgtgtcgtgagatgttgggttaagtcccgcaacgagcgcaacccctatccccagtagccagcagtncggctgggcactctgaggagactgccagggataacctggaggaaggcggggatgacgtcaaatcatcatgccccttatgatttgggctacacacgtgctacaatggcgtaaacaaagggaagcnagcctkcgraggtaagcaaatcccanaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaactgc

SEQ ID NO: 90 - DNA, 1476 bp, Clostridium clostridioforme
  variation 535: note = a, c, t, g, unknown or other
  variation 918: note = a, c, t, g, unknown or other
  variation 955: note = a, c, t, g, unknown or other
  variation 1011: note = a, c, t, g, unknown or other
  variation 1454: note = a, c, t, g, unknown or other
  variation 1472: note = a, c, t, g, unknown or other
  source 1..1476: mol_type = genomic DNA
gaacatgagagtttgatcctggctcaggatcaacgctggcggcgtgcctaacacatgcaagtcgaacgaagcaattaagatgaagttttcggatggaatcttgattgactgagtggcggacgggtgagtaacgcgtggataacctgcctcacactgggggataacagttagaaatgactgctaataccgcataagcgcacagtgccgcatggcagtgtgtgaaaaactccggtagtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcnagcgttatccggatttactgggtgtaaagggagcgtagacggcgaagcaagtctgaagtgaaaacccagggctcaaccctggcactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagccttcggtgccgccgcaaacgcagtaagcattccacctggggagtacgttcgcaagaatgaaactcaaaggaattgacgggganccgcacaagcggtggagcatgtggtttaattcgaagnaacgcgaagaaccttaccaagtcttgacatccccctgacggccggtaacgcggccnttcttcgggacaggggagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcgagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagggagctgccnaaggcggggcaggtaacngggg

SEQ ID NO: 91 - DNA, 1398 bp, Blautia obeum; identical to SEQ ID NO: 89
  variation 1083: note = a, c, t, g, unknown or other
  variation 1207: note = a, c, t, g, unknown or other
  variation 1233: note = a, c, t, g, unknown or other
  source 1..1398: mol_type = genomic DNA

SEQ ID NO: 92 - DNA, 1436 bp, Klebsiella oxytoca
  source 1..1436: mol_type = genomic DNA
atcctggctcagattgaacgctggcggcaggcctaacacatgcaagtcgaacggtagcacagagagcttgctctcgggtgacgagtggcggacgggtgagtaatgtctgggaaactgcccgatggagggggataactactggaaacggtagctaataccgcataacgtcgcaagaccaaagagggggaccttcgggcctcttgccatcggatgtgcccagatgggattagcttgtaggtgaggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtgaggttaataaccttattcattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctggagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcgattcggtcgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcg

SEQ ID NO: 93 - DNA, 1487 bp, Solobacterium moorei
  source 1..1487: mol_type = genomic DNA
agagtttgatcctggctcagtatgaactctgccggcgtgcctaatacttgcaagtcgaacgctgaagatctagcttgctagatcgaaggagtgccgaacgggtgagtaatacataagcaacctacccacgaagactgggataatctctggaaacggggactaataccggataggtaatcggaaggcatcttctggttattaaaggttaaaaacactggtggatgggcttatggcgcattagttagttggtgaggtaacggcccaccaagacgatgatgcgtagccggcctgagagggtgaacggccacattgggactgagacacggcccagactctacgggaggcagcagtagggaattttcggcaataggggcaaccctgaccgagcaacgccgcgtgagtgaagacggccttcgggttgtaaaagctcttgtttgtaagggaagaacggtagatagagaatatctaagtgacggtaccttaccagaaagccacggctaactacgtgccagcagccgcggtaatacgtaggtggcgaccgttatccggaattattggccgtaaagggtgcgtaggcggcctgttaagtaagtggttaaattgttgggctcaacccaatccagccacttaaactggcaggctagagtattggagaggcaagtggaattccatgtgtagcggtaaaatgcgtagatatatggaggaacaccagtggcgaaggcggcttgctagccaaagactgacgctcatgcacgaaaccgtggggagccaataggattagataccctagtagtccacgccgtaaccgatgaatactaagtattggggaaactcagtgctgcactaacgcaataagtattccgcctgtggagtatgcacgcaagtgtgaaacataaaggaattgacgggggcccgcacaagcggtggagtatgtggtttaattcgacgcaacgcgaagaaccttaccaggccttgacatcccttgcaaagctgtagagatacagtagaggttatcaaggagacaggtggttgcatggtgtcgtcagctcgtgtcgtgagatgttgggttaagtccggcaacgagcgcaacccttgccttcagttaccagcatttagttggggactctggagggactgccggtgataaaccggaggaaggtggggatgacgtcaaatcatcatgccccttatggcctgggctacacacgtactacaatggctgttacaacgtgcagcgacctagcgataggaagcgaatcactaaaagacagtctcagttcggattgaagtctgcaactcgacttcatgaagctggaatcgctagtaatcgcggatcagaatgccgcggtgaatacgttctcgggccttgtacacaccgcccgtcataccatgagagctggtaatacccgaggccggtggcctaaccgcaaggaaggagccgtcgaaggtaggactagtgattggggtcaagtcgtaacaaggtaacc

SEQ ID NO: 94 - DNA, 1492 bp, Parabacteroides merdae
  source 1..1492: mol_type = genomic DNA
agagtttgatcctggctcaggatgaacgctagcgacaggcttaacacatgcaagtcgaggggcagcatgatttgtagcaatacagattgatggcgaccggcgcacgggtgagtaacgcgtatgcaacttacctatcagagggggatagcccggcgaaagtcggattaataccccataaaacaggggtcccgcatgggaatatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggaagaaggatctatggtttgtaaacttcttttataggggaataaagtggaggacgtgtccttttttgtatgtaccctatgaataagcatcggctaactccgtgccagcagccgcggtaatacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtggtgtagcggtgaaatgcatagatatcacgcagaactccgattgcgaaggcagcttactaaaccataactgacactgaagcacgaaagcgtggggatcaaacaggattagataccctggtagtccacgcagtaaacgatgattactaggagtttgcgatacaatgtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtctgaccggagtggaaacactccttctagcaatagcagattacgaggtgctgcatggttgtcgtcagctcgtgccgtgaggtgtcggcttaagtgccataacgagcgcaacccttatcactagttactaacaggtgaagctgaggactctggtgagactgccagcgtaagctgtgaggaaggtggggatgacgtcaaatcagcacggcccttacatccggggcgacacacgtgttacaatggcatggacaaagggcagctacctggcgacaggatgctaatctccaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaaggatcggcctagggtaaaactggtgactggggctaagtcgtaacaaggtaacc

SEQ ID NO: 95 - DNA, 1456 bp, Shuttleworthia satelles
  source 1..1456: mol_type = genomic DNA
gatgaacgctggcggcgtgcttaacacatgcaagtcgaacgagaaatgatattacgatctcttcggagtgacggttttatttcgagtggcggacgggtgagtaacgcgtgagcaacctgccctataccgggggataccagttggaaacgactgttaataccgcataagcgcacgataagacatcttgttgtgtgaaaaactccggtggtataggatgggctcgcgtatgattagccagttggcggggtaaaggcccaccaaagcgacgatcattagccgacctgagagggcgaccggccacattgggactgagacacggcccaaactcctacgggaggcagcagtggggaatattgcacaatgggcgaaagcctgatgcagcgacgccgcgtgagtgaggaagtatttcggtatgtaaagctctatcagcagggaagaaagaaatgacggtacctgagtaagaagctccggctaaatacgtgccagcagccgcggtaatacgtatggagcaagcgttatccggatttactgggtgtaaagggagcgtaggcggcagagcaagtctgatgtgaaagcccggggctcaaccccggtagtgcattggaaactgttcagctcgagtgtcggaggggtaagcggaattcctggtgtagcggtgaaatgcgtagatatcaggaagaacaccggaggcgaaggcggcttactggacgattactgacgctgaggctcgaaagcgtggggagcgaacaggattagataccctggtagtccacgctgtaaacgatgaatactaggtgtcggtacccgaagggtatcggtgccgtcgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatcccgatgacaagcgatgtaatgtcgtctctcttcggagcatcggagacaggtggtgcatggttgtcgtcagctcgtgtcgtgagatgttgggttaagtcccgcaacgagcgcaacccttgttcttagtagccagcaggtgagctgggcactctaaggagactgccggggtgaacccggaggaaggtggggatgacgtcaaatcatcatgccccttatgacttgggctacacacgtgctacaatggcgtaaacaaagggaggcgaagccgtgaggccaagcgaatcccaaaaataacgtctcagttcggattgtagtctgcaactcgactacatgaagctggaatcgctagtaatcgcagatcagcatgctgcggtgaatacgttcccgggtcttgtacacaccgcccgtcacaccatgggagttggtaatgcccgaagtcagtgacccaaccgcaaggagggagctgccgaaggcaggactgataactggggtg

SEQ ID NO: 96 - DNA, 1385 bp, Streptococcus sp.
  source 1..1385: mol_type = genomic DNA
tgagagaggagcttgctcttcttggatgagttgcgaacgggtgagtaacgcgtaggtaacctgccttgtagcgggggataactattggaaacgatagctaataccgcataacaatggatgacacatgtcatttatttgaaaggggcaattgctccactacaagatggacctgcgttgtattagctagtaggtgaggtaatggctcacctaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagtcaagaacgggtgtgagagtggaaagttcacactgtgacggtagcttaccagaaagggacggctaactacgtgccagcagccgcggtaatacgtaggtcccgagcgttgtccggatttattgggcgtaaagcgagcgcaggcggtttgataagtctgaagttaaaggctgtggctcaaccatagttcgctttggaaactgtcaaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttggatcctttccgggattcagtgccgaagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctatttctagagatagaaagttacttcggtacatcggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggttggtacaacgagttgcgagtcggtgacggcgagctaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaacc

SEQ ID NO: 97 - DNA, 1500 bp, Clostridium cellulosi
  variation 1380..1381: note = a, c, t, g, unknown or other
  source 1..1500: mol_type = genomic DNA
tttgatcctggctcaggacgaacgctggcggcgtgcctaacacatgcaagtcgagcggagatagtacttcggttctatcttagcggcggacgggtgagtaacgcgtgagcaacctgccctgagcacggggatagcgtctggaaacggacggtaataccgcataatgtacgttggaggcatctccgatgtaccaaaggaggaatccactcaaggatgggctcgcgtccgattaggtagttggtgaggtaacggcccaccaagcctgcgatcggtagccggactgagaggttgtacggccacattgggactgagacacggcccagactcctacgggaggcagcagtggggaatattgcacaatggaggaaactctgatgcagcgacgccgcgtgagggaagaaggtcttcggattgtaaacctctgtctttcgggacgaaggaagtgacggtaccgaaagaggaagccacggctaactacgtgccagcagccgcggtaatacgtaggtggcgagcgttgtccggaattactgggtgtaaagggtgcgtaggcgggttgtcaagttggatgtgaaatctctgggcttaactcagaggttgcattcaaaactggcgatcttgagtgaggtagaggcaggcggaattcccggtgtagcggtgaaatgcgtagatatcgggaggaacaccagtggcgaaggcggcctgctgggccttaactgacgctgaggcgcgaaagcgtggggagcaaacaggattagataccctggtagtccatgctgtaaacgatgattgctaggtgtgggtggactgaccccatccgtgccggagttaacacaataagcatcccacctggggagtacggccgcaaggctgaaactcaaaggaattgacgggggcccgcacaagcagtggagtatgtggtttaattcgaagcaacgcgaagaaccttaccaggtcttgacatccaccgaatccggaagagattctggagtgcccttcggggagcggtgagacaggtggtgcatggttgtcgtcagctcgtgtcgtgagatgttgggttaagtcccgcaacgagcgcaacccttgttaatagttgctacgcaagagcactctattaagactgccgttgataaaacggaggaaggtggggatgacgtcaaatcatcatgccccttatgacctgggctacacacgtactacaatggccgccaacaaagggaagcaataccgcgaggtggagcgaatccccaaaagcggtcccagttcagattgcaggctgcaacccgcctgcatgaagacggaattgctagtaatcgcggatcagcatgccgcggtgaatacgttcccgggccttgtacacaccgcccgtcacaccatgagagnnggcaacacccgaagtcgtttgcgtaaccgaaaggagcgcggcgccgaaggtgggatcggtgattggggtgaagtcgtaacaaggtagccgtatcggaaggtgcggctggatcacctcct

SEQ ID NO: 98 - DNA, 1478 bp, Lactobacillus acidophilus
  source 1..1478: mol_type = genomic DNA
tcctggctcaggacgaacgctggcggcgtgcctaatacatgcaagtcgagcgagctgaaccaacagattcacttcggtgatgacgttgggaacgcgagcggcggatgggtgagtaacacgtggggaacctgccccatagtctgggataccacttggaaacaggtgctaataccggataagaaagcagatcgcatgatcagcttataaaaggcggcgtaagctgtcgctatgggatggccccgcggtgcattagctagttggtagggtaacggcctaccaaggcaatgatgcatagccgagttgagagactgatcggccacattgggactgagacacggcccaaactcctacgggaggcagcagtagggaatcttccacaatggacgaaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagtgcaatccgtagagatacggagttcccttcggggacactaagacaggtggtgcatggctgtcgtcagctcgtgtcgtgagatgttgggttaagtcccgcaacgagcgcaacccttgtcattagttgccagcattaagttgggcactctaatgagactgccggtgacaaaccggaggaaggtggggatgacgtcaagtcatcatgccccttatgacctgggctacacacgtgctacaatggacagtacaacgaggagcaagcctgcgaaggcaagcgaatctcttaaagctgttctcagttcggactgcagtctgcaactcgactgcacgaagctggaatcgctagtaatcgcggatcagcacgccgcggtgaatacgttcccgggccttgtacacaccgcccgtcacaccatgggagtctgcaatgcccaaagccggtggcctaaccttcgggaaggagccgtctaaggc

SEQ ID NO: 99 - DNA, 1455 bp, Bacteroides ovatus
  source 1..1455: mol_type = genomic DNA
gatcatggctcaggatgaacgctagctacaggcttaacacatacaagtcgtggggcagcattttatgtttgtttgcaaactgaagatggcgaccggcgcacgggtgagtaacacgtatccaacctgccgataactccggaatagcctttcgaaagaaagattaataccggatagcatacgatatcgcatgatatttttattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatgggcgagagcctgaaccagccaagtagcgtgaaggatgaaggctctatgggtcgtaaacttcttttatatgggaataaagttttccacgtgtggaattttgtatgtaccatatgaataaggatcggactactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgttactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgagaaccttacccgggcttaaattgcaacagaatatattggaaacagtatagccgtaaggctgttgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctacctggtgacatgatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaa

SEQ ID NO: 100 - DNA, 1445 bp, Clostridium hylemonae
  source 1..1445: mol_type = genomic DNA
aggatgaacgctgccgccgtgcttaacacatgcaagtcgaacgaagcaatactgtgtgaagagattagcttgctaagatcagaactttgtattgactgagtggcggacgggtgagtaacgcgtgggcaacctgccttacacagggggataacagctagaaatggctgctaataccgcataagacctcagtaccgcatggtagaggggtaaaaactccggtggtgtaagatgggcccgcgtctgattaggtagttggtagggtaacggcctaccaagccgacgatcagtagccgacctgagagggtgaccggccacattggactgagacacggcccaaactcctacgggaggcagcagtggggaatattgcacaatgggggaaaccctgatgcagcgacgccgcgtgaaggatgaagtatttcggtatgtaaacttctatcagcagggaagaagatgacggtacctgactaagaagccccggctaactacgtgccagcagccgcggtaatacgtagggggcaagcgttatccggatttactgggtgtaaagggagcgtagacggcatggcaagtctgaagtgaaagcccggggctcaaccccgggactgctttggaaactgtcaggctagagtgtcggagaggcaagtggaattcctagtgtagcggtgaaatgcgtagatattaggaggaacaccagtggcgaagcggcttgctggacgatgactgacgttgaggctcgaaagcgtggggagcaaacaggattagataccctggtagtccacgccgtaaacgatgattactaggtgtcgggaagcaaagcttttcggtgccgcagccaacgcaataagtaatccacctggggagtacgttcgcaagaatgaaactcaaaggaattgacggggacccgcacaagcggtggagcatgtggtttaattcgaagcaacgcgaagaaccttacctgatcttgacatcccggtgacaaagtatgtaacgtactctttcttcggaacaccggtgacaggtggtgcatggttgtcgtcagctcgtgtcgtgagatgttgggttaagtcccgcaacggcgcaacccttatctttagtagccagcatttgaggtgggcactctagagagactgccagggataacctggaggaaggtggggatgacgtcaaatcatcatgccccttatgaccagggctacacacgtgctacaatggcgtaaacaaagggaagcgaccctgtgaaggcaagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatggggtcagtaacgcccgaagccggtgacctaaccgcaaaggaggagccgtcgaaggtg

SEQ ID NO: 101 - DNA, 1497 bp, Veillonella parvula
  source 1..1497: mol_type = genomic DNA
gctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatctaacctcggcatcgaggaaagatgaaaggtggcctctatttataagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaaaggccttcttgcgaacagttagaaggattgacggtaccggaatagaaagccacggctaactacgtgccagcagccgcggtaatacgtaggtggcaagcgttgtccggaattattgggcgtaaagcgcgcgcaggcggatcagtyagtctgtcttaaaagttcggggcttaaccccgtgatgggatggaaactgctgatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaacyagagatrgttcctcttcttcggaagccagaaaacaggtggtgcacggttgtcgtcagctcgtgtcgtgagatgttgggttaagtcccgcaacgagcgcaacccctatcttatgttgccagcactttgggtgggaactcatgagagactgccgcagacaatgcggaggaaggcggggatgacgtcaaatcatcatgccccttatgacctgggctacacacgtactacaatgggagttaatagacggaagcgagatcgcgagatggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtgaagtcgtaacaaggtagcc

SEQ ID NO: 102 - DNA, 1465 bp, Longibaculum muris
  source 1..1465: mol_type = genomic DNA
ctggctcaggatgaacgctggcggcgtgcctaatacatgcaagtcgaacgcaccgcttcggtggtgagtggcgaacgggtgagtaatacataagtaacctggcctttcgagggggataactattggaaacgatagctaagaccgcataggcataattctcgcatgagagttatgttaaatatcccaagggatagcgagaggatggacttatggcgcattagctagttggtgagggtaacggcccaccaaggcgacgatgcgtagccgacctgagagggtggacggccacactgggactgagacacggcccagactcctacgggaggcagcagtagggaattttcggcaatgggcgaaagcctgaccgagcaacgccgcgtgagggaggaagtatttcggtatgtaaacctctgttataaaggaagaacggcgcatgtagggaatgacatgcgagtgacggtactttatgagaaagccacggctaactacgtgccagcagccgcggtaatacgtaggtggcgagcgttatccggaatcattgggcgtaaagagggagcaggcggcaactagggtctgcggtaaaagaccgaagctaaacttcggtaagccgtggaaaccgaggagctagagtgcagtagaggatcgtggaattccatgtgtagcggtgaaatgcgtagatatatggaggaacaccagtggcgaaggcgacgatctgggctgcaactgacgctcagtcccgaaagcgtggggagcaaataggattagataccctagtagtccacgccgtaaacgatgagtactaagtgtttgggggtcagacctcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatacccttaaaggctctagagatagagagatagctataggggatacaggtggtgcatggttgtcgtcagctcgtgtcgtgagatgttgggttaagtcccgcaacgagcgcaacccctgttgccagttgccagcattaagttggggactctggcgagactgcctctgcaaggaggaggaaggcggggatgacgtcaaatcatcatgccccttatgacctgggctacacacgtgctacaatggacggatcagagggaagcgaagccgcgaggtggagcgaaacccagaaacccgttctcagttcggactgcagtctgcaactcgactgcacgaagttggaatcgctagtaatcgcgaatcagcatgtcgcggtgaatacgttctcgggccttgtacacaccgcccgtcacaccatgagagttggtaacacccgaagccggtggcctaaccgcaaggaaggagctgtctaaggtgggactgatgattggggtgaagtcgta

SEQ ID NO: 103 - DNA, 1507 bp, unidentified
  misc_feature 1..1507: note = Description of Unknown: Peptostreptococcaceae bacterium sequence
  source 1..1507: mol_type = genomic DNA
gagtttgatcctggctcaggatgaacgctggcggcgtgcttaacacatgcaagtcgagcgagaatgtatggatgacgcttcggtagattccatacaggaaagcggcggacgggtgagtaacgcgtaggcaacctgccctgtacagagggatagcgtcgggaaaccggcattaaaccctcatgataccacacggtcacatggccgagtggtcaaagattcatcggtatgggatgggcctgcgtctgattagttggttggtggggtaacggctcaccaaggcgatgatcagtagccgacctgagagggtgaacggccacattggaactgagacacggtccaaactcctacgggaggcagcagtggggaatattgcacaatgggcgcaagcctgatgcagcaacgccgcgtgagcgaagaaggcctttgggtcgtaaagctctgtcctggggaaagaaagaaatgacggtacccttggaggaagccccggctaactacgtgccagcagccgcggtaatacgtagggggcaagcgttatccggaattattgggcgtaaagagtacgtaggtggtattttaagcgcagggtttaaggcaatggctcaactattgttcgccctgtgaactggaacacttgagtgcaggaggggaaagcggaattcctagtgtagcggtgaaatgcgtagatattaggaggaacaccggtggcgaaggcggctttctggactgtaactgacactgaggtacgaaagcgtgggtagcaaacaggattagataccctggtagtccacgccgtaaacgatgagcactaggtgtcggggtcgcaagacttcggtgccggagttaacgcaataagtgctccgcctggggagtacggccgcaaggctaaaactcaaaggaattgacggggacccgcacaagcagcggagcatgtggtttaattcgaagcaacgcgaagaaccttaccaggacttgacatccttctgacaggcccttaaccgggccctccttcgggcagaagagacaggtggtgcatggttgtcgtcagctcgtgtcgtgagatgttgggttaagtcccgcaacgagcgcaacccttgtcattagttgccatcattaagttgggcactctagtgagactgccggggataactcggaggaaggtggggatgacgtcaaatcatcatgccccttatgttctgggctacacacgtgctacaatggccggcacaacgagaagcgagaccgcgaggtggagcaaatcttctaaaaccggccccagttcggattgtaggctgcaactcgcctacatgaagccggagttgctagtaatcgcggatcagaatgccgcggtgaatgcgttcccgggtcttgtacacaccgcccgtcacaccatggaagttgggagcgcccgaagttggtcaaagaatcgattacctaaggcgaaaccaatgactggggtgaagtcgtaacaaggtagccgtatcggaaggtgcggctggatcacctcctt

SEQ ID NO: 104 - DNA, 1482 bp, Alloscardovia omnicolens
  source 1..1482: mol_type = genomic DNA
gacgaacgctggcggcgtgcttaacacatgcaagtcgaacgggatcctttctagcttgctaggggggtgagagtggcgaacgggtgagtaacgcgtgattaacctgccgtgtagtttggaatagctcctggaaacgggtggtaatgccgaatgttccacatgctcgcatgggtgtgtgggaaaacgttttagtgctatgcgatgggatcgcgtcctatcagcttgttggtggggtagtggcctaccaaggcttcgacgggtagccggcctgagagggcgaccggccacattgggactgagatacggcccagactcctacgggaggcagcagtggggaatattgcacaatggggggaaccctgatgcagcgacgccgcgtgcgggatgacggccttcgggttgtaaaccgcttttattggggagcaagcgtgagtgagtgtaccttttgaataagcaccggctaactacgtgccagcagccgcggtaatacgtagggtgcaagcgttgtccggaattattgggcgtaaagagctcgtaggcggtttgtcgcgtctggtgtgaaagtccatcgcttaacggtggattggcgctgggtacgggcaggctagagtgtagtaggggagactggaattctcggtgtaacggtggaatgtgtagatatcgggaagaacaccaatggcgaaggcaggtctctgggctattactgacgctgaggagcgaaagcgtggggagcgaacaggattagataccctggtagtccacgccgtaaacggtggatgctggatgtggggccctttccacgggttctgtgtcggagctaacgcgttaagcatcccgcctggggagtacggccgcaaggctaaaactcaaagaaattgacgggggcccgcacaagcggcggagcatgcggattaattcgatgcaacgcgaagaaccttaccaagacttgacatgttaacgatggcctcagagatggggtgtctcttcggagcgttttcacaggtggtgcatggtcgtcgtcagctcgtgtcgtgagatgttgggttaagtcccgcaacgagcgcaacccttgtcttgtgttgccagcacgttatggtgggaactcacaagagaccgccggggttaactcggaggaaggtggggatgacgtcagatcatcatgccccttacgtcttgggcttcacgcatgctacaatggccggtacagcgaggtgcgatagggtgacctggagcggatctctgaaaaccggtctcagttcggatcggagcctgcaactcggctccgtgaaggtggagtcgctagtaatcgcagatcagcaacgctgcggtgaatgcgttcccgggccttgtacacaccgcccgtcaagtcatgaaagtgggcagcacccgaagccggtggcctaacagtttttgctgggggagccgtctaaggtgaggttcgcgattgggactaagtcgtaacaaggtagccgtaccggaagg

SEQ ID NO: 105 - DNA, 1542 bp, Lactobacillus salivarius
  source 1..1542: mol_type = genomic DNA
agagtttgatcctggctcaggacgaacgctggcggcgtgcctaatacatgcaagtcgaacgaaactttcttacaccgaatgcttgcrttcatcgtaagaagttgagtggcggacgggtgagtaacacgtgggtaacctgcctaaaagaaggggataacacttggaaacaggtgctaataccgtatatctctaaggatcgcatgatccttagatgaaagatggttctgctatcgcttttagatggacccgcggcgtattaactagttggtggggtaacggcctaccaaggtgatgatacgtagccgaactgagaggttgatcggccacattgggactgagacacggtccaaactcctacgggaggcagcagtagggaatcttccacaatggacgcaagtctgatggagcaacgccgcgtgagtgaagaaggtcttcggatcgtaaaactctgttgttagagaagaacacgagtgagagtaactgttcattcgatgacggtatctaaccagcaagtcacggctaactacgtgccagcagccgcggtaatacgtaggtggcaagcgttgtccggatttattgggcgtaaagggaacgcaggcggtcttttaagtctgatgtgaaagccttcggcttaaccggagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgaatgctaggtgttggagggtttccgcccttcagtgccgcagctaacgcaataagcattccgcctggggagtacgaccgcaaggttgaaactcaaaggaattgacgggggcccgcacaagcggtggagcatgtggtttaattcgaagcaacgcgaagaaccttaccaggtcttgacatcctttgaccacctaagagattaggctttcccttcggggacaaagtgacaggtggtgcatggctgtcgtcagctcgtgtcgtgagatgttgggttaagtcccgcaacgagcgcaacccttgttgtcagttgccagcattaagttgggcactctggcgagactgccggtgacaaaccggaggaaggtggggacgacgtcaagtcatcatgccccttatgacctgggctacacacgtgctacaatggacggtacaacgagtcgcaagaccgcgaggtttagctaatctcttaaagccgttctcagttcggattgtaggctgcaactcgcctacatgaagtcggaatcgctagtaatcgcgaatcagcatgtcgcggtgaatacgttcccgggccttgtacacaccgcccgtcacaccatgagagtttgtaacacccaaagccggtggggtaaccgcaaggagccagccgtctaaggtgggacagatgattggggtgaagtcgtaacaaggtagccgtaggagaacctgcggctg

SEQ ID NO: 106 - DNA, 1529 bp, Clostridium scindens
  source 1..1529: mol_type = genomic DNA
gagagtttgatcctggctcaggatgaacgctggcggcgtgcctaacacatgcaagtcgaacgaagcgcctggccccgacttcttcggaacgaggagccttgcgactgagtggcggacgggtgagtaacgcgtgggcaacctgccttgcactgggggataacagccagaaatggctgctaataccgcataagaccgaagcgccgcatggcgcggcggccaaagccccggcggtgcaagatgggcccgcgtctgattaggtagttggcggggtaacggcccaccaagccgacgatcagtagccgacctgagagggtgaccggccacattgggactgagacacggcccagactcctacgggaggcagcagtggggaatattgcacaatgggggaaaccctgatgcagcgacgccgcgtgaaggatgaagtatttcggtatgtaaacttctatcagcagggaagaagatgacggtacctgactaagaagccccggctaactacgtgccagcagccgcggtaatacgtagggggcaagcgttatccggatttactgggtgtaaagggagcgtagacggcgatgcaagccagatgtgaaagcccggggctcaaccccgggactgcatttggaactgcgtggctggagtgtcggagaggcaggcggaattcctagtgtagcggtgaaatgcgtagatattaggaggaacaccagtggcgaaggcggcctgctggacgatgactgacgttgaggctcgaaagcgtggggagcaaacaggattagataccctggtagtccacgccgtaaacgatgactactaggtgtcgggtggcaaggccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgatcttgacatcccgatgccaaagcgcgtaacgcgctctttcttcggaacatcggtgacaggtggtgcatggttgtcgtcagctcgtgtcgtgagatgttgggttaagtcccgcaacgagcgcaacccctatcttcagtagccagcattttggatgggcactctggagagactgccagggagaacctggaggaaggtggggatgacgtcaaatcatcatgccccttatgaccagggctacacacgtgctacaatggcgtaaacaaagggaggcgaacccgcgagggtgggcaaatcccaaaaataacgtctcagttcggattgtagtctgcaactcgactacatgaagttggaatcgctagtaatcgcgaatcagaatgtcgcggtgaatacgttcccgggtcttgtacacaccgcccgtcacaccatgggagtcagtaacgcccgaagccggtgacccaacccgtaagggagggagccgtcgaaggtgggaccgataactggggtgaagtcgtaacaaggtagccgtatcggaaggtgcggctggatcacctccttc

SEQ ID NO: 107 - DNA, 1454 bp, Clostridium lavalense
  source 1..1454: mol_type = genomic DNA
gatgaacgctggcggcgtgcctaacacatgcaagtcgaacgaagcattttraatgaagttttcggatggattytgagatgactgagtggcggacgggtgagtaacacgtggataacctgcctcacactgggggacaacagttagaaatgactgctaataccgcataagcgcacagtaccgcatggtacagtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagataatgacggtacctgactaagaagccccggctaactacgtgccagcagccgcggtaatacgtagggggcaagcgttatccggatttactgggtgtaaagggagcgtagacggcatggcaagtctgaagtgaaaacccagggctcaaccctgggactgctttggaaactgtcaagctagagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgagtgctaggtgttggggggcaaagcccttcggtgccgtcgcaaacgcaataagcactccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgcctttccttcgggacaagrgagacaggtggtgcatggttgtcgtcagctcgtgtcgtgagatgttgggttaagtcccgcaacgagcgcaacccttatccttagtagccagcartragatgggcactctagggagactgccagggacaacctggaggaaggtggggatgacgtcaaatcatcatgccccttatgatttgggctacacacgtgctacaatggcgtaaacaaagggaagcgaccctgcgaaggtgagcaaatctcaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcargagagggakctgccgaasgcggcgcakgtaactggggtg

SEQ ID NO: 108 - DNA, 1411 bp, Abiotrophia defectiva
  variation 186: note = a, c, t, g, unknown or other
  variation 271: note = a, c, t, g, unknown or other
  variation 872: note = a, c, t, g, unknown or other
  variation 910: note = a, c, t, g, unknown or other
  variation 1142: note = a, c, t, g, unknown or other
  variation 1270: note = a, c, t, g, unknown or other
  source 1..1411: mol_type = genomic DNA
agagtttgatcatggctcaggacgaacgctggcggcgtgcctaatacatgcaagtcgaacgaaccgcgactaggtgcttgcacttggtcaaggtgagtggcgaacgggtgagtaacacgtgggtaacctacctcatagtgggggataacagtcggaaacgactgctaataccgcataggacatggnatcacatgattcagtgaggaaaggtggcgcaagctatcgctaagagatggacccgcggtgcattagctagttggtagggtaaggncctaccaaggcgatgatgcatagccgacctgagagggtgatcggccacattgggactgagacacggcccaaactcctacgggaggcagcagtagggaatcttccgcaatggacgcaagtctgacggagcaacgccgcgtgagtgaagaaggtcttcggatcgtaaagctctgttgttagagaagaacagcgcatagagtaactgctatgcgtgtgacggtatctaaccagaaagccacggctaactacgtgccagcagccgcggtaatacgtaggtggcgagcgttgtccggatttattgggcgtaaagggagtgtaggcggtcttttaagtctgatgtgaaagcccacggctcaaccgtggagggtcattggaaactgggagacttgagtgcagaagaggagagcggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggaggggttccacccttcagtgctggagttaangcaataagcactccgcctggggagtacggccgcaaggntgaaactcaaaggaattgacggggacccgcacaagcggtggagcatgtggtttaattcgaagcaacgcgaagaaccttaccaggtcttgacatcccgacgaccgctctagagatagagttttttttcggaacgtcggtgacaggtggtgcatggttgtcgtcagctcgtgtcgtgagatgttgggttaagtcccgcaacgagcgcaacccctataactagttgccagcattnagatggggactctagttagactgccggtgacaaaccggaggaaggtggggatgacgtcaaatcatcatgccccttatgacctgggctacacacgtgctacaatggatggtacaacgagcagcgaactngcgagggtaagcgaatctctaaaagccattctcagttcggattgtagtctgcaactcgactacatgaagccggaatcgctagtaatcgcggatcagcacgccgcggtgaatacgttcccgggtcttgtacacaccgcccgt

SEQ ID NO: 109 - DNA, 1468 bp, Alistipes putredinis
  variation 2: note = a, c, t, g, unknown or other
  variation 30: note = a, c, t, g, unknown or other
  variation 202: note = a, c, t, g, unknown or other
  variation 339: note = a, c, t, g, unknown or other
  variation 425: note = a, c, t, g, unknown or other
  variation 439: note = a, c, t, g, unknown or other
  variation 527: note = a, c, t, g, unknown or other
  variation 530: note = a, c, t, g, unknown or other
  variation 544: note = a, c, t, g, unknown or other
  variation 637: note = a, c, t, g, unknown or other
  variation 739: note = a, c, t, g, unknown or other
  variation 761..763: note = a, c, t, g, unknown or other
  variation 771: note = a, c, t, g, unknown or other
  variation 867: note = a, c, t, g, unknown or other
  variation 1127: note = a, c, t, g, unknown or other
  variation 1139: note = a, c, t, g, unknown or other
  variation 1204: note = a, c, t, g, unknown or other
  variation 1425: note = a, c, t, g, unknown or other
  source 1..1468: mol_type = genomic DNA
cnatggagagtttgatcctggctcaggatnaacgctagcggcaggcttaacacatgcaagtcgaggggcagcatcatggatagcaatatctatggtcggcgaccggcgcacgggtgcgtaacgcgtatgcaacctacctttaacagggggataacaccgagaaattggtactaataccccataatatcatagaaggcatctnttatggttgaaaattccgatggttagagatgggcatgcgttgtattagctagttggtggggtaacggctcaccaaggcaacgatacatagggggactgagaggttaaccccccacactggtactgagacacggaccngactcctacgggaggcagcagtgaggaatattggtcaatggacgcaagtctgaaccagccatgccgcgtgcaggatgacggctctntgagttgtaaactncttttgtacgagggtaaacgcagatacgtgtatctgtctgaaagtatcgtacgaataaggatcggctaactccgtgccagcagccgcngtnatacggaggattcnagcgttatccggatttattgggtttaaagggtgcgtaggcggtttgataagttagaggtgaaatttcggggctcaaccctgaacgtgcctctnatactgttgagctagagagtagttgcggtaggcggaatgtatggtgtagcggtgaaatgcttagagatcatacagaacaccgattgcgaaggcagcttaccnaactatatctgacgctgatgcnnnaaagcgtngggagcaaacaggattagataccctggtagtccacgcagtaaacgatgataactcgttgtcggcgatacacagtcggtgactaagcgaaagcgatnagttatccacctggggagtacgttcgcaagaatgaaactcaaaggaattgacgggggcccgcacaagcggaggaacatgtggtttaattcgatgatacgcgaggaaccttacccgggcttgaaagttagcgacgattcttgaaagaggatttccttcggggcgcgaaactaggtgctgcatggttgtcgtcagctcgtgccgtgaggtgtcgggttaagtcccataacgagcgcaacccctaccgttagttgccatcagntgaagctgggcnctctggcgggactgccggtgtaagccgagaggaaggtggggatgacgtcaaatcagcacggcccntacgtccggggctacacacgtgttacaatggtaggtacagagggcagctacccagcgatgggatgcgaatctcgaaagcctatctcagttcggattggaggctgaaacccgcctccatgaagttggattcgctagtaatcgcgcatcagccatggcgcggtgaatacgttcccgggccttgtacacaccgcccgtcaagccatgggagccgggggtgcctnaagttcgtgaccgcaaggagcgacctagggcaaaactggtgac

SEQ ID NO: 110 - DNA, 1408 bp, Salinicoccus qingdaonensis
  source 1..1408: mol_type = genomic DNA
tcgagcgcgagattggggagcttgctccccatgatcgagcggcggacgggtgagtaacacgtaggcaacctacccatcagactgggataaccgcgggaaaccgtggctaataccggatgaccctttatgacaccggtcgtaaagttgaaaggcgcttatggtgccgctgatggatgggcctgcggcgcattagctggttggtggggtaacggcccaccaaggcgacgatgcgtagccgacctgagagggtgatcggccacactgggactgagacacggcccagactcctacgggaggcagcagtagggaatcttccgcaatggacgcaagtctgacggagcaacgccgcgtgagtgaagaagggtttcggctcgtaaaactctgttgttaaggaagaatgtcatgtatttaactgatatgtgagtgacggtacttaaccagaaagccacggctaactacgtgccagcagccgcggtaatacgtaggtggcaagcgttatccggaattattgggcgtaaagcgcgcgtaggcggtttcttaagtctgatgtgaaagcccccggctcaaccggggagggtcattggaaactggggaacttgagtgcagaagaggagagtggaattccatgtgtagcggtgaaatgcgcagagatatggaggaacaccagtggcgaaggcggctctctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgaccgccctggagacagggcttcccttcggggcagagagacaggtggtgcatggttgtcgtcagctcgtgtcgtgagatgttgggttaagtcccgcaacgagcgcaacccttaagcttagttgccagcattcagttgggcactctaagttgactgccggtgacaaaccggaggaaggtggggatgacgtcaaatcatcatgccccttatgatttgggctacacacgtgctacaatggacaggttacaaagggcagcgacgccgcaaggccaagcgaatcccataaaactgttctcagttcggattggagtctgcaactcgactccatgaagctggaatcgctagtaatcgtggatcagcatgccacggtgaatacgttcccgggtcttgtacacaccgcccgtcacaccacgaaagtcagtaacacctgaagccggtgggccaaccattatggaggcagc

SEQ ID NO: 111 - DNA, 1494 bp, Parasutterella excrementihominis
  source 1..1494: mol_type = genomic DNA
attgaacgctggcggaacgctttacacatgcaagtcgaacggtaacgcggagagaagcttgcttctctccggcgacgagtggcgaacgggtgagtaatacatcggaacgtgtccgctcgtgggggacaaccagccgaaaggttggctaataccgcatgagttctacggaagaaagagggggacccgcaagggcctctcgcgagcggagcggccgatgactgattagcctgttggtgaggtaacggctcaccaaagcaacgatcagtagctggtctgagaggacgaccagccacactgggactgagacacggcccagactcctacgggaggcagcagtggggaattttggacaatgggcgcaagcctgatccagctattccgcgtgtgggatgaaggccctcgggttgtaaaccacttttgtagagaacgaaaagacaccttcgaataaagggtgttgctgacggtactctaagaataagcaccggctaactacgtgccagcagccgcggtaatacgtagggtgcgagcgttaatcggaattactgggcgtaaagggtgcgcaggcggttgagtaagacagatgtgaaatccccgagcttaactcgggaatggcatatgtgactgctcgactagagtgtgtcagagggaggtggaattccacgtgtagcagtgaaatgcgtagatatgtggaagaacaccgatggcgaaggcagcctcctgggacataactgacgctcaggcacgaaagcgtggggagcaaacaggattagataccctggtagtccacgccctaaacgatgttaactagttgttgggaagtaaaattctcagtaacgcagccaacgcgagaagttaaccgcctgggaagtacggtcgcaagactaaaactcaaaggaattgacggggacccgcacaagcggtggatgatgtggattaattcgatgcaacgcgaaaaaccttacctacccttgacatgtcaggaagctcttgtaatgagagcgtgcccgcaagggagcctgaacacaggtgctgcatggctgtcgtcagctcgtgtcgtgagatgttgggttaagtcccgcaacgagcgcaacccttgtcactagttgctacgaaagggcactctagtgagactgccggtgacaaaccggaggaaggtggggatgacgtcaagtcctcatggcccttatgggtagggcttcacacgtcatacaatggtcggaacagagggcagcgaagccgtgaggcggagccaatcccagaaaaccgatcgtagtccggattgcagtctgcaactcgactgcatgaagtcggaatcgctagtaatcgcggatcagcatgccgcggtgaatacgttcccgggtcttgtacacaccgcccgtcaaacaatgggagtggtgtttaccagaagtcgttagcctaaccgcaaggagggcggcgaccacggtgagcaccgtgactaatgttaagtcgtaacaaggtagccgtaccggaagg

SEQ ID NO: 112 - DNA, 1422 bp, Bacteroides caccae
  variation 179..181: note = a, c, t, g, unknown or other
  variation 901: note = a, c, t, g, unknown or other
  variation 1006: note = a, c, t, g, unknown or other
  variation 1420: note = a, c, t, g, unknown or other
  source 1..1422: mol_type = genomic DNA
atgaacgctagctacaggcttaacacatgcaagtcgaggggcatcagtttgtttgcttgcaaacaaacgctggcgaccggcgcacgggtgagtaacacgtatccaacctacctcatactcggggatagcctttcgaaagaaagattaatatccgatagcatatatttcccgcatgggtnnnatattaaagaaattcggtatgagatggggatgcgttccattagtttgttgggggggtaacggcccaccaagactacgatggataggggttctgagaggaaggtcccccacattggaactgagacacggtccaaactcctacgggaggcagcagtgaggaatattggtcaatggacgcgagtctgaaccagccaagtagcgtgaaggatgactgccctatgggttgtaaacttcttttatatgggaataaagtggtccacgtgtggacttttgtatgtaccatatgaataaggatcggctaactccgtgccagcagccgcggtaatacggaggatccgagcgttatccggatttattgggtttaaagggagcgtaggcggattgttaagtcagttgtgaaagtttgcggctcaaccgtaaaattgcagttgatactggcagtcttgagtgcagtagaggtgggcggaattcgtggtgtagcggtgaaatgcttagatatcacgaagaactccgattgcgaaggcagctcactggagtgtaactgacgctgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggccngcacaagcggaggaacatgtggtttaattcgatgatacgcgaggaaccttacccgggcttaaattgcaaatgaattatggggaaacccatacgccgcaaggcatntgtgaaggtgctgcatggttgtcgtcagctcgtgccgtgaggtgtcggcttaagtgccataacgagcgcaacccttatcttcagttactaacaggtcatgctgaggactctggagagactgccgtcgtaagatgtgaggaaggtggggatgacgtcaaatcagcacggcccttacgtccggggctacacacgtgttacaatggggggtacagaaggcagctacctggtgacaggatgccaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagngt

SEQ ID NO: 113 - DNA, 1556 bp, Streptococcus anginosus
  variation 5: note = a, c, t, g, unknown or other
  variation 354: note = a, c, t, g, unknown or other
  variation 372..374: note = a, c, t, g, unknown or other
  source 1..1556: mol_type = genomic DNA
ggccngtagagtttgatcctggttcaggacgaacgctggcggcgtgcctaatacatgcaagtaggacgcacagtttataccgtagcttgctacaccatagactgtgagttgcgaacgggtgagtaacgcgtaggtaacctgcctattagagggggataactattggaaacgatagctaataccgcataacagtatgtaacacatgttagatgcttgaaagatgcaattgcatcgctagtagatggacctgcgttgtattagctagtaggtagggtaatggcctacctaggcgacgatacatagccgacctgagagggtgatcggccacactgggactgagacacggcccagacncctacgggaggcagcagnnnggaatcttcggcaatggggggaaccctgaccgagcaacgccgcgtgagtgaagaaggttttcggatcstaaagctctgttgttaaggaagaacgagtgtgagaatggaaagttcatactgtgacggtacttaaccagaaagggacggctaactacgtgccagcagccgcggtaatacgtaggtcccgagcgttgtccggatttattgggcgtaaagcgagcgcaggcggttagaaaagtctgaagtgaaaggcagtggctcaaccattgtaggctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttgggtcctttccgggactcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctatttctagagataggaagtttcttcggaacatcggtgacaggtggtgcatggttgtcgtcagctcgtgtcgtgagatgttgggttaagtcccgcaacgagcgcaacccttattgttagttgccatcattaagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctctgaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccgtaaggagccagccgcctaaggtgggatagatgattggggtgaagtcgtaacaaggtagccgtatcggaaggtgcggctggatcacctccttg

SEQ ID NO: 114 - DNA, 1362 bp, Haemophilus parainfluenzae
  source 1..1362: mol_type = genomic DNA
attgaacgctggcggcaggcttaacacatgcaagtcgaacggtaacatraagaagcttgcttctttgatgacgagtggcggacgggtgagtaatgcttgggaatctrgcttatggagggggataactacgggaaactgtagctaataccgcgtagwatcgrragatgaaagtgtgggaccttcgggccacatgccataggatgagcccaagtgggattaggtagttggtgaggtaaaggctcaccaagccgacgatctctagctggtctgagaggatgaccagccacactgggactgagacacggcccagactcctacgggaggcagcagtggggaatattgcgcaatgggggcaaccctgacgcagccatgccgcgtgaatgaagaaggccttcgggttgtaaagttctttcggtagcgaggaaggcatttagtttaatagactaggtgattgacgttaactacagaagaagcaccggctaactccgtgccagcagccgcggtaatacggagggtgcgagcgttaatcggaataactgggcgtaaagggcacgcaggcggtgacttaagtgaggtgtgaaagccccgggcttaacctgggaattgcatttcatactgggtcgctagagtactttagggaggggtagaattccacgtgtagcggtgaaatgcgtagagatgtggaggaataccgaaggcgaaggcagccccttgggaatgtactgacgctcatgtgcgaaagcgtggggagcaaacaggattagataccctggtagtccacgctgtaaacgatgtcgatttgggggttgagctttaagyttggcgcccgtagctaacgtgataaatcgaccgcctggggagtacggccgcaaggttaaaactcaaatgaattgacgggggcccgcacaagcggtggagcatgtggtttaattcgatgcaacgcgaagaaccttacctactcttgacatccagagaacattccagagatggattggtgccttcgggaactctgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagtagggctacacacgtgctacaatggcgtatacagagggaagcgagagtgcgagctggagcgaatctcacaaagtacgtctaagtccggattggagtctgcaactcgactccatgaagtcggaatcgctagtaatcgcaaatcagaatgttgcggtgaatacgttcccgggcctt

SEQ ID NO: 115 - DNA, 1395 bp, Clostridium aldenense
  source 1..1395: mol_type = genomic DNA
tcctggttgactgagtggcggacgggtgagtaacgcgtggataacctgcctcacactgggggataacagttagaaatggctgctaataccgcataagcgcacagtaccgcatggtacggtgtgaaaaacccaggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgcaagcctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgacgcaagtctggagtgaaagcccggggcccaaccccgggactgctttggaaactgtgttgctggagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatgctaggtgtcggggggcaaagcccttcggtgccgccgctaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggcacgtaacggtgcccttccttcgggacaggggagacaggtggtgcatggttgtcgtcagctcgtgtcgtgagatgttgggttaagtcccgcaacgagcgcaacccttatccttagtagccagcacgtcgtggtgggcactctagggagactgccagggataacctggaggaaggtggggatgacgtcaaatcatcatgccccttatgatttgggctacacacgtgctacaatggcgtaaacaaagggaggcgaccctgcgaaggcaagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagcaacgcccgaagtcagtgacccaaccttaacaggagggagctgccgaaggcggggcaggtaactggggtgaagtcgtaac

SEQ ID NO: 116 - DNA, 331 bp, Klebsiella sp.
  source 1..331: mol_type = genomic DNA
agcgcaggcggtttcttaagtctgatgtgaaatccccgggcttaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgatttggaggttgtgcccttgaggcgtggcttccggagctaacgcgttaaatcgaccgcctggggagtacggccgcaaggttaaa

SEQ ID NO: 117 - DNA, 331 bp, Klebsiella sp.
  source 1..331: mol_type = genomic DNA
agcgcaggcgggaagacaagttggaagtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgatttggaggttgtgcccttgaggcgtggcttccggagctaacgcgttaaatcgaccgcctggggagtacggccgcaaggttaaa

SEQ ID NO: 118 - DNA, 331 bp, Klebsiella sp.
  source 1..331: mol_type = genomic DNA
agcgtagacggtgtggcaagtctgatgtgaaaggcatgggcttaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgatttggaggttgtgcccttgaggcgtggcttccggagctaacgcgttaaatcgaccgcctggggagtacggccgcaaggttaaa

(19) United States
(12) Patent Application Publication
(10) Pub. No.: US 2023/0151430 A1
(43) Pub. Date: May 18, 2023

(54) METHODS AND COMPOSITIONS FOR IDENTIFYING AND TREATING SUBJECTS AT RISK OF POOR CANCER SURVIVAL

(71) Applicant: MEMORIAL SLOAN-KETTERING CANCER CENTER, New York, NY (US)

(72) Inventors: Jonathan U. Peled, Roosevelt Island, NY (US); Marcel R.M. Van Den Brink, New York, NY (US); Antonio Gomes, New York, NY (US)

(73) Assignee: MEMORIAL SLOAN-KETTERING CANCER CENTER, New York, NY (US)

(21) Appl. No.: 17/890,694

(22) Filed: Aug. 18, 2022

Related U.S. Application Data
(63) Continuation of application No. PCT/US2021/018582, filed on Feb. 18, 2021.
(60) Provisional application No. 62/977,908, filed on Feb. 18, 2020.

(51) Int. Cl. C12Q 1/6886; C12Q 1/689

(52) U.S. Cl. CPC C12Q 1/6886; C12Q 1/689

(57) ABSTRACT

The present disclosure relates to compositions and methods for predicting cancer survival in a subject after receiving a treatment (e.g., allogeneic hematopoietic-cell transplantation). The present disclosure further discloses compositions and methods for treating said subject.

### CROSS-REFERENCE TO RELATED APPLICATIONS

This application is a continuation application of International Patent Application No. PCT/US2021/018582, filed Feb. 18, 2021, which claims priority to U.S. Provisional Patent Application Ser. No. 62/977,908, filed Feb. 18, 2020, the contents of which are incorporated by reference herein in their entireties.

### STATEMENT OF GOVERNMENT INTEREST

This invention was made with government support under CA228308 and HL143189 awarded by the National Institutes of Health. The government has certain rights in the invention.

### SEQUENCE LISTING

The specification further incorporates by reference the Sequence Listing submitted herewith via EFS on Aug. 18, 2022. Pursuant to 37 C.F.R. § 1.52(e)(5), the Sequence Listing file, identified as 072734_1391.xml, is 363,511 bytes and was created on Aug. 18, 2022. The Sequence Listing, electronically filed herewith, does not extend beyond the scope of the specification, and thus does not contain new matter.

### 1. INTRODUCTION

The present disclosure relates to compositions and methods for predicting cancer survival in a subject receiving a treatment (e.g., an allogeneic hematopoietic-cell transplantation). The present disclosure further discloses compositions, e.g., pharmaceutical compositions, and methods for treating said subject.

### 2. BACKGROUND

Allogeneic hematopoietic-cell transplantation (allo-HCT) is a curative therapy for hematological malignancies in which a patient receives a cytotoxic conditioning regimen followed by infusion of hematopoietic precursor cells from a genetically matched donor. Complications such as graft-vs-host disease (GVHD) remain a major cause of morbidity and mortality, limiting the broader applicability of allo-HCT. Allo-HCT patients can exhibit microbiota injury characterized by dramatic expansions of potentially pathogenic bacteria and loss of α-diversity—a parameter that considers the number of unique bacterial taxa present and their relative frequencies.

### 3. SUMMARY

The present disclosure is partly based on the discovery that the likelihood of cancer survival after allogeneic hematopoietic-cell transplantation (allo-HCT) is associated with the presence and/or absence and the relative abundance of specific taxonomic groups (e.g., a phylum, a class, an order, a family, a genus, a species, or a strain) of bacteria. Surprisingly, this association is generalizable across transplant centers and geographical location. Accordingly, the present disclosure relates to compositions and methods for determining the likelihood of survival (e.g., cancer survival) in a subject after a treatment (e.g., allo-HCT) as well as compositions and methods for increasing the likelihood of survival in a subject after the treatment. In certain non-limiting embodiments, the present invention further provides for compositions and methods for treating a subject determined to not likely to exhibit survival, e.g., cancer survival. In certain embodiments, the subject has a cancer, e.g., leukemia. In certain embodiments, the subject has a non-cancer disease, such as aplastic anemia, immune-deficiency conditions, inborn errors of metabolism, or sickle-cell disease.

In certain embodiments, the present disclosure provides a method for treating a subject having a cancer, wherein the method comprises: (a) determining a level of a diagnostic bacterium or a spore thereof in a sample of the subject; (b) comparing the level of the diagnostic bacterium or spores thereof to a reference diagnostic bacterium or a spore thereof level; (c) identifying the subject as not likely to exhibit cancer survival if the level of the diagnostic bacterium or spore thereof is lower than the reference diagnostic bacterium or spore thereof; and (d) treating the subject identified as not likely to exhibit cancer survival with a cancer treatment. In certain embodiments, the diagnostic bacterium is a bacterium of the taxonomic group selected from the group consisting of Clostridia, Streptococcaceae, Lactobacillaceae, Actinobacteria, Pediococcus acidilactici, Erysipelotrichales, Coprobacillaceae, Clostridiaceae, Akkermansia muciniphila, Escherichia coli, Streptococcus mitis, Veillonellaceae, Bilophila, Parascardovia, Streptococcus salivarius, Coriobacteriaceae, Peptostreptococcaceae, Phascolarctobacterium faecium, Lactobacillus plantarum, Clostridium tertium, Eubacterium biforme, Alphaproteobacteria, Ruminococcaceae, Gemella haemolysans, Dorea, Coprococcus, Clostridium, Rothia, Megasphaera, Atopobium, Clostridium glycolicum, Coprococcus comes, Clostridium spiroforme, Lactobacillus fermentum, Clostridium sp. 826, Lactobacillus animalis, Ruminococcus (family Lachnospiraceae), Streptococcus parasanguinis, Clostridium sp. ID5, Granulicatella adiacens, Actinomyces graevenitzii, Clostridium bartlettii DSM 16795, S24-7, Clostridium algidixylanolyticum, Lactobacillus homohiochii, Tyzzerella nexilis, Mogibacterium neglectum, Lactobacillus reuteri, Clostridium saccharogumia, [Eubacterium] hallii, Drancourtella massiliensis, Clostridium perfringens, Bacteroides stercoris ATCC 43183, Ruminococcus faecis, Clostridium sp. PPf35E6, Bacteroides uniformis, Weissella confusa, Megasphaera micronuciformis, Propionibacterium, Peptostreptococcus sp. MDA2346-2, Bacteroides fragilis, Oscillospira, Lactobacillus gasseri, Rothia dentocariosa, Clostridium sporosphaeroides, Lactococcus, Clostridium hathewayi, Clostridium methylpentosum DSM 5476, Clostridium sp. cTPY-17, Clostridium sp. Enrichment culture clone NHT38, Ruminococcus torques, Clostridium sp. cf3-PUG, Lactobacillus buchneri, mitochondria, Coprococcus, Bacillaceae, Fusobacterium nucleatum, Eggerthella lenta, Anaerococcus, Clostridium sp. Culture-54, Anaerostipes caccae, Blautia faecis, Actinobaculum massiliense, Enterococcus gallinarum, Clostridium sp. MSTE9, ambiguous Leuconostoc, Paraprevotellaceae, Roseburia, unclassified Peptostreptococcaceae, Fusobacterium, Atopobium rimae, Blautia luti, Propionibacterium propionicum, Streptococcus lutetiensis, Scardovia inopinata, Dorea formicigenerans, Corynebacterium pseudogenitalium, Turicibacter sanguinis, Roseburia faecis, Clostridium paraputrificum, Fusobacteria, Clostridium symbiosum, Propionibacterium freudenreichii, and any combinations thereof; or the diagnostic bacterium or spore thereof comprises a 16S rRNA gene having a nucleotide sequence that has between about 90 and 100% homology to the nucleotide sequence set forth in any one of SEQ ID NOs:1-69.

In certain embodiments, the present disclosure provides a method for treating a subject having a cancer, wherein the method comprises: (a) determining a level of a diagnostic bacterium or a spore thereof in a sample of the subject; (b) comparing the level of the diagnostic bacterium or spores thereof to a reference diagnostic bacterium or a spore thereof level; (c) identifying the subject as not likely to exhibit cancer survival if the level of the diagnostic bacterium or spore thereof is higher than the reference diagnostic bacterium or spore thereof; (d) treating the subject identified as not likely to exhibit cancer survival with a cancer treatment. In certain embodiments, the diagnostic bacterium is a bacterium of the taxonomic group selected from the group consisting of Firmicutes, Bacilli, Enterococcus, Bacillales, ambiguous Klebsiella, Lactobacillus delbrueckii subsp. Bulgaricus, Coprobacillus, Parabacteroides, Ruminococcus, Streptococcus mutans, Enterococcus rivorum, Enterobacter ludwigii, Clostridium leptum, Enterococcus lactis, Bifidobacterium dentium, Eubacterium limosum, Proteobacteria, Mycoplasma, Holdemania filiformis, Lactococcus piscium, Blautia, Bacteroides thetaiotaomicron, Massiliomicrobiota timonensis, Blautia hydrogenotrophica, Enterococcus mundtii, Prevotella melaninogenica, Erwinia chrysanthemi, Clostridium nexile DSM 1787, Veillonella, Clostridium difficile, [Ruminococcus] obeum, Streptophyta, Bacteroidales, Parascardovia, Clostridium clostridioforme, Blautia obeum, Klebsiella oxytoca, Bulleidia moorei, Parabacteroides merdae, Shuttleworthia satelles, Streptococcus sp. DN812, Clostridium cellulosi, Lactobacillus acidophilus, Bacteroides ovatus, Clostridium hylemonae, Veillonella parvula, Longibaculum muris, Butyrivibrio, Peptostreptococcaceae bacterium canine oral taxon 074, Alloscardovia omnicolens, Lactobacillus salivarius, Clostridium scindens, Alistipes, Clostridium lavalense, Anaerostipes, Abiotrophia defectiva, Leuconostocaceae, Dorea, Alistipes putredinis, Salinicoccus qingdaonensis, Parasutterella excrementihominis, Bacteroides caccae, Bifidobacteriaceae, Streptococcus anginosus, Haemophilus parainfluenzae, Oscillospira, Clostridium aldenense, and any combination thereof, or the diagnostic bacterium or spore thereof comprises a 16S rRNA gene sequence that has between about 90 and 100% homology to the nucleotide sequence set forth in any one of SEQ ID NOs:70-118.

In certain embodiments, the present disclosure provides a method for treating a subject having a cancer, wherein the method comprises administering a cancer treatment to the subject, wherein the subject is identified as not likely to exhibit cancer survival by determining that a level of a diagnostic bacterium or a spore thereof is lower than a reference diagnostic bacterium or a spore thereof, wherein the diagnostic bacterium is a bacterium of the taxonomic group selected from the group consisting of Clostridia, Streptococcaceae, Lactobacillaceae, Actinobacteria, Pediococcus acidilactici, Erysipelotrichales, Coprobacillaceae, Clostridiaceae, Akkermansia muciniphila, Escherichia coli, Streptococcus mitis, Veillonellaceae, Bilophila, Parascardovia, Streptococcus salivarius, Coriobacteriaceae, Peptostreptococcaceae, Phascolarctobacterium faecium, Lactobacillus plantarum, Clostridium tertium, Eubacterium biforme, Alphaproteobacteria, Ruminococcaceae, Gemella haemolysans, Dorea, Coprococcus, Clostridium, Rothia, Megasphaera, Atopobium, Clostridium glycolicum, Coprococcus comes, Clostridium spiroforme, Lactobacillus fermentum, Clostridium sp. 826, Lactobacillus animalis, Ruminococcus (family Lachnospiraceae), Streptococcus parasanguinis, Clostridium sp. ID5, Granulicatella adiacens, Actinomyces graevenitzii, Clostridium bartlettii DSM 16795, S24-7, Clostridium algidixylanolyticum, Lactobacillus homohiochii, Tyzzerella nexilis, Mogibacterium neglectum, Lactobacillus reuteri, Clostridium saccharogumia, [Eubacterium] hallii, Drancourtella massiliensis, Clostridium perfringens, Bacteroides stercoris ATCC 43183, Ruminococcus faecis, Clostridium sp. PPf35E6, Bacteroides uniformis, Weissella confusa, Megasphaera micronuciformis, Propionibacterium, Peptostreptococcus sp. MDA2346-2, Bacteroides fragilis, Oscillospira, Lactobacillus gasseri, Rothia dentocariosa, Clostridium sporosphaeroides, Lactococcus, Clostridium hathewayi, Clostridium methylpentosum DSM 5476, Clostridium sp. cTPY-17, Clostridium sp. Enrichment culture clone NHT38, Ruminococcus torques, Clostridium sp. cf3-PUG, Lactobacillus buchneri, mitochondria, Coprococcus, Bacillaceae, Fusobacterium nucleatum, Eggerthella lenta, Anaerococcus, Clostridium sp. Culture-54, Anaerostipes caccae, Blautia faecis, Actinobaculum massiliense, Enterococcus gallinarum, Clostridium sp. MSTE9, ambiguous Leuconostoc, Paraprevotellaceae, Roseburia, unclassified Peptostreptococcaceae, Fusobacterium, Atopobium rimae, Blautia luti, Propionibacterium propionicum, Streptococcus lutetiensis, Scardovia inopinata, Dorea formicigenerans, Corynebacterium pseudogenitalium, Turicibacter sanguinis, Roseburia faecis, Clostridium paraputrificum, Fusobacteria, Clostridium symbiosum, Propionibacterium freudenreichii, and any combinations thereof; or the diagnostic bacterium or spore thereof comprises a 16S rRNA gene having a nucleotide sequence that has between about 90 and 100% homology to the nucleotide sequence set forth in any one of SEQ ID NOs:1-69.

In certain embodiments, the present disclosure provides a method for treating a subject having a cancer, wherein the method comprises administering a cancer treatment (e.g., allo-HCT) to the subject, wherein the subject is identified as not likely to exhibit cancer survival by determining that a level of a diagnostic bacterium or a spore thereof is higher than a reference diagnostic bacterium or a spore thereof, wherein the diagnostic bacterium is a bacterium of the taxonomic group selected from the group consisting of Firmicutes, Bacilli, Enterococcus, Bacillales, ambiguous Klebsiella, Lactobacillus delbrueckii subsp. Bulgaricus, Coprobacillus, Parabacteroides, Ruminococcus, Streptococcus mutans, Enterococcus rivorum, Enterobacter ludwigii, Clostridium leptum, Enterococcus lactis, Bifidobacterium dentium, Eubacterium limosum, Proteobacteria, Mycoplasma, Holdemania filiformis, Lactococcus piscium, Blautia, Bacteroides thetaiotaomicron, Massiliomicrobiota timonensis, Blautia hydrogenotrophica, Enterococcus mundtii, Prevotella melaninogenica, Erwinia chrysanthemi, Clostridium nexile DSM 1787, Veillonella, Clostridium difficile, [Ruminococcus] obeum, Streptophyta, Bacteroidales, Parascardovia, Clostridium clostridioforme, Blautia obeum, Klebsiella oxytoca, Bulleidia moorei, Parabacteroides merdae, Shuttleworthia satelles, Streptococcus sp. DN812, Clostridium cellulosi, Lactobacillus acidophilus, Bacteroides ovatus, Clostridium hylemonae, Veillonella parvula, Longibaculum muris, Butyrivibrio, Peptostreptococcaceae bacterium canine oral taxon 074, Alloscardovia omnicolens, Lactobacillus salivarius, Clostridium scindens, Alistipes, Clostridium lavalense, Anaerostipes, Abiotrophia defectiva, Leuconostocaceae, Dorea, Alistipes putredinis, Salinicoccus qingdaonensis, Parasutterella excrementihominis, Bacteroides caccae, Bifidobacteriaceae, Streptococcus anginosus, Haemophilus parainfluenzae, Oscillospira, Clostridium aldenense, and any combination thereof, or the diagnostic bacterium or spore thereof comprises a 16S rRNA gene sequence that has between about 90 and 100% homology to the nucleotide sequence set forth in any one of SEQ ID NOs:70-118.

In certain embodiments, the subject has previously received a hematopoietic cell transplantation (HCT). In certain embodiments, the cancer treatment is a hematopoietic cell transplantation (HCT). In certain embodiments, the HCT is an allogenic hematopoietic cell transplantation (allo-HCT). In certain embodiments, the allo-HCT is a T-cell replete allo-HCT.

In certain embodiments, the level of the diagnostic bacterium or spore thereof is the relative abundance of the diagnostic bacterium or spores thereof as compared to other bacteria in the sample. In certain embodiments, the sample is a fecal sample or an intestinal content sample of the subject.

In certain embodiments, the cancer treatment comprises administering to the subject a therapeutic bacterium or a spore thereof or a pharmaceutical composition comprising thereof, a hematopoietic cell transplantation (HCT), surgery, radiation therapy, chemotherapy, immunotherapy, stem cell therapy, cellular therapy, a probiotic bacteria, a probiotic yeast, a prebiotic, a postbiotic, an antibiotic, or a combination thereof.

In certain embodiments, the therapeutic bacterium is a bacterium of the taxonomic group selected from the group consisting of Clostridia, Streptococcaceae, Lactobacillaceae, Actinobacteria, Pediococcus acidilactici, Erysipelotrichales, Coprobacillaceae, Clostridiaceae, Akkermansia muciniphila, Escherichia coli, Streptococcus mitis, Veillonellaceae, Bilophila, Parascardovia, Streptococcus salivarius, Coriobacteriaceae, Peptostreptococcaceae, Phascolarctobacterium faecium, Lactobacillus plantarum, Clostridium tertium, Eubacterium biforme, Alphaproteobacteria, Ruminococcaceae, Gemella haemolysans, Dorea, Coprococcus, Clostridium, Rothia, Megasphaera, Atopobium, Clostridium glycolicum, Coprococcus comes, Clostridium spiroforme, Lactobacillus fermentum, Clostridium sp. 826, Lactobacillus animalis, Ruminococcus (family Lachnospiraceae), Streptococcus parasanguinis, Clostridium sp. ID5, Granulicatella adiacens, Actinomyces graevenitzii, Clostridium bartlettii DSM16795, S24-7, Clostridium algidixylanolyticum, Lactobacillus homohiochii, Tyzzerella nexilis, Mogibacterium neglectum, Lactobacillus reuteri, Clostridium saccharogumia, [Eubacterium] hallii, Drancourtella massiliensis, Clostridium perfringens, Bacteroides stercoris ATCC 43183, Ruminococcus faecis, Clostridium sp. PPf35E6, Bacteroides uniformis, Weissella confusa, Megasphaera micronuciformis, Propionibacterium, Peptostreptococcus sp. MDA2346-2, Bacteroides fragilis, Oscillospira, Lactobacillus gasseri, Rothia dentocariosa, Clostridium sporosphaeroides, Lactococcus, Clostridium hathewayi, Clostridium methylpentosum DSM 5476, Clostridium sp. cTPY-17, Clostridium sp. Enrichment culture clone NHT38, Ruminococcus torques, Clostridium sp. cf3-PUG, Lactobacillus buchneri, mitochondria, Coprococcus, Bacillaceae, Fusobacterium nucleatum, Eggerthella lenta, Anaerococcus, Clostridium sp. Culture-54, Anaerostipes caccae, Blautia faecis, Actinobaculum massiliense, Enterococcus gallinarum, Clostridium sp. MSTE9, ambiguous Leuconostoc, Paraprevotellaceae, Roseburia, unclassified Peptostreptococcaceae, Fusobacterium, Atopobium rimae, Blautia luti, Propionibacterium propionicum, Streptococcus lutetiensis, Scardovia inopinata, Dorea formicigenerans, Corynebacterium pseudogenitalium, Turicibacter sanguinis, Roseburia faecis, Clostridium paraputrificum, Fusobacteria, Clostridium symbiosum, Propionibacterium freudenreichii, and any combinations thereof.

In certain embodiments, the therapeutic bacterium or spore thereof is administered in an amount effective to increase the amount of the diagnostic bacterium or spore thereof of Group 2 in the subject.

In certain embodiments, the therapeutic bacterium or spore thereof is administered in an amount effective to decrease the amount of diagnostic bacterium or spore thereof of Group 3 in the subject.

In certain embodiments, the therapeutic bacterium or spore thereof is administered in an amount effective to increase microbiota diversity in the subject.

In certain embodiments, the cancer treatment comprises a combination of administering to the subject the therapeutic bacterium or spore thereof or a pharmaceutical composition comprising thereof; and the HCT. In certain embodiments, the therapeutic bacterium or spore thereof or a pharmaceutical composition comprising thereof is administered to the subject before or after the HCT. In certain embodiments, the HCT is an allogenic hematopoietic cell transplantation (allo-HCT). In certain embodiments, the allo-HCT is a T-cell replete allo-HCT.

In certain embodiments, the cancer treatment comprises administering an antibiotic to the subject.

In certain embodiments, the antibiotic selectively kills or inhibits the growth of the diagnostic bacterium or spore thereof of Group 3. In certain embodiments, the antibiotic does not selectively kill or inhibit the growth of the diagnostic bacterium or spore thereof of Group 2.

In certain embodiments, the method increases the likelihood of cancer survival in the subject.

In certain embodiments, the method: (i) increases the amount of the diagnostic bacterium or spore thereof of Group 2 in the subject; (ii) increases the proliferation or growth of the diagnostic bacterium or spore thereof of Group 2 in the subject; (iii) decreases the amount of the diagnostic bacterium or spore thereof of Group 3 in the subject; and/or (iv) decreases the proliferation or growth of the diagnostic bacterium or spore thereof of Group 3 in the subject.

In certain embodiments, the cancer survival is the survival of the subject at least about 2 years following the cancer treatment.

In certain embodiments, the present disclosure provides a pharmaceutical composition comprising a therapeutic bacterium or a spore thereof, wherein: (i) the bacterium is a bacterium of the taxonomic group selected from the group consisting of Clostridia, Streptococcaceae, Lactobacillaceae, Actinobacteria, Pediococcus acidilactici, Erysipelotrichales, Coprobacillaceae, Clostridiaceae, Akkermansia muciniphila, Escherichia coli, Streptococcus mitis, Veillonellaceae, Bilophila, Parascardovia, Streptococcus salivarius, Coriobacteriaceae, Peptostreptococcaceae, Phascolarctobacterium faecium, Lactobacillus plantarum, Clostridium tertium, Eubacterium biforme, Alphaproteobacteria, Ruminococcaceae, Gemella haemolysans, Dorea, Coprococcus, Clostridium, Rothia, Megasphaera, Atopobium, Clostridium glycolicum, Coprococcus comes, Clostridium spiroforme, Lactobacillus fermentum, Clostridium sp. 826, Lactobacillus animalis, Ruminococcus (family Lachnospiraceae), Streptococcus parasanguinis, Clostridium sp. ID5, Granulicatella adiacens, Actinomyces graevenitzii, Clostridium bartlettii DSM 16795, S24-7, Clostridium algidixylanolyticum, Lactobacillus homohiochii, Tyzzerella nexilis, Mogibacterium neglectum, Lactobacillus reuteri, Clostridium saccharogumia, [Eubacterium] hallii, Drancourtella massiliensis, Clostridium perfringens, Bacteroides stercoris ATCC 43183, Ruminococcus faecis, Clostridium sp. PPf35E6, Bacteroides uniformis, Weissella confusa, Megasphaera micronuciformis, Propionibacterium, Peptostreptococcus sp. MDA2346-2, Bacteroides fragilis, Oscillospira, Lactobacillus gasseri, Rothia dentocariosa, Clostridium sporosphaeroides, Lactococcus, Clostridium hathewayi, Clostridium methylpentosum DSM 5476, Clostridium sp. cTPY-17, Clostridium sp. Enrichment culture clone NHT38, Ruminococcus torques, Clostridium sp. cf3-PUG, Lactobacillus buchneri, mitochondria, Coprococcus, Bacillaceae, Fusobacterium nucleatum, Eggerthella lenta, Anaerococcus, Clostridium sp. Culture-54, Anaerostipes caccae, Blautia faecis, Actinobaculum massiliense, Enterococcus gallinarum, Clostridium sp. MSTE9, ambiguous Leuconostoc, Paraprevotellaceae, Roseburia, unclassified Peptostreptococcaceae, Fusobacterium, Atopobium rimae, Blautia luti, Propionibacterium propionicum, Streptococcus lutetiensis, Scardovia inopinata, Dorea formicigenerans, Corynebacterium pseudogenitalium, Turicibacter sanguinis, Roseburia faecis, Clostridium paraputrificum, Fusobacteria, Clostridium symbiosum, Propionibacterium freudenreichii, and any combinations thereof; or (b) the bacterium or spore thereof comprises a 16S rRNA gene sequence that has between about 90 and 100% homology to the nucleotide sequence set forth in any one of SEQ ID NOs:1-69.

In certain embodiments, the presently disclosed pharmaceutical composition further comprises a biocompatible pharmaceutical carrier. In certain embodiments, the pharmaceutical composition is formulated for oral, nasogastric, rectal, percutaneous (e.g., G tube), orogastric tube, or other enteral routes of administration. In certain embodiments, the presently disclosed pharmaceutical composition further comprises a probiotic bacteria, a probiotic yeast, a prebiotic, a postbiotic, an antibiotic, or a combination thereof. In certain embodiments, the pharmaceutical composition is in a form of a liquid, a suspension, a dried powder, a tablet, a capsule, a food product, or a combination thereof.

In certain embodiments, the bacterium or spore thereof is a recombinant bacterium, or a progeny thereof. In certain embodiments, the bacterium or spore thereof comprises an exogenous nucleic acid encoding a protein that confers antibiotic sensitivity or resistance to the bacterium or spore thereof.

In certain embodiments, the pharmaceutical composition comprises the bacterium or spore thereof in an amount that increases the likelihood of cancer survival in a subject administered the pharmaceutical composition.

In certain embodiments, the present disclosure provides a method for identifying a subject having a cancer as not likely to exhibit cancer survival, wherein the method comprises: (a) determining a level of a diagnostic bacterium or a spore thereof in a sample of the subject; (b) comparing the level of the diagnostic bacterium or spores thereof to a reference diagnostic bacterium or a spore thereof level; (c) identifying the subject as not likely to exhibit cancer survival if the level of the diagnostic bacterium or spore thereof is lower than the reference diagnostic bacterium or spore thereof, wherein the diagnostic bacterium is a bacterium of the taxonomic group selected from the group consisting of Clostridia, Streptococcaceae, Lactobacillaceae, Actinobacteria, Pediococcus acidilactici, Erysipelotrichales, Coprobacillaceae, Clostridiaceae, Akkermansia muciniphila, Escherichia coli, Streptococcus mitis, Veillonellaceae, Bilophila, Parascardovia, Streptococcus salivarius, Coriobacteriaceae, Peptostreptococcaceae, Phascolarctobacterium faecium, Lactobacillus plantarum, Clostridium tertium, Eubacterium biforme, Alphaproteobacteria, Ruminococcaceae, Gemella haemolysans, Dorea, Coprococcus, Clostridium, Rothia, Megasphaera, Atopobium, Clostridium glycolicum, Coprococcus comes, Clostridium spiroforme, Lactobacillus fermentum, Clostridium sp. 826, Lactobacillus animalis, Ruminococcus (family Lachnospiraceae), Streptococcus parasanguinis, Clostridium sp. ID5, Granulicatella adiacens, Actinomyces graevenitzii, Clostridium bartlettii DSM 16795, S24-7, Clostridium algidixylanolyticum, Lactobacillus homohiochii, Tyzzerella nexilis, Mogibacterium neglectum, Lactobacillus reuteri, Clostridium saccharogumia, [Eubacterium] hallii, Drancourtella massiliensis, Clostridium perfringens, Bacteroides stercoris ATCC 43183, Ruminococcus faecis, Clostridium sp. PPf35E6, Bacteroides uniformis, Weissella confusa, Megasphaera micronuciformis, Propionibacterium, Peptostreptococcus sp. MDA2346-2, Bacteroides fragilis, Oscillospira, Lactobacillus gasseri, Rothia dentocariosa, Clostridium sporosphaeroides, Lactococcus, Clostridium hathewayi, Clostridium methylpentosum DSM 5476, Clostridium sp. cTPY-17, Clostridium sp. Enrichment culture clone NHT38, Ruminococcus torques, Clostridium sp. cf3-PUG, Lactobacillus buchneri, mitochondria, Coprococcus, Bacillaceae, Fusobacterium nucleatum, Eggerthella lenta, Anaerococcus, Clostridium sp. Culture-54, Anaerostipes caccae, Blautia faecis, Actinobaculum massiliense, Enterococcus gallinarum, Clostridium sp. MSTE9, ambiguous Leuconostoc, Paraprevotellaceae, Roseburia, unclassified Peptostreptococcaceae, Fusobacterium, Atopobium rimae, Blautia luti, Propionibacterium propionicum, Streptococcus lutetiensis, Scardovia inopinata, Dorea formicigenerans, Corynebacterium pseudogenitalium, Turicibacter sanguinis, Roseburia faecis, Clostridium paraputrificum, Fusobacteria, Clostridium symbiosum, Propionibacterium freudenreichii, and any combinations thereof, or the diagnostic bacterium or spore thereof comprises a 16S rRNA gene having a nucleotide sequence that has between about 90 and 100% homology to the nucleotide sequence set forth in any one of SEQ ID NOs:1-69.

In certain embodiments, the present disclosure provides a method for identifying a subject having a cancer as not likely to exhibit cancer survival, wherein the method comprises: (a) determining a level of a diagnostic bacterium or a spore thereof in a sample of the subject; (b) comparing the level of the diagnostic bacterium or spores thereof to a reference diagnostic bacterium or a spore thereof level; and (c) identifying the subject as not likely to exhibit cancer survival if the level of the diagnostic bacterium or spore thereof is higher than the reference diagnostic bacterium or spore thereof, wherein the diagnostic bacterium is a bacterium of the taxonomic group selected from the group consisting of Firmicutes, Bacilli, Enterococcus, Bacillales, ambiguous Klebsiella, Lactobacillus delbrueckii subsp. Bulgaricus, Coprobacillus, Parabacteroides, Ruminococcus, Streptococcus mutans, Enterococcus rivorum, Enterobacter ludwigii, Clostridium leptum, Enterococcus lactis, Bifidobacterium dentium, Eubacterium limosum, Proteobacteria, Mycoplasma, Holdemania filiformis, Lactococcus piscium, Blautia, Bacteroides thetaiotaomicron, Massiliomicrobiota timonensis, Blautia hydrogenotrophica, Enterococcus mundtii, Prevotella melaninogenica, Erwinia chrysanthemi, Clostridium nexile DSM 1787, Veillonella, Clostridium difficile, [Ruminococcus] obeum, Streptophyta, Bacteroidales, Parascardovia, Clostridium clostridioforme, Blautia obeum, Klebsiella oxytoca, Bulleidia moorei, Parabacteroides merdae, Shuttleworthia satelles, Streptococcus sp. DN812, Clostridium cellulosi, Lactobacillus acidophilus, Bacteroides ovatus, Clostridium hylemonae, Veillonella parvula, Longibaculum muris, Butyrivibrio, Peptostreptococcaceae bacterium canine oral taxon 074, Alloscardovia omnicolens, Lactobacillus salivarius, Clostridium scindens, Alistipes, Clostridium lavalense, Anaerostipes, Abiotrophia defectiva, Leuconostocaceae, Dorea, Alistipes putredinis, Salinicoccus qingdaonensis, Parasutterella excrementihominis, Bacteroides caccae, Bifidobacteriaceae, Streptococcus anginosus, Haemophilus parainfluenzae, Oscillospira, Clostridium aldenense, and any combination thereof, or the diagnostic bacterium or spore thereof comprises a 16S rRNA gene sequence that has between about 90 and 100% homology to the nucleotide sequence set forth in any one of SEQ ID NOs:70-118.

In certain embodiments, the presently disclosed method further comprises treating the subject identified as not likely to exhibit cancer survival with a cancer treatment.

In certain embodiments, the cancer treatment comprises administering to the subject a therapeutic bacterium or a spore thereof or a pharmaceutical composition comprising thereof, a hematopoietic cell transplantation (HCT), surgery, radiation therapy, chemotherapy, immunotherapy, stem cell therapy, cellular therapy, a probiotic bacteria, a probiotic yeast, a prebiotic, a postbiotic, an antibiotic, or a combination thereof. In certain embodiments, the therapeutic bacterium is a bacterium of the taxonomic group selected from the group consisting of Clostridia, Streptococcaceae, Lactobacillaceae, Actinobacteria, Pediococcus acidilactici, Erysipelotrichales, Coprobacillaceae, Clostridiaceae, Akkermansia muciniphila, Escherichia coli, Streptococcus mitis, Veillonellaceae, Bilophila, Parascardovia, Streptococcus salivarius, Coriobacteriaceae, Peptostreptococcaceae, Phascolarctobacterium faecium, Lactobacillus plantarum, Clostridium tertium, Eubacterium biforme, Alphaproteobacteria, Ruminococcaceae, Gemella haemolysans, Dorea, Coprococcus, Clostridium, Rothia, Megasphaera, Atopobium, Clostridium glycolicum, Coprococcus comes, Clostridium spiroforme, Lactobacillus fermentum, Clostridium sp. 826, Lactobacillus animalis, Ruminococcus (family Lachnospiraceae), Streptococcus parasanguinis, Clostridium sp. ID5, Granulicatella adiacens, Actinomyces graevenitzii, Clostridium bartlettii DSM 16795, S24-7, Clostridium algidixylanolyticum, Lactobacillus homohiochii, Tyzzerella nexilis, Mogibacterium neglectum, Lactobacillus reuteri, Clostridium saccharogumia, [Eubacterium] hallii, Drancourtella massiliensis, Clostridium perfringens, Bacteroides stercoris ATCC 43183, Ruminococcus faecis, Clostridium sp. PPf35E6, Bacteroides uniformis, Weissella confusa, Megasphaera micronuciformis, Propionibacterium, Peptostreptococcus sp. MDA2346-2, Bacteroides fragilis, Oscillospira, Lactobacillus gasseri, Rothia dentocariosa, Clostridium sporosphaeroides, Lactococcus, Clostridium hathewayi, Clostridium methylpentosum DSM 5476, Clostridium sp. cTPY-17, Clostridium sp. Enrichment culture clone NHT38, Ruminococcus torques, Clostridium sp. cf3-PUG, Lactobacillus buchneri, mitochondria, Coprococcus, Bacillaceae, Fusobacterium nucleatum, Eggerthella lenta, Anaerococcus, Clostridium sp. Culture-54, Anaerostipes caccae, Blautia faecis, Actinobaculum massiliense, Enterococcus gallinarum, Clostridium sp. MSTE9, ambiguous Leuconostoc, Paraprevotellaceae, Roseburia, unclassified Peptostreptococcaceae, Fusobacterium, Atopobium rimae, Blautia luti, Propionibacterium propionicum, Streptococcus lutetiensis, Scardovia inopinata, Dorea formicigenerans, Corynebacterium pseudogenitalium, Turicibacter sanguinis, Roseburia faecis, Clostridium paraputrificum, Fusobacteria, Clostridium symbiosum, Propionibacterium freudenreichii, and any combinations thereof.

In certain embodiments, the cancer treatment comprises a combination of administering to the subject the therapeutic bacterium or spore thereof or a pharmaceutical composition comprising thereof; and the HCT. In certain embodiments, the therapeutic bacterium or spore thereof or a pharmaceutical composition comprising thereof is administered to the subject before or after the HCT. In certain embodiments, the cancer treatment comprises administering an antibiotic to the subject.

In certain embodiments, the antibiotic administered to the subject selectively kills or inhibits the growth of the diagnostic bacterium or spore thereof of Group 3. In certain embodiments, the antibiotic does not selectively kill or inhibit the growth of the diagnostic bacterium or spore thereof of Group 2.

In certain embodiments, the present disclosure provides a method for identifying a subject having a cancer as likely to exhibit cancer survival, wherein the method comprises: (a) determining a level of a diagnostic bacterium or a spore thereof in a sample of the subject; (b) comparing the level of the diagnostic bacterium or spores thereof to a reference diagnostic bacterium or a spore thereof level; (c) identifying the subject as likely to exhibit cancer survival if the level of the diagnostic bacterium or spore thereof is higher than the reference diagnostic bacterium or spore thereof; wherein the diagnostic bacterium is a bacterium of the taxonomic group selected from the group consisting of Clostridia, Streptococcaceae, Lactobacillaceae, Actinobacteria, Pediococcus acidilactici, Erysipelotrichales, Coprobacillaceae, Clostridiaceae, Akkermansia muciniphila, Escherichia coli, Streptococcus mitis, Veillonellaceae, Bilophila, Parascardovia, Streptococcus salivarius, Coriobacteriaceae, Peptostreptococcaceae, Phascolarctobacterium faecium, Lactobacillus plantarum, Clostridium tertium, Eubacterium biforme, Alphaproteobacteria, Ruminococcaceae, Gemella haemolysans, Dorea, Coprococcus, Clostridium, Rothia, Megasphaera, Atopobium, Clostridium glycolicum, Coprococcus comes, Clostridium spiroforme, Lactobacillus fermentum, Clostridium sp. 826, Lactobacillus animalis, Ruminococcus (family Lachnospiraceae), Streptococcus parasanguinis, Clostridium sp. ID5, Granulicatella adiacens, Actinomyces graevenitzii, Clostridium bartlettii DSM 16795, S24-7, Clostridium algidixylanolyticum, Lactobacillus homohiochii, Tyzzerella nexilis, Mogibacterium neglectum, Lactobacillus reuteri, Clostridium saccharogumia, [Eubacterium] hallii, Drancourtella massiliensis, Clostridium perfringens, Bacteroides stercoris ATCC 43183, Ruminococcus faecis, Clostridium sp. PPf35E6, Bacteroides uniformis, Weissella confusa, Megasphaera micronuciformis, Propionibacterium, Peptostreptococcus sp. MDA2346-2, Bacteroides fragilis, Oscillospira, Lactobacillus gasseri, Rothia dentocariosa, Clostridium sporosphaeroides, Lactococcus, Clostridium hathewayi, Clostridium methylpentosum DSM 5476, Clostridium sp. cTPY-17, Clostridium sp. Enrichment culture clone NHT38, Ruminococcus torques, Clostridium sp. cf3-PUG, Lactobacillus buchneri, mitochondria, Coprococcus, Bacillaceae, Fusobacterium nucleatum, Eggerthella lenta, Anaerococcus, Clostridium sp. Culture-54, Anaerostipes caccae, Blautia faecis, Actinobaculum massiliense, Enterococcus gallinarum, Clostridium sp. MSTE9, ambiguous Leuconostoc, Paraprevotellaceae, Roseburia, unclassified Peptostreptococcaceae, Fusobacterium, Atopobium rimae, Blautia luti, Propionibacterium propionicum, Streptococcus lutetiensis, Scardovia inopinata, Dorea formicigenerans, Corynebacterium pseudogenitalium, Turicibacter sanguinis, Roseburia faecis, Clostridium paraputrificum, Fusobacteria, Clostridium symbiosum, Propionibacterium freudenreichii, and any combinations thereof, or the diagnostic bacterium or spore thereof comprises a 16S rRNA gene having a nucleotide sequence that has between about 90 and 100% homology to the nucleotide sequence set forth in any one of SEQ ID NOs:1-69.

In certain embodiments, the present disclosure provides a method for identifying a subject having a cancer as likely to exhibit cancer survival, wherein the method comprises: (a) determining a level of a diagnostic bacterium or a spore thereof in a sample of the subject; (b) comparing the level of the diagnostic bacterium or spores thereof to a reference diagnostic bacterium or a spore thereof level; and (c) identifying the subject as likely to exhibit cancer survival if the level of the diagnostic bacterium or spore thereof is lower than the reference diagnostic bacterium or spore thereof, wherein the diagnostic bacterium is a bacterium of the taxonomic group selected from the group consisting of Firmicutes, Bacilli, Enterococcus, Bacillales, ambiguous Klebsiella, Lactobacillus delbrueckii subsp. Bulgaricus, Coprobacillus, Parabacteroides, Ruminococcus, Streptococcus mutans, Enterococcus rivorum, Enterobacter ludwigii, Clostridium leptum, Enterococcus lactis, Bifidobacterium dentium, Eubacterium limosum, Proteobacteria, Mycoplasma, Holdemania filformis, Lactococcus piscium, Blautia, Bacteroides thetaiotaomicron, Massiliomicrobiota timonensis, Blautia hydrogenotrophica, Enterococcus mundtii, Prevotella melaninogenica, Erwinia chrysanthemi, Clostridium nexile DSM 1787, Veillonella, Clostridium difficile, [Ruminococcus] obeum, Streptophyta, Bacteroidales, Parascardovia, Clostridium clostridioforme, Blautia obeum, Klebsiella oxytoca, Bulleidia moorei, Parabacteroides merdae, Shuttleworthia satelles, Streptococcus sp. DN812, Clostridium cellulosi, Lactobacillus acidophilus, Bacteroides ovatus, Clostridium hylemonae, Veillonella parvula, Longibaculum muris, Butyrivibrio, Peptostreptococcaceae bacterium canine oral taxon 074, Alloscardovia omnicolens, Lactobacillus salivarius, Clostridium scindens, Alistipes, Clostridium lavalense, Anaerostipes, Abiotrophia defectiva, Leuconostocaceae, Dorea, Alistipes putredinis, Salinicoccus qingdaonensis, Parasutterella excrementihominis, Bacteroides caccae, Bifidobacteriaceae, Streptococcus anginosus, Haemophilus parainfluenzae, Oscillospira, Clostridium aldenense, and any combination thereof, or the diagnostic bacterium or spore thereof comprises a 16S rRNA gene sequence that has between about 90 and 100% homology to the nucleotide sequence set forth in any one of SEQ ID NOs:70-118.

In certain embodiments, the present disclosure provides a kit, wherein the kit comprises the presently disclosed pharmaceutical composition.

In certain embodiments, the presently disclosed kit further comprises instructions for treating a subject having a cancer.

In certain embodiments, the present disclosure provides a kit for identifying a subject having a cancer as not likely to exhibit cancer survival, wherein the kit comprises means for detecting the level of a diagnostic bacterium or a spore thereof, wherein the diagnostic bacterium is a bacterium of the taxonomic group selected from the group consisting of Clostridia, Streptococcaceae, Lactobacillaceae, Actinobacteria, Pediococcus acidilactici, Erysipelotrichales, Coprobacillaceae, Clostridiaceae, Akkermansia muciniphila, Escherichia coli, Streptococcus mitis, Veillonellaceae, Bilophila, Parascardovia, Streptococcus salivarius, Coriobacteriaceae, Peptostreptococcaceae, Phascolarctobacterium faecium, Lactobacillus plantarum, Clostridium tertium, Eubacterium biforme, Alphaproteobacteria, Ruminococcaceae, Gemella haemolysans, Dorea, Coprococcus, Clostridium, Rothia, Megasphaera, Atopobium, Clostridium glycolicum, Coprococcus comes, Clostridium spiroforme, Lactobacillus fermentum, Clostridium sp. 826, Lactobacillus animalis, Ruminococcus (family Lachnospiraceae), Streptococcus parasanguinis, Clostridium sp. ID5, Granulicatella adiacens, Actinomyces graevenitzii, Clostridium bartlettii DSM 16795, S24-7, Clostridium algidixylanolyticum, Lactobacillus homohiochii, Tyzzerella nexilis, Mogibacterium neglectum, Lactobacillus reuteri, Clostridium saccharogumia, [Eubacterium] hallii, Drancourtella massiliensis, Clostridium perfringens, Bacteroides stercoris ATCC 43183, Ruminococcus faecis, Clostridium sp. PPf35E6, Bacteroides uniformis, Weissella confusa, Megasphaera micronuciformis, Propionibacterium, Peptostreptococcus sp. MDA2346-2, Bacteroides fragilis, Oscillospira, Lactobacillus gasseri, Rothia dentocariosa, Clostridium sporosphaeroides, Lactococcus, Clostridium hathewayi, Clostridium methylpentosum DSM 5476, Clostridium sp. cTPY-17, Clostridium sp. Enrichment culture clone NHT38, Ruminococcus torques, Clostridium sp. cf3-PUG, Lactobacillus buchneri, mitochondria, Coprococcus, Bacillaceae, Fusobacterium nucleatum, Eggerthella lenta, Anaerococcus, Clostridium sp. Culture-54, Anaerostipes caccae, Blautia faecis, Actinobaculum massiliense, Enterococcus gallinarum, Clostridium sp. MSTE9, ambiguous Leuconostoc, Paraprevotellaceae, Roseburia, unclassified Peptostreptococcaceae, Fusobacterium, Atopobium rimae, Blautia luti, Propionibacterium propionicum, Streptococcus lutetiensis, Scardovia inopinata, Dorea formicigenerans, Corynebacterium pseudogenitalium, Turicibacter sanguinis, Roseburia faecis, Clostridium paraputrificum, Fusobacteria, Clostridium symbiosum, Propionibacterium freudenreichii, and any combinations thereof, or the diagnostic bacterium or spore thereof comprises a 16S rRNA gene having a nucleotide sequence that has between about 90 and 100% homology to the nucleotide sequence set forth in any one of SEQ ID NOs:1-69

In certain embodiments, the presently disclosed kit instructions for identifying the subjects as not likely to exhibit cancer survival, wherein the instructions comprise: (a) determining the level of the diagnostic bacterium or spore thereof in a sample of the subject; (b) comparing the level of the diagnostic bacterium or spores thereof to a reference diagnostic bacterium or a spore thereof level; and (c) identifying the subject as not likely to exhibit cancer survival if the level of the diagnostic bacterium or spore thereof is lower than the reference diagnostic bacterium or spore thereof.

In certain embodiments, the present disclosure provides a kit for of identifying a subject as not likely to exhibit cancer survival, wherein the kit comprises means for detecting the level of a diagnostic bacterium or a spore thereof, wherein the diagnostic bacterium is a bacterium of the taxonomic group selected from the group consisting of Firmicutes, Bacilli, Enterococcus, Bacillales, ambiguous Klebsiella, Lactobacillus delbrueckii subsp. Bulgaricus, Coprobacillus, Parabacteroides, Ruminococcus, Streptococcus mutans, Enterococcus rivorum, Enterobacter ludwigii, Clostridium leptum, Enterococcus lactis, Bifidobacterium dentium, Eubacterium limosum, Proteobacteria, Mycoplasma, Holdemania filiformis, Lactococcus piscium, Blautia, Bacteroides thetaiotaomicron, Massiliomicrobiota timonensis, Blautia hydrogenotrophica, Enterococcus mundtii, Prevotella melaninogenica, Erwinia chrysanthemi, Clostridium nexile DSM 1787, Veillonella, Clostridium difficile, [Ruminococcus] obeum, Streptophyta, Bacteroidales, Parascardovia, Clostridium clostridioforme, Blautia obeum, Klebsiella oxytoca, Bulleidia moorei, Parabacteroides merdae, Shuttleworthia satelles, Streptococcus sp. DN812, Clostridium cellulosi, Lactobacillus acidophilus, Bacteroides ovatus, Clostridium hylemonae, Veillonella parvula, Longibaculum muris, Butyrivibrio, Peptostreptococcaceae bacterium canine oral taxon 074, Alloscardovia omnicolens, Lactobacillus salivarius, Clostridium scindens, Alistipes, Clostridium lavalense, Anaerostipes, Abiotrophia defectiva, Leuconostocaceae, Dorea, Alistipes putredinis, Salinicoccus qingdaonensis, Parasutterella excrementihominis, Bacteroides caccae, Bifidobacteriaceae, Streptococcus anginosus, Haemophilus parainfluenzae, Oscillospira, Clostridium aldenense, and any combination thereof, or the diagnostic bacterium or spore thereof comprises a 16S rRNA gene sequence that has between about 90 and 100% homology to the nucleotide sequence set forth in any one of SEQ ID NOs:70-118.

In certain embodiments, the presently disclosed kit further comprises instructions for identifying the subjects as not likely to exhibit cancer survival, wherein the instructions comprise: (a) determining the level of the diagnostic bacterium or spore thereof in a sample of the subject; (b) comparing the level of the diagnostic bacterium or spores thereof to a reference diagnostic bacterium or a spore thereof level; and (c) identifying the subject as not likely to exhibit cancer survival if the level of the diagnostic bacterium or spore thereof is higher than the reference diagnostic bacterium or spore thereof.

### 4. BRIEF DESCRIPTION OF THE FIGURES

FIGS. 1A-1E show that intestinal microbiota diversity declines during transplantation and is associated with poor overall cancer survival. FIG. 1A shows intestinal microbiota diversity, as measured by 16S sequencing and the inverse Simpson index, declined comparably during the course of allogeneic hematopoietic cell transplantation (allo-HCT) at all four institutions. Each point represents a stool sample, color coded by institution. Curves are loess-smoothed averages. Median diversity decreased 4.3-, 1.7-, 3.3-, and 2.5-fold respectively at MSK, Regensburg (Reg), Duke (Duk), and Hokkaido (Hok) between each patient's baseline (earliest sample day −30 to −6) to the peri-engraftment period (median value of samples collected day 7-21). See also FIG. 7. FIGS. 1B and 1C show that overall survival is longer in patients with high intestinal diversity in peri-engraftment samples. The median diversity value in the MSK cohort (2.64) was used as the stratification cutoff in both of these day +21 landmark analyses. MSK high-diversity group, 104 events in 354 patients; MSK low-diversity group, 136 events in 350 patients; Reg+Duk+Hok high-diversity group, 18 events in 87 patients; Reg+Duk+Hok low-diversity group, 35 events in 92 patients. Accompanying analyses of diversity as a continuous variable and multivariate models are in Table 2. (D) Cumulative incidences of transplant-related mortality and relapse/progression of disease in the MSK cohort. Transplant-related mortality events: high-diversity group, 52 events in 354 patients; low-diversity group, 82 events in 349 patients. Relapse events: high-diversity group, 84 events in 354 patients; low-diversity group, 81 events in 349 patients. (E) Subset analysis of the MSK recipients of either T-replete (unmodified) vs. T-cell-depleted grafts; see also FIG. 9. Numbers of patients at risk in FIGS. 1D and 1E are tabulated in FIG. 16.

FIGS. 2A-2G show the global spectrum of microbiota injury in allo-HCT. FIGS. 2A-2D show the microbiota composition of 8,768 samples from 1,362 patients from all four institutions visualized via the t-Distributed Stochastic Neighbor Embedding (t-SNE) algorithm. Each point represents a single stool sample and the axes (tSNE1 and tSNE2) have arbitrary units. The more similar samples are, the closer they appear on the t-SNE plot. Earlier samples (left side of FIG. 2A) are enriched for high-diversity samples (left side of FIG. 2B) whereas later samples are enriched for low-diversity configurations (right side p FIG. 2B). FIG. 2C shows samples from all four institutions are well distributed across t-SNE space. FIG. 2D shows color-coding by the most abundant taxon in each sample, which illustrates that the early cluster is characterized by Clostridia (brown, orange, pink), Bacteroidetes (teal) and Actinobacteria (purple). Some low-diversity states are characterized by domination with the genera Enterococcus (dark green), Streptococcus (light green), Klebsiella (reds) and Escherichia (other reds). FIG. 2E shows that among patients with at least one pre-HCT and one post-HCT sample, the fraction of patients who have experienced at least one instance of domination each week, as defined by relative abundance 30% for any taxonomic unit. FIG. 2F shows the fraction of samples with domination in each 7-day sliding window. FIG. 2G shows taxa contributing to domination events in the MSK cohort. Domination was defined at the level of operational taxonomic units; color-coding is at higher taxonomic ranks as in the color legend.

FIGS. 3A-3C show that microbiota injury is observed pre-HCT and is predictive of allo-HCT outcomes. FIG. 3A shows that the diversity of initial patient samples (collected day −30 to −6) is lower than those from 314 participants of the Human Microbiome Project (HMP) and from 34 healthy adult volunteers sampled and sequenced at MSK (healthy control). FIG. 3B shows the fraction of healthy-volunteer and initial patient samples classifiable via the Enterotypes scheme. Pre-HCT samples are less likely to be classifiable as belonging to an Enterotype than healthy volunteers. FIG. 3C shows the overall cancer survival is higher in patients with high intestinal diversity in initial samples from MSK. High-diversity group, 72 events in 250 patients; low-diversity group, 101 events in 251 patients.

FIGS. 4A-4D show risk score taxa. (FIG. 4A) The risk score for post-HCT mortality was computed as a function of the intestinal abundance of 172 bacterial taxa. The score was derived in the training (MSK) cohort using regularized regression. Each point is one of the 172 bacterial taxa, and the diameter of the points is proportional to the mean abundance of the taxon in the intestinal communities. Red indicates an association with increased mortality and blue indicates an association with decreased mortality. The overall risk score was computed as a weighted average of the taxa (where the weights were defined by the regularized Cox model) and was plotted on the horizontal axis. The average magnitude of each taxon's contribution to the overall risk score was plotted on the vertical axis by multiplying the estimated weight by the average abundance in the training cohort. For example, the class Bacilli (large red point near the top of the graph) had a relatively modest small effect size in the direction of predicting increased mortality risk, but due to its high abundance it made a large contribution to the overall risk score. In contrast, Enterococcus gallinarum (small blue point in the lower left) had a large effect size in the direction of predicting reduced mortality risk, but due to its low abundance it made a small contribution to overall risk score. The names of taxa with the largest effect sizes are annotated; the full list is tabulated in FIG. 5. p, phylum; c, class; f, family; g, genus; s, species. (FIG. 4B) The risk score trained in the MSK cohort was tested in a validation cohort combined of patients from Duke+Regensburg+Hokkaido. The risk score association with patient overall survival was evaluated as a continuous variable in a multivariable model adjusted for age, conditioning intensity, graft source, and HCT-CI with stratification by institution. (FIG. 4C) Non-parametric estimate of the hazard rate for categories of the standardized risk score (plotted using the R package bshazard). Patients at the highest risk of death are in the highest-risk tertile; the difference in the hazard rate across the three groups is most prominent in the first-year post-transplant and attenuates over time. 24 events in 59 patients in the highest-risk tertile, 16 events in 59 patients in the middle-risk tertile, 13 events in 60 patients in the lowest-risk tertile. (FIG. 4D) Concordance indices (C-index) for various models of mortality in the validation cohort combined of patients from Duke+Regensburg+Hokkaido. All models were stratified by institution.

FIG. 5 shows the full list of taxa with risk score. Effect size is the coefficient of each term in the model. Positive values indicate increased mortality risk; negative values decreased mortality risk. The abundance columns tabulate the abundance of each taxon at MSK day 7-21. Score contributions are the effect sizes scaled according to taxon abundance. k, kingdom; p, phylum; c, class; o, order; f, family; g, genus; s, species.

FIGS. 6A-6C show distribution of sample collections. (FIG. 6A) Unique patients from each cohort are plotted along the vertical axis against time relative to HCT on the horizontal axis. Each point is a sample, color-coded according to its α-diversity, as measured by the inverse Simpson index. (FIG. 6B) Histograms of sample collection frequency across time relative to HCT in each cohort. (FIG. 6C) Histogram of the number of samples per patient analyzed. FIGS. 6A-6C are associated with Table 1, FIGS. 13 and 14.

FIG. 7 shows that a loss of intestinal diversity was observed at all four centers. Plotted are the inverse Simpson index values of fecal samples collected at baseline (earliest sample collected between day −30 and day −6) and the median values of each patient's samples that were collected in the peri-engraftment period (day 7,21). Only patients with at least one sample in each time period are plotted (N=408, 20, 26, and 19 at MSK, Regensburg, Duke, and Hokkaido, respectively). At MSK, Regensburg, Duke, and Hokkaido diversity decreased 4.3-, 1.7-, 3.3-, and 2.5-fold, respectively. At each center, the reduction in diversity was significant by a paired Wilcoxon test (p<0.001).

FIG. 8 shows that diversity declines comparably in recipients of T-depleted and T-replete grafts. Intestinal microbiota diversity, as measured by 16S sequencing and the inverse Simpson index, declined comparably in 447 recipients of T-cell depleted grafts as in 629 recipients of unmodified grafts (368 PBSC unmodified, 178 cord-blood, and 83 BM unmodified) at MSK.

FIG. 9 shows that peri-neutrophil engraftment predicts TRM and GRM in recipients of T-replete grafts at MSK. In this subset analysis Forest plot of specific clinical outcomes, hazard ratios for the indicated outcome are plotted. The size of the diamond is proportional to the number of patients in each subset. Whiskers indicate 95% confidence intervals. Cumulative incidence curves of the same data are plotted in FIG. 1E.

FIGS. 10A-10B show antibiotic exposures. (FIG. 10A) Heatmap of drug exposures in the four cohorts. Each row is a drug, and each column is one of the four institutions. The values and color-coding indicate the fraction of patients at each center who were exposed to at least one dose in the defined exposure window. The black bracket indicates drugs to which >20% of patients in at least one institution were exposed and were considered for this analysis; asterisks indicate drugs that were employed in prophylactic regimens in this population that were excluded. (FIG. 10B) Schematic of the sampling and exposure windows used to identify the drugs associated with a decrease in diversity from the pre-HCT period to the peri-engraftment period. Association of drug exposure with clinical outcomes (FIG. 17) was analyzed in a landmark fashion following day 21 as in the rest of the manuscript.

FIGS. 11A-11B show statistical analysis of microbiome composition. (FIG. 11A) The variation in microbiota composition between centers is smaller than the magnitude of change observed during HCT. The variation in microbiota composition between centers is smaller than the magnitude of change observed during HCT. The present disclosure defined as a reference point an averaged intestinal microbiota composition among samples collected pre-HCT at MSK (day −30 to day −1) Each point represents the distance of a single stool sample from this reference point, as measured by the Bray-Curtis (beta-diversity) distance. Among pre-HCT samples, MSK and Duke had comparable distance to the reference (median distance of 4.52 and 4.60, respectively, p>0.05), Regensburg and Hokkaido samples were moderately farther (median distance of 6.77 and 7.66, respectively, p<0.005). In contrast, the median distance of post-HCT (day 0 to day +20) samples from the reference was markedly farther (11.31, 10.02, 8.53 and 13.82 for MSK, Duke, Regensburg and Hokkaido, respectively, p<0.005). Thus, the pre-HCT variation in microbiota composition across geography is smaller than the changes that occur over the course of transplantation. NS, not-significant. (FIG. 11B) In a generalized estimating equation with an independence working correlation structure for the binary endpoint of sample dominance, the odds of a observing a dominated sample from Regensburg or Duke was comparable to MSK. The odds of a sample from Hokkaido being dominated was higher than at MSK.

FIG. 12 shows survival analysis by pre-HCT and peri-engraftment diversity. In this analysis within the MSK cohort, these four curves were not statistically different overall (p=0.2), but the high-high group (blue) had statistically significantly lower risk of mortality than the low-low group (red) (HR 0.62 [0.39-0.99] p=0.045). High-high group: 29 events in 113 patients. In the low-high group: 26 events in 75 patients. In the high-low group: 31 events in 86 patients. In the low-low group: 45 events in 111 patients. The inverse Simpson diversity cutoff thresholds to define pre-HCT and peri-engraftment groups were 11.2 and 2.66, respectively, as in the rest of the analysis. This analysis is accompanied by a multivariate Cox proportional hazards analysis in FIG. 19.

FIG. 13 provides patient flow through the study (CONSORT Table).

FIG. 14 shows numbers of samples per patient. Summary of the number of serial samples collected per patient in the overall cohort and at each center. For example, 2 serial samples were analyzed from each of 200 patients (15% of the overall cohort), of whom 133 were from MSK (12% of the MSK cohort). When the overall cohort is ranked according to the number of samples per patient, the minimum number of samples per patient was 1 sample/patient. The 25th percentile was 2 samples/patient. The median (50th percentile) was 4 samples/patient. The 75th percentile was 8 samples/patient. The mean number of samples per patient was 6.4.

FIG. 15 shows institutional antibiotic clinical practices.

FIG. 16 shows numbers of patients at risk. Number at risk at 3-month intervals for Kaplan-Meier and cumulative-incidence plots.

FIG. 17 shows diversity and survival remain significantly associated in multivariable models adjusted for exposure to key antibiotics. Multivariable Cox proportional hazards analyses of the association of peri-engraftment intestinal diversity (median of samples collected day +7 to +21) with overall survival at each institution. The multivariate models were stratified by institution and adjusted for age, conditioning intensity, graft source, the hematopoietic cell transplantation comorbidity index (HCT-CI), and duration of exposure to the two key antibiotics identified in FIGS. 10A-10. Intestinal diversity was measured by the inverse Simpson (S) index and is considered here separately as either a log 10-transformed continuous variable or a median-stratified binary variable. See Table 2 for univariate results.

FIG. 18 shows multivariate analysis of pre-HCT diversity at MSK. Multivariate Cox proportional hazards analysis of the association of pre-HCT (first sample collected day −30 to day −6) intestinal diversity with overall survival at MSK. Intestinal diversity was measured by the inverse Simpson (S) index and is considered here separately as either a log-transformed continuous variable or a median-stratified binary variable. The model was adjusted for age, conditioning intensity, graft source, and the hematopoietic cell transplantation comorbidity index (HCT-CI).

FIG. 19 shows multivariate analysis of pre-HCT diversity at MSK. Multivariate Cox proportional hazards analysis of the association of pre-HCT (first sample collected day −30 to day −6) intestinal diversity with overall survival at MSK. Intestinal diversity was measured by the inverse Simpson (S) index and is considered here separately as either a log-transformed continuous variable or a median-stratified binary variable. The model was adjusted for age, conditioning intensity, graft source, and the hematopoietic cell transplantation comorbidity index (HCT-CI). For the continuous-variable analysis there were 131 events in 385 patients. For the binary analysis: in the high-diversity peri-engraftment group there were 55 events in 188 patients and in low-diversity peri-engraftment group there were 76 events in 197 patients. This table is accompanied by the survival curves in FIG. 12.

FIG. 20 shows clinical characteristics of patients by high- and low-diversity groups. Clinical characteristics of patients in the survival analysis according to high- and low-diversity groups day 7-21. For survival analysis, patients were grouped into high- and low-diversity groups according to the institution-specific median diversity. * median Simpson reciprocal diversity index value per institution.

FIG. 21 shows sample-collection periods.

### 5. DETAILED DESCRIPTION

In the following description, details are set forth by way of example to facilitate discussion of the disclosed subject matter. It should be apparent to a person of ordinary skill in the field, however, that the disclosed embodiments are exemplary and not exhaustive of all possible embodiments.

The present disclosure relates to methods for determining the likelihood of cancer survival for a subject following treatment, for example, following allo-HCT, and also to compositions, e.g., pharmaceutical compositions, and methods for increasing the likelihood of cancer survival in a subject. The compositions and methods are based, in part, on Applicants' discovery that there is an association between microbiota diversity and cancer survival for at least about two years following allo-HCT. In particular, these compositions and methods are applicable across populations, and can increase the overall cancer survival of patients following allo-HCT in all geographical locations. The compositions and methods of the present disclosure improve upon current compositions and methods by utilizing microbiota-composition risk scores for the presently disclosed bacteria taxonomic units that are predictors for cancer survival. These predictors are independent of treatment center and geographical location. The compositions and methods of the present disclosure are related specifically to the likelihood of cancer survival, and not to relapse risk. Cancer survival is distinct from cancer relapse, and in the current disclosure refers to overall survival of cancer patients for at least about 2 years following treatment. In contrast, the term “cancer relapse” refers to a return or recurrence of cancer, or the signs and symptoms of cancer, after a period of improvement, for example, after a period of reduction in the presence of cancer, or the signs and symptoms thereof, following treatment.

For clarity of description, and not by way of limitation, this section is divided into the following subsections:

5.1. Definitions;

5.2. Methods of predicting cancer survival;

5.3. Bacteria for predicting likelihood of cancer survival;

5.4. Recombinant cells;

5.5. Compositions;

5.6. Methods of treatments; and

5.7. Kits

### 5.1. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them. The following are terms relevant to the present disclosure:

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s),” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

An “individual” or “subject” or “patient” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

An “effective amount” of a substance as that term is used herein is that amount sufficient to effect beneficial or desired results, including clinical results, and, as such, an “effective amount” depends upon the context in which it is being applied. In the context of administering a composition to increase the likelihood of cancer survival, and/or administering a composition to reduce at least one sign or symptom of TRM and/or GRM, an effective amount of a composition described herein is an amount sufficient to improve clinical outcomes and/or promote cancer survival, as well as decrease the symptoms and/or reduce the likelihood of TRM and/or GRM. The increase in the likelihood of cancer survival can be a 10%, 20%, 30%, 40%, 50%, 60%, 70%, 80%, 90%, 95%, 98% or 99% increase. An effective amount can be administered in one or more administrations.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more signs or symptoms, diminishment of extent of disease, stabilized (i.e., not worsening) state of disease, prevention of disease, delay or slowing of disease progression, remission of the disease (e.g., cancer remission) and/or amelioration or palliation of the disease state. The decrease can be a 10%, 20%, 30%, 40%, 50%, 60%, 70%, 80%, 90%, 95%, 98% or 99% decrease in severity of complications, signs or symptoms. “Treatment” can also mean prolonging cancer survival as compared to expected cancer survival if not receiving treatment. “Treatment” can also refer to increasing the likelihood of cancer survival.

The term “expression vector” is used to denote a nucleic acid molecule that is either linear or circular, into which another nucleic acid sequence fragment of appropriate size can be integrated. Such nucleic acid fragment(s) can include additional segments that provide for transcription of a gene encoded by the nucleic acid sequence fragment. The additional segments can include and are not limited to: promoters, transcription terminators, enhancers, internal ribosome entry sites, untranslated regions, polyadenylation signals, selectable markers, origins of replication and such, as known in the art. Expression vectors are often derived from plasmids, cosmids, viral vectors and yeast artificial chromosomes; vectors are often recombinant molecules containing nucleic acid sequences from several sources.

A “nucleic acid molecule” is a single or double stranded covalently-linked sequence of nucleotides in which the 3′ and 5′ ends on each nucleotide are joined by phosphodiester bonds. The polynucleotide can be made up of deoxyribonucleotide bases or ribonucleotide bases. Polynucleotides include DNA and RNA, and can be manufactured synthetically in vitro or isolated from natural sources.

The term “promoter” as used herein denotes a region within a gene to which transcription factors and/or RNA polymerase can bind so as to control expression of an associated coding sequence. Promoters are commonly, but not always, located in the 5′ non-coding regions of genes, upstream of the translation initiation codon. The promoter region of a gene can include one or more consensus sequences that act as recognizable binding sites for sequence specific nucleic acid binding domains of nucleic acid binding proteins. Nevertheless, such binding sites can also be located in regions outside of the promoter, for example in enhancer regions located in introns or downstream of the coding sequence.

An “Operational Taxonomic Unit”, or “OTU”, and also referred to in this disclosure as a “taxonomic unit” are used to categorize bacteria based on sequence similarity, and are clusters of similar sequence variants of the 16S rRNA gene sequence. For example, typically OTU clusters are defined by a 97% identity threshold to distinguish bacteria at the genus level.

A “cluster,” or “cluster of related bacteria” can include two or more bacterial species or strains that are related by rRNA sequences, for example 16S rRNA gene (e.g., a variable region of the 16S rRNA gene sequence, such as V1, V2, V3, V4 or V5), similarity, and/or evolutionary distance.

A “probiotic” is a microorganism or group of microorganisms that provides health benefits, or that is intended to be non-pathogenic, to a subject when consumed, ingested, or otherwise administered to a subject, for example, an increased likelihood of cancer survival following cancer treatment. As used herein, the term probiotic can be used to describe, for example, probiotic bacteria and/or a probiotic yeast, and can include the bacteria described herein as well as other bacteria.

A “prebiotic” is a substance that promotes the growth, proliferation and/or survival of one or more bacteria or yeast. As used herein, the term prebiotic can be used to describe, for example, a nutritional supplement including plant fiber, or one or more of poorly-absorbed complex carbohydrates, oligosaccharides, inulin-type fructans or arabinoxylans.

A “postbiotic” is a substance derived from a probiotic organism. As used herein, the term postbiotic can be used to describe, for example, a protein expressed by one or more bacteria or yeast, a metabolic product of one or more bacteria or yeast, or media from a culture of one or more strains of bacteria or yeast.

As used herein, the term “microbiota diversity” refers to the number of and abundance distribution of distinct types of microbe organisms within a given body habitat and unless otherwise stated, it is measured in terms of Simpson reciprocal.

As used herein, the term “microbiota injury” refers to loss of diversity in the microbiota composition, or a composition that is different from baseline, or a composition that is different from that of a healthy person, or a composition that is dominated by a single taxon.

As used herein, the term “survival” refers to the survival of a subject having a disease, e.g., a non-cancer disease or cancer, for a certain amount of time (e.g., at least about 6 months, at least about 1 year, at least about 2 years, at least about 3 years, at least about 4 years, at least about 5 years, at least about 6 years, at least about 7 years, at least about 8 years, at least about 9 years, at least about 10 years, at least about 11 years, at least about 12 years, at least about 13 years, at least about 14 years, at least about 15 years or more) following a treatment. In certain embodiments, the treatment is allo-HCT.

As used herein, the term “cancer survival” refers to the survival of a subject having a cancer for a certain amount of time (e.g., at least about 6 months, at least about 1 year, at least about 2 years, at least about 3 years, at least about 4 years, at least about 5 years, at least about 6 years, at least about 7 years, at least about 8 years, at least about 9 years, at least about 10 years, at least about 11 years, at least about 12 years, at least about 13 years, at least about 14 years, at least about 15 years or more) following a cancer treatment. In certain embodiments, the treatment is allo-HCT. In contrast, the term “cancer relapse” refers to a return or recurrence of cancer, or the signs and symptoms of cancer, after a period of improvement, for example, after a period of reduction in the presence of cancer, or the signs and symptoms thereof, following treatment.

In certain non-limiting embodiments, survival or cancer survival can be determined by measuring the proportion of subjects surviving during a two-year period after a treatment, which can be allo-HCT.

As used herein, the term “transplant-related mortality”, or “TRM” refers to deaths that are not preceded by relapse or progression of disease. Transplant-related mortalities within the first 100 days after a transplant can be due to the toxicities of radiation cancer treatments (including immunosuppressive drugs, conditioning chemotherapy and radiation, anti-infectives or other drugs), GVHD, or infections.

As used herein, the term “Graft-versus-host-disease-related mortality”, “GVHD-related mortality”, or “GRM” refers to deaths that occur following a diagnosis of GVHD (e.g., an infection). In certain embodiments, the death can be caused by donor cells attacking the patient's healthy tissue and organs.

As used herein, the term “recombinant cell” refers to cells which have some genetic modification from the original parent cells from which they are derived. Such cells can also be referred to as “genetically-engineered cells.” Such a genetic modification can be the result of an introduction of a heterologous gene (or nucleic acid) for expression of the gene product, e.g., a recombinant protein.

### 5.2. Methods of Predicting Cancer Survival

In certain non-limiting embodiments, the present disclosure provides for methods of determining whether a subject having a cancer is likely or not likely to exhibit cancer survival. In certain embodiments, cancer survival refers to the survival of the subject at least about 2 years following a cancer treatment (e.g., allo-HCT). In certain embodiments, cancer survival refers to the survival of the subject at least 6 months, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years or more following a cancer treatment. In certain embodiments, the cancer survival refers to the survival of the subject at least about 2 years following a cancer treatment.

In certain non-limiting embodiments, the present disclosure provides for methods of determining whether a subject having a non-cancer disease is likely or not likely to exhibit survival after a treatment (e.g., allo-HCT). In certain embodiments, survival refers to the survival of the subject at least 6 months, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years or more following the treatment. In certain embodiments, survival refers to the survival of the subject at least about 2 years following the treatment.

Non-limiting examples of cancers referred to in the present disclosure include, but are not limited to, leukemia, e.g., acute leukemia, acute myeloid leukemia, chronic myeloid leukemia, chronic leukemia, acute lymphoid leukemia, chronic lymphocytic leukemia, biphenotypic acute leukemia and natural killer-cell large granular lymphocyte leukemia, lymphoid malignancies, Non-Hodgkin's lymphoma, plasma cell disorders, myelodysplastic syndrome/myeloproliferative neoplasms and plasmacytoid dendritic cell neoplasms. In certain embodiments, the cancer is leukemia.

Non-limiting examples of non-cancer diseases referred to in the present disclosure include aplastic anemia, and non-malignant hematologic disorders including familial hemophagocytic lymphohistiocytosis, X-linked lymphoproliferative disease and paroxysmal nocturnal hemoglobinuria.

In certain non-limiting embodiments, a subject determined to have a reduced likelihood of survival, e.g., from cancer, can be monitored with increased frequency and for an extended period of time following treatment, and can be administered therapeutic regimes in addition to, or as an alternative to, allo-HCT, as described further herein.

In certain non-limiting embodiments, the treatment, e.g., cancer treatment, comprises hematopoietic cell transplantation (HCT). In certain non-limiting embodiments, the hematopoietic cell transplant comprises allogeneic cells from a donor that is different than the treated patient (allo-HCT).

In certain non-limiting embodiments, the treatment, e.g., cancer treatment, comprises an allogenic cord blood transplant, or allogenic cord stem cell transplant.

In certain non-limiting embodiments, the treatment, e.g., cancer treatment, comprises a T-cell replete transplant or a T-cell depleted transplant. In certain embodiments, the T-cell replete transplant includes an unmodified graft. In certain embodiments, the T-cell depleted transplant includes a graft that has been depleted of T cells before its infusion into the recipient.

In certain non-limiting embodiments, the treatment, e.g., cancer treatment, comprises a bone marrow transplant.

In certain non-limiting embodiments, the methods comprise determining the abundance of a species of bacteria, OTU, or cluster (also referred to herein as “bacterium”) in an intestinal microbiota sample of the subject that is indicative of the likelihood of survival, e.g., cancer survival.

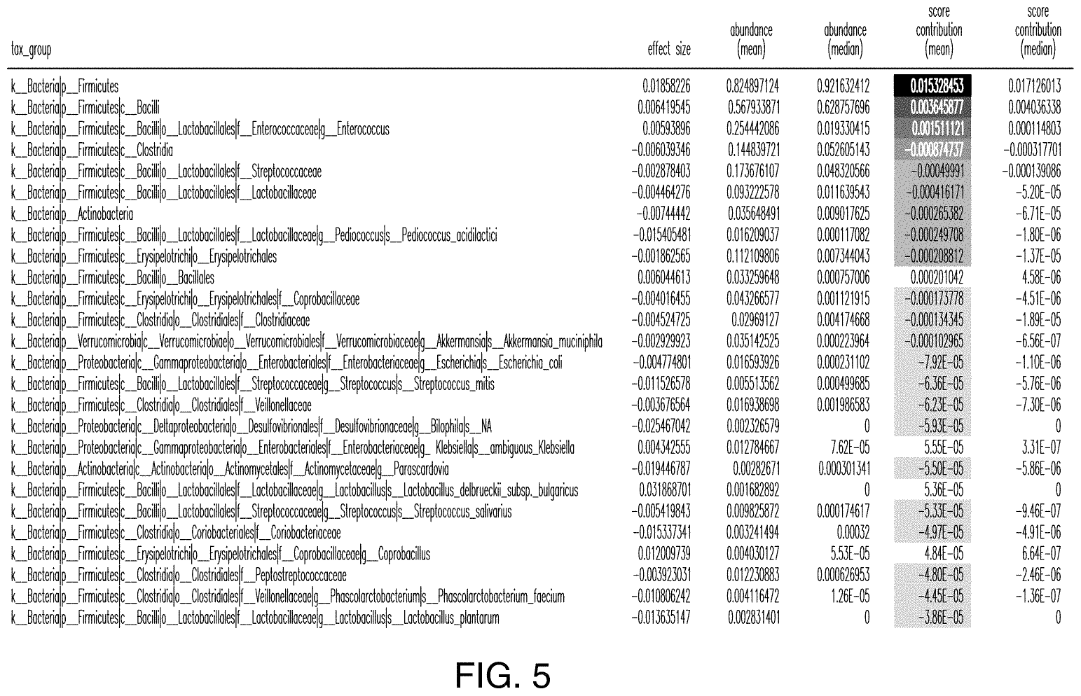

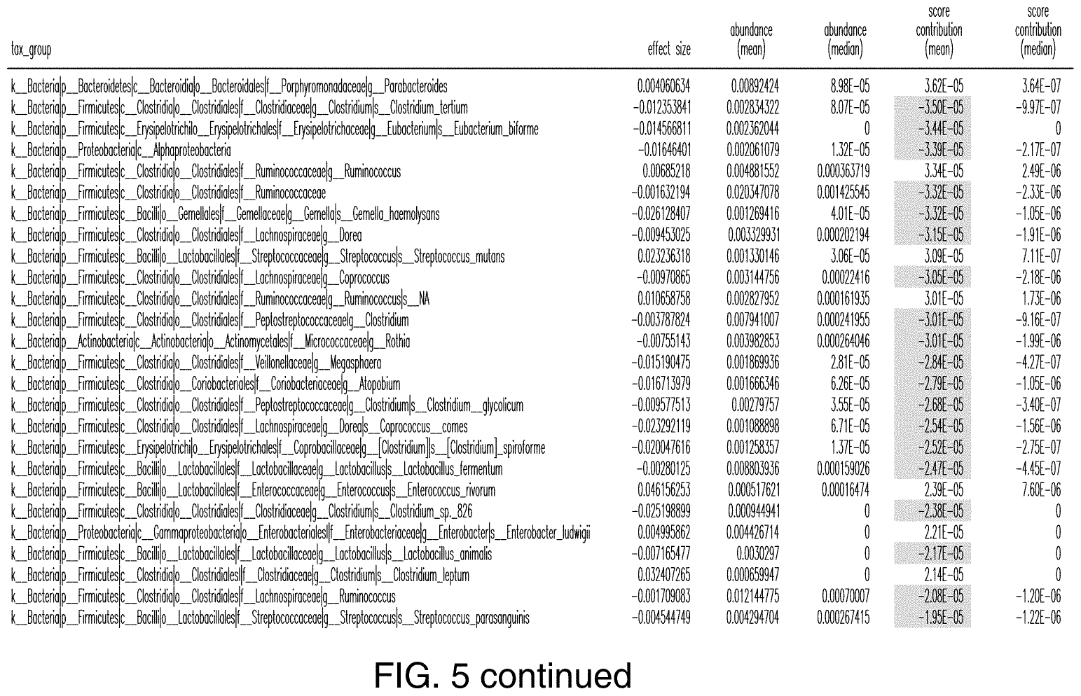

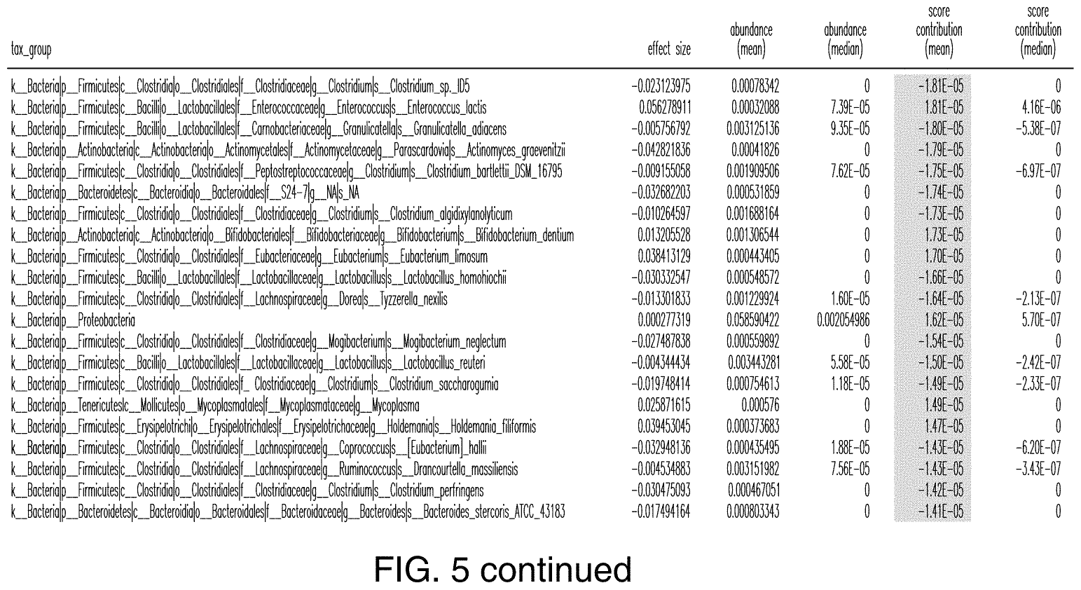

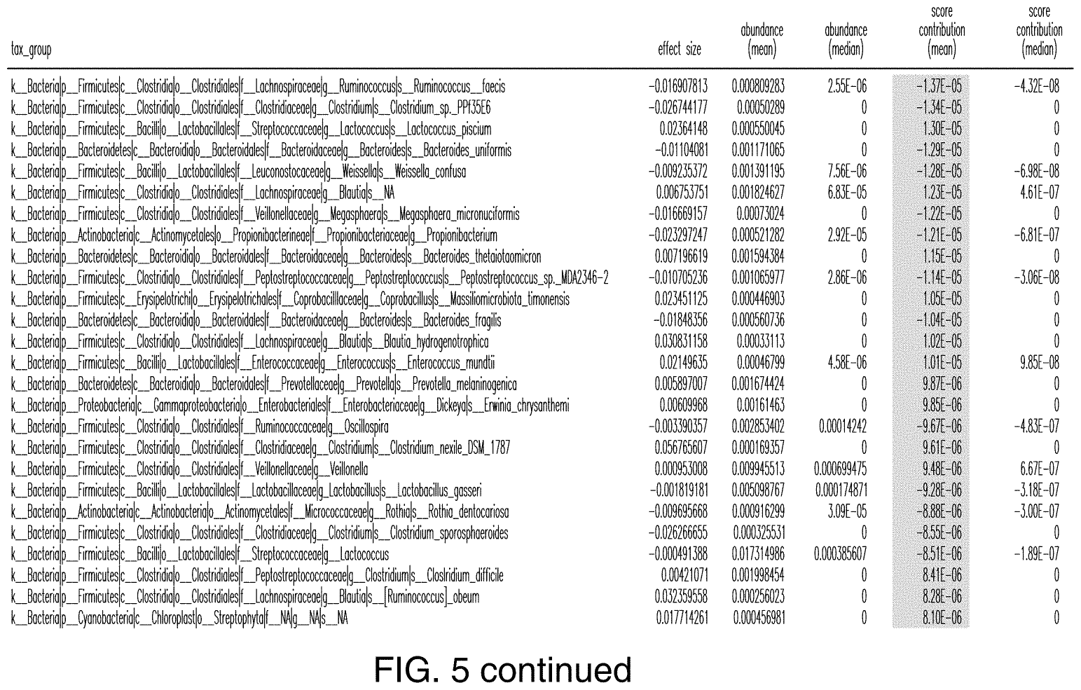

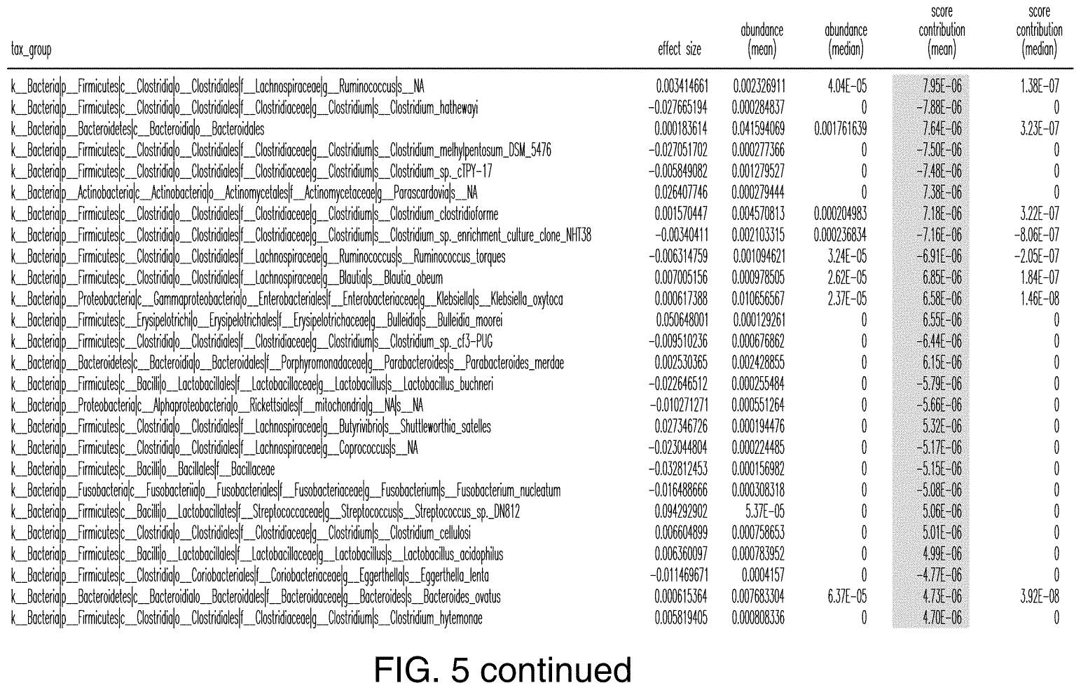

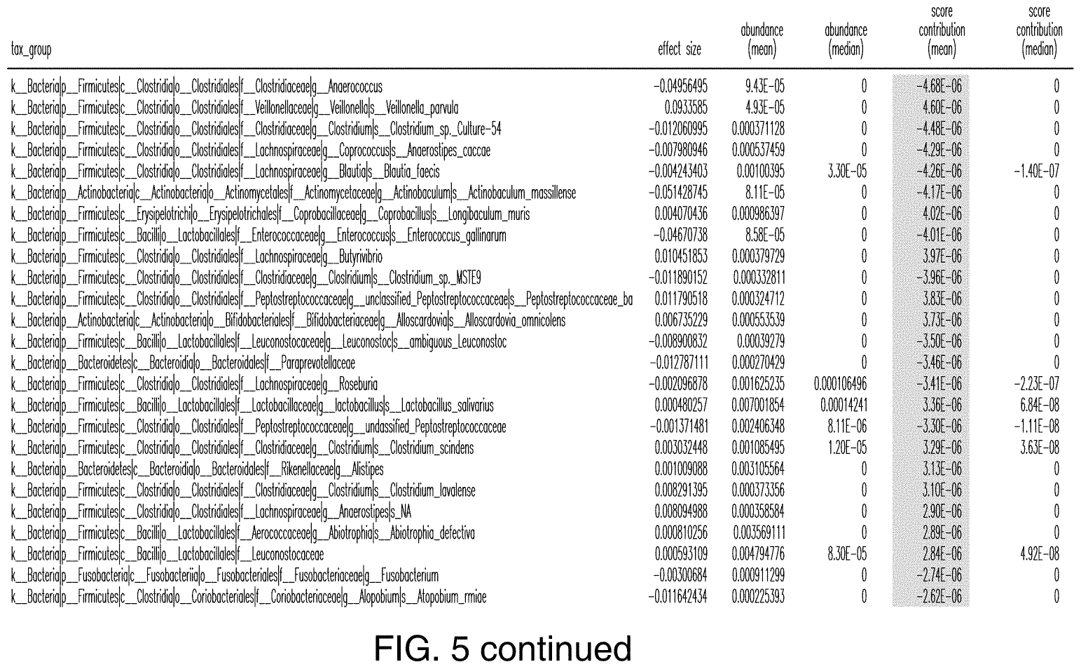

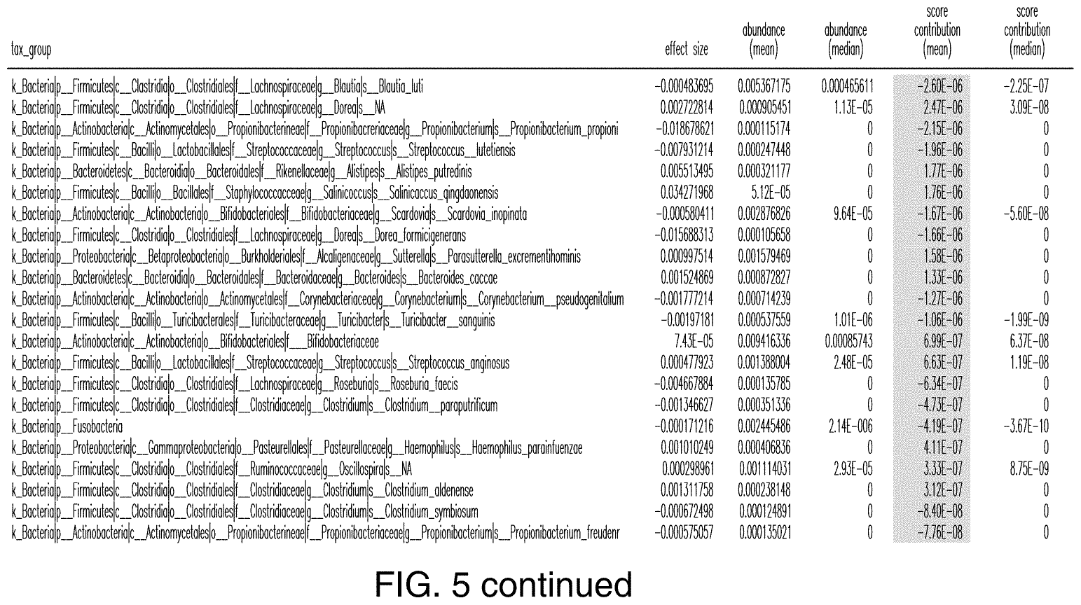

In certain non-limiting embodiments, the bacteria detected, e.g., in an intestinal microbiota sample of the subject, can be associated with the likelihood of survival, e.g., cancer survival. In certain embodiments, such bacteria can be selected from the bacteria listed in FIG. 5. For example, but not by way of limitation, such bacteria can be selected from the taxonomic groups found within super kingdom of bacteria consisting of Firmicutes, Bacilli, Enterococcus, Clostridia, Streptococcaceae, Lactobacillaceae, Actinobacteria, Pediococcus acidilactici, Erysipelotrichales, Bacillales, Coprobacillaceae, Clostridiaceae, Akkermansia muciniphila, Escherichia coli, Streptococcus mitis, Veillonellaceae, Bilophila, ambiguous Klebsiella, Parascardovia, Lactobacillus delbrueckii subsp. Bulgaricus, Streptococcus salivarius, Coriobacteriaceae, Coprobacillus, Peptostreptococcaceae, Phascolarctobacterium faecium, Lactobacillus plantarum, Parabacteroides, Clostridium tertium, Eubacterium biforme, Alphaproteobacteria, Ruminococcus, Ruminococcaceae, Gemella haemolysans, Dorea, Streptococcus mutans, Coprococcus, Clostridium, Rothia, Megasphaera, Atopobium, Clostridium glycolicum, Coprococcus comes, Clostridium spiroforme, Lactobacillus fermentum, Enterococcus rivorum, Clostridium sp. 826, Enterobacter ludwigii, Lactobacillus animalis, Clostridium leptum, Ruminococcus (family Lachnospiraceae), Streptococcus parasanguinis, Clostridium sp. ID5, Enterococcus lactis, Granulicatella adiacens, Actinomyces graevenitzii, Clostridium bartlettii DSM 16795, S24-7, Clostridium algidixylanolyticum, Bifidobacterium dentium, Eubacterium limosum, Lactobacillus homohiochii, Tyzzerella nexilis, Proteobacteria, Mogibacterium neglectum, Lactobacillus reuteri, Clostridium saccharogumia, Mycoplasma, Holdemania filiformis, [Eubacterium] hallii, Drancourtella massiliensis, Clostridium perfringens, Bacteroides stercoris ATCC 43183, Ruminococcus faecis, Clostridium sp. PPf35E6, Lactococcus piscium, Bacteroides uniformis, Weissella confusa, Blautia, Megasphaera micronuciformis, Propionibacterium, Bacteroides thetaiotaomicron, Peptostreptococcus sp. MDA2346-2, Massiliomicrobiota timonensis, Bacteroides fragilis, Blautia hydrogenotrophica, Enterococcus mundtii, Prevotella melaninogenica, Erwinia chrysanthemi, Oscillospira, Clostridium nexile DSM 1787, Veillonella, Lactobacillus gasseri, Rothia dentocariosa, Clostridium sporosphaeroides, Lactococcus, Clostridium difficile, [Ruminococcus] obeum, Streptophyta, Clostridium hathewayi, Bacteroidales, Clostridium methylpentosum DSM 5476, Clostridium sp. cTPY-17, Parascardovia, Clostridium clostridioforme, Clostridium sp. Enrichment culture clone NHT38, Ruminococcus torques, Blautia obeum, Klebsiella oxytoca, Bulleidia moorei, Clostridium sp. cf3-PUG, Parabacteroides merdae, Lactobacillus buchneri, mitochondria, Shuttleworthia satelles, Coprococcus, Bacillaceae, Fusobacterium nucleatum, Streptococcus sp. DN812, Clostridium cellulosi, Lactobacillus acidophilus, Eggerthella lenta, Bacteroides ovatus, Clostridium hylemonae, Anaerococcus, Veillonella parvula, Clostridium sp. Culture-54, Anaerostipes caccae, Blautia faecis, Actinobaculum massiliense, Longibaculum muris, Enterococcus gallinarum, Butyrivibrio, Clostridium sp. MSTE9, Peptostreptococcaceae bacterium canine oral taxon 074, Alloscardovia omnicolens, ambiguous Leuconostoc, Paraprevotellaceae, Roseburia, Lactobacillus salivarius, unclassified Peptostreptococcaceae, Clostridium scindens, Alistipes, Clostridium lavalense, Anaerostipes, Abiotrophia defectiva, Leuconostocaceae, Fusobacterium, Atopobium rimae, Blautia luti, Dorea, Propionibacterium propionicum, Streptococcus lutetiensis, Alistipes putredinis, Salinicoccus qingdaonensis, Scardovia inopinata, Dorea formicigenerans, Parasutterella excrementihominis, Bacteroides caccae, Corynebacterium pseudogenitalium, Turicibacter sanguinis, Bifidobacteriaceae, Streptococcus anginosus, Roseburia faecis, Clostridium paraputrificum, Fusobacteria, Haemophilus parainfluenzae, Oscillospira, Clostridium aldenense, Clostridium symbiosum, Propionibacterium freudenreichii, a combination thereof, or a cluster comprising any one or more of the foregoing bacteria. These taxonomic groups can be a phylum, a class, an order, a family, a genus, a species, or a strain of bacteria. As used herein, the term “Group 1” refers to the above list of taxonomic groups of therapeutic bacteria, or spores thereof, that can be used for predicting survival, e.g., cancer survival, in a subject.

In certain non-limiting embodiments, the bacteria detected, e.g., in an intestinal microbiota sample of the subject, can be associated with an increased likelihood of survival, e.g., cancer survival. In certain embodiments, such bacteria can be selected from the bacteria listed in FIG. 5 and have a negative effect size value. For example, but not by way of limitation, such bacteria can be selected from the taxonomic groups found within super kingdom of bacteria comprising Clostridia, Streptococcaceae, Lactobacillaceae, Actinobacteria, Pediococcus acidilactici, Erysipelotrichales, Coprobacillaceae, Clostridiaceae, Akkermansia muciniphila, Escherichia coli, Streptococcus mitis, Veillonellaceae, Bilophila, Parascardovia, Streptococcus salivarius, Coriobacteriaceae, Peptostreptococcaceae, Phascolarctobacterium faecium, Lactobacillus plantarum, Clostridium tertium, Eubacterium biforme, Alphaproteobacteria, Ruminococcaceae, Gemella haemolysans, Dorea, Coprococcus, Clostridium, Rothia, Megasphaera, Atopobium, Clostridium glycolicum, Coprococcus comes, Clostridium spiroforme, Lactobacillus fermentum, Clostridium sp. 826, Lactobacillus animalis, Ruminococcus (family Lachnospiraceae), Streptococcus parasanguinis, Clostridium sp. ID5, Granulicatella adiacens, Actinomyces graevenitzii, Clostridium bartlettii DSM 16795, S24-7, Clostridium algidixylanolyticum, Lactobacillus homohiochii, Tyzzerella nexilis, Mogibacterium neglectum, Lactobacillus reuteri, Clostridium saccharogumia, [Eubacterium] hallii, Drancourtella massiliensis, Clostridium perfringens, Bacteroides stercoris ATCC 43183, Ruminococcus faecis, Clostridium sp. PPf35E6, Bacteroides uniformis, Weissella confusa, Megasphaera micronuciformis, Propionibacterium, Peptostreptococcus sp. MDA2346-2, Bacteroides fragilis, Oscillospira, Lactobacillus gasseri, Rothia dentocariosa, Clostridium sporosphaeroides, Lactococcus, Clostridium hathewayi, Clostridium methylpentosum DSM 5476, Clostridium sp. cTPY-17, Clostridium sp. Enrichment culture clone NHT38, Ruminococcus torques, Clostridium sp. cf3-PUG, Lactobacillus buchneri, mitochondria, Coprococcus, Bacillaceae, Fusobacterium nucleatum, Eggerthella lenta, Anaerococcus, Clostridium sp. Culture-54, Anaerostipes caccae, Blautia faecis, Actinobaculum massiliense, Enterococcus gallinarum, Clostridium sp. MSTE9, ambiguous Leuconostoc, Paraprevotellaceae, Roseburia, unclassified Peptostreptococcaceae, Fusobacterium, Atopobium rimae, Blautia luti, Propionibacterium propionicum, Streptococcus lutetiensis, Scardovia inopinata, Dorea formicigenerans, Corynebacterium pseudogenitalium, Turicibacter sanguinis, Roseburia faecis, Clostridium paraputrificum, Fusobacteria, Clostridium symbiosum, Propionibacterium freudenreichii, a combination thereof, or a cluster comprising any one or more of foregoing bacteria. These taxonomic groups can be a phylum, a class, an order, a family, a genus, a species, or a strain of bacteria. As used herein, the term “Group 2” refers to the above list of taxonomic groups of therapeutic bacteria, or spores thereof, that can be used for predicting a subject as likely to exhibit cancer survival.

In certain non-limiting embodiments, the bacteria detected, e.g., in an intestinal microbiota sample of the subject, can be associated with a decreased likelihood of survival, e.g., cancer survival. In certain embodiments, such bacteria can be selected from the bacteria listed in FIG. 5 and have a positive effect size value. For example, but not by way of limitation, such bacteria can be selected from the taxonomic groups found within super kingdom of bacteria comprising Firmicutes, Bacilli, Enterococcus, Bacillales, ambiguous Klebsiella, Lactobacillus delbrueckii subsp. Bulgaricus, Coprobacillus, Parabacteroides, Ruminococcus, Streptococcus mutans, Enterococcus rivorum, Enterobacter ludwigii, Clostridium leptum, Enterococcus lactis, Bifidobacterium dentium, Eubacterium limosum, Proteobacteria, Mycoplasma, Holdemania filiformis, Lactococcus piscium, Blautia, Bacteroides thetaiotaomicron, Massiliomicrobiota timonensis, Blautia hydrogenotrophica, Enterococcus mundtii, Prevotella melaninogenica, Erwinia chrysanthemi, Clostridium nexile DSM 1787, Veillonella, Clostridium difficile, [Ruminococcus] obeum, Streptophyta, Bacteroidales, Parascardovia, Clostridium clostridioforme, Blautia obeum, Klebsiella oxytoca, Bulleidia moorei, Parabacteroides merdae, Shuttleworthia satelles, Streptococcus sp. DN812, Clostridium cellulosi, Lactobacillus acidophilus, Bacteroides ovatus, Clostridium hylemonae, Veillonella parvula, Longibaculum muris, Butyrivibrio, Peptostreptococcaceae bacterium canine oral taxon 074, Alloscardovia omnicolens, Lactobacillus salivarius, Clostridium scindens, Alistipes, Clostridium lavalense, Anaerostipes, Abiotrophia defectiva, Leuconostocaceae, Dorea, Alistipes putredinis, Salinicoccus qingdaonensis, Parasutterella excrementihominis, Bacteroides caccae, Bifidobacteriaceae, Streptococcus anginosus, Haemophilus parainfluenzae, Oscillospira, Clostridium aldenense, a combination thereof, or a cluster comprising any one or more of foregoing bacteria. These taxonomic groups can be a phylum, a class, an order, a family, a genus, a species, or a strain of bacteria. As used herein, the term “Group 3” refers to the above list of taxonomic groups of therapeutic bacteria, or spores thereof, that can be used for predicting a subject as likely to not exhibit cancer survival.

In certain non-limiting embodiments, the bacteria can be detected prior to treating the subject, for example, prior to an allo-HCT.

In certain non-limiting embodiments, the bacteria can be detected after treating the subject, for example, after an allo-HCT.

In certain non-limiting embodiments, the bacteria can be detected in the peri-engraftment period. For example, but not by way of limitation, the bacteria can be detected about 7 days after an allo-HCT. In certain embodiments, the bacteria can be detected between about 7 days to about 21 days after an allo-HCT.

In certain non-limiting embodiments, detecting an abundance, e.g., increased abundance, of any one or more species of bacteria that are associated with an increased likelihood of survival, e.g., cancer survival, for example, a bacteria from the taxonomic groups listed in Group 2, can indicate that the subject is likely to exhibit survival, e.g., cancer survival. In certain non-limiting embodiments, detecting an abundance, e.g., increased abundance, of any one or more bacteria that are associated with an increased likelihood of survival, e.g., cancer survival, for example, a bacterial species from the taxonomic groups listed in Group 2, can indicate that the subject has a reduced risk of mortality.

In certain non-limiting embodiments, detecting an abundance, e.g., increased abundance, of any one or more bacteria that are associated with a decreased likelihood of survival, e.g., cancer survival, for example, a bacterial species from the taxonomic groups listed in Group 3, can indicate that the subject is not likely to exhibit survival, e.g., cancer survival. In certain non-limiting embodiments, detecting an abundance, e.g., increased abundance, of any one or more bacteria that are associated with a decreased likelihood of survival, e.g., cancer survival, for example, a bacterial species from the taxonomic groups listed in Group 3, can indicate that the subject has an increased risk of mortality.

In certain non-limiting embodiments, detecting a lower abundance of any one or more species of bacteria that are associated with an increased likelihood of cancer survival, for example, a bacteria from the taxonomic groups listed in Group 2, in a subject with cancer compared to a healthy subject can indicate that the subject with cancer has a decreased likelihood of cancer survival. In certain non-limiting embodiments, detecting a lower abundance of any one or more bacteria that are associated with an increased likelihood of cancer survival, for example, a bacteria from the taxonomic groups listed in Group 2, in a subject with cancer compared to a healthy subject can indicate that the subject with cancer has an increased risk of mortality.

In certain non-limiting embodiments, detecting a lower abundance of any one or more species of bacteria that are associated with a decreased likelihood of cancer survival, for example, a bacteria from the taxonomic groups listed in Group 3, in a subject with cancer compared to a healthy subject can indicate that the subject with cancer has an increased likelihood of cancer survival. In certain non-limiting embodiments, detecting a lower abundance of any one or more bacteria that are associated with a decreased likelihood of cancer survival, for example, a bacteria from the taxonomic groups listed in Group 3, in a subject with cancer compared to a healthy subject an indicate that the subject with cancer has a decreased risk of mortality.

In certain non-limiting embodiments, the methods of the present disclosure comprise determining the abundance of one more bacteria present in an intestinal microbiota sample of a subject, for example, a bacteria from the taxonomic groups listed in Group 2, a combination thereof, or a cluster comprising any one or more of the foregoing bacteria, wherein the subject is diagnosed or identified as likely to exhibit cancer survival, when the abundance or amount of the one or more bacteria in the subject's microbiota is greater than a bacteria reference level. In certain non-limiting embodiments, a bacteria reference level is an abundance of bacteria, for example, a bacteria from the taxonomic groups listed in Group 2, a combination thereof, or a cluster comprising any one or more of the foregoing bacteria, present in intestinal microbiota, a level above which is indicative of likely to exhibit cancer survival, as determined by a medical doctor or person of skill in the art.

In certain non-limiting embodiments, such a reference level is the abundance of said bacteria in the microbiota of a subject with cancer who has survived for at least about 2 years following a cancer treatment.

In certain non-limiting embodiments, such a reference level is the abundance of said bacteria in the microbiota of a healthy subject who does not have cancer, has not been diagnosed with cancer, or has a reduced risk for having cancer.

In certain embodiments, the level or abundance of the bacterium or spores thereof is the relative abundance of the bacterium or spores thereof as compared to other bacteria in the sample of the subject.

In certain embodiments, the level or the abundance of a bacterium can be determined by quantification of bacterial nucleic acid molecules (e.g., DNA or RNA molecules) in the sample. In certain embodiments, the bacterial nucleic acid molecule encodes 16S rRNA gene unique to the bacterial species. In certain embodiments, the level of the bacterial nucleic acid molecule 16S rRNA gene is determined by a sequencing method, e.g., metagenomic sequencing or shotgun metagenomic sequencing. In certain embodiments, the sequencing is performed using an Illumina MiSeq platform or Illumina HiSeq 2000 platform. In certain embodiments, the bacterial nucleic acid molecule level (e.g., copy number) is determined by an amplification-based method, e.g., by polymerase chain reaction (PCR), including reverse transcription-polymerase chain reaction (RT-PCR) for RNA quantitative analysis. In certain embodiments, amplification of the bacterial nucleic acid molecules in a sample can be accomplished by any known method, including but not limited to ligase chain reaction (LCR), transcription-mediated amplification, and self-sustained sequence replication or nucleic acid sequence-based amplification (NASBA). In certain embodiments, the level of a bacterial nucleic acid molecule level can be determined by size fractionation (e.g., gel electrophoresis), whether or not proceeded by an amplification step. In certain embodiments, the level of a bacterial nucleic acid molecule level can be determined by sequence-specific probe hybridization. In certain embodiments, the level of a bacterial nucleic acid molecule level can be determined by mass spectroscopy, PCR, microarray hybridization, thermal sequencing, capillary array sequencing, or solid phase sequencing.

In certain embodiments, the level or the abundance of the bacterium is determined by quantification of one or more proteins unique to the bacteria. In certain embodiments, the protein that is indicative of a bacterium's identity, can be detected but not limited using Western Blot, microarray, gel electrophoresis (such as 2-dimensional gel electrophoresis), and immunohistochemical assays.

In certain embodiments, the level or the abundance of the bacterium refers to a relative abundance of the bacterium in a sample. The relative abundance of a bacterium refers to the proportion occupied by the particular bacterium in the whole bacterial flora in the sample. The relative abundance of a bacterium can be determined from, for example, the total number of bacterial cells constituting the bacterial flora and the number of the particular bacterial cells included in the bacterial flora. More specifically, for example, genes having a nucleotide sequence that is common in the bacteria included in the bacterial flora and nucleotide sequences characteristic to each bacterial species (for example, 16S rRNA gene) are comprehensively decoded, and the relative abundance of a particular bacterium can be determined by designating the total number of decoded genes and the total number of genes belonging to particular bacterial species as the total number of bacterial cells constituting the bacterial flora and the number of particular bacterial cells, respectively.

In certain embodiments, the level of a bacterial gene is determined by measuring a level of a bacterial nucleic acids include DNA and RNA including at least a portion of the bacterial gene, a bacterial mRNA or cDNA that is transcribed from the bacterial gene, or a sequence complementary or homologous thereto (including but not limited to antisense or small interfering RNA). Said nucleic acid can be included of natural nucleotides and can optionally include nucleotide bases which are not naturally occurring. In certain embodiments, the level of a bacterial gene is determined by measuring a level of a bacterial protein that is encoded by the bacterial gene.

Any suitable methods known in the art for measuring nucleic acid and protein levels can be used with the presently disclosed methods. In certain embodiments, methods for measuring nucleic acid levels include, but not limited to, real-time PCR (RT-PCR), quantitative PCR, quantitative real-time polymerase chain reaction (qRT-PCR), fluorescent PCR, RT-MSP (RT methylation specific polymerase chain reaction), PicoGreen™ (Molecular Probes, Eugene, Oreg.) detection of DNA, radioimmunoassay or direct radio-labeling of DNA, in situ hybridization visualization, fluorescent in situ hybridization (FISH), microarray, sequencing.

In certain embodiments, methods for measuring protein levels include, but are not limited to, mass spectrometry techniques, 1-D or 2-D gel-based analysis systems, chromatography, enzyme linked immunosorbent assays (ELISAs), radioimmunoassays (RIA), enzyme immunoassays (EIA), Western Blotting, immunoprecipitation and immunohistochemistry.

In certain non-limiting embodiments, the methods of the present disclosure comprise determining the abundance of one more bacteria present in an intestinal microbiota sample of a subject, for example, a bacterial species from the taxonomic groups listed in Group 2, a combination thereof, or a cluster comprising any one or more of the foregoing bacteria, wherein the subject is diagnosed or identified as not likely to exhibit cancer survival, when the abundance or amount of the one or more bacteria in the subject's microbiota is lower than a bacteria reference level. In certain non-limiting embodiments, a bacteria reference level is an abundance of bacteria, for example, a bacterial species from the taxonomic groups listed in Group 2, a combination thereof, or a cluster comprising any one or more of the foregoing bacteria, present in intestinal microbiota, a level below which indicate the subject is not likely to exhibit cancer survival, as determined by a medical doctor or person of skill in the art.

In certain non-limiting embodiments, such a reference level can be the abundance of said bacteria in the microbiota of a subject with cancer who did not survive two years following treatment.

In certain non-limiting embodiments, the methods of the present disclosure comprise determining the abundance of bacteria in an intestinal microbiota sample of a subject that is indicative of a decreased likelihood of cancer survival. In certain non-limiting embodiments, the bacteria belong to a species from the taxonomic groups listed in Group 3.

In certain non-limiting embodiments, detecting an abundance of a bacterial species from the taxonomic groups listed in Group 3 in the subject that is greater than the abundance of said bacteria in an intestinal microbiota sample of a second subject that did not survive two years following treatment indicates that the subject is not likely to exhibit cancer survival.

In certain non-limiting embodiments, detecting an abundance of a bacterial species from the taxonomic groups listed in Group 3 in the subject that is greater than the abundance of said bacteria in an intestinal microbiota sample of a second subject that did survive two years following treatment indicates that the subject is not likely to exhibit cancer survival.

In certain non-limiting embodiments, detecting an abundance of a bacterial species from the taxonomic groups listed in Group 3 in the subject that is lower than the abundance of said bacteria in an intestinal microbiota sample of a second subject that did not survive two years following treatment indicates that the subject is likely to exhibit cancer survival.

In certain non-limiting embodiments, detecting an abundance of a bacterial species from the taxonomic groups listed in Group 3 in the subject that is lower than the abundance of said bacteria in an intestinal microbiota sample of a second subject that did survive two years following treatment indicates that the subject is likely to exhibit cancer survival.

In certain non-limiting embodiments, the methods of the present disclosure comprise determining the abundance of a bacterial species from the taxonomic groups listed in Group 3 present in an intestinal microbiota sample of a subject, wherein the subject is diagnosed or identified as not likely to exhibit cancer survival, when the abundance or amount of the bacterial species from the taxonomic groups listed in Group 3 in the subject's microbiota is greater than a bacteria reference level. In certain non-limiting embodiments, a bacteria reference level is an abundance of a bacterial species from the taxonomic groups listed in Group 3 present in intestinal microbiota, a level above which indicate the subject is not likely to exhibit cancer survival, as determined by a medical doctor or person of skill in the art.

In certain non-limiting embodiments, such a reference level can be the abundance of said bacteria in the microbiota of a subject with cancer who did not survive two years following treatment.

In certain non-limiting embodiments, the methods of the present disclosure comprise determining the abundance of a bacterial species from the taxonomic groups listed in Group 3 present in an intestinal microbiota sample of a subject, wherein the subject is diagnosed or identified as likely to exhibit cancer survival, when the abundance or amount of the bacterial species from the taxonomic groups listed in Group 3 in the subject's microbiota is less than a bacteria reference level. In certain non-limiting embodiments, a bacteria reference level is an abundance of a bacterial species from the taxonomic groups listed in Group 3, present in intestinal microbiota, at a level below which indicates that the subject is likely to exhibit cancer survival, as determined by a medical doctor or person of skill in the art.

In certain non-limiting embodiments, such a reference level can be the abundance of said bacteria in the microbiota of a subject with cancer who has survived for at least about 2 years following treatment.

In certain non-limiting embodiments, such a reference level can be the abundance of said bacteria in the microbiota of a healthy subject who has not been diagnosed with cancer, or has a reduced risk for having cancer.

In certain non-limiting embodiments, the microbiota sample is a fecal sample or an intestinal content sample, for example, a rectal swab.

In certain non-limiting embodiments, the abundance or amount of bacteria present in a sample is determined by measuring the abundance or amount of bacterial nucleic acid present in the sample, for example, 16S rRNA gene.

In certain non-limiting embodiments, the abundance or amount of bacteria present in a sample is determined by shotgun sequencing of bacterial DNA, PCR amplification of specific genes carried by the bacteria, quantitative PCR of transcripts expressed specifically by the bacteria, antibody-based methods of bacterial detection, metabolomic detection of bacterial metabolites, proteomic detection of bacterial proteins, and/or by methods of culturing the microbiota sample.

In certain non-limiting embodiments, the microbiota sample is collected from the subject up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more days after the subject has received cancer treatment, for example, allo-HCT. In certain non-limiting embodiments, the microbiota sample is collected from the subject up to 1, 2, 3, 4 or more weeks, or up to 1, 2, 3, 4, 5, 6, 7, 8, 9, 10, 11, 12 or more months, after the subject has received a cancer treatment. In certain non-limiting embodiments, the microbiota sample is collected from the subject up to 1, 2, 3, 4, 5, 6, 7 or more days, or up to 1, 2, 3, 4 or more weeks, or up to 1, 2, 3, 4, 5, 6, 7, 8, 9, 10, 11, 12 or more months, before the subject receives a cancer treatment, for example, allo-HCT.

### 5.3. Bacteria for Predicting Likelihood of Cancer Survival

The present disclosure provides bacteria and compositions comprising thereof for predicting likelihood of cancer survival. In certain embodiments, the presently disclosed bacteria can be used as diagnostic bacteria for predicting the likelihood of survival, e.g., cancer survival, in a subject. In certain embodiments, the presently disclosed bacteria can be used as therapeutic bacteria for the treatment of cancer and/or for increasing the likelihood of cancer survival in a subject being treated or to be treated for cancer, e.g., has received or will receive allo-HCT.

In certain non-limiting embodiments, the one or more bacteria, or spores thereof, associated with the likelihood of cancer survival, include a bacterial species from the taxonomic groups listed in Group 1.

In certain non-limiting embodiments, the one or more bacteria, or spores thereof, associated with an increased likelihood of cancer survival, include a bacterial species from the taxonomic groups listed in Group 2.

In certain non-limiting embodiments, the one or more bacteria, or spores thereof, associated with a decreased likelihood of cancer survival, include a bacterial species from the taxonomic groups listed in Group 3.

In certain non-limiting embodiments, the one or more bacteria can comprise one or more species from the bacteria class Clostridia, for example, found within the taxonomic group having National Center for Biotechnology Information taxonomy ID (NCBI:txid) 186801.

In certain non-limiting embodiments, the one or more bacteria can comprise one or more species from the bacteria family Streptococcaceae, for example, found within the taxonomic group having NCBI:txid 1300.

In certain non-limiting embodiments, the one or more bacteria can comprise one or more species from the bacteria family Lactobacillaceae, for example, found within the taxonomic group having NCBI:txid 33958.

In certain non-limiting embodiments, the one or more bacteria can comprise one or more species from the bacteria phylum Actinobacteria, for example, found within the taxonomic group having NCBI:txid 201174.

In certain non-limiting embodiments, the one or more bacteria can comprise one or more species from the bacterial species Pediococcus acidilactici, for example, found within the taxonomic group having NCBI:txid 1254, or bacteria having at least 90%, at least about 95%, at least about 97%, or at least about 99% identity with one or more 16S rRNA gene sequences, for example, as described by GenBank Accession number NR_041640 (SEQ ID NO:1), or a variable region of one or more 16S rRNA gene sequences such as the V4 region, from said Pediococcus acidilactici.

In certain non-limiting embodiments, the one or more bacteria can comprise one or more species from the bacteria order Erysipelotrichales, for example, found within the taxonomic group having NCBI:txid 526525.

In certain non-limiting embodiments, the one or more bacteria can comprise one or more species from the bacteria family Coprobacillaceae.

In certain non-limiting embodiments, the one or more bacteria can comprise one or more species from the bacteria family Clostridiaceae, for example, found within the taxonomic group having NCBI:txid 31979.

In certain non-limiting embodiments, the one or more bacteria can comprise one or more species from the bacterial species Akkermansia muciniphila, for example, found within the taxonomic group having NCBI:txid 239935, or bacteria having at least about 90%, at least about 95%, at least about 97%, or at least about 99% identity with one or more 16S rRNA gene sequences, for example, as described by GenBank Accession number NR_042817 (SEQ ID NO:2), or a variable region of one or more 16S rRNA gene sequences such as the V4 region, from said Akkermansia muciniphila.

In certain non-limiting embodiments, the one or more bacteria can comprise one or more species from the bacterial species Escherichia coli, for example, found within the taxonomic group having NCBI:txid 562, or bacteria having at least about 90%, at least about 95%, at least about 97%, or at least about 99% identity with one or more 16S rRNA gene sequences, for example, as described by GenBank Accession number NR_024570 (SEQ ID NO:3), or a variable region of one or more 16S rRNA gene sequences such as the V4 region, from said Escherichia co/i.

In certain non-limiting embodiments, the one or more bacteria can comprise one or more species from the bacterial species Streptococcus mitis, for example, found within the taxonomic group having NCBI:txid 28037, or bacteria having at least about 90%, at least about 95%, at least about 97%, or at least about 99% identity with one or more 16S rRNA gene sequences, for example, as described by GenBank Accession number NR_028664 (SEQ ID NO:4), or a variable region of one or more 16S rRNA gene sequences such as the V4 region, from said Streptococcus mitis.

In certain non-limiting embodiments, the one or more bacteria can comprise one or more species from the bacteria family Veillonellaceae, for example, found within the taxonomic group having NCBI:txid 31977.

In certain non-limiting embodiments, the one or more bacteria can comprise one or more species from the bacteria genus Bilophila, for example, found within the taxonomic group having NCBI:txid 35832.

In certain non-limiting embodiments, the one or more bacteria can comprise one or more species from the bacteria genus Parascardovia, for example, found within the taxonomic group having NCBI:txid 196082.

In certain non-limiting embodiments, the one or more bacteria can comprise one or more species from the bacterial species Streptococcus salivarius, for example, found within the taxonomic group having NCBI:txid 1304, or bacteria having at least about 90%, at least about 95%, at least about 97%, or at least about 99% identity with one or more 16S rRNA gene sequences, for example, as described by GenBank Accession number NR_042776 (SEQ ID NO:5), or a variable region of one or more 16S rRNA gene sequences such as the V4 region, from said Streptococcus salivarius.

In certain non-limiting embodiments, the one or more bacteria can comprise one or more species from the bacteria family Coriobacteriaceae, for example, found within the taxonomic group having NCBI:txid 84107.

In certain non-limiting embodiments, the one or more bacteria can comprise one or more species from the bacteria family Peptostreptococcaceae, for example, found within the taxonomic group having NCBI:txid 186804.

In certain non-limiting embodiments, the one or more bacteria can comprise one or more species from the bacterial species Phascolarctobacterium faecium, for example, found within the taxonomic group having NCBI:txid 33025, or bacteria having at least about 90%, at least about 95%, at least about 97%, or at least about 99% identity with one or more 16S rRNA gene sequences, for example, as described by GenBank Accession number NR_026111 (SEQ ID NO:6), or a variable region of one or more 16S rRNA gene sequences such as the V4 region, from said Phascolarctobacterium faecium.

In certain non-limiting embodiments, the one or more bacteria can comprise one or more species from the bacterial species Lactobacillus plantarum, for example, found within the taxonomic group having NCBI:txid 1590, or bacteria having at least about 90%, at least about 95%, at least about 97%, or at least about 99% identity with one or more 16S rRNA gene sequences, for example, as described by GenBank Accession number NR_042254 (SEQ ID NO:7), or a variable region of one or more 16S rRNA gene sequences such as the V4 region, from said Lactobacillus plantarum.

In certain non-limiting embodiments, the one or more bacteria can comprise one or more species from the bacterial species Clostridium tertium, for example, found within the taxonomic group having NCBI:txid 1559, or bacteria having at least about 90%, at least about 95%, at least about 97%, or at least about 99% identity with one or more 16S rRNA gene sequences, for example, as described by GenBank Accession number NR_037086 (SEQ ID NO:8), or a variable region of one or more 16S rRNA gene sequences such as the V4 region, from said Clostridium tertium.

In certain non-limiting embodiments, the one or more bacteria can comprise one or more species from the bacterial species Eubacterium biforme, for example, found within the taxonomic group having NCBI:txid 1735, or bacteria having at least about 90%, at least about 95%, at least about 97%, or at least about 99% identity with one or more 16S rRNA gene sequences, for example, as described by GenBank Accession number NR_044731 (SEQ ID NO:9), or a variable region of one or more 16S rRNA gene sequences such as the V4 region, from said Eubacterium biforme.

In certain non-limiting embodiments, the one or more bacteria can comprise one or more species from the bacteria class Alphaproteobacteria, for example, found within the taxonomic group having NCBI:txid 28211.

In certain non-limiting embodiments, the one or more bacteria can comprise one or more species from the bacteria family Ruminococcaceae, for example, found within the taxonomic group having NCBI:txid 541000.

In certain non-limiting embodiments, the one or more bacteria can comprise one or more species from the bacterial species Gemella haemolysans, for example, found within the taxonomic group having NCBI:txid 1379, or bacteria having at least about 90%, at least about 95%, at least about 97%, or at least about 99% identity with one or more 16S rRNA gene sequences, for example, as described by GenBank Accession number NR_025903 (SEQ ID NO:10), or a variable region of one or more 16S rRNA gene sequences such as the V4 region, from said Gemella haemolysans.

In certain non-limiting embodiments, the one or more bacteria can comprise one or more species from the bacteria genus Dorea, for example, found within the taxonomic group having NCBI:txid 189330.

In certain non-limiting embodiments, the one or more bacteria can comprise one or more species from the bacteria genus Coprococcus, for example, found within the taxonomic group having NCBI:txid 33042.

In certain non-limiting embodiments, the one or more bacteria can comprise one or more species from the bacteria genus Clostridium, for example, found within the taxonomic group having NCBI:txid 1485.

In certain non-limiting embodiments, the one or more bacteria can comprise one or more species from the bacteria genus Rothia, for example, found within the taxonomic group having NCBI:txid 32207.

In certain non-limiting embodiments, the one or more bacteria can comprise one or more species from the bacteria genus Megasphaera, for example, found within the taxonomic group having NCBI:txid 906.

In certain non-limiting embodiments, the one or more bacteria can comprise one or more species from the bacteria genus Atopobium, for example, found within the taxonomic group having NCBI:txid 1380.

In certain non-limiting embodiments, the one or more bacteria can comprise one or more species from the bacterial species Clostridium glycolicum, for example, found within the taxonomic group having NCBI:txid 36841, or bacteria having at least about 90%, at least about 95%, at least about 97%, or at least about 99% identity with one or more 16S rRNA gene sequences, for example, as described by GenBank Accession number NR_119074 (SEQ ID NO:11), or a variable region of one or more 16S rRNA gene sequences such as the V4 region, from said Clostridium glycolicum.

In certain non-limiting embodiments, the one or more bacteria can comprise one or more species from the bacterial species Coprococcus comes, for example, found within the taxonomic group having NCBI:txid 410072, or bacteria having at least about 90%, at least about 95%, at least about 97%, or at least about 99% identity with one or more 16S rRNA gene sequences, for example, as described by GenBank Accession number NR_044048 (SEQ ID NO:12), or a variable region of one or more 16S rRNA gene sequences such as the V4 region, from said Coprococcus comes.

In certain non-limiting embodiments, the one or more bacteria can comprise one or more species from the bacterial species Clostridium spiroforme, for example, found within the taxonomic group having NCBI:txid 29348, or bacteria having at least about 90%, at least about 95%, at least about 97%, or at least about 99% identity with one or more 16S rRNA gene sequences, for example, as described by GenBank Accession number NR_027592 (SEQ ID NO:13), or a variable region of one or more 16S rRNA gene sequences such as the V4 region, from said Clostridium spiroforme.

In certain non-limiting embodiments, the one or more bacteria can comprise one or more species from the bacterial species Lactobacillus fermentum, for example, found within the taxonomic group having NCBI:txid 1613, or bacteria having at least about 90%, at least about 95%, at least about 97%, or at least about 99% identity with one or more 16S rRNA gene sequences, for example, as described by GenBank Accession number NR_104927 (SEQ ID NO:14), or a variable region of one or more 16S rRNA gene sequences such as the V4 region, from said Lactobacillus fermentum.

In certain non-limiting embodiments, the one or more bacteria can comprise one or more species from the bacterial species Clostridium sp. 826, for example, found within the taxonomic group having NCBI:txid 1217284, or bacteria having at least about 90%, at least about 95%, at least about 97%, or at least about 99% identity with one or more 16S rRNA gene sequences, for example, as described by GenBank Accession number AB739699 (SEQ ID NO:15), or a variable region of one or more 16S rRNA gene sequences such as the V4 region, from said Clostridium sp. 826

In certain non-limiting embodiments, the one or more bacteria can comprise one or more species from the bacterial species Lactobacillus animalis, for example, found within the taxonomic group having NCBI:txid 1605, or bacteria having at least about 90%, at least about 95%, at least about 97%, or at least about 99% identity with one or more 16S rRNA gene sequences, for example, as described by GenBank Accession number NR_041610 (SEQ ID NO:16), or a variable region of one or more 16S rRNA gene sequences such as the V4 region, from said Lactobacillus animalis.

In certain non-limiting embodiments, the one or more bacteria can comprise one or more species from the bacteria genus Ruminococcus (family Lachnospiraceae).

In certain non-limiting embodiments, the one or more bacteria can comprise one or more species from the bacterial species Streptococcus parasanguinis, for example, found within the taxonomic group having NCBI:txid 1318, or bacteria having at least about 90%, at least about 95%, at least about 97%, or at least about 99% identity with one or more 16S rRNA gene sequences, for example, as described by GenBank Accession number NR_024842 (SEQ ID NO:17), or a variable region of one or more 16S rRNA gene sequences such as the V4 region, from said Streptococcus parasanguinis.

In certain non-limiting embodiments, the one or more bacteria can comprise one or more species from the bacterial species Clostridium sp. ID5, for example, found within the taxonomic group having NCBI:txid 320882, or bacteria having at least about 90%, at least about 95%, at least about 97%, or at least about 99% identity with one or more 16S rRNA gene sequences, for example, as described by GenBank Accession number AY960574 (SEQ ID NO:18), or a variable region of one or more 16S rRNA gene sequences such as the V4 region, from said Clostridium sp. ID5.

In certain non-limiting embodiments, the one or more bacteria can comprise one or more species from the bacterial species Granulicatella adiacens, for example, found within the taxonomic group having NCBI:txid 46124, or bacteria having at least about 90%, at least about 95%, at least about 97%, or at least about 99% identity with one or more 16S rRNA gene sequences, for example, as described by GenBank Accession number NR_025862 (SEQ ID NO:19), or a variable region of one or more 16S rRNA gene sequences such as the V4 region, from said Granulicatella adiacens.

In certain non-limiting embodiments, the one or more bacteria can comprise one or more species from the bacterial species Actinomyces graevenitzii, for example, found within the taxonomic group having NCBI:txid 55565, or bacteria having at least about 90%, at least about 95%, at least about 97%, or at least about 99% identity with one or more 16S rRNA gene sequences, for example, as described by GenBank Accession number NR_042167 (SEQ ID NO:20), or a variable region of one or more 16S rRNA gene sequences such as the V4 region, from said Actinomyces graevenitzii.

In certain non-limiting embodiments, the one or more bacteria can comprise one or more species from the bacterial species Clostridium bartlettii DSM 16795, for example, found within the taxonomic group having NCBI:txid 445973, or bacteria having at least about 90%, at least about 95%, at least about 97%, or at least about 99% identity with one or more 16S rRNA gene sequences, for example, as described by GenBank Accession number AY438672 (SEQ ID NO:21), or a variable region of one or more 16S rRNA gene sequences such as the V4 region, from said Clostridium bartlettiii DSM 16795.

In certain non-limiting embodiments, the one or more bacteria can comprise one or more species from the bacteria family S24-7, for example, found within the taxonomic group having NCBI:txid 2005473.

In certain non-limiting embodiments, the one or more bacteria can comprise one or more species from the bacterial species Clostridium algidixylanolyticum, for example, found within the taxonomic group having NCBI:txid 94868, or bacteria having at least about 90%, at least about 95%, at least about 97%, or at least about 99% identity with one or more 16S rRNA gene sequences, for example, as described by GenBank Accession number NR_028726 (SEQ ID NO:22), or a variable region of one or more 16S rRNA gene sequences such as the V4 region, from said Clostridium algidixylanolyticum.

In certain non-limiting embodiments, the one or more bacteria can comprise one or more species from the bacterial species Lactobacillus homohiochii, for example, found within the taxonomic group having NCBI:txid 33961, or bacteria having at least about 90%, at least about 95%, at least about 97%, or at least about 99% identity with one or more 16S rRNA gene sequences, for example, as described by GenBank Accession number LC311740 (SEQ ID NO:23), or a variable region of one or more 16S rRNA gene sequences such as the V4 region, from said Lactobacillus homohiochii.

In certain non-limiting embodiments, the one or more bacteria can comprise one or more species from the bacterial species Tyzzerella nexilis, for example, found within the taxonomic group having NCBI:txid 29361, or bacteria having at least about 90%, at least about 95%, at least about 97%, or at least about 99% identity with one or more 16S rRNA gene sequences, for example, as described by GenBank Accession number NR_029248 (SEQ ID NO:24), or a variable region of one or more 16S rRNA gene sequences such as the V4 region, from said Tyzzerella nexilis.

In certain non-limiting embodiments, the one or more bacteria can comprise one or more species from the bacterial species Mogibacterium neglectum, for example, found within the taxonomic group having NCBI:txid 114528, or bacteria having at least about 90%, at least about 95%, at least about 97%, or at least about 99% identity with one or more 16S rRNA gene sequences, for example, as described by GenBank Accession number NR_027203 (SEQ ID NO:25), or a variable region of one or more 16S rRNA gene sequences such as the V4 region, from said Mogibacterium neglectum.

In certain non-limiting embodiments, the one or more bacteria can comprise one or more species from the bacterial species Lactobacillus reuteri, for example, found within the taxonomic group having NCBI:txid 1598, or bacteria having at least about 90%, at least about 95%, at least about 97%, or at least about 99% identity with one or more 16S rRNA gene sequences, for example, as described by GenBank Accession number NR_025911 (SEQ ID NO:26), or a variable region of one or more 16S rRNA gene sequences such as the V4 region, from said Lactobacillus reuteri.

In certain non-limiting embodiments, the one or more bacteria can comprise one or more species from the bacterial species Clostridium saccharogumia, for example, found within the taxonomic group having NCBI:txid 341225, or bacteria having at least about 90%, at least about 95%, at least about 97%, or at least about 99% identity with one or more 16S rRNA gene sequences, for example, as described by GenBank Accession number NR_043550 (SEQ ID NO:27), or a variable region of one or more 16S rRNA gene sequences such as the V4 region, from said Clostridium saccharogumia.

In certain non-limiting embodiments, the one or more bacteria can comprise one or more species from the bacterial species [Eubacterium] hallii, for example, found within the taxonomic group having NCBI:txid 39488, or bacteria having at least about 90%, at least about 95%, at least about 97%, or at least about 99% identity with one or more 16S rRNA gene sequences, for example, as described by GenBank Accession number L34621 (SEQ ID NO:28), or a variable region of one or more 16S rRNA gene sequences such as the V4 region, from said [Eubacterium] hallii.

In certain non-limiting embodiments, the one or more bacteria can comprise one or more species from the bacterial species Drancourtella massiliensis, for example, found within the taxonomic group having NCBI:txid 1632013, or bacteria having at least about 90%, at least about 95%, at least about 97%, or at least about 99% identity with one or more 16S rRNA gene sequences, for example, as described by GenBank Accession number NR_144722 (SEQ ID NO:29), or a variable region of one or more 16S rRNA gene sequences such as the V4 region, from said Drancourtella massiliensis.

In certain non-limiting embodiments, the one or more bacteria can comprise one or more species from the bacterial species Clostridium perfringens, for example, found within the taxonomic group having NCBI:txid 1502, or bacteria having at least about 90%, at least about 95%, at least about 97%, or at least about 99% identity with one or more 16S rRNA gene sequences, for example, as described by GenBank Accession number NR_121697 (SEQ ID NO:30), or a variable region of one or more 16S rRNA gene sequences such as the V4 region, from said Clostridium perfringens.

In certain non-limiting embodiments, the one or more bacteria can comprise one or more species from the bacterial species Bacteroides stercoris ATCC 43183, for example, found within the taxonomic group having NCBI:txid 449673, or bacteria having at least about 90%, at least about 95%, at least about 97%, or at least about 99% identity with one or more 16S rRNA gene sequences, for example, as described by GenBank Accession number EU136684 (SEQ ID NO:31), or a variable region of one or more 16S rRNA gene sequences such as the V4 region, from said Bacteroides stercoris ATCC 43183.

In certain non-limiting embodiments, the one or more bacteria can comprise one or more species from the bacterial species Ruminococcus faecis, for example, found within the taxonomic group having NCBI:txid 592978, or bacteria having at least about 90%, at least about 95%, at least about 97%, or at least about 99% identity with one or more 16S rRNA gene sequences, for example, as described by GenBank Accession number NR_116747 (SEQ ID NO:32), or a variable region of one or more 16S rRNA gene sequences such as the V4 region, from said Ruminococcus faecis.

In certain non-limiting embodiments, the one or more bacteria can comprise one or more species from the bacterial species Clostridium sp. PPf35E6, for example, found within the taxonomic group having NCBI:txid 265482, or bacteria having at least about 90%, at least about 95%, at least about 97%, or at least about 99% identity with one or more 16S rRNA gene sequences, for example, as described by GenBank Accession number AY548783 (SEQ ID NO:33), or a variable region of one or more 16S rRNA gene sequences such as the V4 region, from said Clostridium sp. PPf35E6.

In certain non-limiting embodiments, the one or more bacteria can comprise one or more species from the bacterial species Bacteroides uniformis, for example, found within the taxonomic group having NCBI:txid 820, or bacteria having at least about 90%, at least about 95%, at least about 97%, or at least about 99% identity with one or more 16S rRNA gene sequences, for example, as described by GenBank Accession number AB247146 (SEQ ID NO:34), or a variable region of one or more 16S rRNA gene sequences such as the V4 region, from said Bacteroides uniformis.

In certain non-limiting embodiments, the one or more bacteria can comprise one or more species from the bacterial species Weissella confusa, for example, found within the taxonomic group having NCBI:txid 1583, or bacteria having at least about 90%, at least about 95%, at least about 97%, or at least about 99% identity with one or more 16S rRNA gene sequences, for example, as described by GenBank Accession number AB596944 (SEQ ID NO:35), or a variable region of one or more 16S rRNA gene sequences such as the V4 region, from said Weissella confusa.

In certain non-limiting embodiments, the one or more bacteria can comprise one or more species from the bacterial species Megasphaera micronuciformis, for example, found within the taxonomic group having NCBI:txid 187326, or bacteria having at least about 90%, at least about 95%, at least about 97%, or at least about 99% identity with one or more 16S rRNA gene sequences, for example, as described by GenBank Accession number NR_025230 (SEQ ID NO:36), or a variable region of one or more 16S rRNA gene sequences such as the V4 region, from said Megasphaera micronuciformis.

In certain non-limiting embodiments, the one or more bacteria can comprise one or more species from the bacteria genus Propionibacterium for example, found within the taxonomic group having NCBI:txid 1743.

In certain non-limiting embodiments, the one or more bacteria can comprise one or more species from the bacterial species Peptostreptococcus sp. MDA2346-2, for example, found within the taxonomic group having NCBI:txid 231367, or bacteria having at least about 90%, at least about 95%, at least about 97%, or at least about 99% identity with one or more 16S rRNA gene sequences, for example, as described by GenBank Accession number AY286545 (SEQ ID NO:37), or a variable region of one or more 16S rRNA gene sequences such as the V4 region, from said Peptostreptococcus sp. MDA2346-2.

In certain non-limiting embodiments, the one or more bacteria can comprise one or more species from the bacterial species Bacteroides fragilis, for example, found within the taxonomic group having NCBI:txid 817, or bacteria having at least about 90%, at least about 95%, at least about 97%, or at least about 99% identity with one or more 16S rRNA gene sequences, for example, as described by GenBank Accession number NR_074784 (SEQ ID NO:38), or a variable region of one or more 16S rRNA gene sequences such as the V4 region, from said Bacteroides fragilis.

In certain non-limiting embodiments, the one or more bacteria can comprise one or more species from the bacteria genus Oscillospira for example, found within the taxonomic group having NCBI:txid 119852.

In certain non-limiting embodiments, the one or more bacteria can comprise one or more species from the bacterial species Lactobacillus gasseri, for example, found within the taxonomic group having NCBI:txid 1596, or bacteria having at least about 90%, at least about 95%, at least about 97%, or at least about 99% identity with one or more 16S rRNA gene sequences, for example, as described by GenBank Accession number NR_075051 (SEQ ID NO:39), or a variable region of one or more 16S rRNA gene sequences such as the V4 region, from said Lactobacillus gasseri.

In certain non-limiting embodiments, the one or more bacteria can comprise one or more species from the bacterial species Rothia dentocariosa, for example, found within the taxonomic group having NCBI:txid 2047, or bacteria having at least about 90%, at least about 95%, at least about 97%, or at least about 99% identity with one or more 16S rRNA gene sequences, for example, as described by GenBank Accession number NR_074568 (SEQ ID NO:40), or a variable region of one or more 16S rRNA gene sequences such as the V4 region, from said Rothia dentocariosa.

In certain non-limiting embodiments, the one or more bacteria can comprise one or more species from the bacterial species Clostridium sporosphaeroides, for example, found within the taxonomic group having NCBI:txid 1549, or bacteria having at least about 90%, at least about 95%, at least about 97%, or at least about 99% identity with one or more 16S rRNA gene sequences, for example, as described by GenBank Accession number NR_044835 (SEQ ID NO:41), or a variable region of one or more 16S rRNA gene sequences such as the V4 region, from said Clostridium sporosphaeroides.

In certain non-limiting embodiments, the one or more bacteria can comprise one or more species from the bacteria genus Lactococcus for example, found within the taxonomic group having NCBI:txid 1357.

In certain non-limiting embodiments, the one or more bacteria can comprise one or more species from the bacterial species Clostridium hathewayi, for example, found within the taxonomic group having NCBI:txid 154046, or bacteria having at least about 90%, at least about 95%, at least about 97%, or at least about 99% identity with one or more 16S rRNA gene sequences, for example, as described by GenBank Accession number NR_036928 (SEQ ID NO:42), or a variable region of one or more 16S rRNA gene sequences such as the V4 region, from said Clostridium hathewayi.

In certain non-limiting embodiments, the one or more bacteria can comprise one or more species from the bacterial species Clostridium methylpentosum DSM 5476, for example, found within the taxonomic group having NCBI:txid 537013, or bacteria having at least about 90%, at least about 95%, at least about 97%, or at least about 99% identity with one or more 16S rRNA gene sequences, for example, as described by GenBank Accession number Y18181 (SEQ ID NO:43), or a variable region of one or more 16S rRNA gene sequences such as the V4 region, from said Clostridium methylpentosum DSM 5476.

In certain non-limiting embodiments, the one or more bacteria can comprise one or more species from the bacterial species Clostridium sp. cTPY-17, for example, found within the taxonomic group having NCBI:txid 245292, or bacteria having at least about 90%, at least about 95%, at least about 97%, or at least about 99% identity with one or more 16S rRNA gene sequences, for example, as described by GenBank Accession number AY239462 (SEQ ID NO:44), or a variable region of one or more 16S rRNA gene sequences such as the V4 region, from said Clostridium sp. cTPY-17.

In certain non-limiting embodiments, the one or more bacteria can comprise one or more species from the bacterial species Clostridium sp. Enrichment culture clone NHT38, for example, found within the taxonomic group having NCBI:txid 986803, or bacteria having at least about 90%, at least about 95%, at least about 97%, or at least about 99% identity with one or more 16S rRNA gene sequences, for example, as described by GenBank Accession number JF312678 (SEQ ID NO:45), or a variable region of one or more 16S rRNA gene sequences such as the V4 region, from said Clostridium sp. Enrichment culture clone NHT38.

In certain non-limiting embodiments, the one or more bacteria can comprise one or more species from the bacterial species Ruminococcus torques, for example, found within the taxonomic group having NCBI:txid 33039, or bacteria having at least about 90%, at least about 95%, at least about 97%, or at least about 99% identity with one or more 16S rRNA gene sequences, for example, as described by GenBank Accession number NR_036777 (SEQ ID NO:46), or a variable region of one or more 16S rRNA gene sequences such as the V4 region, from said Ruminococcus torques.

In certain non-limiting embodiments, the one or more bacteria can comprise one or more species from the bacterial species Clostridium sp. cf3-PUG, for example, found within the taxonomic group having NCBI:txid 999944, or bacteria having at least about 90%, at least about 95%, at least about 97%, or at least about 99% identity with one or more 16S rRNA gene sequences, for example, as described by GenBank Accession number HQ324121 (SEQ ID NO:47), or a variable region of one or more 16S rRNA gene sequences such as the V4 region, from said Clostridium sp. cf3-PUG.

In certain non-limiting embodiments, the one or more bacteria can comprise one or more species from the bacterial species Lactobacillus buchneri, for example, found within the taxonomic group having NCBI:txid 1581, or bacteria having at least about 90%, at least about 95%, at least about 97%, or at least about 99% identity with one or more 16S rRNA gene sequences, for example, as described by GenBank Accession number NR_041293 (SEQ ID NO:48), or a variable region of one or more 16S rRNA gene sequences such as the V4 region, from said Lactobacillus buchneri.

In certain non-limiting embodiments, the one or more bacteria can comprise one or more species from the bacteria family mitochondria.

In certain non-limiting embodiments, the one or more bacteria can comprise one or more species from the bacteria genus Coprococcus for example, found within the taxonomic group having NCBI:txid 33042.

In certain non-limiting embodiments, the one or more bacteria can comprise one or more species from the bacteria family Bacillaceae for example, found within the taxonomic group having NCBI:txid 186817.

In certain non-limiting embodiments, the one or more bacteria can comprise one or more species from the bacterial species Fusobacterium nucleatum, for example, found within the taxonomic group having NCBI:txid 851, or bacteria having at least about 90%, at least about 95%, at least about 97%, or at least about 99% identity with one or more 16S rRNA gene sequences, for example, as described by GenBank Accession number KU726670 (SEQ ID NO:49), or a variable region of one or more 16S rRNA gene sequences such as the V4 region, from said Fusobacterium nucleatum.

In certain non-limiting embodiments, the one or more bacteria can comprise one or more species from the bacterial species Eggerthella lenta, for example, found within the taxonomic group having NCBI:txid 84112, or bacteria having at least about 90%, at least about 95%, at least about 97%, or at least about 99% identity with one or more 16S rRNA gene sequences, for example, as described by GenBank Accession number NR_074377 (SEQ ID NO:50), or a variable region of one or more 16S rRNA gene sequences such as the V4 region, from said Eggerthella lenta.

In certain non-limiting embodiments, the one or more bacteria can comprise one or more species from the bacteria genus Anaerococcus for example, found within the taxonomic group having NCBI:txid 165779.

In certain non-limiting embodiments, the one or more bacteria can comprise one or more species from the bacterial species Clostridium sp. Culture-54, for example, found within the taxonomic group having NCBI:txid 1003352, or bacteria having at least about 90%, at least about 95%, at least about 97%, or at least about 99% identity with one or more 16S rRNA gene sequences, for example, as described by GenBank Accession number AB622823 (SEQ ID NO:51), or a variable region of one or more 16S rRNA gene sequences such as the V4 region, from said Clostridium sp. Culture-54.

In certain non-limiting embodiments, the one or more bacteria can comprise one or more species from the bacterial species Anaerostipes caccae, for example, found within the taxonomic group having NCBI:txid 105841, or bacteria having at least about 90%, at least about 95%, at least about 97%, or at least about 99% identity with one or more 16S rRNA gene sequences, for example, as described by GenBank Accession number AB243986 (SEQ ID NO:52), or a variable region of one or more 16S rRNA gene sequences such as the V4 region, from said Anaerostipes caccae.

In certain non-limiting embodiments, the one or more bacteria can comprise one or more species from the bacterial species Blautia faecis, for example, found within the taxonomic group having NCBI:txid 871665, or bacteria having at least about 90%, at least about 95%, at least about 97%, or at least about 99% identity with one or more 16S rRNA gene sequences, for example, as described by GenBank Accession number NR_109014 (SEQ ID NO:53), or a variable region of one or more 16S rRNA gene sequences such as the V4 region, from said Blautia faecis.

In certain non-limiting embodiments, the one or more bacteria can comprise one or more species from the bacterial species Actinobaculum massiliense, for example, found within the taxonomic group having NCBI:txid 202789, or bacteria having at least about 90%, at least about 95%, at least about 97%, or at least about 99% identity with one or more 16S rRNA gene sequences, for example, as described by GenBank Accession number MH645801 (SEQ ID NO:54), or a variable region of one or more 16S rRNA gene sequences such as the V4 region, from said Actinobaculum massiliense.

In certain non-limiting embodiments, the one or more bacteria can comprise one or more species from the bacterial species Enterococcus gallinarum, for example, found within the taxonomic group having NCBI:txid 1353, or bacteria having at least about 90%, at least about 95%, at least about 97%, or at least about 99% identity with one or more 16S rRNA gene sequences, for example, as described by GenBank Accession number NR_113924 (SEQ ID NO:55), or a variable region of one or more 16S rRNA gene sequences such as the V4 region, from said Enterococcus gallinarum.

In certain non-limiting embodiments, the one or more bacteria can comprise one or more species from the bacterial species Clostridium sp. MSTE9, for example, found within the taxonomic group having NCBI:txid 1105031, or bacteria having at least about 90%, at least about 95%, at least about 97%, or at least about 99% identity with one or more 16S rRNA gene sequences, for example, as described by GenBank Accession number JN091083 (SEQ ID NO:56), or a variable region of one or more 16S rRNA gene sequences such as the V4 region, from said Clostridium sp. MSTE9.

In certain non-limiting embodiments, the one or more bacteria can comprise one or more species from the bacterial species ambiguous Leuconostoc, or the bacteria having at least about 90%, at least about 95%, at least about 97%, or at least about 99% identity with the 16S rRNA gene sequence having the sequence set forth in SEQ ID NO:57, or a variable region of one or more 16S rRNA gene sequences such as the V4 region, from said ambiguous Leuconostoc.

[SEQ ID NO: 57]
AGCGCAGACGGTTGATTAAGTCTGATGTGAAAGCC
CGGAGCTCAACTCCGGAAAGGCATTGGAAACTGGT
CAACTTGAGTGCAGTAGAGGTAAGTGGAACTCCAT
GTGTAGCGGTGGAATGCGTAGATATATGGAAGAAC
ACCAGCGGCGAAGGCGGCTTACTGGACTGTAACTG
ACGTTGAGGCTCGAAAGTGTGGGTAGCAAACAGGA
TTAGATACCCTGGTAGTCCACACCGTAAACGATGA
ACACTAGGTGTTAGGAGGTTTCCGCCTCTTAGTGC
CGAAGCTAACGCATTAAGTGTTCCGCCTGGGGAGT
ACGACCGCAAGGTTGAA

In certain non-limiting embodiments, the one or more bacteria can comprise one or more species from the bacteria family Paraprevotellaceae.

In certain non-limiting embodiments, the one or more bacteria can comprise one or more species from the bacteria genus Roseburia for example, found within the taxonomic group having NCBI:txid 841.

In certain non-limiting embodiments, the one or more bacteria can comprise one or more species from the bacteria genus unclassified Peptostreptococcaceae for example, found within the taxonomic group having NCBI:txid 200630.

In certain non-limiting embodiments, the one or more bacteria can comprise one or more species from the bacteria genus Fusobacterium for example, found within the taxonomic group having NCBI:txid 848.

In certain non-limiting embodiments, the one or more bacteria can comprise one or more species from the bacterial species Atopobium rimae, for example, found within the taxonomic group having NCBI:txid 1383, or bacteria having at least about 90%, at least about 95%, at least about 97%, or at least about 99% identity with one or more 16S rRNA gene sequences, for example, as described by GenBank Accession number NR_036819 (SEQ ID NO:58), or a variable region of one or more 16S rRNA gene sequences such as the V4 region, from said Atopobium rimae.

In certain non-limiting embodiments, the one or more bacteria can comprise one or more species from the bacterial species Blautia luti, for example, found within the taxonomic group having NCBI:txid 89014, or bacteria having at least about 90%, at least about 95%, at least about 97%, or at least about 99% identity with one or more 16S rRNA gene sequences, for example, as described by GenBank Accession number NR_041960 (SEQ ID NO:59), or a variable region of one or more 16S rRNA gene sequences such as the V4 region, from said Blautia luti.

In certain non-limiting embodiments, the one or more bacteria can comprise one or more species from the bacterial species Propionibacterium propionicum, for example, found within the taxonomic group having NCBI:txid 1750, or bacteria having at least about 90%, at least about 95%, at least about 97%, or at least about 99% identity with one or more 16S rRNA gene sequences, for example, as described by GenBank Accession number NR_025277 (SEQ ID NO:60), or a variable region of one or more 16S rRNA gene sequences such as the V4 region, from said Propionibacterium propionicum.

In certain non-limiting embodiments, the one or more bacteria can comprise one or more species from the bacterial species Streptococcus lutetiensis, for example, found within the taxonomic group having NCBI:txid 150055, or bacteria having at least about 90%, at least about 95%, at least about 97%, or at least about 99% identity with one or more 16S rRNA gene sequences, for example, as described by GenBank Accession number NR_037096 (SEQ ID NO:61), or a variable region of one or more 16S rRNA gene sequences such as the V4 region, from said Streptococcus lutetiensis.

In certain non-limiting embodiments, the one or more bacteria can comprise one or more species from the bacterial species Scardovia inopinata, for example, found within the taxonomic group having NCBI:txid 78259, or bacteria having at least about 90%, at least about 95%, at least about 97%, or at least about 99% identity with one or more 16S rRNA gene sequences, for example, as described by GenBank Accession number NR_043451 (SEQ ID NO:62), or a variable region of one or more 16S rRNA gene sequences such as the V4 region, from said Scardovia inopinata.

In certain non-limiting embodiments, the one or more bacteria can comprise one or more species from the bacterial species Dorea formicigenerans, for example, found within the taxonomic group having NCBI:txid 39486, or bacteria having at least about 90%, at least about 95%, at least about 97%, or at least about 99% identity with one or more 16S rRNA gene sequences, for example, as described by GenBank Accession number NR_044645 (SEQ ID NO:63), or a variable region of one or more 16S rRNA gene sequences such as the V4 region, from said Dorea formicigenerans.

In certain non-limiting embodiments, the one or more bacteria can comprise one or more species from the bacterial species Corynebacterium pseudogenitalium, for example, found within the taxonomic group having NCBI:txid 38303, or bacteria having at least about 90%, at least about 95%, at least about 97%, or at least about 99% identity with one or more 16S rRNA gene sequences, for example, as described by GenBank Accession number X81872 (SEQ ID NO:64), or a variable region of one or more 16S rRNA gene sequences such as the V4 region, from said Corynebacterium pseudogenitalium.

In certain non-limiting embodiments, the one or more bacteria can comprise one or more species from the bacterial species Turicibacter sanguinis, for example, found within the taxonomic group having NCBI:txid 154288, or bacteria having at least about 90%, at least about 95%, at least about 97%, or at least about 99% identity with one or more 16S rRNA gene sequences, for example, as described by GenBank Accession number NR_028816 (SEQ ID NO:65), or a variable region of one or more 16S rRNA gene sequences such as the V4 region, from said Turicibacter sanguinis.

In certain non-limiting embodiments, the one or more bacteria can comprise one or more species from the bacterial species Roseburia faecis, for example, found within the taxonomic group having NCBI:txid 301302, or bacteria having at least about 90%, at least about 95%, at least about 97%, or at least about 99% identity with one or more 16S rRNA gene sequences, for example, as described by GenBank Accession number NR_042832 (SEQ ID NO:66), or a variable region of one or more 16S rRNA gene sequences such as the V4 region, from said Roseburia faecis.

In certain non-limiting embodiments, the one or more bacteria can comprise one or more species from the bacterial species Clostridium paraputrificum, for example, found within the taxonomic group having NCBI:txid 29363, or bacteria having at least about 90%, at least about 95%, at least about 97%, or at least about 99% identity with one or more 16S rRNA gene sequences, for example, as described by GenBank Accession number NR_026135 (SEQ ID NO:67), or a variable region of one or more 16S rRNA gene sequences such as the V4 region, from said Clostridium paraputrificum.

In certain non-limiting embodiments, the one or more bacteria can comprise one or more species from the bacteria phylum Fusobacteria, for example, found within the taxonomic group having NCBI:txid 32066.

In certain non-limiting embodiments, the one or more bacteria can comprise one or more species from the bacterial species Clostridium symbiosum, for example, found within the taxonomic group having NCBI:txid 1512, or bacteria having at least about 90%, at least about 95%, at least about 97%, or at least about 99% identity with one or more 16S rRNA gene sequences, for example, as described by GenBank Accession number NR_118730 (SEQ ID NO:68), or a variable region of one or more 16S rRNA gene sequences such as the V4 region, from said Clostridium symbiosum.

In certain non-limiting embodiments, the one or more bacteria can comprise one or more species from the bacterial species Propionibacterium freudenreichii, for example, found within the taxonomic group having NCBI:txid 1744, or bacteria having at least about 90%, at least about 95%, at least about 97%, or at least about 99% identity with one or more 16S rRNA gene sequences, for example, as described by GenBank Accession number X53217 (SEQ ID NO:69), or a variable region of one or more 16S rRNA gene sequences such as the V4 region, from said Propionibacterium freudenreichii.

In certain non-limiting embodiments, the one or more bacteria can comprise one or more species from the bacteria phylum Firmicutes for example, found within the taxonomic group having NCBI:txid 1239.

In certain non-limiting embodiments, the one or more bacteria can comprise one or more species from the bacteria class Bacilli, for example, found within the taxonomic group having NCBI:txid 91061.

In certain non-limiting embodiments, the one or more bacteria can comprise one or more species from the bacteria genus Enterococcus, for example, found within the taxonomic group having NCBI:txid 1350.

In certain non-limiting embodiments, the one or more bacteria can comprise one or more species from the bacteria order Bacillales, for example, found within the taxonomic group having NCBI:txid 1385.

In certain non-limiting embodiments, the one or more bacteria can comprise one or more species from the bacterial species ambiguous Klebsiella, for example bacteria having at least about 90%, at least about 95%, at least about 97%, or at least about 99% identity with a 16S rRNA gene sequence having the sequence set forth in SEQ ID NO:70, SEQ ID NO: 116, SEQ ID NO: 117 or SEQ ID NO: 118, or a variable region of one or more 16S rRNA gene sequences such as the V4 region, from said ambiguous Klebsiella.

[SEQ ID NO: 70]
CACGCAGGCGGTCTGTCAAGTCGGATGTGAAATCC
CCGGGCTCAACCTGGGAACTGCATTCGAAACTGGC
AGGCTAGAGTCTTGTAGAGGGGGGTAGAATTCCAG
GTGTAGCGGTGAAATGCGTAGAGATCTGGAGGAAT
ACCGGTGGCGAAGGCGGCCCCCTGGACAAAGACTG
ACGCTCAGGTGCGAAAGCGTGGGGAGCAAACAGGA
TTAGATACCCTGGTAGTCCACGCCGTAAACGATGT
CGATTTGGAGGTTGTGCCCTTGAGGCGTGGCTTCC
GGAGCTAACGCGTTAAATCGACCGCCTGGGGAGTA
CGTTCGCAAGAATGAA
[SEQ ID NO: 116]
AGCGCAGGCGGTTTCTTAAGTCTGATGTGAAATCC
CCGGGCTTAACCTGGGAACTGCATTCGAAACTGGC
AGGCTAGAGTCTTGTAGAGGGGGGTAGAATTCCAG
GTGTAGCGGTGAAATGCGTAGAGATCTGGAGGAAT
ACCGGTGGCGAAGGCGGCCCCCTGGACAAAGACTG
ACGCTCAGGTGCGAAAGCGTGGGGAGCAAACAGGA
TTAGATACCCTGGTAGTCCACGCCGTAAACGATGT
CGATTTGGAGGTTGTGCCCTTGAGGCGTGGCTTCC
GGAGCTAACGCGTTAAATCGACCGCCTGGGGAGTA
CGGCCGCAAGGTTAAA
[SEQ ID NO: 117]
AGCGCAGGCGGGAAGACAAGTTGGAAGTGAAATCC
CCGGGCTCAACCTGGGAACTGCATTCGAAACTGGC
AGGCTAGAGTCTTGTAGAGGGGGGTAGAATTCCAG
GTGTAGCGGTGAAATGCGTAGAGATCTGGAGGAAT
ACCGGTGGCGAAGGCGGCCCCCTGGACAAAGACTG
ACGCTCAGGTGCGAAAGCGTGGGGAGCAAACAGGA
TTAGATACCCTGGTAGTCCACGCCGTAAACGATGT
CGATTTGGAGGTTGTGCCCTTGAGGCGTGGCTTCC
GGAGCTAACGCGTTAAATCGACCGCCTGGGGAGTA
CGGCCGCAAGGTTAAA
[SEQ ID NO: 118]
AGCGTAGACGGTGTGGCAAGTCTGATGTGAAAGGC
ATGGGCTTAACCTGGGAACTGCATTCGAAACTGGC
AGGCTAGAGTCTTGTAGAGGGGGGTAGAATTCCAG
GTGTAGCGGTGAAATGCGTAGAGATCTGGAGGAAT
ACCGGTGGCGAAGGCGGCCCCCTGGACAAAGACTG
ACGCTCAGGTGCGAAAGCGTGGGGAGCAAACAGGA
TTAGATACCCTGGTAGTCCACGCCGTAAACGATGT
CGATTTGGAGGTTGTGCCCTTGAGGCGTGGCTTCC
GGAGCTAACGCGTTAAATCGACCGCCTGGGGAGTA
CGGCCGCAAGGTTAAA

In certain non-limiting embodiments, the one or more bacteria can comprise one or more species from the bacterial species Lactobacillus delbrueckii subsp. Bulgaricus, for example, found within the taxonomic group having NCBI:txid 1585, or bacteria having at least about 90%, at least about 95%, at least about 97%, or at least about 99% identity with one or more 16S rRNA gene sequences, for example, as described by GenBank Accession number NR_075019 (SEQ ID NO:71), or a variable region of one or more 16S rRNA gene sequences such as the V4 region, from said Lactobacillus delbrueckii subsp. Bulgaricus.

In certain non-limiting embodiments, the one or more bacteria can comprise one or more species from the bacteria genus Coprobacillus for example, found within the taxonomic group having NCBI:txid 100883.

In certain non-limiting embodiments, the one or more bacteria can comprise one or more species from the bacteria genus Parabacteroides, for example, found within the taxonomic group having NCBI:txid 375288.

In certain non-limiting embodiments, the one or more bacteria can comprise one or more species from the bacteria genus Ruminococcus, for example, found within the taxonomic group having NCBI:txid 1263.

In certain non-limiting embodiments, the one or more bacteria can comprise one or more species from the bacterial species Streptococcus mutans, for example, found within the taxonomic group having NCBI:txid 1309, or bacteria having at least about 90%, at least about 95%, at least about 97%, or at least about 99% identity with one or more 16S rRNA gene sequences, for example, as described by GenBank Accession number NR_042772 (SEQ ID NO:72), or a variable region of one or more 16S rRNA gene sequences such as the V4 region, from said Streptococcus mutans.

In certain non-limiting embodiments, the one or more bacteria can comprise one or more species from the bacterial species Streptococcus mutans, for example, found within the taxonomic group having NCBI:txid 1309, or bacteria having at least about 90%, at least about 95%, at least about 97%, or at least about 99% identity with one or more 16S rRNA gene sequences, for example, as described by GenBank Accession number NR_042772 (SEQ ID NO:72), or a variable region of one or more 16S rRNA gene sequences such as the V4 region, from said Streptococcus mutans.

In certain non-limiting embodiments, the one or more bacteria can comprise one or more species from the bacterial species Enterococcus rivorum, for example, found within the taxonomic group having NCBI:txid 762845, or bacteria having at least about 90%, at least about 95%, at least about 97%, or at least about 99% identity with one or more 16S rRNA gene sequences, for example, as described by GenBank Accession number NR_117043 (SEQ ID NO:73), or a variable region of one or more 16S rRNA gene sequences such as the V4 region, from said Enterococcus rivorum.

In certain non-limiting embodiments, the one or more bacteria can comprise one or more species from the bacterial species Enterobacter ludwigii, for example, found within the taxonomic group having NCBI:txid 299767, or bacteria having at least about 90%, at least about 95%, at least about 97%, or at least about 99% identity with one or more 16S rRNA gene sequences, for example, as described by GenBank Accession number KF528827 (SEQ ID NO:74), or a variable region of one or more 16S rRNA gene sequences such as the V4 region, from said Enterobacter ludwigii.

In certain non-limiting embodiments, the one or more bacteria can comprise one or more species from the bacterial species Clostridium leptum, for example, found within the taxonomic group having NCBI:txid 1535, or bacteria having at least about 90%, at least about 95%, at least about 97%, or at least about 99% identity with one or more 16S rRNA gene sequences, for example, as described by GenBank Accession number NR_114789 (SEQ ID NO:75), or a variable region of one or more 16S rRNA gene sequences such as the V4 region, from said Clostridium leptum.

In certain non-limiting embodiments, the one or more bacteria can comprise one or more species from the bacterial species Enterococcus lactis, for example, found within the taxonomic group having NCBI:txid 357441, or bacteria having at least about 90%, at least about 95%, at least about 97%, or at least about 99% identity with one or more 16S rRNA gene sequences, for example, as described by GenBank Accession number NR_117562 (SEQ ID NO:76), or a variable region of one or more 16S rRNA gene sequences such as the V4 region, from said Enterococcus lactis.

In certain non-limiting embodiments, the one or more bacteria can comprise one or more species from the bacterial species Bifidobacterium dentium, for example, found within the taxonomic group having NCBI:txid 1689, or bacteria having at least about 90%, at least about 95%, at least about 97%, or at least about 99% identity with one or more 16S rRNA gene sequences, for example, as described by GenBank Accession number NR_037115 (SEQ ID NO:77), or a variable region of one or more 16S rRNA gene sequences such as the V4 region, from said Bifidobacterium dentium.

In certain non-limiting embodiments, the one or more bacteria can comprise one or more species from the bacterial species Eubacterium limosum, for example, found within the taxonomic group having NCBI:txid 1736, or bacteria having at least about 90%, at least about 95%, at least about 97%, or at least about 99% identity with one or more 16S rRNA gene sequences, for example, as described by GenBank Accession number NR_113248 (SEQ ID NO:78), or a variable region of one or more 16S rRNA gene sequences such as the V4 region, from said Eubacterium limosum.

In certain non-limiting embodiments, the one or more bacteria can comprise one or more species from the bacteria phylum Proteobacteria, for example, found within the taxonomic group having NCBI:txid 1224.

In certain non-limiting embodiments, the one or more bacteria can comprise one or more species from the bacteria genus Mycoplasma, for example, found within the taxonomic group having NCBI:txid 2093.

In certain non-limiting embodiments, the one or more bacteria can comprise one or more species from the bacterial species Holdemania filiformis, for example, found within the taxonomic group having NCBI:txid 61171, or bacteria having at least about 90%, at least about 95%, at least about 97%, or at least about 99% identity with one or more 16S rRNA gene sequences, for example, as described by GenBank Accession number NR_029335 (SEQ ID NO:79), or a variable region of one or more 16S rRNA gene sequences such as the V4 region, from said Holdemania filiformis.

In certain non-limiting embodiments, the one or more bacteria can comprise one or more species from the bacterial species Lactococcus piscium, for example, found within the taxonomic group having NCBI:txid 1364, or bacteria having at least about 90%, at least about 95%, at least about 97%, or at least about 99% identity with one or more 16S rRNA gene sequences, for example, as described by GenBank Accession number JN226415 (SEQ ID NO:80), or a variable region of one or more 16S rRNA gene sequences such as the V4 region, from said Lactococcus piscium.

In certain non-limiting embodiments, the one or more bacteria can comprise one or more species from the bacteria genus Blautia, for example, found within the taxonomic group having NCBI:txid 572511.

In certain non-limiting embodiments, the one or more bacteria can comprise one or more species from the bacterial species Bacteroides thetaiotaomicron, for example, found within the taxonomic group having NCBI:txid 818, or bacteria having at least about 90%, at least about 95%, at least about 97%, or at least about 99% identity with one or more 16S rRNA gene sequences, for example, as described by GenBank Accession number NR_074277 (SEQ ID NO:81), or a variable region of one or more 16S rRNA gene sequences such as the V4 region, from said Bacteroides thetaiotaomicron.

In certain non-limiting embodiments, the one or more bacteria can comprise one or more species from the bacterial species Massiliomicrobiota timonensis, for example, found within the taxonomic group having NCBI:txid 1776392, or bacteria having at least about 90%, at least about 95%, at least about 97%, or at least about 99% identity with one or more 16S rRNA gene sequences, for example, as described by GenBank Accession number NR_144738 (SEQ ID NO:82), or a variable region of one or more 16S rRNA gene sequences such as the V4 region, from said Massiliomicrobiota timonensis.

In certain non-limiting embodiments, the one or more bacteria can comprise one or more species from the bacterial species Blautia hydrogenotrophica, for example, found within the taxonomic group having NCBI:txid 53443, or bacteria having at least about 90%, at least about 95%, at least about 97%, or at least about 99% identity with one or more 16S rRNA gene sequences, for example, as described by GenBank Accession number NR_026314 (SEQ ID NO:83), or a variable region of one or more 16S rRNA gene sequences such as the V4 region, from said Blautia hydrogenotrophica.

In certain non-limiting embodiments, the one or more bacteria can comprise one or more species from the bacterial species Enterococcus mundtii, for example, found within the taxonomic group having NCBI:txid 53346, or bacteria having at least about 90%, at least about 95%, at least about 97%, or at least about 99% identity with one or more 16S rRNA gene sequences, for example, as described by GenBank Accession number NR_024906 (SEQ ID NO:84), or a variable region of one or more 16S rRNA gene sequences such as the V4 region, from said Enterococcus mundtii.

In certain non-limiting embodiments, the one or more bacteria can comprise one or more species from the bacterial species Prevotella melaninogenica, for example, found within the taxonomic group having NCBI:txid 28132, or bacteria having at least about 90%, at least about 95%, at least about 97%, or at least about 99% identity with one or more 16S rRNA gene sequences, for example, as described by GenBank Accession number NR_113113 (SEQ ID NO:85), or a variable region of one or more 16S rRNA gene sequences such as the V4 region, from said Prevotella melaninogenica.

In certain non-limiting embodiments, the one or more bacteria can comprise one or more species from the bacterial species Erwinia chrysanthemi, for example, found within the taxonomic group having NCBI:txid 556, or bacteria having at least about 90%, at least about 95%, at least about 97%, or at least about 99% identity with one or more 16S rRNA gene sequences, for example, as described by GenBank Accession number AF373175 (SEQ ID NO:86), or a variable region of one or more 16S rRNA gene sequences such as the V4 region, from said Erwinia chrysanthemi.

In certain non-limiting embodiments, the one or more bacteria can comprise one or more species from the bacterial species Clostridium nexile DSM1787, for example, found within the taxonomic group having NCBI:txid 500632, or bacteria having at least about 90%, at least about 95%, at least about 97%, or at least about 99% identity with one or more 16S rRNA gene sequences, for example, as described by GenBank Accession number NR_029248 (SEQ ID NO:87), or a variable region of one or more 16S rRNA gene sequences such as the V4 region, from said Clostridium nexile DSM1787.

In certain non-limiting embodiments, the one or more bacteria can comprise one or more species from the bacteria genus Veillonella, for example, found within the taxonomic group having NCBI:txid 29465.

In certain non-limiting embodiments, the one or more bacteria can comprise one or more species from the bacterial species Clostridium difficile, for example, found within the taxonomic group having NCBI:txid 1496, or bacteria having at least about 90%, at least about 95%, at least about 97%, or at least about 99% identity with one or more 16S rRNA gene sequences, for example, as described by GenBank Accession number NR_113132 (SEQ ID NO:88), or a variable region of one or more 16S rRNA gene sequences such as the V4 region, from said Clostridium difficile.

In certain non-limiting embodiments, the one or more bacteria can comprise one or more species from the bacterial species [Ruminococcus] obeum, for example, found within the taxonomic group having NCBI:txid 40520, or bacteria having at least about 90%, at least about 95%, at least about 97%, or at least about 99% identity with one or more 16S rRNA gene sequences, for example, as described by GenBank Accession number NR_118692 (SEQ ID NO:89), or a variable region of one or more 16S rRNA gene sequences such as the V4 region, from said [Ruminococcus] obeum.

In certain non-limiting embodiments, the one or more bacteria can comprise one or more species from the bacteria order Streptophyta, for example, found within the taxonomic group having NCBI:txid 35493.

In certain non-limiting embodiments, the one or more bacteria can comprise one or more species from the bacteria order Bacteroidales, for example, found within the taxonomic group having NCBI:txid 171549.

In certain non-limiting embodiments, the one or more bacteria can comprise one or more species from the bacteria genus Parascardovia, for example, found within the taxonomic group having NCBI:txid 196082.

In certain non-limiting embodiments, the one or more bacteria can comprise one or more species from the bacterial species Clostridium clostridioforme, for example, found within the taxonomic group having NCBI:txid 1531, or bacteria having at least about 90%, at least about 95%, at least about 97%, or at least about 99% identity with one or more 16S rRNA gene sequences, for example, as described by GenBank Accession number NR_044715 (SEQ ID NO:90), or a variable region of one or more 16S rRNA gene sequences such as the V4 region, from said Clostridium clostridioforme.

In certain non-limiting embodiments, the one or more bacteria can comprise one or more species from the bacterial species Blautia obeum, for example, found within the taxonomic group having NCBI:txid 40520, or bacteria having at least about 90%, at least about 95%, at least about 97%, or at least about 99% identity with one or more 16S rRNA gene sequences, for example, as described by GenBank Accession number NR_118692 (SEQ ID NO:91), or a variable region of one or more 16S rRNA gene sequences such as the V4 region, from said Blautia obeum.

In certain non-limiting embodiments, the one or more bacteria can comprise one or more species from the bacterial species Klebsiella oxytoca, for example, found within the taxonomic group having NCBI:txid 571, or bacteria having at least about 90%, at least about 95%, at least about 97%, or at least about 99% identity with one or more 16S rRNA gene sequences, for example, as described by GenBank Accession number NR_041749 (SEQ ID NO:92), or a variable region of one or more 16S rRNA gene sequences such as the V4 region, from said Klebsiella oxytoca.

In certain non-limiting embodiments, the one or more bacteria can comprise one or more species from the bacterial species Bulleidia moorei, for example, found within the taxonomic group having NCBI:txid 102148, or bacteria having at least about 90%, at least about 95%, at least about 97%, or at least about 99% identity with one or more 16S rRNA gene sequences, for example, as described by GenBank Accession number NR_036882 (SEQ ID NO:93), or a variable region of one or more 16S rRNA gene sequences such as the V4 region, from said Bulleidia moorei.

In certain non-limiting embodiments, the one or more bacteria can comprise one or more species from the bacterial species Parabacteroides merdae, for example, found within the taxonomic group having NCBI:txid 46503, or bacteria having at least about 90%, at least about 95%, at least about 97%, or at least about 99% identity with one or more 16S rRNA gene sequences, for example, as described by GenBank Accession number NR_041343 (SEQ ID NO:94), or a variable region of one or more 16S rRNA gene sequences such as the V4 region, from said Parabacteroides merdae.

In certain non-limiting embodiments, the one or more bacteria can comprise one or more species from the bacterial species Shuttleworthia satelles, for example, found within the taxonomic group having NCBI:txid 177972, or bacteria having at least about 90%, at least about 95%, at least about 97%, or at least about 99% identity with one or more 16S rRNA gene sequences, for example, as described by GenBank Accession number NR_028827 (SEQ ID NO:95), or a variable region of one or more 16S rRNA gene sequences such as the V4 region, from said Shuttleworthia satelles.

In certain non-limiting embodiments, the one or more bacteria can comprise one or more species from the bacterial species Streptococcus sp. DN812, for example, found within the taxonomic group having NCBL:txid 1244110, or bacteria having at least about 90%, at least about 95%, at least about 97%, or at least about 99% identity with one or more 16S rRNA gene sequences, for example, as described by GenBank Accession number JX681143 (SEQ ID NO:96), or a variable region of one or more 16S rRNA gene sequences such as the V4 region, from said Streptococcus sp. DN812.

In certain non-limiting embodiments, the one or more bacteria can comprise one or more species from the bacterial species Clostridium cellulosi, for example, found within the taxonomic group having NCBI:txid 29343, or bacteria having at least about 90%, at least about 95%, at least about 97%, or at least about 99% identity with one or more 16S rRNA gene sequences, for example, as described by GenBank Accession number NR_044624 (SEQ ID NO:97), or a variable region of one or more 16S rRNA gene sequences such as the V4 region, from said Clostridium cellulosi.

In certain non-limiting embodiments, the one or more bacteria can comprise one or more species from the bacterial species Lactobacillus acidophilus, for example, found within the taxonomic group having NCBI:txid 1579, or bacteria having at least about 90%, at least about 95%, at least about 97%, or at least about 99% identity with one or more 16S rRNA gene sequences, for example, as described by GenBank Accession number NR_043182 (SEQ ID NO:98), or a variable region of one or more 16S rRNA gene sequences such as the V4 region, from said Lactobacillus acidophilus.

In certain non-limiting embodiments, the one or more bacteria can comprise one or more species from the bacterial species Bacteroides ovatus, for example, found within the taxonomic group having NCBI:txid 28116, or bacteria having at least about 90%, at least about 95%, at least about 97%, or at least about 99% identity with one or more 16S rRNA gene sequences, for example, as described by GenBank Accession number NR_040865 (SEQ ID NO:99), or a variable region of one or more 16S rRNA gene sequences such as the V4 region, from said Bacteroides ovatus.

In certain non-limiting embodiments, the one or more bacteria can comprise one or more species from the bacterial species Clostridium hylemonae, for example, found within the taxonomic group having NCBI:txid 89153, or bacteria having at least about 90%, at least about 95%, at least about 97%, or at least about 99% identity with one or more 16S rRNA gene sequences, for example, as described by GenBank Accession number NR_024719 (SEQ ID NO:100), or a variable region of one or more 16S rRNA gene sequences such as the V4 region, from said Clostridium hylemonae.

In certain non-limiting embodiments, the one or more bacteria can comprise one or more species from the bacterial species Veillonella parvula, for example, found within the taxonomic group having NCBI:txid 29466, or bacteria having at least about 90%, at least about 95%, at least about 97%, or at least about 99% identity with one or more 16S rRNA gene sequences, for example, as described by GenBank Accession number NR_043332 (SEQ ID NO:101), or a variable region of one or more 16S rRNA gene sequences such as the V4 region, from said Veillonella parvula.

In certain non-limiting embodiments, the one or more bacteria can comprise one or more species from the bacterial species Longibaculum muris, for example, found within the taxonomic group having NCBI:txid 1796628, or bacteria having at least about 90%, at least about 95%, at least about 97%, or at least about 99% identity with one or more 16S rRNA gene sequences, for example, as described by GenBank Accession number NR_144615 (SEQ ID NO:102), or a variable region of one or more 16S rRNA gene sequences such as the V4 region, from said Longibaculum muris.

In certain non-limiting embodiments, the one or more bacteria can comprise one or more species from the bacteria genus Butyrivibrio, for example, found within the taxonomic group having NCBI:txid 830.

In certain non-limiting embodiments, the one or more bacteria can comprise one or more species from the bacterial species Peptostreptococcaceae bacterium canine oral taxon 074, for example, found within the taxonomic group having NCBI:txid 1151692, or bacteria having at least about 90%, at least about 95%, at least about 97%, or at least about 99% identity with one or more 16S rRNA gene sequences, for example, as described by GenBank Accession number JN713238 (SEQ ID NO:103), or a variable region of one or more 16S rRNA gene sequences such as the V4 region, from said Peptostreptococcaceae bacterium canine oral taxon 074.

In certain non-limiting embodiments, the one or more bacteria can comprise one or more species from the bacterial species Alloscardovia omnicolens, for example, found within the taxonomic group having NCBI:txid 419015, or bacteria having at least about 90%, at least about 95%, at least about 97%, or at least about 99% identity with one or more 16S rRNA gene sequences, for example, as described by GenBank Accession number NR_042583 (SEQ ID NO:104), or a variable region of one or more 16S rRNA gene sequences such as the V4 region, from said Alloscardovia omnicolens.

In certain non-limiting embodiments, the one or more bacteria can comprise one or more species from the bacterial species Lactobacillus salivarius, for example, found within the taxonomic group having NCBI:txid 1624, or bacteria having at least about 90%, at least about 95%, at least about 97%, or at least about 99% identity with one or more 16S rRNA gene sequences, for example, as described by GenBank Accession number NR_028725 (SEQ ID NO:105), or a variable region of one or more 16S rRNA gene sequences such as the V4 region, from said Lactobacillus salivarius.

In certain non-limiting embodiments, the one or more bacteria can comprise one or more species from the bacterial species Clostridium scindens, for example, found within the taxonomic group having NCBI:txid 29347, or bacteria having at least about 90%, at least about 95%, at least about 97%, or at least about 99% identity with one or more 16S rRNA gene sequences, for example, as described by GenBank Accession number NR_028785 (SEQ ID NO:106), or a variable region of one or more 16S rRNA gene sequences such as the V4 region, from said Clostridium scindens.

In certain non-limiting embodiments, the one or more bacteria can comprise one or more species from the bacteria genus Alistipes, for example, found within the taxonomic group having NCBI:txid 239759.

In certain non-limiting embodiments, the one or more bacteria can comprise one or more species from the bacterial species Clostridium lavalense, for example, found within the taxonomic group having NCBI:txid 460384, or bacteria having at least about 90%, at least about 95%, at least about 97%, or at least about 99% identity with one or more 16S rRNA gene sequences, for example, as described by GenBank Accession number NR_044289 (SEQ ID NO:107), or a variable region of one or more 16S rRNA gene sequences such as the V4 region, from said Clostridium lavalense.

In certain non-limiting embodiments, the one or more bacteria can comprise one or more species from the bacteria genus Anaerostipes, for example, found within the taxonomic group having NCBI:txid 207244.

In certain non-limiting embodiments, the one or more bacteria can comprise one or more species from the bacterial species Abiotrophia defectiva, for example, found within the taxonomic group having NCBI:txid 46125, or bacteria having at least about 90%, at least about 95%, at least about 97%, or at least about 99% identity with one or more 16S rRNA gene sequences, for example, as described by GenBank Accession number NR_025863 (SEQ ID NO:108), or a variable region of one or more 16S rRNA gene sequences such as the V4 region, from said Abiotrophia defectiva.

In certain non-limiting embodiments, the one or more bacteria can comprise one or more species from the bacteria family Leuconostocaceae, for example, found within the taxonomic group having NCBI:txid 81850.

In certain non-limiting embodiments, the one or more bacteria can comprise one or more species from the bacteria genus Dorea, for example, found within the taxonomic group having NCBI:txid 189330.

In certain non-limiting embodiments, the one or more bacteria can comprise one or more species from the bacteria genus Klebsiella, for example, found within the taxonomic group having NCBI:txid 570.

In certain non-limiting embodiments, the one or more bacteria can comprise one or more species from the bacteria genus Escherichia, for example, found within the taxonomic group having NCBI:txid 561.

In certain non-limiting embodiments, the one or more bacteria can comprise one or more species from the bacteria genus Staphylococcus, for example, found within the taxonomic group having NCBI:txid 1279.

In certain non-limiting embodiments, the one or more bacteria can comprise one or more species from the bacteria genus Streptococcus, for example, found within the taxonomic group having NCBI:txid 1301.

In certain non-limiting embodiments, the one or more bacteria can comprise one or more species from the bacterial species Alistipes putredinis, for example, found within the taxonomic group having NCBI:txid 28117, or bacteria having at least about 90%, at least about 95%, at least about 97%, or at least about 99% identity with one or more 16S rRNA gene sequences, for example, as described by GenBank Accession number NR_025909 (SEQ ID NO:109), or a variable region of one or more 16S rRNA gene sequences such as the V4 region, from said Alistipes putredinis.

In certain non-limiting embodiments, the one or more bacteria can comprise one or more species from the bacterial species Salinicoccus qingdaonensis, for example, found within the taxonomic group having NCBI:txid 576118, or bacteria having at least about 90%, at least about 95%, at least about 97%, or at least about 99% identity with one or more 16S rRNA gene sequences, for example, as described by GenBank Accession number NR_108325 (SEQ ID NO:110), or a variable region of one or more 16S rRNA gene sequences such as the V4 region, from said Salinicoccus qingdaonensis.

In certain non-limiting embodiments, the one or more bacteria can comprise one or more species from the bacterial species Parasutterella excrementihominis, for example, found within the taxonomic group having NCBI:txid 487175, or bacteria having at least about 90%, at least about 95%, at least about 97%, or at least about 99% identity with one or more 16S rRNA gene sequences, for example, as described by GenBank Accession number NR_041667 (SEQ ID NO:111), or a variable region of one or more 16S rRNA gene sequences such as the V4 region, from said Parasutterella excrementihominis.

In certain non-limiting embodiments, the one or more bacteria can comprise one or more species from the bacterial species Bacteroides caccae, for example, found within the taxonomic group having NCBI:txid 47678, or bacteria having at least about 90%, at least about 95%, at least about 97%, or at least about 99% identity with one or more 16S rRNA gene sequences, for example, as described by GenBank Accession number NR_026242 (SEQ ID NO:112), or a variable region of one or more 16S rRNA gene sequences such as the V4 region, from said Bacteroides caccae.

In certain non-limiting embodiments, the one or more bacteria can comprise one or more species from the bacteria family Bifidobacteriaceae, for example, found within the taxonomic group having NCBI:txid 31953.

In certain non-limiting embodiments, the one or more bacteria can comprise one or more species from the bacterial species Streptococcus anginosus, for example, found within the taxonomic group having NCBI:txid 1328, or bacteria having at least about 90%, at least about 95%, at least about 97%, or at least about 99% identity with one or more 16S rRNA gene sequences, for example, as described by GenBank Accession number NR_041722 (SEQ ID NO:113), or a variable region of one or more 16S rRNA gene sequences such as the V4 region, from said Streptococcus anginosus.

In certain non-limiting embodiments, the one or more bacteria can comprise one or more species from the bacterial species Haemophilus parainfluenzae, for example, found within the taxonomic group having NCBI:txid 729, or bacteria having at least about 90%, at least about 95%, at least about 97%, or at least about 99% identity with one or more 16S rRNA gene sequences, for example, as described by GenBank Accession number NR_042878 (SEQ ID NO:114), or a variable region of one or more 16S rRNA gene sequences such as the V4 region, from said Haemophilus parainfluenzae.

In certain non-limiting embodiments, the one or more bacteria can comprise one or more species from the bacteria genus Oscillospira, for example, found within the taxonomic group having NCBI:txid 119852.

In certain non-limiting embodiments, the one or more bacteria can comprise one or more species from the bacterial species Clostridium aldenense, for example, found within the taxonomic group having NCBI:txid 358742, or bacteria having at least about 90%, at least about 95%, at least about 97%, or at least about 99% identity with one or more 16S rRNA gene sequences, for example, as described by GenBank Accession number NR_043680 (SEQ ID NO:115), or a variable region of one or more 16S rRNA gene sequences such as the V4 region, from said Clostridium aldenense.

In certain non-limiting embodiments, the bacteria described herein can be modified, for example, by introducing one or more exogenous nucleic acids into the bacteria, thereby producing recombinant bacteria. Such nucleic acids can comprise, for example, an antibiotic resistance gene and/or an antibiotic susceptibility gene. Such recombinant bacteria can be prepared as described herein.

In certain non-limiting embodiments, the present disclosure provides compositions comprising one or more bacteria, or spores thereof, associated with the likelihood of cancer survival, for example, a bacterial species from the taxonomic groups listed in Group 1.

In certain non-limiting embodiments, the present disclosure provides compositions comprising one or more bacteria, or spores thereof, associated with an increased likelihood of cancer survival, for example, a bacterial species from the taxonomic groups listed in Group 2.

In certain non-limiting embodiments, the present disclosure provides compositions comprising one or more bacteria, associated with a decreased likelihood of cancer survival, for example, a bacterial species from the taxonomic groups listed in Group 3.

### 5.4. Recombinant Cells

The present disclosure provides for therapeutic compositions, and therapeutic uses thereof, as described herein, which increase the likelihood of cancer survival in a subject. Such therapeutic compositions can comprise, for example, therapeutic bacteria, small molecules, polypeptides, or nucleic acid molecules.

In certain non-limiting embodiments, the therapeutic compositions reduce the amount of bacterial species from the taxonomic groups listed in Group 3, and/or inhibit proliferation and/or growth of bacterial species from the taxonomic groups listed in Group 3 in a subject.

In certain non-limiting embodiments, the therapeutic composition comprises a recombinant species of bacteria from the taxonomic groups listed in Group 2, a combination thereof, or a cluster comprising any one or more of the foregoing bacteria, or progeny thereof.

In certain non-limiting embodiments, the therapeutic composition comprises a recombinant cell, or progeny thereof, for example, a recombinant cell expressing one or more proteins endogenously expressed by a species of bacteria from the taxonomic groups listed in Group 2, a combination thereof, or a cluster comprising any one or more of the foregoing bacteria.

In certain non-limiting embodiments, expression of an antibiotic resistance gene by the recombinant cell, or progeny thereof, reduces the inhibition in growth or survival of the recombinant cell caused by exposure to said antibiotic such as, but not limited to, an antibiotic selected from the group consisting of a β-lactam antibiotic, clindamycin, a cephalosporin, a quinolone antibiotic, levofloxacin, fluoroquinolone, a macrolide antibiotic, trimethoprim, and a sulfonamide antibiotic. In certain non-limiting embodiments, the recombinant cell is resistant to an antibiotic other than the foregoing antibiotics.

In certain non-limiting embodiments, expression of an antibiotic susceptibility gene by the recombinant cell increases the inhibition in growth or survival of the recombinant cell caused by exposure to said antibiotic. In certain non-limiting embodiments, such antibiotics can include, but are not limited to, an antibiotic selected from the group consisting of a β-lactam antibiotic, clindamycin, a cephalosporin, a quinolone antibiotic, levofloxacin, fluoroquinolone, a macrolide antibiotic, trimethoprim, and a sulfonamide antibiotic. In certain non-limiting embodiments, the recombinant cell is susceptible to an antibiotic other than the foregoing antibiotics.

In certain non-limiting embodiments, the recombinant cells described herein express one or more recombinant genes that increase the synthesis and secretion of a metabolite that modulates a subject's likelihood of cancer survival, for example, a protein from a species of bacteria from the taxonomic groups listed in Group 2 that increases the subject's likelihood for cancer survival.

Delivery of nucleic acid into a subject or cell, e.g., bacterial cells of the intestinal microbiota, can be either direct, in which case the subject or cell, e.g., bacterial cells of a subject's intestinal microbiota, is directly exposed to the nucleic acid or nucleic acid-carrying vectors, or indirect, in which case, cells, e.g., a host cell, such as isolated bacterial cells of the intestinal microbiota, are first transformed with the nucleic acids in vitro, then transplanted into the subject. These two approaches are known, respectively, as in situ or ex vivo gene therapy.

For general reviews of the methods of gene therapy, see Kron and Kreppel, Curr. Gene Ther. 12(5):362-73 (2012); Yi et al. Curr. Gene Ther. 11(3):218-28 (2011); Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); Morgan and Anderson, Ann. Rev. Biochem. 62:191-217 (1993); and May, TIBTECH 11(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In certain non-limiting embodiments, the nucleic acid can be introduced into cells, e.g., bacterial host cells, prior to administration in vivo of the resulting recombinant cell by any method known in the art, including but not limited to transfection, electroporation, microinjection, lipofection, calcium phosphate mediated transfection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985)), and can be used in accordance with the present disclosure, provided that the necessary developmental and physiological functions of the recipient cells are not disrupted. Usually, the method of transfer includes the transfer of a selectable marker to the host cells. The cells are then placed under selection to isolate those host cells that have taken up and are expressing the transferred gene. Those host cells are then delivered to a patient.

The resulting recombinant cells, or progeny thereof, can be delivered to a patient by various methods known in the art. The number of cells envisioned for use depends on the desired effect, patient state, etc., and can be determined by one skilled in the art.

In certain non-limiting embodiments, the terms “vector” and “expression vector” mean the vehicle by which a DNA or RNA sequence (e.g., a foreign gene) can be introduced into a host cell, so as to transform the host and promote expression (e.g., transcription and translation) of the introduced sequence. Vectors include plasmids, phages, viruses, etc. A “therapeutic vector” as used herein refers to a vector which is acceptable for administration to an animal, and particularly to a human.

Vectors typically include the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can accept additional (foreign) DNA and which can be introduced into a suitable host cell. A plasmid vector can contain coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA can be from the same gene or from different genes, and can be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET plasmids (Invitrogen, San Diego, Calif.), pCDNA3 plasmids (Invitrogen), pREP plasmids (Invitrogen), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g., antibiotic resistance, and one or more expression cassettes.

Suitable vectors include, for example, bacteriophages, cosmids, plasmids, naked DNA, DNA lipid complexes, and other recombination vehicles typically used in the art which have been described for expression in a variety of eukaryotic and prokaryotic hosts, and can be used for gene therapy as well as for simple protein expression.

### 5.5. Compositions

In certain non-limiting embodiments, the present disclosure provides for compositions, e.g. pharmaceutical compositions, and therapeutic uses thereof as described herein. In certain embodiments, the present disclosure provides compositions, e.g., pharmaceutical compositions, comprising bacteria, e.g., therapeutic bacteria, for treating a cancer and/or for increasing the likelihood of cancer survival in a subject being treated or to be treated for cancer, e.g., has received or will receive allo-HCT. In certain embodiments, the present disclosure provides compositions, e.g., pharmaceutical compositions, for treating a cancer subject that has a decreased likelihood of cancer survival.

In certain embodiments, the present disclosure provides for a composition, e.g., a pharmaceutical composition, comprising an isolated bacterial species from the taxonomic groups disclosed herein. For example, but not by way of limitation, the compositions disclosed herein can reduce the amount of bacterial species from the taxonomic groups listed in Group 3, and/or inhibit proliferation and/or growth of bacterial species from the taxonomic groups listed in Group 3 in a subject. Alternatively or additionally, the compositions disclosed herein can increase the amount of bacterial species from the taxonomic groups listed in Group 2, and/or increase proliferation and/or growth of bacterial species from the taxonomic groups listed in Group 2 in a subject.

In certain embodiments, the present disclosure provides for a composition, e.g., a pharmaceutical composition, comprising an isolated bacterial species from the taxonomic groups listed in Group 2, a combination thereof, or a cluster comprising any one or more of the foregoing bacteria. In certain embodiments, the composition, e.g., a pharmaceutical composition, comprises one, two, three, four, five, six, seven, eight, nine, ten, eleven, twelve, thirteen, fourteen, fifteen, or sixteen bacteria selected from the bacterial species from the taxonomic groups listed in Group 2.

In certain non-limiting embodiments, a composition of the present disclosure can includes one or more of the bacterial species from the taxonomic groups listed in Group 2, but alternate or additional bacteria can also be comprised in the compositions described herein, for example, bacteria which can be naturally occurring, bacteria that are in a cluster comprising any one or more of the bacterial species from the taxonomic groups listed in Group 2, or bacteria engineered to express proteins from a bacterial species in the taxonomic groups listed in Group 2.

In certain non-limiting embodiments of the disclosure, bacteria of a composition disclosed herein can be administered in the vegetative or dormant state, or as spores, or a mixture thereof. In certain embodiments, the bacteria of a composition disclosed herein can be lyophilized or in freeze-dried form.

In certain embodiments, compositions, e.g., pharmaceutical compositions, of the present disclosure can further include at least one other agent, such as a stabilizing compound or additional therapeutic agent, for example, a probiotic, prebiotic, postbiotic, and/or antibiotic, and can be administered in any sterile, biocompatible pharmaceutical carrier, including, but not limited to, saline, buffered saline, dextrose, glycerol, polyethylene glycol, and water. In certain embodiments, the composition can be in a liquid or lyophilized or freeze-dried form.

In certain non-limiting embodiments, a composition, e.g., a pharmaceutical composition, includes a diluent (for example, a buffer such as Tris, citrate, acetate or phosphate buffers) having suitable pH values and ionic strengths, solubilizer such as polysorbate (e.g., Tween®), carriers such as human serum albumin or gelatin. In certain embodiments, a preservative can be included that does not affect viability of the organisms in the composition. Examples of preservatives include thimerosal, parabens, benzylalconium chloride or benzyl alcohol, antioxidants such as ascorbic acid or sodium metabisulfite, and other components such as lysine or glycine. Selection of a particular composition will depend upon a number of factors, including the condition being treated, the route of administration and the pharmacokinetic parameters desired. A more extensive survey of components suitable for pharmaceutical compositions is found in Remington's Pharmaceutical Sciences, 18th ed. A. R. Gennaro, ed. Mack, Easton, Pa. (1980).

In certain non-limiting embodiments, the methods and compositions of the present disclosure find use in increasing the likelihood of cancer survival in a subject.

Such therapeutic bacteria are administered to the patient in a pharmaceutically acceptable carrier. The route of administration eventually chosen will depend upon a number of factors and can be ascertained by one skilled in the art.

In certain non-limiting embodiments, the pharmaceutical compositions of the present disclosure can be formulated using pharmaceutically acceptable carriers well known in the art in dosages suitable for oral, nasogastric, rectal, percutaneous (e.g., G tube), orogastric tube, or other enteral routes administration. Such carriers enable the pharmaceutical compositions to be formulated as tablets, pills, capsules, liquids, gels, syrups, slurries, suspensions and the like, for oral, rectal or nasal ingestion by a patient to be treated. In certain non-limiting embodiments, the formulation comprises a capsule or tablet formulated for gastrointestinal delivery, e.g., an enteric coated capsule or pill.

In certain non-limiting embodiments, the bacteria are administered in a food product, for example, a yogurt food product. In certain non-limiting embodiments, a “food product” means a product or composition that is intended for consumption by a human or a non-human animal. Such food products include any food, feed, snack, food supplement, liquid, beverage, treat, toy (chewable and/or consumable toys), meal substitute or meal replacement.

In certain non-limiting embodiments, a bacterial species from the taxonomic groups listed in Group 2, a combination thereof, or a cluster comprising any one or more of the foregoing bacteria, can be administered in the form of purified bacteria or spores or other progenitors thereof, or alternatively can be administered as a constituent in a mixture of types of bacteria, optionally including one or more species or cluster of additional bacteria, for example, probiotic bacteria, a probiotic yeast, prebiotic, postbiotic and/or antibiotic.

In certain non-limiting embodiments, a pharmaceutical composition of the present disclosure comprises bacteria from the taxonomic groups listed in Group 2, a combination thereof, or a cluster comprising any one or more of the foregoing bacteria, and optionally including one or more species or cluster of additional bacteria, for example, probiotic bacteria, a probiotic yeast, prebiotic, postbiotic and/or antibiotic. In certain embodiments, such bacteria can be administered in the form of a liquid, a suspension, a dried (e.g., lyophilized) powder, a tablet, a capsule, or a suppository, and can be administered orally, nasogastrically, rectally, percutaneously (e.g., G tube), or through other enteral routes.

Pharmaceutical compositions suitable for use in the present disclosure include, in certain non-limiting embodiments, compositions where the active ingredients are contained in an effective amount to achieve the intended purpose. For example, but not by way of limitation, a pharmaceutical composition can include an effective amount of a bacteria, e.g., bacteria from the taxonomic groups listed in Group 2, for increasing the likelihood of cancer survival in a subject being treated or to be treated for cancer. The amount will vary from one individual to another and will depend upon a number of factors, including the overall physical condition of the patient, e.g., severity and degree of cancer cell growth and/or tumor growth. In certain embodiments, a composition, e.g., pharmaceutical composition, can include at least 10⁵ bacteria, or at least 10⁶ bacteria, or at least 10⁷ bacteria, or at least 10⁸ bacteria, or at least 10⁹ bacteria from the taxonomic groups listed in Group 2. In certain embodiments, a pharmaceutical composition can include from about 10⁵ bacteria to about 10¹⁰ bacteria from the taxonomic groups listed in Group 2.

In certain non-limiting embodiments, the compositions of the present disclosure can be administered for prophylactic and/or therapeutic treatments. For example, in alternative non-limiting embodiments, pharmaceutical compositions of the present disclosure are administered in an amount sufficient to increase the likelihood of cancer survival, for example, by treating, preventing and/or ameliorating cancer cell growth and/or cancer cell presence and/or tumor growth and/or tumor presence and/or tumor volume. As is well known in the medical arts, dosages for any one patient depends upon many factors, including stage of the disease or condition, the severity of the disease or condition, the patient's size, body surface area, age, the particular compound to be administered, sex, time and route of administration, general health, and interaction with other drugs being concurrently administered.

Accordingly, in certain non-limiting embodiments, a therapeutic bacteria can be administered to a patient alone, or in combination with one or more other drugs, nucleotide sequences, lifestyle changes, etc. used in the treatment or prevention of cancer, or symptoms thereof, or in pharmaceutical compositions where it is mixed with excipient(s) or other pharmaceutically acceptable carriers.

Single or multiple administrations of formulations can be given depending on the dosage and frequency as required and tolerated by the patient. In certain non-limiting embodiments, the formulations should provide a sufficient quantity of active agent to effectively increase the likelihood of cancer survival by treating, preventing or ameliorating the cancer, or symptoms or complications thereof as described herein.

### 5.6. Methods of Treatment

In certain non-limiting embodiments, the present disclosure provides for a method for treating a subject having a cancer. In certain embodiments, the method disclosed herein increases the likelihood of cancer survival in the subject. In certain embodiments, the method disclosed herein increases the amount of bacterial species from the taxonomic groups listed in Group 2 in the subject. In certain embodiments, the method disclosed herein increases proliferation and/or growth of bacterial species from the taxonomic groups listed in Group 2 in a subject. In certain embodiments, the method disclosed herein reduces the amount of bacterial species from the taxonomic groups listed in Group 3 in the subject. In certain embodiments, the method disclosed herein inhibits proliferation and/or growth of bacterial species from the taxonomic groups listed in Group 3 in a subject.

In certain embodiments, the method comprises administering, to a subject in need of such treatment, an effective amount of a composition described herein, for example, a recombinant cell and/or a composition comprising one or more therapeutic bacteria, for example, the bacterial species from the taxonomic groups listed in Group 2, a combination thereof, or a cluster comprising any one or more of the foregoing bacteria.

Subjects in need of such treatment or compositions to increase the likelihood of cancer survival include subjects who have undergone treatment for cancer, for example, who have undergone allo-HCT, or are about to receive treatment for cancer, for example, who are about the receive allo-HCT, as described herein. In certain embodiments, the subject has undergone allo-HCT. In certain embodiments, the subject is about to undergo allo-HCT.

Subjects at decreased likelihood of cancer survival include individuals who have received a hematopoietic cell transplantation (HCT) (for example, an allogeneic), a bone marrow transplant, and/or a cord blood or cord stem cell transplant. In certain non-limiting embodiments, the transplant is T-cell replete. In certain non-limiting embodiments, the transplant is T-cell depleted.

In certain non-limiting embodiments, the present disclosure provides for a method for increasing the likelihood of cancer survival, comprising administering, to a subject in need of such treatment, an effective amount of a composition or a therapeutic bacteria described herein, for example, a bacterial species from the taxonomic groups listed in Group 2, a combination thereof, or a cluster comprising any one or more of the foregoing bacteria.

In certain non-limiting embodiments, an effective amount of a composition or a therapeutic bacteria described herein is administered prior to a cancer treatment. In certain non-limiting embodiments, the cancer treatment is allo-HCT and the composition is administered prior to transplantation.

In certain non-limiting embodiments, an effective amount of a composition or a therapeutic bacteria described herein is administered following a cancer treatment.

The cancer treatment can be allo-HCT and the composition can be administered in the peri-engraftment period.

In certain non-limiting embodiments, an effective amount of a composition or a therapeutic bacteria described herein is an amount which reduces the amount of bacterial species from the taxonomic groups listed in Group 3, and/or inhibit proliferation and/or growth of bacterial species from the taxonomic groups listed in Group 3 in a subject.

In certain non-limiting embodiments, the present disclosure provides for a method of increasing the likelihood of cancer survival, and/or increasing the amount of bacterial species from the taxonomic groups listed in Group 2, and/or increasing proliferation and/or growth of bacterial species from the taxonomic groups listed in Group 2 in a subject, and/or reducing the amount of bacterial species from the taxonomic groups listed in Group 3, and/or inhibiting proliferation and/or growth of bacterial species from the taxonomic groups listed in Group 3 in a subject, comprising administering, to a subject in need of such treatment, an effective amount of a probiotic. In certain non-limiting embodiments, the probiotic comprises a bacterial species from the taxonomic groups listed in Group 2, a combination thereof, or a cluster comprising any one or more of the foregoing bacteria. In certain non-limiting embodiments, the probiotic comprises endogenous flora (for example, an autologous fecal microbiota transplant) that are re-introduced into the subject.

In certain non-limiting embodiments, the present disclosure provides for a method of increasing the likelihood of cancer survival, and/or increasing the amount of bacterial species from the taxonomic groups listed in Group 2, and/or increasing proliferation and/or growth of bacterial species from the taxonomic groups listed in Group 2 in a subject, and/or reducing the amount of bacterial species from the taxonomic groups listed in Group 3, and/or inhibiting proliferation and/or growth of bacterial species from the taxonomic groups listed in Group 3 in a subject, comprising administering, to a subject in need of such treatment, an effective amount of a prebiotic. In certain non-limiting embodiments, the prebiotic promotes the growth, proliferation and/or survival of bacterial species from the taxonomic groups listed in Group 2, a combination thereof, or a cluster comprising any one or more of the foregoing bacteria, in the subject.

In certain non-limiting embodiments, the therapy comprises administering a prebiotic to the subject, wherein the prebiotic comprises one or more agents, for example, a nutritional supplement, that increases growth and survival of bacterial species from the taxonomic groups listed in Group 2, a combination thereof, or a cluster comprising any one or more of the foregoing bacteria. In certain non-limiting embodiments, the prebiotic comprises one or more of poorly-absorbed complex carbohydrates, oligosaccharides, inulin-type fructans or arabinoxylans.

In certain non-limiting embodiments, the present disclosure provides for a method of increasing the likelihood of cancer survival, and/or increasing the amount of bacterial species from the taxonomic groups listed in Group 2, and/or increasing proliferation and/or growth of bacterial species from the taxonomic groups listed in Group 2 in a subject, and/or reducing the amount of bacterial species from the taxonomic groups listed in Group 3, and/or inhibiting proliferation and/or growth of bacterial species from the taxonomic groups listed in Group 3 in a subject, comprising administering, to a subject in need of such treatment, an effective amount of a postbiotic. In certain non-limiting embodiments, the postbiotic comprises one or more agents, such as a protein, expressed by a bacterial species from the taxonomic groups listed in Group 2, a combination thereof, or a cluster comprising any one or more of the foregoing bacteria. In certain non-limiting embodiments, the postbiotic comprises bacterial metabolites, for example, metabolites that promote anti-inflammatory effects. In certain non-limiting embodiments, the postbiotic comprises media from a culture of a bacterial species from the taxonomic groups listed in Group 2, a combination thereof, or a cluster comprising any one or more of the foregoing bacteria. In certain non-limiting embodiments, the postbiotic comprises a short-chain fatty acid such as butyrate or similar acids, or secondary bile acids.

In certain non-limiting embodiments, the present disclosure provides for a method of increasing the likelihood of cancer survival, and/or increasing the amount of bacterial species from the taxonomic groups listed in Group 2, and/or increasing proliferation and/or growth of bacterial species from the taxonomic groups listed in Group 2 in a subject, and/or reducing the amount of bacterial species from the taxonomic groups listed in Group 3, and/or inhibiting proliferation and/or growth of bacterial species from the taxonomic groups listed in Group 3 in a subject, comprising administering, to a subject in need of such treatment, an effective amount of an antibiotic. In certain embodiments, the antibiotic is selective for a bacterial species from the taxonomic groups listed in Group 3. In certain non-limiting embodiments, the antibiotic does not target the bacterial species from the taxonomic groups listed in Group 2, a combination thereof, or a cluster comprising any one or more of the foregoing bacteria. For example, in certain non-limiting embodiments, the methods of the present disclosure comprise administering an antibiotic to the subject along with recombinant bacterial species from the taxonomic groups listed in Group 2, a combination thereof, or a cluster comprising any one or more of the foregoing bacteria, wherein the recombinant cells express antibiotic resistance gene such that the cells are resistant to the antibiotic administered with the recombinant cells. In certain non-limiting embodiments, the antibiotic comprises a penicillin, vancomycin, and/or linezolid antibiotic.

In certain non-limiting embodiments, the present disclosure provides for a method of increasing the likelihood of cancer survival, and/or increasing the amount of bacterial species from the taxonomic groups listed in Group 2, and/or increasing proliferation and/or growth of bacterial species from the taxonomic groups listed in Group 2 in a subject, and/or reducing the amount of bacterial species from the taxonomic groups listed in Group 3, and/or inhibiting proliferation and/or growth of bacterial species from the taxonomic groups listed in Group 3 in a subject, comprising administering, to a subject in need of such treatment, an effective amount of a cancer therapy, for example surgery to remove cancerous cells or tissue, radiation therapy, chemotherapy, immunotherapy (for example, but not limited to, antibodies directed to CTLA-4, PD-1, CD52, and/or CD20; and cytokines such as interferons and interleukins), stem cell therapy and/or cellular therapies (for example, but not limited to, CAR-modified T cells and other antigen-specific T cells).

In certain non-limiting embodiments, such methods comprise determining the abundance of one more bacteria present in an intestinal microbiota sample of a subject diagnosed with cancer, for example, a bacterial species from the taxonomic groups listed in Group 2, a combination thereof, or a cluster comprising any one or more of the foregoing bacteria, wherein the subject is diagnosed or identified as having a decreased likelihood of cancer survival, when the abundance or amount of the one or more bacteria in the subject's microbiota is lower than a bacteria reference level. In certain non-limiting embodiments, a bacteria reference level is an abundance of bacteria, for example, a bacterial species from the taxonomic groups listed in Group 2, a combination thereof, or a cluster comprising any one or more of the foregoing bacteria, present in intestinal microbiota, a level below which is indicative of having a decreased likelihood of cancer survival, as determined by a medical doctor or person of skill in the art.

In certain non-limiting embodiments, such methods comprise determining the abundance of a bacterial species from the taxonomic groups listed in Group 3 present in an intestinal microbiota sample of a subject diagnosed with cancer, wherein the subject is diagnosed or identified as having a decreased likelihood of cancer survival, when the abundance or amount of the bacteria in the subject's microbiota is greater than a bacteria reference level. In certain non-limiting embodiments, a bacteria reference level is an abundance of a bacteria, for example, a bacterial species from the taxonomic groups listed in Group 3, present in intestinal microbiota, a level above which is indicative of a decreased likelihood of cancer survival, as determined by a medical doctor or person of skill in the art.

In certain non-limiting embodiments, a subject determined to have a decreased likelihood of cancer survival can be monitored more frequently and/or for an extended period of time following treatment, and can be administered therapeutic regimens in addition to, or as an alternative to, a hematopoietic cell transplantation, for example, but not limited to, surgery to remove cancerous cells or tissue, radiation therapy, chemotherapy, immunotherapy (for example, but not limited to, antibodies directed to CTLA-4, PD-1, CD52, and/or CD20; and cytokines such as interferons and interleukins), stem cell therapy and/or cellular therapies (for example, but not limited to, CAR-modified T cells and other antigen-specific T cells).

### 5.7. Kits

The presently disclosed subject matter provides for kits for diagnosing a subject as having an increased and/or decreased likelihood of cancer survival. In certain embodiments, the kit comprises one or more agents for detecting the presence of a bacterial species from the taxonomic groups listed in Group 2, a bacterial species from the taxonomic groups listed in Group 3, a combination thereof, or a cluster comprising any one or more of the foregoing bacteria. In certain non-limiting embodiments, the agent comprises nucleic acid primers specific for said bacteria. In certain non-limiting embodiments, the nucleic acid primers are specific for 16S rRNA gene sequencing.

The presently disclosed subject matter provides for kits for treating a subject with a decreased likelihood of cancer survival. In certain non-limiting embodiments, the kit comprises one or more therapeutic composition or cells described herein, for example, therapeutic bacteria selected from a bacterial species from the taxonomic groups listed in Group 2, a combination thereof, or a cluster comprising any one or more of the foregoing bacteria.

In certain non-limiting embodiments, the kit comprises instructions for administering the therapeutic composition or cells. The instructions can comprise information about the use of the composition or cells for increasing the likelihood of cancer survival. In certain non-limiting embodiments, the instructions can comprise at least one of the following: description of the therapeutic composition or cells; dosage schedule and administration; precautions; warnings; indications; counter-indications; over dosage information; adverse reactions; animal pharmacology; clinical studies; and/or references. The instructions can be printed directly on a container (when present) comprising the cells, or as a label applied to the container, or as a separate sheet, pamphlet, card, or folder supplied in or with the container.

### 6. EXAMPLES

The presently disclosed subject matter will be better understood by reference to the following Examples, which are provided as exemplary of the disclosure, and not by way of limitation. Examples 1-4 describe the identification of intestinal flora that are associated with the likelihood of cancer survival.

### Example 1: Microbiota as a Predictor of Mortality in Allogeneic HCT

Allo-HCT patients can exhibit microbiota injury characterized by dramatic expansions of potentially pathogenic bacteria and loss of α-diversity—a parameter that considers the number of unique bacterial taxa present and their relative frequencies. Intestinal microbiota diversity has previously been linked with inflammatory bowel disease and response to cancer immunotherapy. The major adverse outcomes following allo-HCT are relapse, GVHD, infection, and organ toxicity—each of these, as well as overall cancer survival, have been associated in single-center studies with features of the intestinal microbiota in the post-HCT period. For example, domination of intestinal communities by a single bacterial taxon can lead to an increased risk of bloodstream infection by a bacterium of that same taxon, and exposure to certain antibiotics at specific times is associated with transplant-related mortality (TRM) and GVHD-related mortality (GRM). Preclinical models have shown that commensal bacteria can influence GVHD pathophysiology.

These findings in small, single-center studies raise the possibility of developing clinical strategies to manipulate the microbiota with the goal of improving allo-HCT outcomes. A question remains, however, as to whether the relationships between microbiota composition and allo-HCT outcomes are generalizable. Practice patterns vary across transplant centers, particularly with respect to antibiotics and nutrition-two critical determinants of microbiota injury. Moreover, geographic variations in intestinal microbiota composition have been described and implicated in the development of autoimmunity.

The present disclosure profiled intestinal communities in 8,767 fecal samples from 1,362 allo-HCT patients at four centers on three continents using 16S ribosomal sequencing. The associations between microbiota diversity and mortality were examined using Cox proportional hazards analysis in an observational study.

Briefly, the present disclosure observed consistent patterns of microbiota disruption characterized by loss of diversity and domination by single taxa. High intestinal microbiota diversity was associated with a lower risk of death in independent cohorts (First cohort: multivariate HR 0.71 [0.55-0.92], 136 events in 350 patients in the low-diversity group, 104 events in 354 patients in the high-diversity group. Second multicenter cohort: multivariate HR 0.49 [0.27-0.90], 35 events in 92 patients in the low-diversity group, 18 events in 87 patients in the high-diversity group). Subset analyses identified an association between low diversity and an increased risk of transplant-related mortality and graft-vs-host disease. Baseline pre-HCT samples already bore evidence of microbiome disruption, and low diversity prior to transplantation was associated with poor survival.

Comparable patterns of microbiota disruption during allo-HCT across transplant centers and geographic locations were observed, which were characterized by loss of diversity and domination by single taxa. High intestinal microbial diversity at the time of neutrophil engraftment was consistently associated with lower mortality. Thus, it was demonstrated that low diversity is consistently predictive of poor post-HCT outcomes.

### Methods

Stool samples were collected prospectively at each center using similar procedures. Written informed consent was received prior to collection per institutional review boards at each center. DNA extraction, PCR-amplification of genomic 16S ribosomal RNA V4/V5 regions, and sequencing were performed in a central laboratory.

The primary outcome was overall survival (OS). Alpha-diversity, calculated here using the inverse Simpson index, is a single value that summarizes a microbiome community according to the count of unique species and how evenly their frequencies are distributed, but this value does not provide any information about the actual species present. For the primary analysis of clinical outcomes, patients were stratified into high- and low-diversity groups using the median diversity value in the peri-engraftment samples from MSK. When diversity was additionally analyzed as a continuous variable, hazard ratios refer to risk of the outcome per 1 log₁₀ change in inverse Simpson values.

Associations between microbial diversity and outcomes were assessed with overall and cause-specific Cox-proportional hazards multivariable regression models. The cumulative incidences of TRM, GRM, and relapse were estimated while accounting for respective competing endpoints. The study was designed by the authors, who also gathered and analyzed the data. The first author wrote the first draft of the paper; all authors decided to publish the manuscript and vouch for its contents.

### Transplantation Characteristics and Clinical Outcomes

Inclusion criteria for this study were patients with an evaluable stool sample (successfully 16S-amplified and sequenced with >200 reads) that had been collected after day −30 of a first allo-HCT at any of the four centers. Patients who had received an autologous hematopoietic cell transplantation prior to allogeneic HCT were considered eligible for inclusion. Samples from patients who received second allografts were excluded if they were collected after day −10 relative to the second transplant.

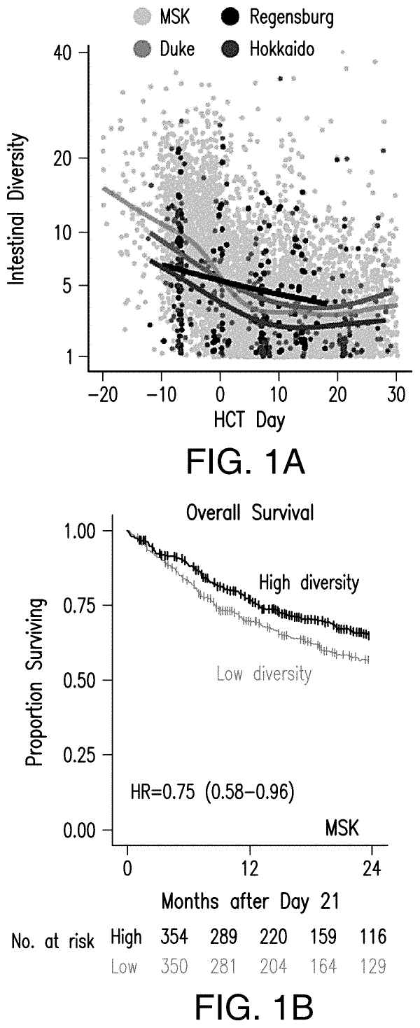

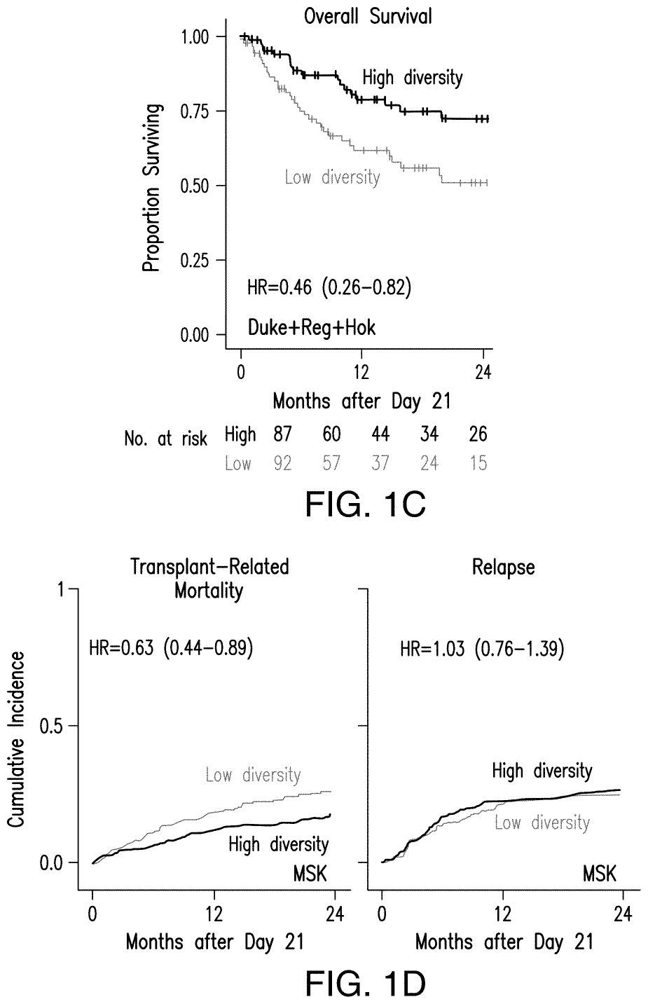

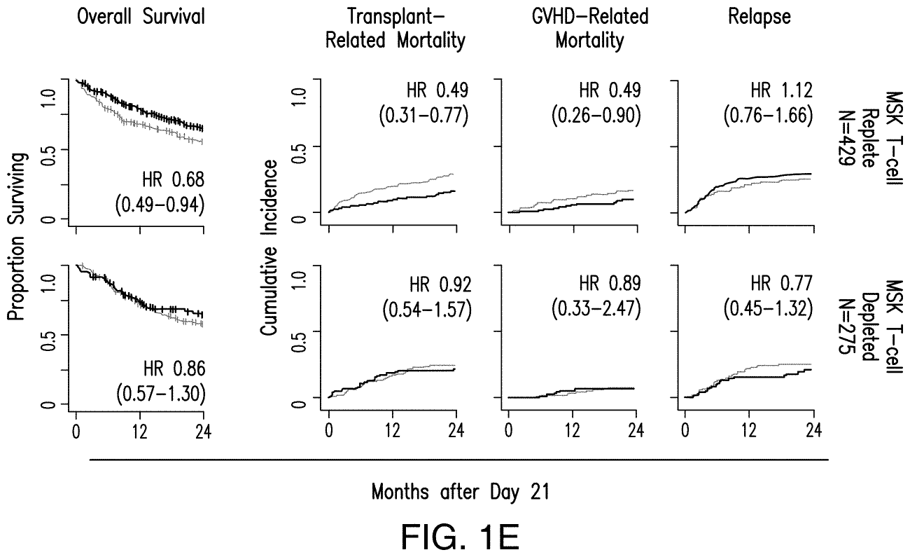

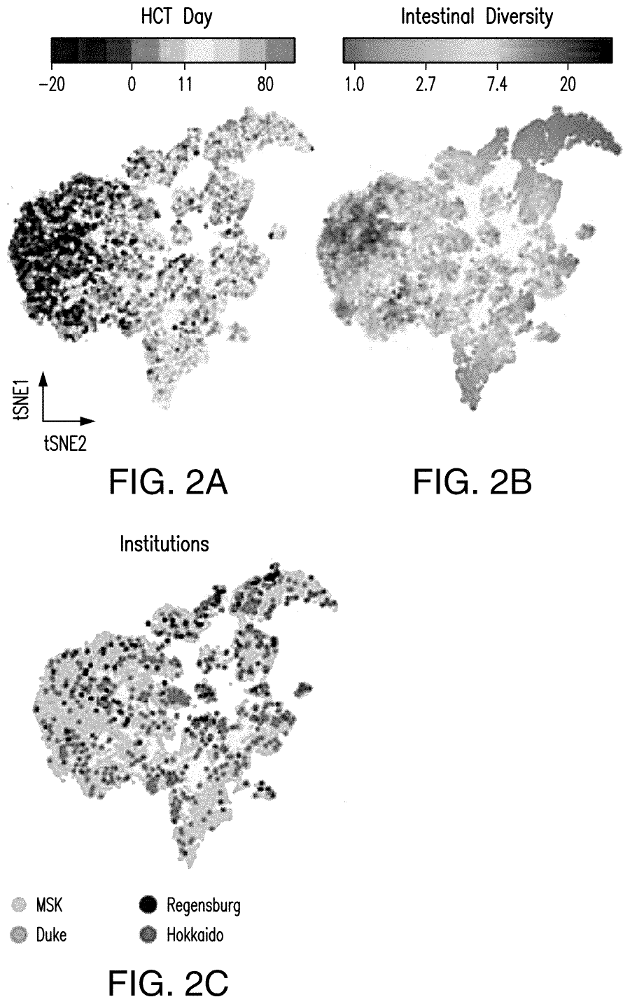

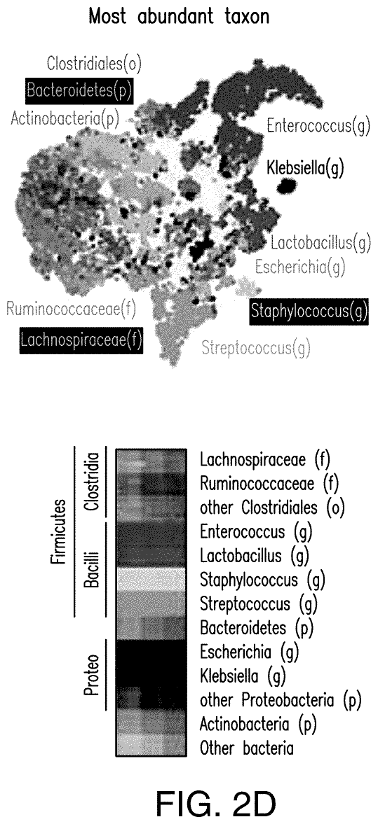

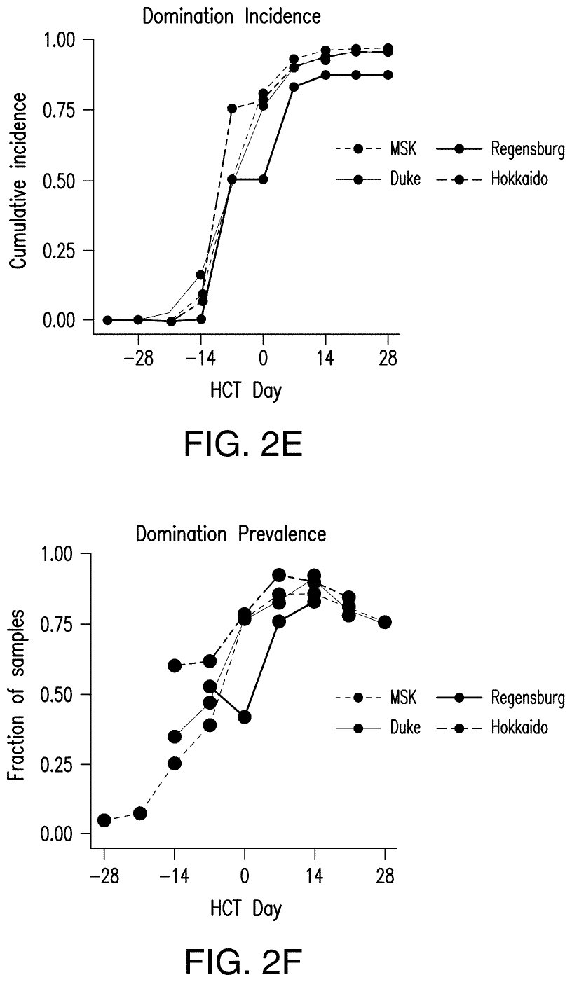

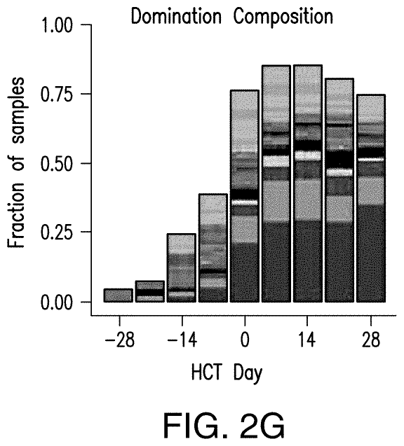

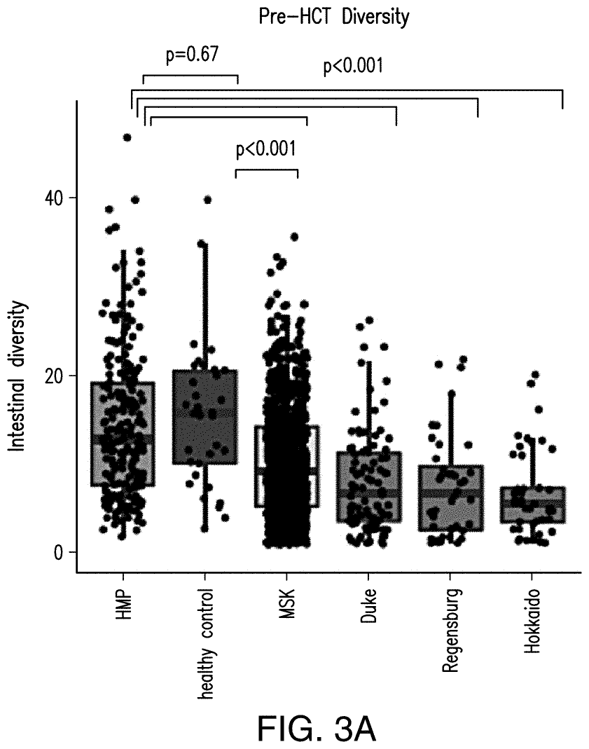

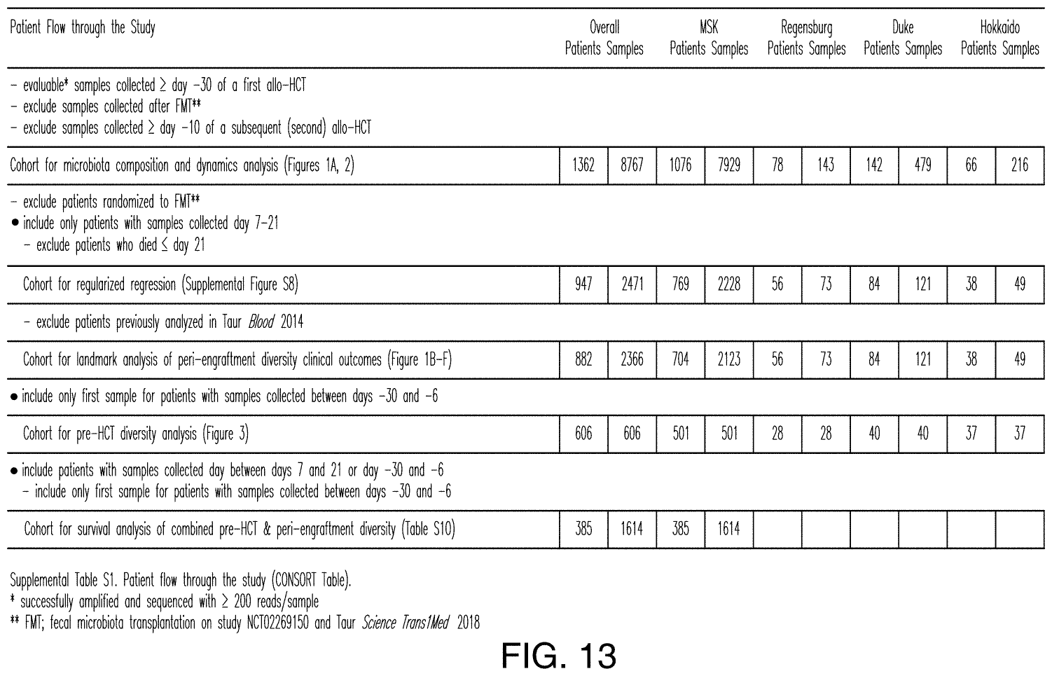

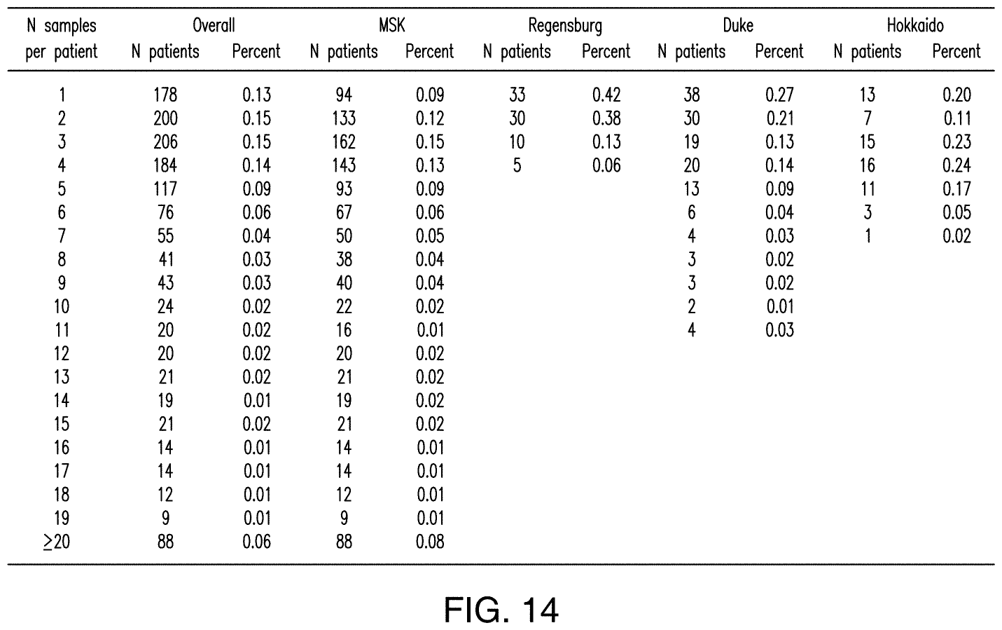

Patient exclusions from various stages of analysis are tabulated in FIG. 13. Importantly, several subjects at one of the centers (MSK) had participated in a randomized clinical trial of fecal microbiota transplantation (FMT, NCT02269150). Patients who were randomized to the control arm (no FMT) were included in all analyses in this study. For patients randomized to receive FMT, samples collected after the FMT procedure were completely excluded from this study, but the pre-FMT samples from these patients were included in analyses of microbiota composition and dynamics (FIG. 1A and FIG. 2). Patients randomized to the FMT arm were excluded from analysis of clinical outcomes (FIGS. 1B-1E, FIG. 3C and FIG. 4A-4D).

Similarly, patients who were analyzed in a prior study of diversity and survival (Taur et al., Blood 2014; 124:1174-82) were included in analyses of microbiota composition and dynamics (FIG. 1A and FIG. 2) but were excluded from analysis of association between clinical outcomes and diversity in the peri-engraftment period (FIGS. 1B-1E) so that the MSK cohort described here is independent of the prior study. These patients were included in the analysis of clinical outcomes and diversity in the pre-HCT period. Patients who participated in a trial randomizing the empiric antibiotic regimens for febrile neutropenia (NCT03078010) were included in the analysis because both arms are within standard practice.

Conditioning regimens were categorized by intensity of myeloablation. Clinical data were obtained from institutional clinical research databases and from dedicated chart reviews. In Table 1, the “other” disease category includes biphenotypic acute leukemia, natural killer-cell large granular lymphocyte leukemia, plasmacytoid dendritic cell neoplasms, and non-malignant hematologic disorders including familial hemophagocytic lymphohistiocytosis, X-linked lymphoproliferative disease, and paroxysmal nocturnal hemoglobinuria. Among the 447 recipients of T-cell depleted grafts transplanted at MSK, 437 (97.8%) received grafts that were CD34-selected on CliniMACS CD34 Reagent system (Miltenyi Biotec, Gladbach, Germany). For ten of the TCD (2.2%) recipients, TCD grafts were prepared via sheep-erythrocyte rosetting based methods. For patients who had transferred their care outside of the four centers within two years of follow-up, outcomes were assessed by telephone interviews with the patients' treating physicians. For the variables reported herein, there were no missing clinical data except in the case of a single patient at MSK who was not evaluable for the hematopoietic cell-transplantation comorbidity index (HCT-CI) because pulmonary function testing was not performed prior to transplantation, and in the case of 38 samples whose time of collection was available only as “pre-transplant” for which a value of day −7 was assigned. Healthy volunteers who provided stool samples provided written informed consent according to a biospecimen collection protocol approved by the MSKCC Institutional Review Board. Raw sequence files from the Human Microbiome Project were downloaded from the human microbiome project website (https://hmpdacc.org) and processed computationally on the same pipeline as samples in this study.

### Samples

Written informed consent was received prior to collection per institutional review boards at each center. DNA extraction, PCR-amplification of genomic 16S ribosomal RNA V4/V5 regions, and sequencing were performed in a central laboratory. Bias was minimized by (a) collecting, aliquoting, and freezing samples at each center according to harmonized procedures, and (b) by performing DNA extraction, PCR amplification, sequencing, and computational analysis centrally at MSK. Stool samples were aliquoted and stored frozen without additives at each center. Aliquots were shipped frozen to a central laboratory (MSK) where bacterial cell walls were disrupted by silica bead-beating, nucleic acids isolated, and the genomic 16S ribosomal-RNA gene V4-V5 variable region was amplified and sequenced on the Illumina MiSeq platform. PCR products were purified either using Qiagen PCR Purification Kit or Agencourt AMPure PCR purification system following the manufacturers' instructions.

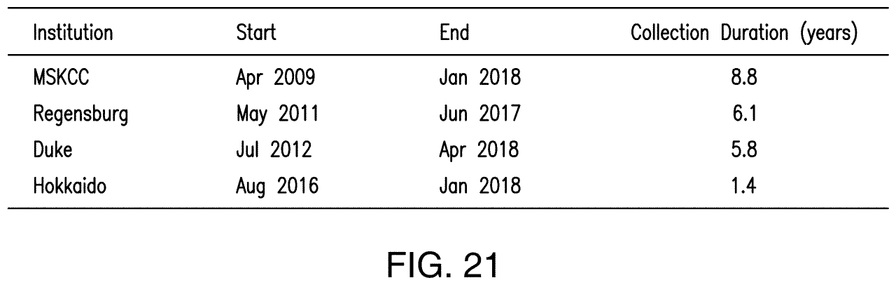

Samples were collected during different time periods lasting 1.4-8.8 years within the years 2009-2018 (FIG. 21). At Regensburg, Duke, and Hokkaido, and for the majority of the MSK patients, weekly samples were requested during the duration of the patients' inpatient admissions, or for outpatient transplants from the start of conditioning through engraftment. At MSK, there were additional collection efforts that yielded additional samples beyond the weekly samples, including upon readmission, in the outpatient setting at scheduled timepoints, and in a subset of patients, near-daily collections while admitted. For the analysis of microbiota composition dynamics (FIGS. 2A-2D) the present disclosure included any evaluable sample collected on or after day −30 relative to a first allo-HCT. Overall, one quarter of the samples were collected between day −30 and 0, 50% of the samples were collected between day −30 and day 10, 75% of the samples were collected between day −30 and day +25.

Stool samples were aliquoted and stored frozen without additives at each center. Aliquots were shipped frozen to a central laboratory (MSK) where bacterial cell walls were disrupted by silica bead-beating, nucleic acids isolated, and the genomic 16S ribosomal-RNA gene V4-V5 variable region was amplified and sequenced on the Illumina MiSeq platform. PCR products were purified either using Qiagen PCR Purification Kit or Agencourt AMPure PCR purification system following the manufacturers' instructions. In cases of poor PCR amplification, the standard PCR buffer was replaced with Ampdirect Plus PCR buffer (Nacalai USA, San Diego, Calif.). In particular, when amplifying samples from Hokkaido, the present disclosure observed that 8 of the initial 13 samples (62%) failed to amplify, in comparison with <25% of failed amplifications from the other cohorts. PCR inhibitor removal using Zymo Research OneStep PCR inhibitor removal kit allowed amplification from only one additional sample. Following other microbiota-profiling studies of Japanese populations, the present disclosure was able to amplify 13 of 13 initial Hokkaido samples using Nacalai AmpDirect Plus buffer. All subsequent Hokkaido samples were amplified according to this protocol.

### Identification of Operational Taxonomic Units

Operational Taxonomic Units (OTUs, as described in Edgar R C, Nat. Meth. 2013; 10:996-8, and also referred to in this disclosure as taxonomic units) were called using a hybrid approach combining de novo and closed-reference OTU-calling. Quality-filtered sequences with >97% identity were grouped into operational taxonomic units (OTUs) as described by Rognes T., et al., Peer J. 2016; 4:e2584. For de novo calling, the search algorithm to dereplicate sequence reads was used. Reads were filtered to sequences of length between 200-350 nucleotides and abundance size of at least two. The search algorithm was used to cluster OTUs (-cluster_otus flag) with parameter -uparse_break. The option uchime_ref was further used to filter for chimeras according to a dereplicated version of NCBI 16S Microbial database, as described by Tatusova T., et al., The NCBI Handbook [Internet]. 2nd ed: National Center for Biotechnology Information (US); 2014. OTUs were clustered at 97% identity. For closed-reference OTU calling, the qiime command pick_closed_reference_otus.py was used. A combined set of over 140M reads from approximately 10000 samples were used for de novo OTU calling to define the closed-reference set of OTUs. Reads from subsequent independent sequencing runs were then identified by closed-reference OTU-calling against the reference set.

OTUs were classified to the species level against the Greengenes database, as described in DeSantis T. Z., et al. Appl. Environ. Microbiol. 2006; 72:5069-72, with gaps in taxonomic annotation filled in by classification against the NCBI 16S ribosomal RNA sequence database (release Dec. 7, 2016). Intestinal diversity was calculated using the inverse Simpson index at the level of OTUs.

### Intestinal Microbiota Diversity

Alpha-diversity is a mathematical value that summarizes an ecological (e.g. microbial) community according to the count of unique species and how evenly their frequencies are distributed. The higher the number of unique species (richness) and the more evenly they are distributed (evenness), the higher the α-diversity. Notably, α-diversity values do not convey any information about the actual species present. Thus, two completely different communities might have identical α-diversity values and share no species in common. Here, the present disclosure calculated α-diversity using the inverse Simpson index at the level of OTUs. An alternative and commonly used method for α-diversity is the Shannon index. These two metrics are highly correlated with one another, but the Simpson index is slightly less sensitive to the long tail of rare bacteria than the Shannon index.

### Taxonomic Color Scheme

The taxonomic color schemes used in FIG. 2D were modified from those used in the R package yingtools2 (https://github.com/ying14/yingtools2) and those described in Taur Y., et al. Sci Transl Med 2018; 10. These color schemes have been customized to highlight common taxonomic patterns in microbiota community in allo-HCT patients. Each genus is assigned to a distinct color shade derived from a basal color that is assigned to a higher-rank taxonomic group in the dataset. This allows visualization of both genus-level and higher-rank taxonomic information. For example, genera from phylum Actinobacteria are in shades of purple, genera from phylum Bacteroidetes are in shades of teal, and most of phylum Firmicutes is depicted in shades of brown. Certain taxonomic groups of biological interest are highlighted separately. For example, genus Enterococcus is in green and family Lachnospiraceae (including genus Blautia) is in shades of pink. The reds of phylum Proteobacteria are variegated to allow resolution between genus Klebsiella and genus Escherichia.

### Enterotypes Classification

Several large-scale studies of healthy human intestinal communities have discerned recurring patterns in which some configurations of relative microbial abundance are observed more frequently than others. A collaborative report by several workers in the microbiota field recently acknowledged the limitations of the enterotypes approach but recommended it as a standard first-step in the classification of human intestinal microbiota datasets. While each enterotype is complex, it is named according to the dominant taxonomic group that contributes to enterotype clustering (Bacteroides, Prevotella, and Firmicutes). In this analysis, genus-level abundances were classified into Enterotypes using the online tool at http://www.enterotypes.org/.

### Statistical Analysis of Clinical Outcomes

A statistical analysis of clinical outcomes was undertaken. A Survival analysis was performed using R package survival, described by Core R Team, R: A Language and Environment for Statistical Computing, Vienna, Australia, R Foundation for Statistical Computing; 2015. When peri-engraftment samples (day 7-21) were analyzed, outcomes were considered in landmark analyses of survivors beyond day 21. When pre-HCT samples were analyzed, outcomes were analyzed from HCT day 0. All survivors were censored at two years of follow-up.

The cumulative incidences of transplant-related mortality (TRM), relapse (defined here as relapse or progression of disease) and GVHD-related mortality (GRM) were estimated with the competing-risks method. The competing risk for relapse was death without relapse. The competing risk for TRM was relapse. The competing risks for GRM were relapse and death without GVHD. Cox proportional hazards multivariable regression models (coxph) were used to assess associations between microbiota and overall cancer survival. For TRM, GRM, and relapse cause-specific Cox regression was used. Hazard ratios (HR) are presented in the Results with square brackets indicating the 95% confidence interval. Time-to-event curves for clinical outcomes were estimated using competing-risks cumulative-incidence function cuminc.

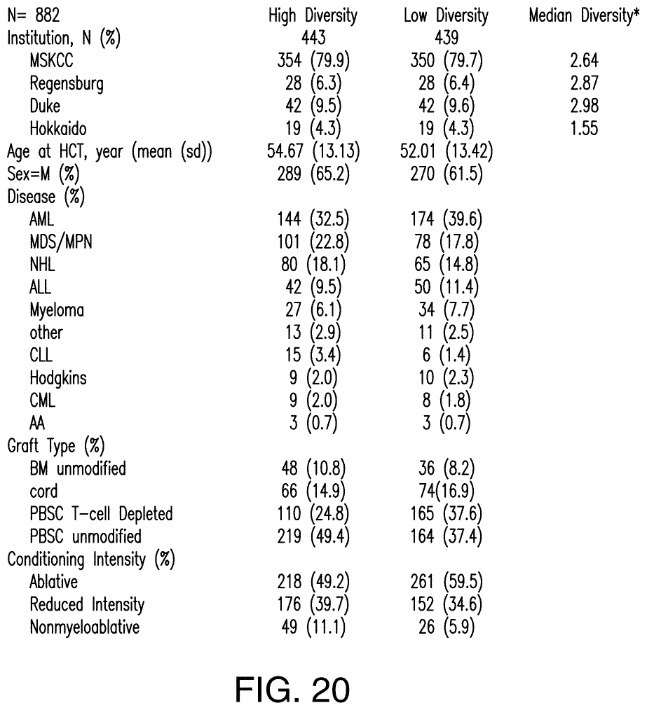

When diversity was considered as a binary (high vs. low) variable, patients were split into above-median and below-median groups using institution-specific median diversity cutoff values. FIG. 20 also tabulates the clinical characteristics of patients in the high- and low-diversity groups.

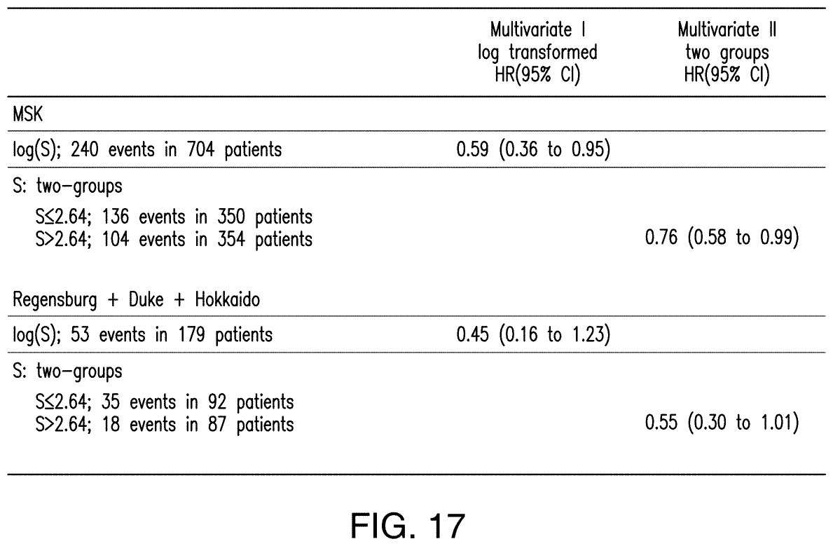

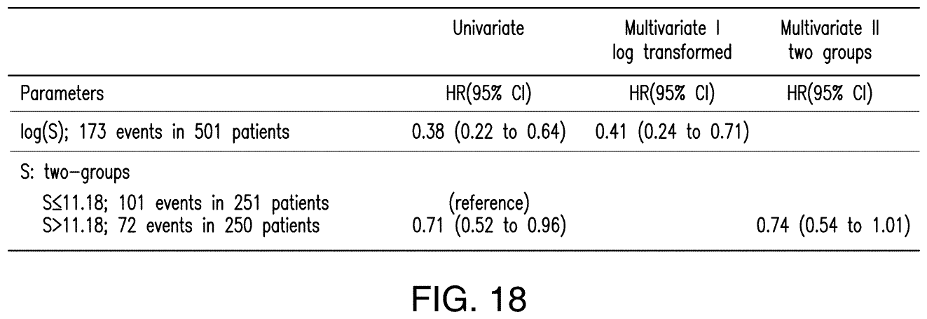

As tabulated in Table 2, and FIGS. 17-19, diversity was also considered as a continuous variable (log 10-transformed). In the analysis of peri-engraftment diversity, when patients had more than one sample in the day 721 sampling window, the per-patient median value was considered. For pre-HCT analysis, the diversity of the first sample for each patient collected in a sampling window of day −30 to day −6 was used. The present disclosure considered only the first sample in the pre-HCT analysis because the present disclosure reasoned that this would be the best estimate of baseline microbiota composition. In multivariate analysis, diversity, age, graft type, conditioning regimen, and the HCT-CI were considered. When samples from more than one institution were analyzed together, the institution of origin was stratified in the coxph formula with parameter strata (institution).

TABLE 2

| | | | Multivariate I | | Multivariate II | |
|---|---|---|---|---|---|---|
| | Univariate | | log transformed | | two groups | |
| | HR (95% CI) | P | HR (95% CI) | P | HR (95% CI) | P |
| MSK | | | | | | |
| log(S); 240 events in 704 | 0.58 (0.37 to | 0.02 | 0.50(0.31 to | 0.004 | | |
| patients | 0.91) | | 0.80) | | | |
| S: two-groups | | | | | | |
| S < 2.64; 136 events in | (reference) | | | | | |
| 350 patients | | | | | | |
| S > 2.64; 104 events in | 0.75 (0.58 to | 0.02 | | | 0.71 (0.55 to | 0.01 |
| 354 patients | 0.96) | | | | 0.92) | |
| Regensburg + Duke + | | | | | | |
| Hokkaido | | | | | | |
| log(S); 53 events in 179 | 0.33 (0.13 to | 0.02 | 0.38 (0.14 to | 0.06 | | |
| patients | 0.84) | | 1.04) | | | |
| S: two-groups | | | | | | |
| S < 2.64; 35 events in 92 | (reference) | | | | | |
| patients | | | | | | |
| S > 2.64; 18 events in 87 | 0.46 (0.26 to | 0.008 | | | 0.49 (0.27 to | 0.02 |
| patients | 0.82) | | | | 0.90) | |

Table 2 shows that intestinal microbiota diversity is associated with survival after allo-HCT. Multivariable Cox proportional hazards analyses of the association of peri-engraftment intestinal diversity (median of samples collected day +7 to +21) with overall survival. The multivariate models were adjusted for age, conditioning intensity, graft source, and the hematopoietic cell transplantation comorbidity index (HCT-CI) and stratified by institution. Intestinal diversity was measured by the inverse Simpson index (S) and is considered here separately as either a log 10-transformed continuous variable or a median-stratified binary variable.

Identification of Bacteria Signature Associated with Patient Outcome

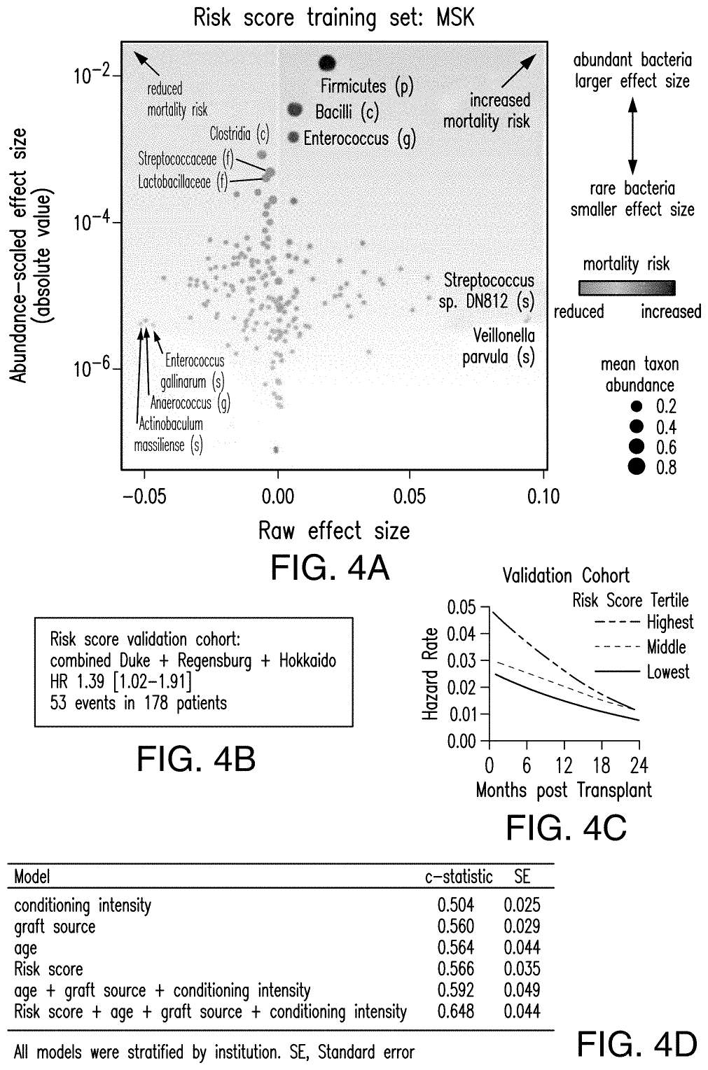

The R package glmnet was used to perform regularized regression and identify a signature of median bacterial abundances during the peri-engraftment period (day 7-21) to predict patient outcomes. To remove spurious signals from the dataset, rare and highly correlated taxonomic groups were eliminated. Considering taxonomy from the phylum to the OTU levels, the dataset initially contained 8,461 taxonomic groups. The analysis was restricted to taxonomic groups that appeared in more than 10% of the present samples with relative abundance above 10⁻⁴. In addition, if the abundance of a parental clade had Pearson correlation greater than 75% to a hierarchically lower clade, the parental clade was removed. The removal of parental clades was prioritized in order to favor higher-resolution taxonomic identification. After these filters, 172 taxonomic groups were used as input features in regularized regression (FIGS. 4A-4D and FIG. 5).

The signature of bacterial effect sizes was identified after 10-fold cross-validation using the function cva.glmnet, with parameters alpha=c(0, 0.1, 0.25, 0.5, 1.0), family=“cox” and maxit=10000. This function identifies optimal lambda and alpha parameters by minimizing cross-validation loss. The abundance of each taxonomic group was log transformed and a pseudo count of 2·10⁻³ was given to eliminate the possibility of −Infinity values, i.e. log(abundance+2e-3). The bacterial signature was identified using a penalty threshold equal to parameter lambda.min in the cross-validation output. The signature of bacterial effect sizes was trained in the MSK cohort and used to compute a risk score in the combined cohort of Regensburg, Duke, and Hokkaido (FIGS. 4A-4D and 5. This risk score was standardized to assure mean=0 and variance=1.

Mapping High-Dimensional Data into tSNE Protections

Mapping high-dimensional data into tSNE projections was done using the Rtsne package to perform t-distributed stochastic neighbor embedding (t-SNE) dimensionality reduction. The Bray-Curtis dissimilarity index computed at the genus level was used as input to the t-SNE algorithm. The perplexity, theta, and iteration parameters were selected by systematic visual inspection to illustrate patterns in the data such as the large cluster of early/diverse samples and the later/low-diversity dominated clusters.

### Compositional Domination Analysis

In FIGS. 2E-2G, domination was defined as any OTU with relative abundance>0.3. The samples were binned into 7-day sliding windows in accordance with the approximately weekly collection schedule. The domination cumulative incidence plot (FIG. 2E) considers patients with at least one evaluable sample in the pre-HCT period and at least one sample in the post-HCT period. The fraction of patients in whom at least one instance of domination was detected by the given time is plotted in FIG. 2F.

### Estimating Geographic and Temporal Variation in Microbiota Compositions

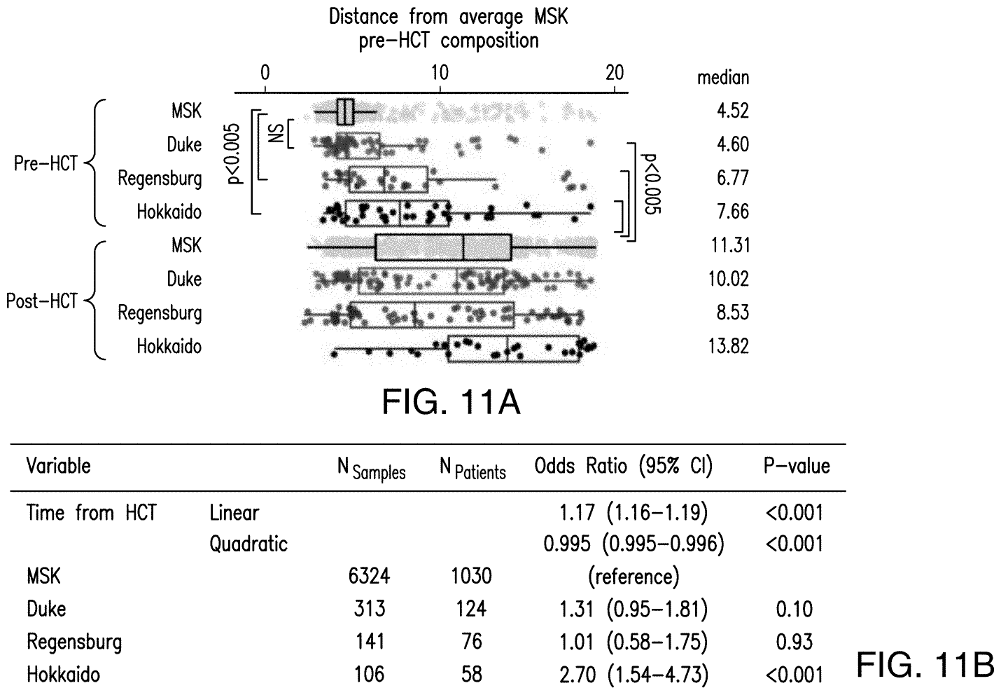

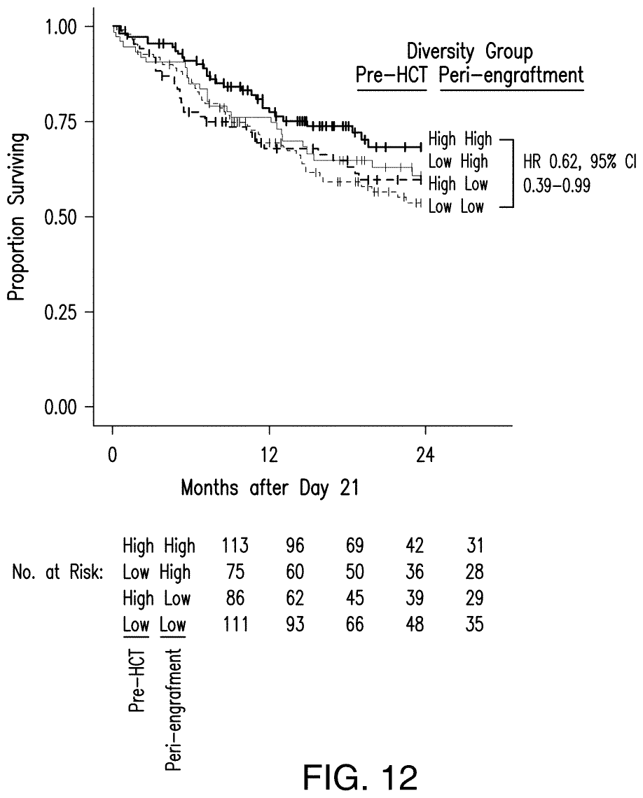

Estimating geographic and temporal variation in microbiota compositions was done by quantified the variation of microbiota composition among the four institutions in comparison to the temporal variation that occurs over time relative to HCT by using a Bray-Curtis beta-diversity matrix (FIGS. 11A-11B). A reference centroid was defined using pre-HCT samples from MSK patients and computed the distance of each sample in the dataset to the pre-HCT MSK centroid. A mean distance value per patient was computed in cases where patients had multiple samples. The Wilcoxon test was used to compare distances of groups of samples from the centroid.

### Antibiotic Analysis

Antibiotics can be associated with microbiome disruption and clinical outcomes in allo-HCT patients. The present disclosure provided an opportunity to explore the association of antibiotic exposures both with (a) microbiota composition and (b) clinical outcomes. The present disclosure sought first to identify key antibiotics most strongly associated with a decline in diversity during allo-HCT, and then to consider exposure to those drugs in a multivariate model of survival.

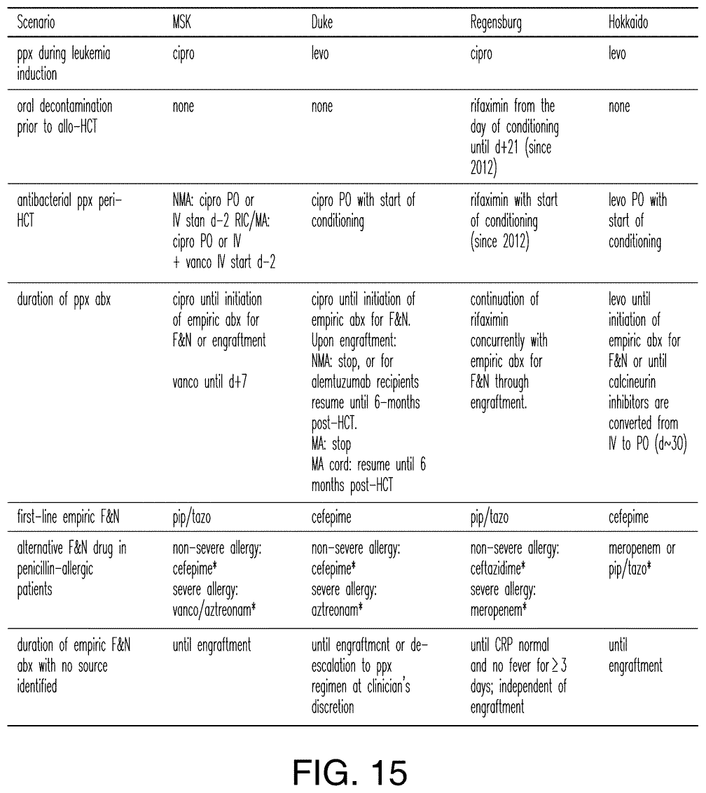

To identify key drugs associated with a decline in intestinal microbiota diversity, the present disclosure considered the change in diversity between the pre-HCT period and the peri-engraftment period, using the same definitions for these sampling periods as elsewhere in this study: for pre-HCT the earliest sample available per patient between day −30 and day −6, and for the peri-engraftment period the median per-patient diversity of samples collected between day 7 and 21. The present disclosure defined an antibiotic exposure window of day −7 and 14 relative to HCT to capture the bulk of antibiotics administered in this population. Among the 31 unique antibiotic drugs administered to the patients during their transplant courses, the present disclosure considered only those to which >20% of patients were exposed during this window in at least one institution. The present disclosure also excluded drugs employed in prophylactic regimens (FIG. 15), among which were fluoroquinolones, trimethoprim-sulfamethoxazole, rifaximin, and intravenous vancomycin. This yielded a set of five antibiotics for further evaluation: cefepime, doripenem, meropenem, piperacillin-tazobactam and meropenem.

The present disclosure modeled variation in microbiota diversity, ΔS, between the peri-engraftment and pre-HCT periods as a function of the time span, Δt, and the effect of antibiotic exposures. The present disclosure assumed that the impact of antibiotics to microbiota diversity is proportional to burden of exposure. The present disclosure defined the exposure burden to antibiotic i, ai, as the number of days of exposure between days −7 and 14 relative to HCT. The time span was computed as day 14 minus the day of the first pre-transplant sample. Formally, the association of drug exposure with diversity was evaluated using linear regression with the following equation in the stats package of R using the function lm( ):

[symbol]

=β₀·Δt+β₁·a₁+ . . . +βₙ·aₙ

where subscripts 1 to n correspond to each drug considered in the model and β represents the regression coefficients. No parameter was used to represent the intercept as it was forced to be 0. Two of the five antibiotics evaluated, piperacillin/tazobactam and meropenem, were significantly associated with variation in microbiota, consistent with the observation that exposure to piperacillin/tazobactam and carbapenems is associated with microbiota disruption. The present disclosure then modified the multivariable model of microbiota diversity and overall survival that is presented in Table 2 to include exposure to piperacillin-tazobactam and meropenem (as continuous variables of exposure duration in days). The present disclosure found that the association between intestinal microbiota diversity and survival remained significant in the MSK and Regensburg cohorts (FIG. 17).

### Observational Study Design Considerations

This manuscript describes an observational cohort study and was prepared, where applicable, according to the reporting recommendations for observational studies (STROBE Statement). The stool samples were collected prospectively (since 2009 at MSK, since 2011 at Regensburg, since 2012 at Duke, and since 2016 at Hokkaido, as detailed in FIG. 21 with the goal of assembling biospecimen banks that would facilitate many different analyses. Once the present disclosure observed that the association between peri-engraftment intestinal microbiota diversity and survival was reproducible (FIGS. 1B-1D, Table 2), the present disclosure commenced additional exploratory analyses that comprise the balance of the figures and tables herein.

The primary outcome was overall cancer survival (OS). α-diversity was calculated using the inverse Simpson index. Associations between microbial diversity and outcomes were assessed with overall and cause-specific Cox-proportional hazards multivariable regression models.

### Results

### Patient Characteristics

Stool samples were prospectively collected at four institutions: Memorial Sloan Kettering Cancer Center (MSK) and Duke University Medical Center in the United States; University Medical Center, University Hospital Regensburg, Germany; and Hokkaido University Hospital, Japan (Table 1). Samples were requested weekly, resulting in a median of four samples per patient. A cohort of 1,362 subjects was identified, each of whom had at least one evaluable sample collected after day −30 of first allo-HCT. Acute leukemia was the most common among a range of indications for transplantation. Variable intensities of pre-HCT conditioning were employed. Unmodified peripheral-blood stem cells were the most common graft type (43%). One center infused grafts that were ex vivo T-cell depleted (TCD, 42% of MSK recipients). Prophylactic and empiric antibiotic practices varied across centers (FIG. 15).

TABLE 1
Clinical Characteristics

| | Overall | | MSK | | Regensburg | | Duke | | Hokkaido | |
|---|---|---|---|---|---|---|---|---|---|---|
| Institution, N (%) | 1362 | (100) | | | | | | | | |
| MSK | 1076 | (79.0) | 1076 | | | | | | | |
| Regensburg | 78 | (5.7) | | | 78 | | | | | |
| Duke | 142 | (10.4) | | | | | 142 | | | |
| Hokkaido | 66 | (4.8) | | | | | | | 66 | |
| Age at HCT, year (mean (sd)) | 53 | (13.0) | 54 | (12.9) | 51 | (11.7) | 51 | (13.2) | 49 | (14.5) |
| Sex = M (%) | 833 | (61.2) | 650 | (60.4) | 47 | (60.3) | 99 | (69.7) | 37 | (56.1) |
| Disease (%) | | | | | | | | | | |
| AML | 490 | (36.0) | 373 | (34.7) | 43 | (55.1) | 42 | (29.6) | 32 | (48.5) |
| MDS/MPN | 264 | (19.4) | 208 | (19.3) | 8 | (10.3) | 41 | (28.9) | 7 | (10.6) |
| NHL | 229 | (16.8) | 187 | (17.4) | 12 | (15.4) | 25 | (17.6) | 5 | (7.6) |
| ALL | 128 | (9.4) | 94 | (8.7) | 6 | (7.7) | 13 | (9.2) | 15 | (22.7) |
| Myeloma | 111 | (8.1) | 97 | (9.0) | 3 | (3.8) | 10 | (7.0) | 1 | (1.5) |
| CLL | 33 | (2.4) | 30 | (2.8) | 3 | (3.8) | | | | |
| Hodgkin's Lymphoma | 31 | (2.3) | 27 | (2.5) | | | 3 | (2.1) | 1 | (1.5) |
| CML | 29 | (2.1) | 23 | (2.1) | 1 | (1.3) | 4 | (2.8) | 1 | (1.5) |
| AA | 10 | (0.7) | 5 | (0.5) | 2 | (2.6) | 2 | (1.4) | 1 | (1.5) |
| other | 37 | (2.7) | 32 | (3.0) | | | 2 | (1.4) | 3 | (4.5) |
| Graft Type (%) | | | | | | | | | | |
| BM unmodified | 113 | (8.3) | 83 | (7.7) | 11 | (14.1) | 13 | (9.2) | 6 | (9.1) |
| cord blood | 211 | (15.5) | 178 | (16.5) | | | 19 | (13.4) | 14 | (21.2) |
| PBSC T-cell Depleted | 447 | (32.8) | 447 | (41.5) | | | | | | |
| PBSC unmodified | 591 | (43.4) | 368 | (34.2) | 67 | (85.9) | 110 | (77.5) | 46 | (69.7) |
| Conditioning Intensity (%) | | | | | | | | | | |
| Ablative | 771 | (56.6) | 598 | (55.6) | 10 | (12.8) | 116 | (81.7) | 47 | (71.2) |
| Reduced Intensity | 468 | (34.4) | 367 | (34.1) | 68 | (87.2) | 14 | (9.9) | 19 | (28.8) |
| Nonmyeloablative | 123 | (9.0) | 111 | (10.3) | | | 12 | (8.5) | | |
| Follow-up of survivors, (months) | | | | | | | | | | |

| | | | | | |
|---|---|---|---|---|---|
| median | 25.2 | 34.2 | 32.5 | 15.0 | 8.3 |
| IQR | (12.8, 49.9) | (15.5, 56.5) | (26.0, 44.4) | (5.8, 23.5) | (4.5, 12.9) |

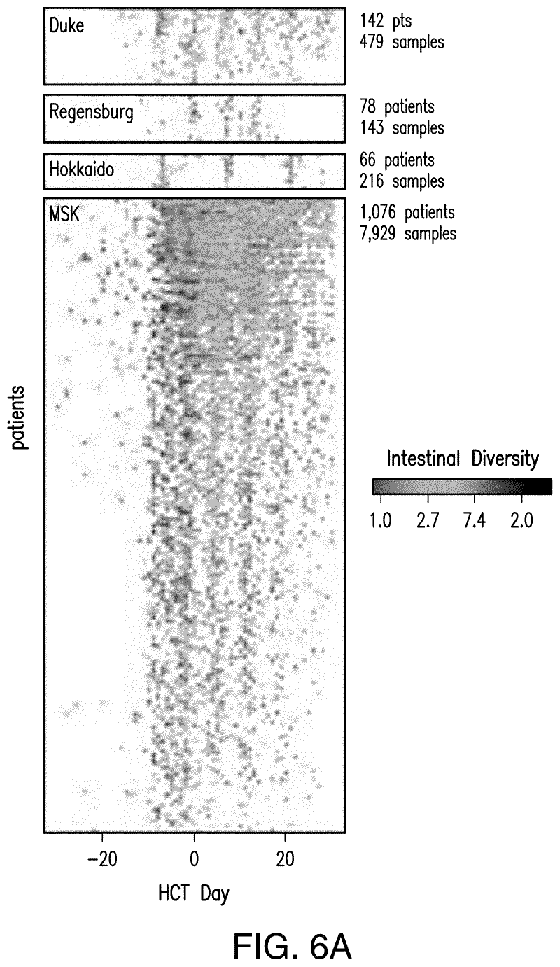

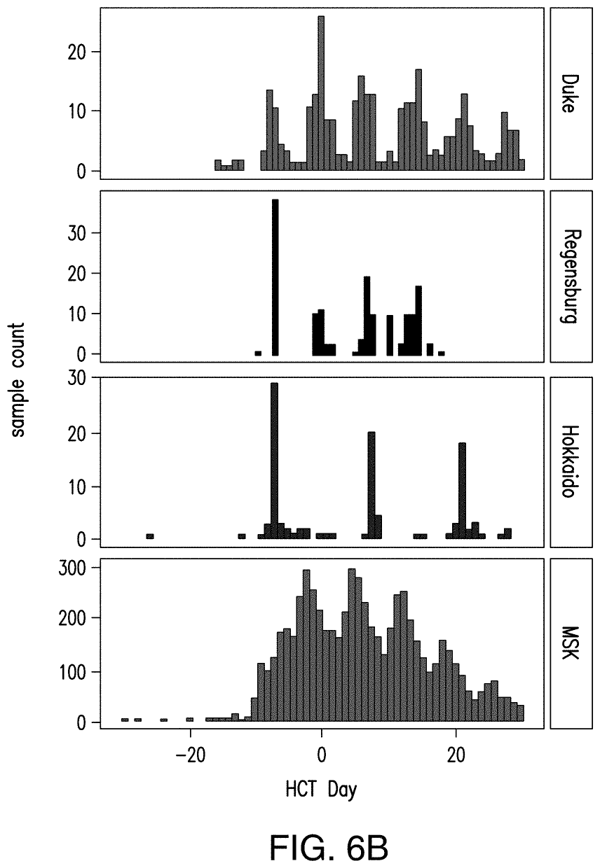

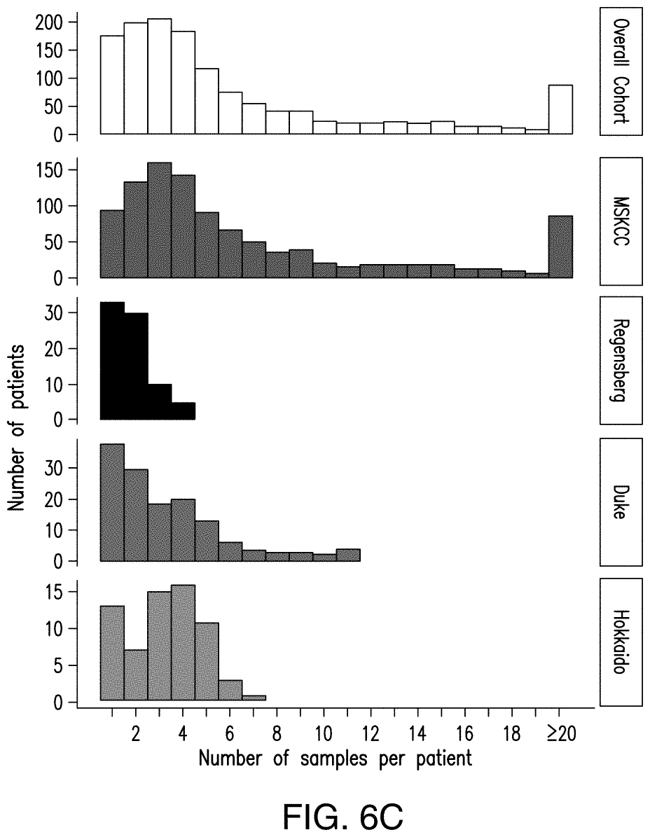

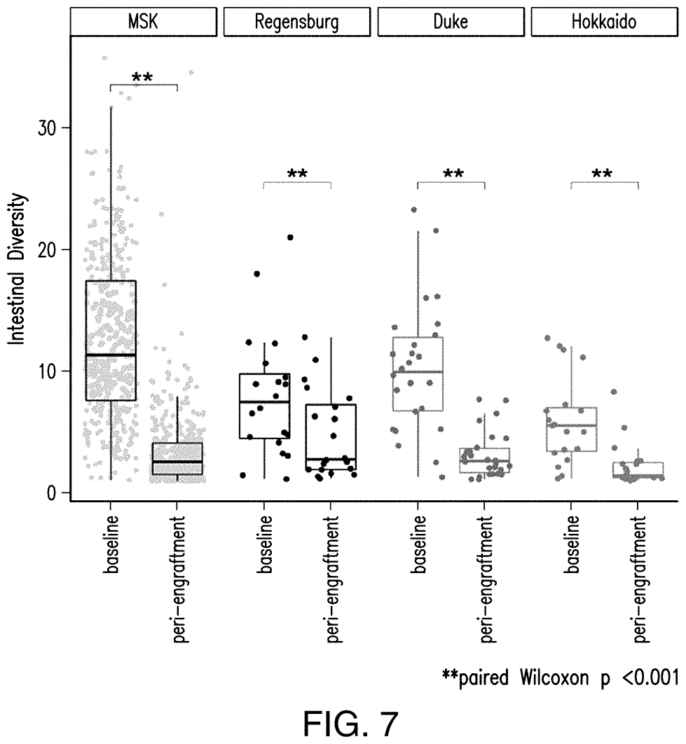

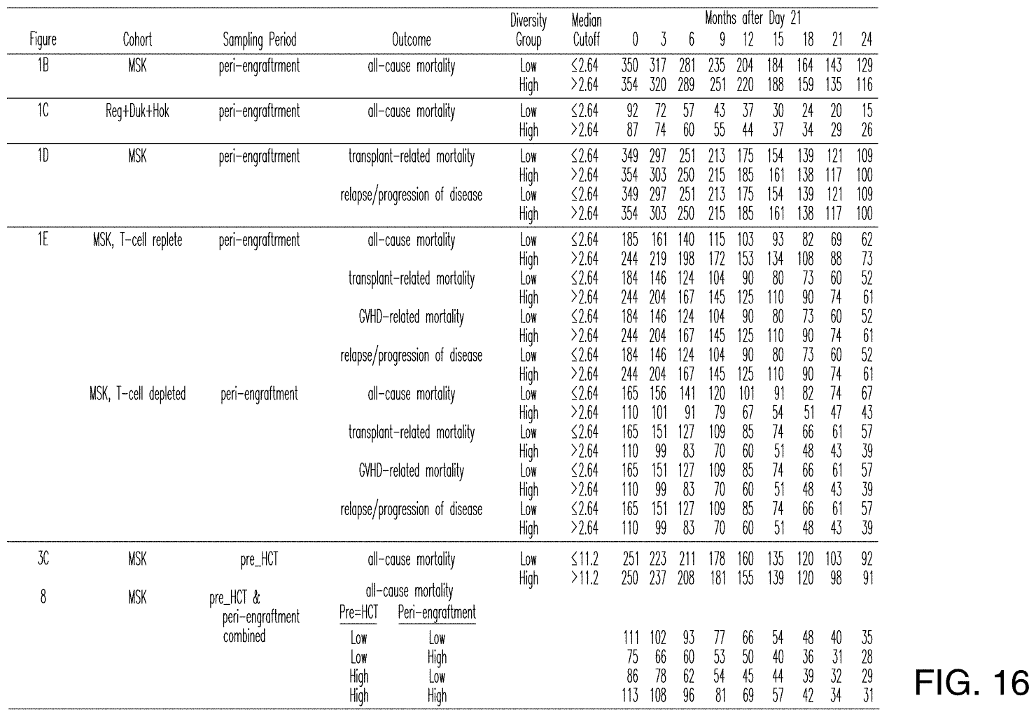

AML, acute myeloid leukemia; MDS/MPN, myelodysplastic syndrome/myeloproliferative neoplasm; NLH, Non-Hodgkin's Lymphoma; ALL, acute lymphoid leukemia; CLL, chronic lymphocytic leukemia; CML, chronic myeloid leukemia; AA, aplastic anemia; BM, bone marrow; PBSC, peripheral blood stem cells; IQR, interquartile range. For the values tabulated herein there were no missing values. Diseases categorized as “other” Diseases categorized as “other” are listed in the Supplemental Appendix. Additional details are in FIG. 13, FIG. 16, and FIGS. 6A-6C.

### Association Between Survival and Intestinal Microbiota Diversity in the Peri-Neutrophil Engraftment Period

To characterize patterns of microbiota injury across different geographies, 8,767 stool samples from 1,362 patients from all four centers were analyzed by 16S sequencing. A loss of diversity was observed at all four centers during the course of transplantation (p<0.001 for each center, FIG. 1A).

To evaluate the association between diversity and OS in a multicenter fashion, the present disclosure considered patients who had evaluable stool samples collected between day 7 and 21 and who survived to day 21. For patients with more than one stool specimen in the sampling window, the median diversity value of those samples was used. At MSK, patients with above-median diversity had a reduced risk of mortality compared with those with below-median diversity (HR 0.75 [0.58-0.96] p=0.02, 136 events in 350 patients in the low-diversity group, 104 events in 354 patients in the high-diversity group, FIG. 1B, Table 2). This association was also observed after multivariable adjustment for age, conditioning intensity, graft source, and the hematopoietic-cell transplantation comorbidity index (HCT-CI) (HR 0.71 [0.55-0.92]p=0.01), and also when diversity was considered as a continuous variable (multivariate HR 0.50 [0.31-0.80] p=0.004, 240 events in 704 patients).

The same association between high peri-engraftment diversity and survival was observed in a combined cohort of the other three centers. Patients with high diversity (>2.64) had a reduced risk of death compared to those with low diversity in both univariate (HR 0.46 [0.26-0.82] p=0.008, 35 events in 92 patients in the low-diversity group, 18 events in 87 patients in the high-diversity group, FIG. 1C) and multivariate analyses (HR 0.49 [0.27-0.90] p=0.02, Table 2). In this cohort, diversity considered as a continuous variable was predictive of mortality only in the univariate analysis (HR 0.33 [0.13-0.84]p=0.02, 53 events in 179 patients) but not in the multivariable model. These data show that high intestinal microbial diversity in the peri-engraftment period (day 7-21) is associated with a reduced risk of death after allo-HCT.

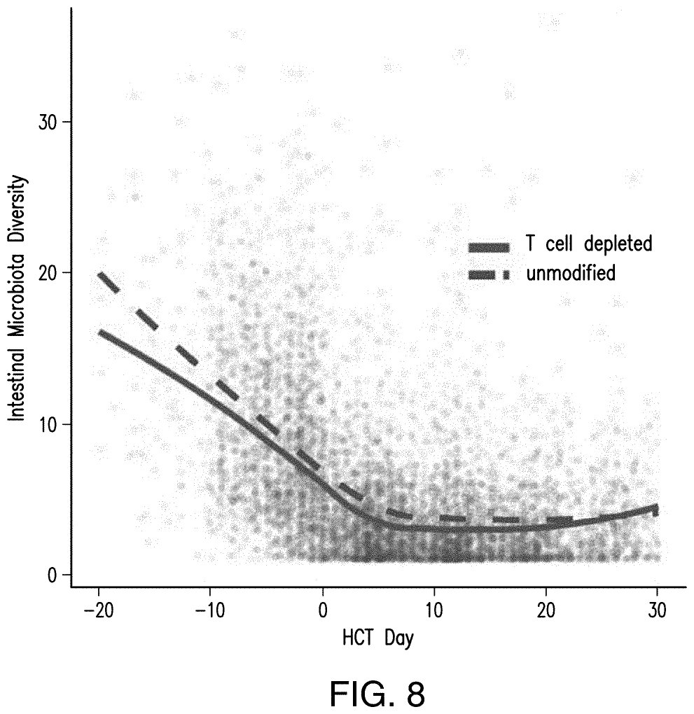

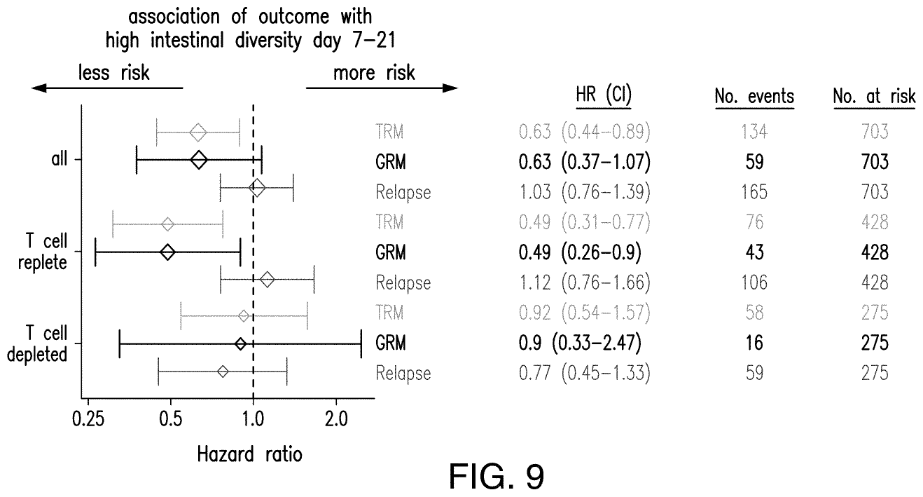

In the MSK cohort, high diversity was associated with a reduced risk of TRM (HR 0.63 [0.44-0.89] p=0.009, 82 events in 349 patients in the low-diversity group, 52 events in 354 patients in the high-diversity group) and not with relapse risk (HR 1.03 [0.76-1.39] p>0.8, 81 events in 349 patients in the low-diversity group, 84 events in 354 patients in the high-diversity group, FIG. 1D). The recipients of TCD grafts in the MSK cohort provided an opportunity to explore the influence of graft composition in the association between diversity and clinical outcomes. Strikingly, while diversity declined comparably in recipients of TCD and unmodified (T-replete) grafts (FIG. 8), the association of microbiota diversity with survival and TRM was observed in recipients of unmodified grafts (HR 0.49 [0.31-0.77] p=0.002, 46 events in 184 patients in the low-diversity group, 30 events in 244 patients in the high-diversity group) and not observed in recipients of TCD grafts (FIG. 1E). Among recipients of unmodified grafts, a reduced risk of GRM in patients with high diversity was observed (HR 0.49 [0.26-0.90], p=0.02, 26 events in 184 patients in the low-diversity group, 17 events in 244 patients in the high-diversity group).

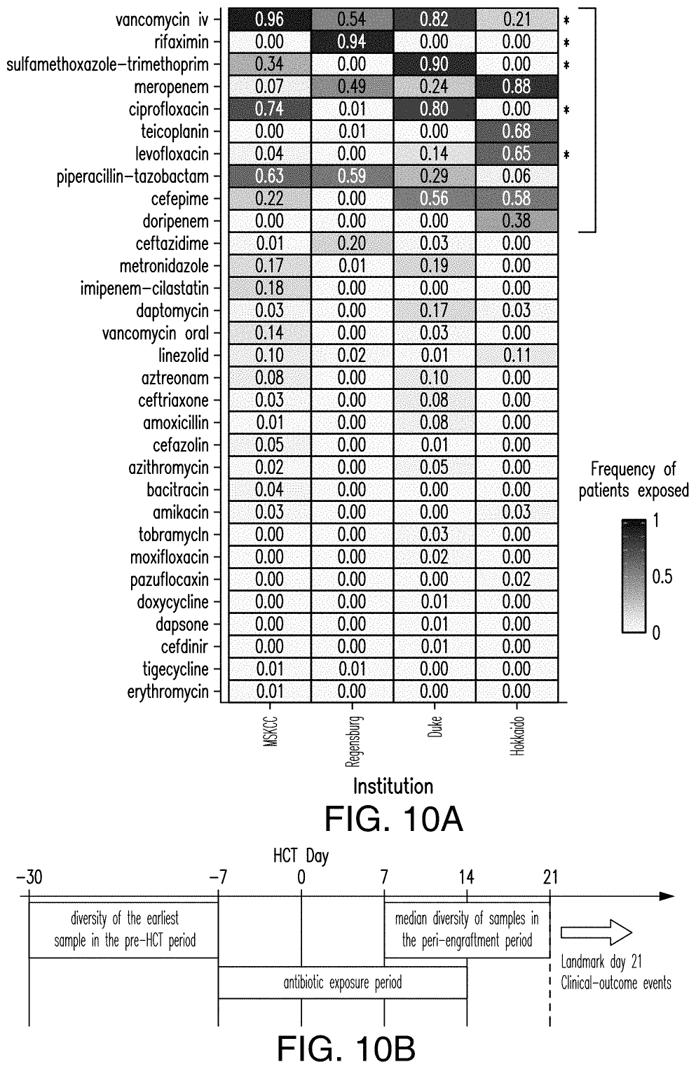

In further exploratory analysis, the association between diversity and survival was also observed in the MSK cohort when multivariable models were modified to include exposure to two antibiotics (piperacillin-tazobactam and meropenem) that the present disclosure identified as associated with a decline in diversity during HCT (FIGS. 10A-10B, and 17). This shows antibiotics are an important determinant of microbiota composition in the setting of allo-HCT.

### Spectrum of Microbiota Disruption in Allo-HCT

Differences in microbiota composition between samples can be visualized using the t-distributed Stochastic Neighbor Embedding (t-SNE) algorithm, such that each point represents a single stool sample. Similar samples are located relatively close to each other, and clusters of distinct microbiota compositions can be appreciated (FIGS. 2A-2G). Color coding a t-SNE projection by time of sample collection demonstrated a large cluster of early samples and several clusters of samples collected mostly at later timepoints (FIG. 2A). As expected from the known decline in microbiota diversity over time (FIG. 1A), the samples in the early cluster tended to be highly diverse, while later timepoints were enriched for several distinct lower-diversity compositions (FIG. 2B).

Samples from all four centers spread across the different composition clusters, revealing no obvious transplant-center-specific effect (FIG. 2C). To quantitatively assess microbiota differences between institutions (over geography) vs. within institutions (over time) the present disclosure compared pre-HCT and post-HCT samples at each institution. The pre-HCT variation in microbiota composition between institutions was smaller than the changes that occur over the course of transplantation, as measured by Bray-Curtis β-diversity distances (p<0.005; FIG. 17).

Color-coding the t-SNE according to the most abundant taxon (FIG. 2D) demonstrated that several of the low diversity compositions were characterized by an abundance of Enterococcus, Klebsiella, Escherichia, Staphylococcus, and Streptococcus. Notably, Enterococcus domination, a low-diversity state that was previously shown to confer an increased risk of vancomycin-resistant enterococcal bacteremia as well as a higher risk of GVHD, occurred at all four centers.

It was observed that a pattern of intestinal domination characterized by dramatic expansions of single taxonomic units is common in allo-HCT patients. To better understand these microbiota disruption patterns across geography, the present disclosure quantified the occurrence of domination during HCT at each institution, as defined by a relative-abundance threshold of any single taxonomic unit 30%. The cumulative incidence of domination by any taxonomic unit rose comparably at the four institutions; the fraction of samples with domination peaked a week following transplantation and decreased moderately thereafter (FIGS. 2E-2F). A variety of taxonomic groups contributed to domination events, the most common belonging to the genera Enterococcus and Streptococcus both at MSK (FIG. 2G) and in the other cohorts (not shown). Taken together, these data demonstrate that microbiota disruption frequently accompanies allo-HCT, and that patterns of microbiota injury are observed consistently across transplant centers, although not in all patients.

### Microbiota Disruption Occurs Prior to HCT and Predicts Survival

Given the link between intestinal microbiota integrity and allo-HCT outcomes, the present disclosure asked whether this association was evident earlier than the peri-engraftment period. To characterize the intestinal communities of patients arriving at transplantation units for allo-HCT, the present disclosure compared the composition of the first sample collected from patients (within a day −30 to −6 sampling window) with those from two sources of healthy volunteers: 313 samples from 212 participants in the Human Microbiome Project whose publicly-available raw sequences were processed on a computational pipeline, and 34 healthy adult volunteers sampled and sequenced at MSK.

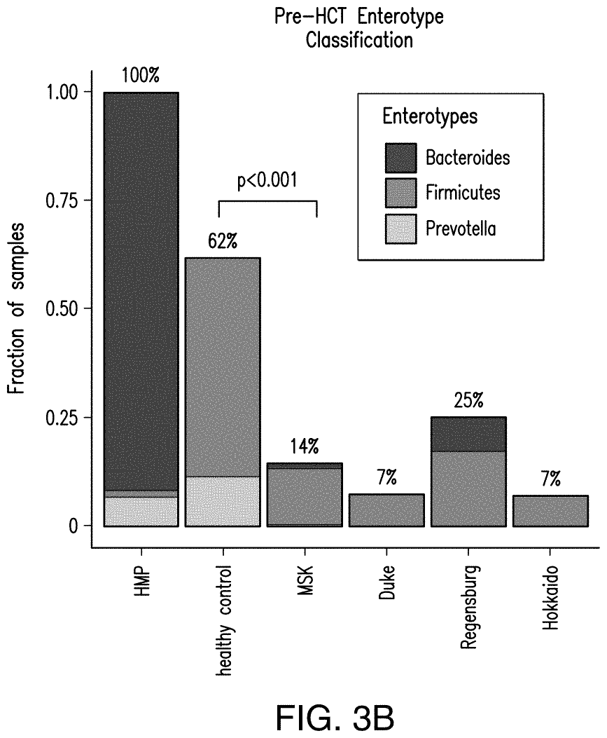

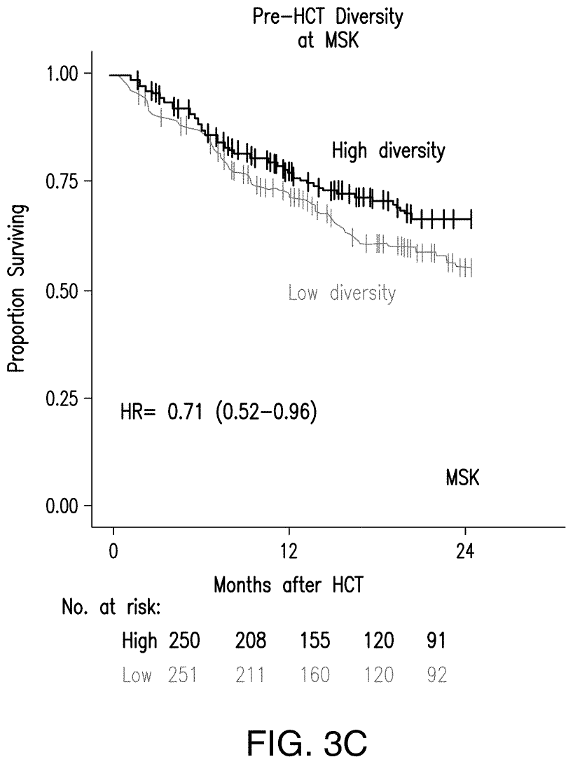

The initial samples from 606 patients at all four institutions had lower diversity than those of the healthy volunteers (p<0.001), and the diversity between the two cohorts of healthy volunteers was not statistically different (FIG. 3A). The present disclosure also evaluated the extent to which initial microbiota compositions could be described by Enterotypes, a reference classifier of healthy human intestinal communities. The initial compositions of patient stool samples were distinct from those of healthy volunteers, as assessed by the fraction of samples that could be categorized to an Enterotype (p<0.02 for Regensburg, p<0.001 for all others, FIG. 3B). This indicates that patients arrived for transplantation with microbiota compositions that were already distinct from those of healthy volunteers.

In the MSK cohort, higher diversity in the pre-HCT period was associated with a lower risk of death (continuous-variable multivariate HR 0.41 [0.24-0.71], p<0.001, 173 events in 501 patients; two-group analyses are in FIG. 3C) and a reduced risk of TRM (HR 0.44 [0.22-0.87] p=0.02 with diversity analyzed as a continuous variable, 103 events in 501 patients). The present disclosure observed a weak correlation between diversity values measured at baseline and during the peri-engraftment period (r=0.22, p<0.001). The association between pre-HCT diversity and survival was not observed in the other three cohorts (Duke, Regensburg, and Hokkaido), however ≤15 events had occurred among the patients with available baseline samples at each center.

### Predictive Microbiota Signature

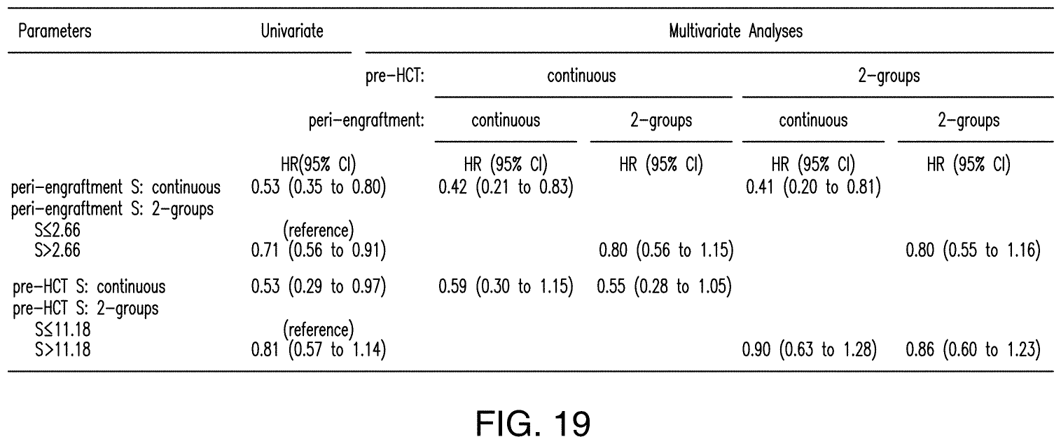

Since α-diversity metrics provide no information about which specific species are present, the present disclosure next sought to identify a signature of bacterial abundances in the peri-engraftment period that could predict mortality risk. From the set of taxonomic levels (species through phylum), the present disclosure selected as candidates those taxa which were present in ≥10% of samples with a minimum relative abundance of 10⁻⁴. The present disclosure also removed features that were highly correlated (Pearson r>0.75) with a hierarchically lower taxonomic rank, yielding an input set of 172 candidate taxonomic features. Regularized Cox regression with cross-validation was performed in the MSK cohort to derive a risk score. Higher values of the score were associated with an increased risk of mortality in the combined Duke+Regensburg+Hokkaido cohort (multivariate-adjusted HR 1.39 [1.02-1.91], p=0.04; FIG. 19, 53 events in 178 patients), indicating that not only a diversity metric but also a signature of specific bacterial abundances is informative about post-HCT mortality risk across independent institutions.

### Discussion

The present disclosure reports the first multi-center study of the intestinal microbiota of allo-HCT patients, where the present disclosure analyzed patterns of microbiota disruption in prospectively-collected stool samples in relation to clinical outcomes. This analysis is the largest and most detailed longitudinal profiling of the intestinal microbiome in patients with hematologic diseases. The diversity of clinical practices across institutions and differences in underlying diseases, conditioning regimens, and graft sources imposed considerable heterogeneity in the study population, yet despite this the present disclosure observed parallel microbiota injury patterns and associations with clinical outcomes.

Pre-transplant microbiota compositions are relatively similar across geography but distinct from those of healthy individuals. Profound microbiota injuries—namely loss of diversity and domination by single taxa—are common events that occur with strikingly convergent kinetics worldwide. These microbiota disruptions are clinically relevant because low diversity at the time of neutrophil engraftment consistently predicts poor OS, particularly in recipients of T-cell-replete grafts. This multicenter, international analysis extends prior observations made in smaller single-center cohorts.

The association of low microbiota diversity with poor survival was partly explained by higher TRM and was most prominently observed in recipients of T-replete (unmodified) grafts, even though intestinal microbiota diversity declined comparably in recipients of TCD grafts. Among recipients of unmodified grafts, higher intestinal microbiome diversity was associated with less GRM, consistent with prior observations. This raises a hypothesis that the correlation between microbiota injury and mortality requires early alloreactivity (e.g. that driven by mature T cells in the graft), however, it can also reflect other clinical parameters that differ in these two patient groups, which include indications for transplant, comorbidities, conditioning intensity and GVHD prophylaxis.

The present disclosure found that by the day of cell infusion, many patients already harbored an intestinal community that was markedly different from those of healthy volunteers and characterized by loss of diversity and domination by single taxa. This demonstrated that the risk of bloodstream infections can be predicted by pre-HCT microbiota composition, and that TRM risk can be predicted by the timing of antibiotic exposure and by pre-HCT colonization by antibiotic-resistant bacteria. Taken together, these results highlight two specific times relative to transplantation in which strategies to remediate or prevent microbiota injury could be evaluated: prior to transplantation or in the peri-engraftment period.

The presently disclosed results extend beyond diversity to provide multicenter evidence that microbiota compositions—specifically the relative abundances of bacterial taxa—offer clinically relevant information about allo-HCT outcomes. Microbiota-based classification algorithms that distinguish cases from controls and can be extrapolated across geography have been previously described for colorectal cancer and inflammatory bowel disease. However, these studies did not consider clinical outcomes beyond diagnosis, and geographic microbiome variation has limited other attempts to apply classifiers across populations. Despite the clinical heterogeneity of the subjects in this study, the present disclosure found that a microbiota-composition risk score trained in one cohort could predict survival in an independent international cohort. It can be of interest to integrate microbiota classifiers into prospective trials of GVHD-predictive biomarkers.

Strengths of this study include its international multicenter design, longitudinal serial sampling, and the uniform tracking of standardized clinical outcomes by transplant centers. Many large-scale observational human microbiome studies have detailed the interactions between microbial communities and their human hosts but few have examined an outcome as important as mortality. The central analysis of samples in this study was another important element, as both wet-lab processing and computational pipelines are critical factors in the reproducibility and quality of microbiota data.

One limitation of this study is that samples were analyzed by targeted sequencing of the 16S gene, which allows reliable genus-level annotation, or in some cases species information and limits the scope of the analysis to bacteria. The alternative of whole metagenomic shotgun sequencing would expand the scope to viruses and fungi and allow identification of encoded metabolic pathways, but with the support of less-well annotated reference databases. Also, samples were not obtained at uniform timepoints relative to transplantation owing to the inherently unscheduled nature of stool collection. A key limitation of an observational study such as this is that it can only demonstrate correlations and not causative relationships. The presently disclosed findings, however, are consistent with preclinical models of allogeneic transplantation and GVHD, which have provided mechanisms by which microbial communities can indeed modulate GVHD.

This study reveals potential opportunities to restore integrity to the intestinal microbiota, for example with fecal microbiota transplantation or other strategies, which could also be evaluated in clinical settings beyond allo-HCT. The similarity between injury patterns and their associations with clinical outcomes raise the possibility that approaches to manipulate the intestinal microbiota with the aim of improving allo-HCT clinical outcomes will be generalizable across graft sources, conditioning regimens, and around the world despite local variations in microbiota composition and in clinical practice.

Although the presently disclosed subject matter and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. All technical features can be individually combined in all possible combinations of such features. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods or steps.

Patents, patent applications, publications, product descriptions, and protocols are cited throughout, the disclosures of which are incorporated herein by reference in their entireties for all purposes.

1. A method for treating a subject having a cancer, comprising:
(a) determining a level of a diagnostic bacterium or a spore thereof in a sample of the subject;
(b) comparing the level of the diagnostic bacterium or spores thereof to a reference diagnostic bacterium or a spore thereof level;
(c) identifying the subject as not likely to exhibit cancer survival:
(i) if the level of the diagnostic bacterium or spore thereof is lower than the reference diagnostic bacterium or spore thereof, wherein the diagnostic bacterium is a bacterium of the taxonomic group selected from the group consisting of Clostridia, Streptococcaceae, Lactobacillaceae, Actinobacteria, Pediococcus acidilactici, Erysipelotrichales, Coprobacillaceae, Clostridiaceae, Akkermansia muciniphila, Escherichia coli, Streptococcus mitis, Veillonellaceae, Bilophila, Parascardovia, Streptococcus salivarius, Coriobacteriaceae, Peptostreptococcaceae, Phascolarctobacterium faecium, Lactobacillus plantarum, Clostridium tertium, Eubacterium biforme, Alphaproteobacteria, Ruminococcaceae, Gemella haemolysans, Dorea, Coprococcus, Clostridium, Rothia, Megasphaera, Atopobium, Clostridium glycolicum, Coprococcus comes, Clostridium spiroforme, Lactobacillus fermentum, Clostridium sp. 826, Lactobacillus animalis, Ruminococcus (family Lachnospiraceae), Streptococcus parasanguinis, Clostridium sp. ID5, Granulicatella adiacens, Actinomyces graevenitzii, Clostridium bartlettii DSM 16795, S24-7, Clostridium algidixylanolyticum, Lactobacillus homohiochii, Tyzzerella nexilis, Mogibacterium neglectum, Lactobacillus reuteri, Clostridium saccharogumia, [Eubacterium] hallii, Drancourtella massiliensis, Clostridium perfringens, Bacteroides stercoris ATCC 43183, Ruminococcus faecis, Clostridium sp. PPf35E6, Bacteroides uniformis, Weissella confusa, Megasphaera micronuciformis, Propionibacterium, Peptostreptococcus sp. MDA2346-2, Bacteroides fragilis, Oscillospira, Lactobacillus gasseri, Rothia dentocariosa, Clostridium sporosphaeroides, Lactococcus, Clostridium hathewayi, Clostridium methylpentosum DSM 5476, Clostridium sp. cTPY-17, Clostridium sp. Enrichment culture clone NHT38, Ruminococcus torques, Clostridium sp. cf3-PUG, Lactobacillus buchneri, mitochondria, Coprococcus, Bacillaceae, Fusobacterium nucleatum, Eggerthella lenta, Anaerococcus, Clostridium sp. Culture-54, Anaerostipes caccae, Blautia faecis, Actinobaculum massiliense, Enterococcus gallinarum, Clostridium sp. MSTE9, ambiguous Leuconostoc, Paraprevotellaceae, Roseburia, unclassified Peptostreptococcaceae, Fusobacterium, Atopobium rimae, Blautia luti, Propionibacterium propionicum, Streptococcus lutetiensis, Scardovia inopinata, Dorea formicigenerans, Corynebacterium pseudogenitalium, Turicibacter sanguinis, Roseburia faecis, Clostridium paraputrificum, Fusobacteria, Clostridium symbiosum, Propionibacterium freudenreichii, and any combinations thereof or the diagnostic bacterium or spore thereof comprises a 16S rRNA gene having a nucleotide sequence that has between about 90 and 100% homology to the nucleotide sequence set forth in any one of SEQ ID NOs:1-69; and/or
(ii) if the level of the diagnostic bacterium or spore thereof is higher than the reference diagnostic bacterium or spore thereof, wherein the diagnostic bacterium is a bacterium of the taxonomic group selected from the group consisting of Firmicutes, Bacilli, Enterococcus, Bacillales, ambiguous Klebsiella, Lactobacillus delbrueckii subsp. Bulgaricus, Coprobacillus, Parabacteroides, Ruminococcus, Streptococcus mutans, Enterococcus rivorum, Enterobacter ludwigii, Clostridium leptum, Enterococcus lactis, Bifidobacterium dentium, Eubacterium limosum, Proteobacteria, Mycoplasma, Holdemania filiformis, Lactococcus piscium, Blautia, Bacteroides thetaiotaomicron, Massiliomicrobiota timonensis, Blautia hydrogenotrophica, Enterococcus mundtii, Prevotella melaninogenica, Erwinia chrysanthemi, Clostridium nexile DSM 1787, Veillonella, Clostridium difficile, [Ruminococcus] obeum, Streptophyta, Bacteroidales, Parascardovia, Clostridium clostridioforme, Blautia obeum, Klebsiella oxytoca, Bulleidia moorei, Parabacteroides merdae, Shuttleworthia satelles, Streptococcus sp. DN812, Clostridium cellulosi, Lactobacillus acidophilus, Bacteroides ovatus, Clostridium hylemonae, Veillonella parvula, Longibaculum muris, Butyrivibrio, Peptostreptococcaceae bacterium canine oral taxon 074, Alloscardovia omnicolens, Lactobacillus salivarius, Clostridium scindens, Alistipes, Clostridium lavalense, Anaerostipes, Abiotrophia defectiva, Leuconostocaceae, Dorea, Alistipes putredinis, Salinicoccus qingdaonensis, Parasutterella excrementihominis, Bacteroides caccae, Bifidobacteriaceae, Streptococcus anginosus, Haemophilus parainfluenzae, Oscillospira, Clostridium aldenense, and any combination thereof or the diagnostic bacterium or spore thereof comprises a 16S rRNA gene sequence that has between about 90 and 100% homology to the nucleotide sequence set forth in any one of SEQ ID NOs:70-118; and
(d) treating the subject identified as not likely to exhibit cancer survival with a cancer treatment.

2. (canceled)

3. A method for treating a subject having a cancer comprising administering a cancer treatment to the subject,
(i) wherein the subject is identified as not likely to exhibit cancer survival by determining that a level of a diagnostic bacterium or a spore thereof is lower than a reference diagnostic bacterium or a spore thereof, wherein the diagnostic bacterium is a bacterium of the taxonomic group selected from the group consisting of Clostridia, Streptococcaceae, Lactobacillaceae, Actinobacteria, Pediococcus acidilactici, Erysipelotrichales, Coprobacillaceae, Clostridiaceae, Akkermansia muciniphila, Escherichia coli, Streptococcus mitis, Veillonellaceae, Bilophila, Parascardovia, Streptococcus salivarius, Coriobacteriaceae, Peptostreptococcaceae, Phascolarctobacterium faecium, Lactobacillus plantarum, Clostridium tertium, Eubacterium biforme, Alphaproteobacteria, Ruminococcaceae, Gemella haemolysans, Dorea, Coprococcus, Clostridium, Rothia, Megasphaera, Atopobium, Clostridium glycolicum, Coprococcus comes, Clostridium spiroforme, Lactobacillus fermentum, Clostridium sp. 826, Lactobacillus animalis, Ruminococcus (family Lachnospiraceae), Streptococcus parasanguinis, Clostridium sp. ID5, Granulicatella adiacens, Actinomyces graevenitzii, Clostridium bartlettii DSM 16795, S24-7, Clostridium algidixylanolyticum, Lactobacillus homohiochii, Tyzzerella nexilis, Mogibacterium neglectum, Lactobacillus reuteri, Clostridium saccharogumia, [Eubacterium] hallii, Drancourtella massiliensis, Clostridium perfringens, Bacteroides stercoris ATCC 43183, Ruminococcus faecis, Clostridium sp. PPf35E6, Bacteroides uniformis, Weissella confusa, Megasphaera micronuciformis, Propionibacterium, Peptostreptococcus sp. MDA2346-2, Bacteroides fragilis, Oscillospira, Lactobacillus gasseri, Rothia dentocariosa, Clostridium sporosphaeroides, Lactococcus, Clostridium hathewayi, Clostridium methylpentosum DSM 5476, Clostridium sp. cTPY-17, Clostridium sp. Enrichment culture clone NHT38, Ruminococcus torques, Clostridium sp. cf3-PUG, Lactobacillus buchneri, mitochondria, Coprococcus, Bacillaceae, Fusobacterium nucleatum, Eggerthella lenta, Anaerococcus, Clostridium sp. Culture-54, Anaerostipes caccae, Blautia faecis, Actinobaculum massiliense, Enterococcus gallinarum, Clostridium sp. MSTE9, ambiguous Leuconostoc, Paraprevotellaceae, Roseburia, unclassified Peptostreptococcaceae, Fusobacterium, Atopobium rimae, Blautia luti, Propionibacterium propionicum, Streptococcus lutetiensis, Scardovia inopinata, Dorea formicigenerans, Corynebacterium pseudogenitalium, Turicibacter sanguinis, Roseburia faecis, Clostridium paraputrificum, Fusobacteria, Clostridium symbiosum, Propionibacterium freudenreichii, and any combinations thereof or the diagnostic bacterium or spore thereof comprises a 16S rRNA gene having a nucleotide sequence that has between about 90 and 100% homology to the nucleotide sequence set forth in any one of SEQ ID NOs:1-69; and/or
(ii) wherein the subject is identified as not likely to exhibit cancer survival by determining that a level of a diagnostic bacterium or a spore thereof is higher than a reference diagnostic bacterium or a spore thereof, wherein the diagnostic bacterium is a bacterium of the taxonomic group selected from the group consisting of Firmicutes, Bacilli, Enterococcus, Bacillales, ambiguous Klebsiella, Lactobacillus delbrueckii subsp. Bulgaricus, Coprobacillus, Parabacteroides, Ruminococcus, Streptococcus mutans, Enterococcus rivorum, Enterobacter ludwigii, Clostridium leptum, Enterococcus lactis, Bifidobacterium dentium, Eubacterium limosum, Proteobacteria, Mycoplasma, Holdemania filiformis, Lactococcus piscium, Blautia, Bacteroides thetaiotaomicron, Massiliomicrobiota timonensis, Blautia hydrogenotrophica, Enterococcus mundtii, Prevotella melaninogenica, Erwinia chrysanthemi, Clostridium nexile DSM 1787, Veillonella, Clostridium difficile, [Ruminococcus] obeum, Streptophyta, Bacteroidales, Parascardovia, Clostridium clostridioforme, Blautia obeum, Klebsiella oxytoca, Bulleidia moorei, Parabacteroides merdae, Shuttleworthia satelles, Streptococcus sp. DN812, Clostridium cellulosi, Lactobacillus acidophilus, Bacteroides ovatus, Clostridium hylemonae, Veillonella parvula, Longibaculum muris, Butyrivibrio, Peptostreptococcaceae bacterium canine oral taxon 074, Alloscardovia omnicolens, Lactobacillus salivarius, Clostridium scindens, Alistipes, Clostridium lavalense, Anaerostipes, Abiotrophia defectiva, Leuconostocaceae, Dorea, Alistipes putredinis, Salinicoccus qingdaonensis, Parasutterella excrementihominis, Bacteroides caccae, Bifidobacteriaceae, Streptococcus anginosus, Haemophilus parainfluenzae, Oscillospira, Clostridium aldenense, and any combination thereof or the diagnostic bacterium or spore thereof comprises a 16S rRNA gene sequence that has between about 90 and 100% homology to the nucleotide sequence set forth in any one of SEQ ID NOs:70-118.

4. (canceled)

5. The method of claim 1, wherein the subject has previously received a hematopoietic cell transplantation (HCT) and/or the cancer treatment is a hematopoietic cell transplantation (HCT).

6. (canceled)

7. The method of claim 5, wherein the HCT is an allogenic hematopoietic cell transplantation (allo-HCT).

8. (canceled)

9. The method of claim 1, wherein the level of the diagnostic bacterium or spore thereof is the relative abundance of the diagnostic bacterium or spores thereof as compared to other bacteria in the sample.

10. The method of claim 1, wherein the sample is a fecal sample or an intestinal content sample of the subject.

11. The method of claim 1, wherein the cancer treatment comprises administering to the subject a therapeutic bacterium or a spore thereof or a pharmaceutical composition comprising thereof, a hematopoietic cell transplantation (HCT), surgery, radiation therapy, chemotherapy, immunotherapy, stem cell therapy, cellular therapy, a probiotic bacteria, a probiotic yeast, a prebiotic, a postbiotic, an antibiotic, or a combination thereof.

12. The method of claim 11, wherein the therapeutic bacterium is a bacterium of the taxonomic group selected from the group consisting of Clostridia, Streptococcaceae, Lactobacillaceae, Actinobacteria, Pediococcus acidilactici, Erysipelotrichales, Coprobacillaceae, Clostridiaceae, Akkermansia muciniphila, Escherichia coli, Streptococcus mitis, Veillonellaceae, Bilophila, Parascardovia, Streptococcus salivarius, Coriobacteriaceae, Peptostreptococcaceae, Phascolarctobacterium faecium, Lactobacillus plantarum, Clostridium tertium, Eubacterium biforme, Alphaproteobacteria, Ruminococcaceae, Gemella haemolysans, Dorea, Coprococcus, Clostridium, Rothia, Megasphaera, Atopobium, Clostridium glycolicum, Coprococcus comes, Clostridium spiroforme, Lactobacillus fermentum, Clostridium sp. 826, Lactobacillus animalis, Ruminococcus (family Lachnospiraceae), Streptococcus parasanguinis, Clostridium sp. ID5, Granulicatella adiacens, Actinomyces graevenitzii, Clostridium bartlettii DSM 16795, S24-7, Clostridium algidixylanolyticum, Lactobacillus homohiochii, Tyzzerella nexilis, Mogibacterium neglectum, Lactobacillus reuteri, Clostridium saccharogumia, [Eubacterium] hallii, Drancourtella massiliensis, Clostridium perfringens, Bacteroides stercoris ATCC 43183, Ruminococcus faecis, Clostridium sp. PPf35E6, Bacteroides uniformis, Weissella confusa, Megasphaera micronuciformis, Propionibacterium, Peptostreptococcus sp. MDA2346-2, Bacteroides fragilis, Oscillospira, Lactobacillus gasseri, Rothia dentocariosa, Clostridium sporosphaeroides, Lactococcus, Clostridium hathewayi, Clostridium methylpentosum DSM 5476, Clostridium sp. cTPY-17, Clostridium sp. Enrichment culture clone NHT38, Ruminococcus torques, Clostridium sp. cf3-PUG, Lactobacillus buchneri, mitochondria, Coprococcus, Bacillaceae, Fusobacterium nucleatum, Eggerthella lenta, Anaerococcus, Clostridium sp. Culture-54, Anaerostipes caccae, Blautia faecis, Actinobaculum massiliense, Enterococcus gallinarum, Clostridium sp. MSTE9, ambiguous Leuconostoc, Paraprevotellaceae, Roseburia, unclassified Peptostreptococcaceae, Fusobacterium, Atopobium rimae, Blautia luti, Propionibacterium propionicum, Streptococcus lutetiensis, Scardovia inopinata, Dorea formicigenerans, Corynebacterium pseudogenitalium, Turicibacter sanguinis, Roseburia faecis, Clostridium paraputrificum, Fusobacteria, Clostridium symbiosum, Propionibacterium freudenreichii, and any combinations thereof.

13. The method of claim 12, wherein the therapeutic bacterium or spore thereof is administered in an amount effective (i) to increase the amount of the diagnostic bacterium or spore thereof of (c)(i) in the subject and/or (ii) to decrease the amount of diagnostic bacterium or spore thereof of (c)(ii) in the subject.

14. (canceled)

15. (canceled)

16. The method of claim 1, wherein the cancer treatment comprises a combination of administering to the subject the therapeutic bacterium or spore thereof or a pharmaceutical composition comprising thereof, and the HCT.

17.-22. (canceled)

23. The method of claim 1, wherein the method:
(a) increases the amount of the diagnostic bacterium or spore thereof of (c)(i) in the subject;
(b) increases the proliferation or growth of the diagnostic bacterium or spore thereof of (c)(i) in the subject;
(c) decreases the amount of the diagnostic bacterium or spore thereof of (c)(ii) in the subject;
(d) decreases the proliferation or growth of the diagnostic bacterium or spore thereof of (c)(ii) in the subject; and/or
(e) increases the likelihood of cancer survival in the subject.

24. The method of claim 1, wherein cancer survival is the survival of the subject at least about 2 years following the cancer treatment.

25. A pharmaceutical composition comprising a therapeutic bacterium or a spore thereof, wherein:
(i) the bacterium is a bacterium of the taxonomic group selected from the group consisting of Clostridia, Streptococcaceae, Lactobacillaceae, Actinobacteria, Pediococcus acidilactici, Erysipelotrichales, Coprobacillaceae, Clostridiaceae, Akkermansia muciniphila, Escherichia coli, Streptococcus mitis, Veillonellaceae, Bilophila, Parascardovia, Streptococcus salivarius, Coriobacteriaceae, Peptostreptococcaceae, Phascolarctobacterium faecium, Lactobacillus plantarum, Clostridium tertium, Eubacterium biforme, Alphaproteobacteria, Ruminococcaceae, Gemella haemolysans, Dorea, Coprococcus, Clostridium, Rothia, Megasphaera, Atopobium, Clostridium glycolicum, Coprococcus comes, Clostridium spiroforme, Lactobacillus fermentum, Clostridium sp. 826, Lactobacillus animalis, Ruminococcus (family Lachnospiraceae), Streptococcus parasanguinis, Clostridium sp. ID5, Granulicatella adiacens, Actinomyces graevenitzii, Clostridium bartlettii DSM 16795, S24-7, Clostridium algidixylanolyticum, Lactobacillus homohiochii, Tyzzerella nexilis, Mogibacterium neglectum, Lactobacillus reuteri, Clostridium saccharogumia, [Eubacterium] hallii, Drancourtella massiliensis, Clostridium perfringens, Bacteroides stercoris ATCC 43183, Ruminococcus faecis, Clostridium sp. PPf35E6, Bacteroides uniformis, Weissella confusa, Megasphaera micronuciformis, Propionibacterium, Peptostreptococcus sp. MDA2346-2, Bacteroides fragilis, Oscillospira, Lactobacillus gasseri, Rothia dentocariosa, Clostridium sporosphaeroides, Lactococcus, Clostridium hathewayi, Clostridium methylpentosum DSM 5476, Clostridium sp. cTPY-17, Clostridium sp. Enrichment culture clone NHT38, Ruminococcus torques, Clostridium sp. cf3-PUG, Lactobacillus buchneri, mitochondria, Coprococcus, Bacillaceae, Fusobacterium nucleatum, Eggerthella lenta, Anaerococcus, Clostridium sp. Culture-54, Anaerostipes caccae, Blautia faecis, Actinobaculum massiliense, Enterococcus gallinarum, Clostridium sp. MSTE9, ambiguous Leuconostoc, Paraprevotellaceae, Roseburia, unclassified Peptostreptococcaceae, Fusobacterium, Atopobium rimae, Blautia luti, Propionibacterium propionicum, Streptococcus lutetiensis, Scardovia inopinata, Dorea formicigenerans, Corynebacterium pseudogenitalium, Turicibacter sanguinis, Roseburia faecis, Clostridium paraputrificum, Fusobacteria, Clostridium symbiosum, Propionibacterium freudenreichii, and any combinations thereof, or
(b) the bacterium or spore thereof comprises a 16S rRNA gene sequence that has between about 90 and 100% homology to the nucleotide sequence set forth in any one of SEQ ID NOs:1-69.

26. (canceled)

27. The pharmaceutical composition of claim 25, wherein the pharmaceutical composition:
(i) is formulated for oral, nasogastric, rectal, percutaneous (e.g., G tube), orogastric tube, or other enteral routes administration;
(ii) further comprises a probiotic bacteria, a probiotic yeast, a prebiotic, a postbiotic, an antibiotic, or a combination thereof;
(iii) is in a form of a liquid, a suspension, a dried powder, a tablet, a capsule, a food product, or a combination thereof; and/or
(iv) comprises the bacterium or spore thereof in an amount that increases the likelihood of cancer survival in a subject administered the pharmaceutical composition.

28. (canceled)

29. (canceled)

30. The pharmaceutical composition of claim 25, wherein the bacterium or spore thereof is a recombinant bacterium, or a progeny thereof; and/or comprises an exogenous nucleic acid encoding a protein that confers antibiotic sensitivity or resistance to the bacterium or spore thereof.

31. (canceled)

32. (canceled)

33. A method for identifying a subject having a cancer as not likely to exhibit cancer survival comprising:
(a) determining a level of a diagnostic bacterium or a spore thereof in a sample of the subject;
(b) comparing the level of the diagnostic bacterium or spores thereof to a reference diagnostic bacterium or a spore thereof level; and
(c) identifying the subject as not likely to exhibit cancer survival:
(i) if the level of the diagnostic bacterium or spore thereof is lower than the reference diagnostic bacterium or spore thereof; wherein the diagnostic bacterium is a bacterium of the taxonomic group selected from the group consisting of Clostridia, Streptococcaceae, Lactobacillaceae, Actinobacteria, Pediococcus acidilactici, Erysipelotrichales, Coprobacillaceae, Clostridiaceae, Akkermansia muciniphila, Escherichia coli, Streptococcus mitis, Veillonellaceae, Bilophila, Parascardovia, Streptococcus salivarius, Coriobacteriaceae, Peptostreptococcaceae, Phascolarctobacterium faecium, Lactobacillus plantarum, Clostridium tertium, Eubacterium biforme, Alphaproteobacteria, Ruminococcaceae, Gemella haemolysans, Dorea, Coprococcus, Clostridium, Rothia, Megasphaera, Atopobium, Clostridium glycolicum, Coprococcus comes, Clostridium spiroforme, Lactobacillus fermentum, Clostridium sp. 826, Lactobacillus animalis, Ruminococcus (family Lachnospiraceae), Streptococcus parasanguinis, Clostridium sp. ID5, Granulicatella adiacens, Actinomyces graevenitzii, Clostridium bartlettii DSM 16795, S24-7, Clostridium algidixylanolyticum, Lactobacillus homohiochii, Tyzzerella nexilis, Mogibacterium neglectum, Lactobacillus reuteri, Clostridium saccharogumia, [Eubacterium] hallii, Drancourtella massiliensis, Clostridium perfringens, Bacteroides stercoris ATCC 43183, Ruminococcus faecis, Clostridium sp. PPf35E6, Bacteroides uniformis, Weissella confusa, Megasphaera micronuciformis, Propionibacterium, Peptostreptococcus sp. MDA2346-2, Bacteroides fragilis, Oscillospira, Lactobacillus gasseri, Rothia dentocariosa, Clostridium sporosphaeroides, Lactococcus, Clostridium hathewayi, Clostridium methylpentosum DSM 5476, Clostridium sp. cTPY-17, Clostridium sp. Enrichment culture clone NHT38, Ruminococcus torques, Clostridium sp. cf3-PUG, Lactobacillus buchneri, mitochondria, Coprococcus, Bacillaceae, Fusobacterium nucleatum, Eggerthella lenta, Anaerococcus, Clostridium sp. Culture-54, Anaerostipes caccae, Blautia faecis, Actinobaculum massiliense, Enterococcus gallinarum, Clostridium sp. MSTE9, ambiguous Leuconostoc, Paraprevotellaceae, Roseburia, unclassified Peptostreptococcaceae, Fusobacterium, Atopobium rimae, Blautia luti, Propionibacterium propionicum, Streptococcus lutetiensis, Scardovia inopinata, Dorea formicigenerans, Corynebacterium pseudogenitalium, Turicibacter sanguinis, Roseburia faecis, Clostridium paraputrificum, Fusobacteria, Clostridium symbiosum, Propionibacterium freudenreichii, and any combinations thereof, or the diagnostic bacterium or spore thereof comprises a 16S rRNA gene having a nucleotide sequence that has between about 90 and 100% homology to the nucleotide sequence set forth in any one of SEQ ID NOs:1-69; and/or
(ii) if the level of the diagnostic bacterium or spore thereof is higher than the reference diagnostic bacterium or spore thereof; wherein the diagnostic bacterium is a bacterium of the taxonomic group selected from the group consisting of Firmicutes, Bacilli, Enterococcus, Bacillales, ambiguous Klebsiella, Lactobacillus delbrueckii subsp. Bulgaricus, Coprobacillus, Parabacteroides, Ruminococcus, Streptococcus mutans, Enterococcus rivorum, Enterobacter ludwigii, Clostridium leptum, Enterococcus lactis, Bifidobacterium dentium, Eubacterium limosum, Proteobacteria, Mycoplasma, Holdemania filiformis, Lactococcus piscium, Blautia, Bacteroides thetaiotaomicron, Massiliomicrobiota timonensis, Blautia hydrogenotrophica, Enterococcus mundtii, Prevotella melaninogenica, Erwinia chrysanthemi, Clostridium nexile DSM 1787, Veillonella, Clostridium difficile, [Ruminococcus] obeum, Streptophyta, Bacteroidales, Parascardovia, Clostridium clostridioforme, Blautia obeum, Klebsiella oxytoca, Bulleidia moorei, Parabacteroides merdae, Shuttleworthia satelles, Streptococcus sp. DN812, Clostridium cellulosi, Lactobacillus acidophilus, Bacteroides ovatus, Clostridium hylemonae, Veillonella parvula, Longibaculum muris, Butyrivibrio, Peptostreptococcaceae bacterium canine oral taxon 074, Alloscardovia omnicolens, Lactobacillus salivarius, Clostridium scindens, Alistipes, Clostridium lavalense, Anaerostipes, Abiotrophia defectiva, Leuconostocaceae, Dorea, Alistipes putredinis, Salinicoccus qingdaonensis, Parasutterella excrementihominis, Bacteroides caccae, Bifidobacteriaceae, Streptococcus anginosus, Haemophilus parainfluenzae, Oscillospira, Clostridium aldenense, and any combination thereof; or the diagnostic bacterium or spore thereof comprises a 16S rRNA gene sequence that has between about 90 and 100% homology to the nucleotide sequence set forth in any one of SEQ ID NOs:70-118.

34.-42. (canceled)

43. A method for identifying a subject having a cancer as likely to exhibit cancer survival comprising:
(a) determining a level of a diagnostic bacterium or a spore thereof in a sample of the subject;
(b) comparing the level of the diagnostic bacterium or spores thereof to a reference diagnostic bacterium or a spore thereof level;
(c) identifying the subject as likely to exhibit cancer survival:
(i) if the level of the diagnostic bacterium or spore thereof is higher than the reference diagnostic bacterium or spore thereof, wherein the diagnostic bacterium is a bacterium of the taxonomic group selected from the group consisting of Clostridia, Streptococcaceae, Lactobacillaceae, Actinobacteria, Pediococcus acidilactici, Erysipelotrichales, Coprobacillaceae, Clostridiaceae, Akkermansia muciniphila, Escherichia coli, Streptococcus mitis, Veillonellaceae, Bilophila, Parascardovia, Streptococcus salivarius, Coriobacteriaceae, Peptostreptococcaceae, Phascolarctobacterium faecium, Lactobacillus plantarum, Clostridium tertium, Eubacterium biforme, Alphaproteobacteria, Ruminococcaceae, Gemella haemolysans, Dorea, Coprococcus, Clostridium, Rothia, Megasphaera, Atopobium, Clostridium glycolicum, Coprococcus comes, Clostridium spiroforme, Lactobacillus fermentum, Clostridium sp. 826, Lactobacillus animalis, Ruminococcus (family Lachnospiraceae), Streptococcus parasanguinis, Clostridium sp. ID5, Granulicatella adiacens, Actinomyces graevenitzii, Clostridium bartlettii DSM 16795, S24-7, Clostridium algidixylanolyticum, Lactobacillus homohiochii, Tyzzerella nexilis, Mogibacterium neglectum, Lactobacillus reuteri, Clostridium saccharogumia, [Eubacterium] hallii, Drancourtella massiliensis, Clostridium perfringens, Bacteroides stercoris ATCC 43183, Ruminococcus faecis, Clostridium sp. PPf35E6, Bacteroides uniformis, Weissella confusa, Megasphaera micronuciformis, Propionibacterium, Peptostreptococcus sp. MDA2346-2, Bacteroides fragilis, Oscillospira, Lactobacillus gasseri, Rothia dentocariosa, Clostridium sporosphaeroides, Lactococcus, Clostridium hathewayi, Clostridium methylpentosum DSM 5476, Clostridium sp. cTPY-17, Clostridium sp. Enrichment culture clone NHT38, Ruminococcus torques, Clostridium sp. cf3-PUG, Lactobacillus buchneri, mitochondria, Coprococcus, Bacillaceae, Fusobacterium nucleatum, Eggerthella lenta, Anaerococcus, Clostridium sp. Culture-54, Anaerostipes caccae, Blautia faecis, Actinobaculum massiliense, Enterococcus gallinarum, Clostridium sp. MSTE9, ambiguous Leuconostoc, Paraprevotellaceae, Roseburia, unclassified Peptostreptococcaceae, Fusobacterium, Atopobium rimae, Blautia luti, Propionibacterium propionicum, Streptococcus lutetiensis, Scardovia inopinata, Dorea formicigenerans, Corynebacterium pseudogenitalium, Turicibacter sanguinis, Roseburia faecis, Clostridium paraputrificum, Fusobacteria, Clostridium symbiosum, Propionibacterium freudenreichii, and any combinations thereof or the diagnostic bacterium or spore thereof comprises a 16S rRNA gene having a nucleotide sequence that has between about 90 and 100% homology to the nucleotide sequence set forth in any one of SEQ ID NOs:1-69; and/or
(ii) if the level of the diagnostic bacterium or spore thereof is lower than the reference diagnostic bacterium or spore thereof, wherein the diagnostic bacterium is a bacterium of the taxonomic group selected from the group consisting of Firmicutes, Bacilli, Enterococcus, Bacillales, ambiguous Klebsiella, Lactobacillus delbrueckii subsp. Bulgaricus, Coprobacillus, Parabacteroides, Ruminococcus, Streptococcus mutans, Enterococcus rivorum, Enterobacter ludwigii, Clostridium leptum, Enterococcus lactis, Bifidobacterium dentium, Eubacterium limosum, Proteobacteria, Mycoplasma, Holdemania filiformis, Lactococcus piscium, Blautia, Bacteroides thetaiotaomicron, Massiliomicrobiota timonensis, Blautia hydrogenotrophica, Enterococcus mundtii, Prevotella melaninogenica, Erwinia chrysanthemi, Clostridium nexile DSM 1787, Veillonella, Clostridium difficile, [Ruminococcus] obeum, Streptophyta, Bacteroidales, Parascardovia, Clostridium clostridioforme, Blautia obeum, Klebsiella oxytoca, Bulleidia moorei, Parabacteroides merdae, Shuttleworthia satelles, Streptococcus sp. DN812, Clostridium cellulosi, Lactobacillus acidophilus, Bacteroides ovatus, Clostridium hylemonae, Veillonella parvula, Longibaculum muris, Butyrivibrio, Peptostreptococcaceae bacterium canine oral taxon 074, Alloscardovia omnicolens, Lactobacillus salivarius, Clostridium scindens, Alistipes, Clostridium lavalense, Anaerostipes, Abiotrophia defectiva, Leuconostocaceae, Dorea, Alistipes putredinis, Salinicoccus qingdaonensis, Parasutterella excrementihominis, Bacteroides caccae, Bifidobacteriaceae, Streptococcus anginosus, Haemophilus parainfluenzae, Oscillospira, Clostridium aldenense, and any combination thereof or the diagnostic bacterium or spore thereof comprises a 16S rRNA gene sequence that has between about 90 and 100% homology to the nucleotide sequence set forth in any one of SEQ ID NOs:70-118.

44. (canceled)

45. A kit comprising the pharmaceutical composition of claim 25.

46.-49. (canceled)

50. A kit for performing the method of claim 1.

51. (canceled)

52. The kit of claim 49, wherein the kit comprises means for detecting the level of a diagnostic bacterium or a spore thereof,
wherein the diagnostic bacterium is a bacterium of the taxonomic group selected from the group consisting of Firmicutes, Bacilli, Enterococcus, Bacillales, ambiguous Klebsiella, Lactobacillus delbrueckii subsp. Bulgaricus, Coprobacillus, Parabacteroides, Ruminococcus, Streptococcus mutans, Enterococcus rivorum, Enterobacter ludwigii, Clostridium leptum, Enterococcus lactis, Bifidobacterium dentium, Eubacterium limosum, Proteobacteria, Mycoplasma, Holdemania filiformis, Lactococcus piscium, Blautia, Bacteroides thetaiotaomicron, Massiliomicrobiota timonensis, Blautia hydrogenotrophica, Enterococcus mundtii, Prevotella melaninogenica, Erwinia chrysanthemi, Clostridium nexile DSM 1787, Veillonella, Clostridium difficile, [Ruminococcus] obeum, Streptophyta, Bacteroidales, Parascardovia, Clostridium clostridioforme, Blautia obeum, Klebsiella oxytoca, Bulleidia moorei, Parabacteroides merdae, Shuttleworthia satelles, Streptococcus sp. DN812, Clostridium cellulosi, Lactobacillus acidophilus, Bacteroides ovatus, Clostridium hylemonae, Veillonella parvula, Longibaculum muris, Butyrivibrio, Peptostreptococcaceae bacterium canine oral taxon 074, Alloscardovia omnicolens, Lactobacillus salivarius, Clostridium scindens, Alistipes, Clostridium lavalense, Anaerostipes, Abiotrophia defectiva, Leuconostocaceae, Dorea, Alistipes putredinis, Salinicoccus qingdaonensis, Parasutterella excrementihominis, Bacteroides caccae, Bifidobacteriaceae, Streptococcus anginosus, Haemophilus parainfluenzae, Oscillospira, Clostridium aldenense, Clostridia, Streptococcaceae, Lactobacillaceae, Actinobacteria, Pediococcus acidilactici, Erysipelotrichales, Coprobacillaceae, Clostridiaceae, Akkermansia muciniphila, Escherichia coli, Streptococcus mitis, Veillonellaceae, Bilophila, Parascardovia, Streptococcus salivarius, Coriobacteriaceae, Peptostreptococcaceae, Phascolarctobacterium faecium, Lactobacillus plantarum, Clostridium tertium, Eubacterium biforme, Alphaproteobacteria, Ruminococcaceae, Gemella haemolysans, Dorea, Coprococcus, Clostridium, Rothia, Megasphaera, Atopobium, Clostridium glycolicum, Coprococcus comes, Clostridium spiroforme, Lactobacillus fermentum, Clostridium sp. 826, Lactobacillus animalis, Ruminococcus (family Lachnospiraceae), Streptococcus parasanguinis, Clostridium sp. ID5, Granulicatella adiacens, Actinomyces graevenitzii, Clostridium bartlettii DSM 16795, S24-7, Clostridium algidixylanolyticum, Lactobacillus homohiochii, Tyzzerella nexilis, Mogibacterium neglectum, Lactobacillus reuteri, Clostridium saccharogumia, [Eubacterium] hallii, Drancourtella massiliensis, Clostridium perfringens, Bacteroides stercoris ATCC 43183, Ruminococcus faecis, Clostridium sp. PPf35E6, Bacteroides uniformis, Weissella confusa, Megasphaera micronuciformis, Propionibacterium, Peptostreptococcus sp. MDA2346-2, Bacteroides fragilis, Oscillospira, Lactobacillus gasseri, Rothia dentocariosa, Clostridium sporosphaeroides, Lactococcus, Clostridium hathewayi, Clostridium methylpentosum DSM 5476, Clostridium sp. cTPY-17, Clostridium sp. Enrichment culture clone NHT38, Ruminococcus torques, Clostridium sp. cf3-PUG, Lactobacillus buchneri, mitochondria, Coprococcus, Bacillaceae, Fusobacterium nucleatum, Eggerthella lenta, Anaerococcus, Clostridium sp. Culture-54, Anaerostipes caccae, Blautia faecis, Actinobaculum massiliense, Enterococcus gallinarum, Clostridium sp. MSTE9, ambiguous Leuconostoc, Paraprevotellaceae, Roseburia, unclassified Peptostreptococcaceae, Fusobacterium, Atopobium rimae, Blautia luti, Propionibacterium propionicum, Streptococcus lutetiensis, Scardovia inopinata, Dorea formicigenerans, Corynebacterium pseudogenitalium, Turicibacter sanguinis, Roseburia faecis, Clostridium paraputrificum, Fusobacteria, Clostridium symbiosum, Propionibacterium freudenreichii and any combination thereof or the diagnostic bacterium or spore thereof comprises a 16S rRNA gene sequence that has between about 90 and 100% homology to the nucleotide sequence set forth in any one of SEQ ID NOs: 1-118.
